SEQUENCE LISTING (96 sequences)

SEQ ID NO: 1 - protein, 119 aa, synthetic construct
  VARIANT 2: note = may be replaced with Ile
  VARIANT 11: note = may be replaced with Ile
  VARIANT 12: note = may be replaced with Lys
  VARIANT 38: note = may be replaced with Arg
  VARIANT 48: note = may be replaced with Ile
  VARIANT 68: note = may be replaced with Ala
  VARIANT 70: note = may be replaced with Leu
  VARIANT 72: note = may be replaced with Val
  VARIANT 81: note = may be replaced with Ile
  source 1..119: note = Heavy chain variable domain VH
QVQLVQSGAEVVKPGASVKVSCKASGYSFTDYNMYWVKQAPGQRLEWMGYIDPYNGDTTYNQKFKGRVTITRDTSASTAYMELSSLRSEDTAVYYCARSPYGHYAMDYWGQGTLVTVSS

SEQ ID NO: 2 - protein, 119 aa, synthetic construct; identical to SEQ ID NO: 1
  source 1..119: note = Heavy chain variable domain VH [AH02871_HC]

SEQ ID NO: 3 - protein, 119 aa, synthetic construct
  source 1..119: note = Heavy chain variable domain VH [AH02875_HC]
QIQLVQSGAELKKPGASVKVSCKASGYSFTDYNMYWVRQAPGQRLEWMGYIDPYNGDTTYNQKFKGRATITRDTSASTAYIELSSLRSEDTAVYYCARSPYGHYAMDYWGQGTLVTVSS

SEQ ID NO: 4 - protein, 119 aa, synthetic construct
  source 1..119: note = Heavy chain variable domain VH [AH02877_HC]
QVQLVQSGAEVVKPGASVKVSCKASGYSFTDYNMYWVKQAPGQRLEWMGYIDPYNGDTTYNQKFKGRATLTVDTSASTAYIELSSLRSEDTAVYYCARSPYGHYAMDYWGQGTLVTVSS

SEQ ID NO: 5 - protein, 119 aa, synthetic construct
  source 1..119: note = Heavy chain variable domain VH [AH02879_HC]
QIQLVQSGAEVVKPGASVKVSCKASGYSFTDYNMYWVKQAPGQRLEWIGYIDPYNGDTTYNQKFKGRATLTRDTSASTAYMELSSLRSEDTAVYYCARSPYGHYAMDYWGQGTLVTVSS

SEQ ID NO: 6 - protein, 119 aa, synthetic construct
  source 1..119: note = Heavy chain variable domain VH [AH02886_HC]
QVQLVQSGAELKKPGASVKVSCKASGYSFTDYNMYWVKQAPGQRLEWMGYIDPYNGDTTYNQKFKGRVTLTVDTSASTAYMELSSLRSEDTAVYYCARSPYGHYAMDYWGQGTLVTVSS

SEQ ID NO: 7 - protein, 119 aa, synthetic construct
  source 1..119: note = Heavy chain variable domain VH [AH02895_HC]
QIQLVQSGAEVVKPGASVKVSCKASGYSFTDYNMYWVKQAPGQRLEWMGYIDPYNGDTTYNQKFKGRATITRDTSASTAYIELSSLRSEDTAVYYCARSPYGHYAMDYWGQGTLVTVSS

SEQ ID NO: 8 - protein, 112 aa, synthetic construct
  VARIANT 13: note = may be replaced with Thr
  VARIANT 43: note = may be replaced with Ser
  VARIANT 49: note = may be replaced with Tyr
  VARIANT 71: note = may be replaced with Phe
  VARIANT 78: note = may be replaced with Leu
  VARIANT 106: note = may be replaced with Val or Met
  VARIANT 110: note = may be replaced with Asp
  source 1..112: note = light chain variable domain (VL)
DIQMTQSPSSLSASVGDRVTITCKASQDVSTAVDWYQQKPGKAPKLLINWASTRHTGVPSRFSGSGSGTDYTLTISSMQPEDFATYYCRQHYSTPFTFGQGTKLEIKRTVAA

SEQ ID NO: 9 - protein, 112 aa, synthetic construct
  source 1..112: note = light chain variable domain (VL) [AH02875_LC]
DIQMTQSPSSLSASVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAA

SEQ ID NO: 10 - protein, 112 aa, synthetic construct
  source 1..112: note = light chain variable domain (VL) [H02877_LC]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEIKRTVAA

SEQ ID NO: 11 - protein, 112 aa, synthetic construct
  source 1..112: note = light chain variable domain (VL) [H02879_LC]
DIQMTQSPSSLSTSVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAA

SEQ ID NO: 12 - protein, 112 aa, synthetic construct
  source 1..112: note = light chain variable domain (VL) [H02886_LC]
DIQMTQSPSSLSTSVGDRVTITCKASQDVSTAVDWYQQKPGKAPKLLINWASTRHTGVPSRFSGSGSGTDYTLTISSLQPEDFATYYCRQHYSTPFTFGQGTKLEIKRTVAA

SEQ ID NO: 13 - protein, 112 aa, synthetic construct; identical to SEQ ID NO: 8
  source 1..112: note = light chain variable domain (VL) [H02895_LC]

SEQ ID NO: 14 - protein, 112 aa, synthetic construct
  source 1..112: note = light chain variable domain (VL) [H02877_I106M]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEMKRTVAA

SEQ ID NO: 15 - protein, 112 aa, synthetic construct
  source 1..112: note = light chain variable domain (VL) [AH02877_I106V]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEVKRTVAA

SEQ ID NO: 16 - protein, 112 aa, synthetic construct
  source 1..112: note = light chain variable domain (VL) [AH02877_V110D]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEIKRTDAA

SEQ ID NO: 17 - protein, 112 aa, synthetic construct
  source 1..112: note = light chain variable domain (VL) [AH02877_I106M_V110D]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEMKRTDAA

SEQ ID NO: 18 - protein, 112 aa, synthetic construct
  source 1..112: note = light chain variable domain (VL) [AH02877_I106V_V110D]
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEVKRTDAA

SEQ ID NO: 19 - protein, 119 aa, synthetic construct
  source 1..119: note = Variable heavy chain (VH) of Lilotomab
EIQLQQSGPELVKPGASVKVSCKASGYSFTDYNMYWVKQSHGKSLEWIGYIDPYNGDTTYNQKFKGKATLTVDKSSSTAFIHLNSLTSEDSAVYYCARSPYGHYAMDYWGQGTSVTVSS

SEQ ID NO: 20 - protein, 112 aa, synthetic construct
  source 1..112: note = Variable light chain (VL) of Lilotomab
DIVMTQSHKLLSTSVGDRVSITCKASQDVSTAVDWYQQKPGQSPKLLINWASTRHTGVPDRFTGSGSGTDYTLTISSMQAEDLALYYCRQHYSTPFTFGSGTKLEIKRADAA

SEQ ID NO: 21 - protein, 330 aa, Homo sapiens [sequence omitted]

SEQ ID NO: 22 - protein, 214 aa, synthetic construct
  source 1..214: note = NNV003 Light chain
DIVMTQSHKLLSTSVGDRVSITCKASQDVSTAVDWYQQKPGQSPKLLINWASTRHTGVPDRFTGSGSGTDYTLTISSMQAEDLALYYCRQHYSTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 449 aa, synthetic construct
  source 1..449: note = NNV003 heavy chain
EIQLQQSGPELVKPGASVKVSCKASGYSFTDYNMYWVKQSHGKSLEWIGYIDPYNGDTTYNQKFKGKATLTVDKSSSTAFIHLNSLTSEDSAVYYCARSPYGHYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, synthetic construct
  source 1..214: note = NNV023 light chain [AH02877_V110D] light chain
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEIKRTDAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 214 aa, synthetic construct
  source 1..214: note = AH02877_I106M light chain
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, synthetic construct
  source 1..214: note = AH02877_I106V light chain
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, synthetic construct
  source 1..214: note = AH02877_I106M_V110D light chain
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEMKRTDAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, synthetic construct
  source 1..214: note = AH02877_I106V_V110D light chain
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEVKRTDAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 449 aa, synthetic construct
  source 1..449: note = NNV023 heavy chain [NONA2871HC] heavy chain
QVQLVQSGAEVVKPGASVKVSCKASGYSFTDYNMYWVKQAPGQRLEWMGYIDPYNGDTTYNQKFKGRVTITRDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 448 aa, synthetic construct
  source 1..448: note = NNV030 heavy chain [NNV023_HC_dK330]
QVQLVQSGAEVVKPGASVKVSCKASGYSFTDYNMYWVKQAPGQRLEWMGYIDPYNGDTTYNQKFKGRVTITRDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, Homo sapiens
  source 1..214: note = kappa light chain constant domain, Uniprot ID P0DOX7
DIQMTQSPSTLSASVGDRVTITCRASQSINTWLAWYQQKPGKAPKLLMYKASSLESGVPSRFIGSGSGTEFTLTISSLQPDDFATYYCQQYNSDSKMFGQGTKVEVKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 1407 bp, synthetic construct
  source 1..1407: mol_type = other DNA; note = DNA encoding AH02871_HC with leader sequence
atgggctggagctggatcctgctgttcctcctgagcgtgacagcaggagtgcacagccaggtccagctggtgcagtcaggagcagaagtggtcaaacccggcgcatccgtgaaggtgtcttgtaaggcatcaggctactcatttaccgactacaacatgtattgggtgaagcaggctccaggacagcgactggagtggatggggtacatcgacccttataacggagataccacatacaatcagaagttcaaaggccgggtgactattaccagagacacatctgccagtactgcttatatggagctgagctccctgaggagcgaagatacagcagtctactattgcgcccgctctccttacgggcactatgctatggactattgggggcagggcactctggtcactgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 33 - DNA, 1407 bp, synthetic construct
  source 1..1407: mol_type = other DNA; note = DNA encoding AH02875_HC with leader sequence
atgggctggagctggatcctgctgttcctcctgagcgtgacagcaggagtgcacagccagattcagctggtgcagagcggggcagaactgaagaagcctggggcaagcgtcaaggtgtcatgtaaagcaagtggatactcattcaccgactacaacatgtattgggtgaggcaggctccaggacagcgcctggagtggatgggatacatcgacccttataacggagataccacatacaatcagaagttcaaaggccgggccactatcaccagagacacatctgccagtactgcttatattgagctgagctccctgcgaagcgaagatacagcagtctactattgcgccagatctccttatggacattacgctatggattattggggacagggaactctggtcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - DNA, 1407 bp, synthetic construct
  source 1..1407: mol_type = other DNA; note = DNA encoding AH02877_HC
atgggctggagctggatcctgctgttcctcctgagcgtgacagcaggagtgcacagccaggtgcagctggtccagagcggagcagaagtcgtcaagcccggagcatcagtcaaggtgtcatgtaaagcatcagggtattcatttaccgactacaacatgtattgggtgaagcaggctccaggacagaggctggagtggatggggtacatcgacccttataacggagataccacatacaatcagaagttcaaaggccgggccactctgaccgtggacacatctgccagtactgcttatattgagctgagctccctgcggagcgaagatacagcagtctactattgcgccagatctccttatggacattacgctatggattattggggacagggcactctggtcacagtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 35 - DNA, 1407 bp, synthetic construct
  source 1..1407: mol_type = other DNA; note = DNA encoding AH02879_HC with leader sequence
atgggctggagctggatcctgctgttcctcctgagcgtgacagcaggagtgcacagccagattcagctggtgcagagcggggcagaagtcgtcaaacctggggcaagtgtcaaagtcagttgtaaagcaagcggatactccttcaccgactacaacatgtattgggtgaagcaggctccaggacagcgactggagtggatcgggtacattgacccttataacggagataccacatacaatcagaagttcaaaggccgggccactctgaccagagacacatctgccagtactgcttatatggagctgagctccctgaggagcgaagatacagcagtctactattgcgcccgctctccttatggacattatgctatggactactgggggcagggcacactggtgaccgtgagttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 36 - DNA, 1407 bp, synthetic construct
  source 1..1407: mol_type = other DNA; note = DNA encoding AH02886 _HC with leader sequence
atgggctggagctggatcctgctgttcctcctgagcgtgacagcaggagtgcacagccaggtgcagctggtccagagcggggcagaactgaaaaagccaggggcaagcgtcaaagtctcatgtaaagcaagcgggtattccttcaccgactacaacatgtattgggtgaagcaggctccaggacagaggctggagtggatggggtacatcgacccttataacggagataccacatacaatcagaagttcaaaggccgggtgactctgaccgtcgacacatctgccagtactgcttatatggagctgagctccctgcggagcgaagatacagcagtctactattgcgccagatctccttatggacattatgctatggattattgggggcagggcactctggtgaccgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - DNA, 1407 bp, synthetic construct
  source 1..1407: mol_type = other DNA; note = DNA encoding AH02895_HC with leader sequence
atgggctggagctggatcctgctgttcctcctgagcgtgacagcaggagtgcacagccagattcagctggtgcagagcggagcagaggtggtcaagcctggggcaagcgtcaaagtcagttgtaaagcaagcggatactcatttaccgactacaacatgtattgggtgaagcaggctccaggacagcgactggagtggatggggtacatcgacccttataacggagataccacatacaatcagaagttcaaaggccgggccactatcaccagagacacatctgccagtactgcttatattgagctgagctccctgaggagcgaagatacagcagtctactattgcgcccgctctccttacgggcactacgcaatggattactgggggcaggggactctggtcactgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 38 - DNA, 702 bp, synthetic construct
  source 1..702: mol_type = other DNA; note = DNA encoding AH02871_LC with leader sequence
atgggctggagctggatcctgctgttcctcctgagcgtgacagcaggagtgcacagcgatattcagatgacccagtctcccagtagtctgtcagcttcagtcggcgatagagtcaccatcacttgtaaggcttcccaggacgtgagtaccgcagtggactggtaccagcagaagcccggcaaagcccctaagctgctgatcaactgggctagcactcgacacaccggagtcccatccagattctctggcagtgggtcaggaacagactataccctgacaattagctccatgcagcccgaggattttgccacttactattgtagacagcattactcaaccccattcaccttcggacagggaactaaactggaaatcaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 39 - DNA, 702 bp, synthetic construct
  source 1..702: mol_type = other DNA; note = DNA encoding AH02875_LC with leader sequence
atgggctggagctggatcctgctgttcctcctgagcgtgacagcaggagtgcacagcgatattcagatgacccagagccctagttcactgtcagccagcgtgggagatagagtcacaatcacatgcaaagccagccaggacgtgtctaccgccgtggactggtaccagcagaagcccggcaaaagccctaagctgctgatcaactgggctagcactcgacacaccggagtcccatccagattctctggcagtgggtcaggaacagactataccctgacaattagctccatgcagcccgaggattttgccacttactattgcaggcagcactattctaccccattcacattcgggcagggaactaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - DNA, 702 bp, synthetic construct
  source 1..702: mol_type = other DNA; note = DNA encoding AH02877_LC with leader sequence
atgggctggagctggatcctgctgttcctcctgagcgtgacagcaggagtgcacagcgatattcagatgacccagagcccttccagcctgtccgccagcgtcggagaccgcgtgaccattacttgcaaagcttcccaggatgtgtctaccgccgtggactggtaccagcagaagcccggcaaaagccctaagctgctgatcaactgggctagcactcgacacaccggagtcccatccagattctctggcagtgggtcaggaacagactataccctgacaattagctccctgcagcccgaggattttgccacttactattgtaggcagcattactcaaccccattcacattcggacagggcaccaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - DNA, 702 bp, synthetic construct
  source 1..702: mol_type = other DNA; note = DNA encoding AH02879_LC with leader sequence
atgggctggagctggatcctgctgttcctcctgagcgtgacagcaggagtgcacagcgatattcagatgacccagtccccctcaagcctgtccacaagcgtcggcgatagagtgaccattacctgtaaagcaagccaggatgtctctaccgccgtggactggtaccagcagaagcccggcaaaagccctaagctgctgatcaactgggctagcactcgacacaccggagtcccatccagattctctggcagtgggtcaggaacagactataccctgacaattagctccatgcagcccgaggattttgccacttactattgtagacagcattactcaacaccattcacattcgggcagggaaccaaactggaaatcaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 42 - DNA, 702 bp, synthetic construct
  misc_feature 1..702: note = DNA encoding AH02886_LC with leader sequence
  source 1..702: mol_type = other DNA; note = DNA encoding AH02886_LC with leader sequence
atgggctggagctggatcctgctgttcctcctgagcgtgacagcaggagtgcacagcgatattcagatgacccagagcccttcttccctgtcaacttcagtcggagatagagtcacaatcacctgtaaagccagccaggacgtgagtaccgcagtggactggtaccagcagaagcccggcaaagcccctaagctgctgatcaactgggctagcactcgacacaccggagtcccatccagattctctggcagtgggtcaggaacagactataccctgacaattagctccctgcagcccgaggattttgccacttactattgtagacagcactattcaacaccttttaccttcggacagggcaccaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 43 - DNA, 702 bp, synthetic construct; identical to SEQ ID NO: 38
  source 1..702: mol_type = other DNA; note = DNA encoding AH02895_LC with leader sequence

SEQ ID NO: 44 - protein, 468 aa, synthetic construct
  source 1..468: note = heavy chain of AH02871_HC with Leader peptide
MGWSWILLFLLSVTAGVHSQVQLVQSGAEVVKPGASVKVSCKASGYSFTDYNMYWVKQAPGQRLEWMGYIDPYNGDTTYNQKFKGRVTITRDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 468 aa, synthetic construct
  source 1..468: note = heavy chain of AH02875_HC with Leader peptide
MGWSWILLFLLSVTAGVHSQIQLVQSGAELKKPGASVKVSCKASGYSFTDYNMYWVRQAPGQRLEWMGYIDPYNGDTTYNQKFKGRATITRDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 468 aa, synthetic construct
  source 1..468: note = heavy chain of AH02877_HC with Leader peptide
MGWSWILLFLLSVTAGVHSQVQLVQSGAEVVKPGASVKVSCKASGYSFTDYNMYWVKQAPGQRLEWMGYIDPYNGDTTYNQKFKGRATLTVDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 468 aa, synthetic construct
  source 1..468: note = heavy chain of AH02879_HC with Leader peptide
MGWSWILLFLLSVTAGVHSQIQLVQSGAEVVKPGASVKVSCKASGYSFTDYNMYWVKQAPGQRLEWIGYIDPYNGDTTYNQKFKGRATLTRDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 468 aa, synthetic construct
  source 1..468: note = heavy chain of AH02886_HC with Leader peptide
MGWSWILLFLLSVTAGVHSQVQLVQSGAELKKPGASVKVSCKASGYSFTDYNMYWVKQAPGQRLEWMGYIDPYNGDTTYNQKFKGRVTLTVDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 468 aa, synthetic construct
  source 1..468: note = heavy chain of AH02895_HC with Leader peptide
MGWSWILLFLLSVTAGVHSQIQLVQSGAEVVKPGASVKVSCKASGYSFTDYNMYWVKQAPGQRLEWMGYIDPYNGDTTYNQKFKGRATITRDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 233 aa, synthetic construct
  source 1..233: note = light chain of AH02871_LC with Leader peptide
MGWSWILLFLLSVTAGVHSDIQMTQSPSSLSASVGDRVTITCKASQDVSTAVDWYQQKPGKAPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 233 aa, synthetic construct
  source 1..233: note = light chain of AH02875_LC with Leader peptide
MGWSWILLFLLSVTAGVHSDIQMTQSPSSLSASVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 233 aa, synthetic construct
  source 1..233: note = light chain of AH02877_LC with Leader peptide
MGWSWILLFLLSVTAGVHSDIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 233 aa, synthetic construct
  source 1..233: note = Light chain of AH02879_LC with Leader peptide
MGWSWILLFLLSVTAGVHSDIQMTQSPSSLSTSVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 233 aa, synthetic construct
  source 1..233: note = light chain of AH02886_LC with Leader peptide
MGWSWILLFLLSVTAGVHSDIQMTQSPSSLSTSVGDRVTITCKASQDVSTAVDWYQQKPGKAPKLLINWASTRHTGVPSRFSGSGSGTDYTLTISSLQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 233 aa, synthetic construct; identical to SEQ ID NO: 50
  source 1..233: note = light chain of AH02895_LC with Leader peptide

SEQ ID NO: 56 - protein, 443 aa, synthetic construct
  source 1..443: note = Heavy chain of Lilotomab
EIQLQQSGPELVKPGASVKVSCKASGYSFTDYNMYWVKQSHGKSLEWIGYIDPYNGDTTYNQKFKGKATLTVDKSSSTAFIHLNSLTSEDSAVYYCARSPYGHYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 57 - protein, 214 aa, synthetic construct
  source 1..214: note = light chain of Lilotomab
DIVMTQSHKLLSTSVGDRVSITCKASQDVSTAVDWYQQKPGQSPKLLINWASTRHTGVPDRFTGSGSGTDYTLTISSMQAEDLALYYCRQHYSTPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 451 aa, synthetic construct
  source 1..451: note = Rituximab_chimeric_HC
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 213 aa, synthetic construct
  source 1..213: note = Rituximab_chimeric_LC
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 98 aa, Homo sapiens
  source 1..98: note = Human germline VH domain, IGHV1-3*01
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 61 - protein, 96 aa, Homo sapiens
  source 1..96: note = Human gernline VL domain, IGKV1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTPP

SEQ ID NO: 62 - protein, 167 aa, Homo sapiens
  source 1..167: note = immunoglobulin mu heavy chain, partial [NCBI ref: AEB00269.1]
LVAAATGAHSQVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREQWLVRENFDYWGQGTLVTVSSGSASAPTLFPLVSCENSPSDTSSVAVGCLAQDFLPDS

SEQ ID NO: 63 - protein, 107 aa, Homo sapiens
  source 1..107: note = immunoglobulin kappa light chain, partial [NCBI ref. BAH04867.1]
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 64 - protein, 119 aa, synthetic construct
  source 1..119: note = Heavy chain variable domain VH_1
QVQLVQSGAEVKKPGASVKVSCKASGYSFTDYNMYWVRQAPGQRLEWMGYIDPYNGDTTYNQKFKGRVTITRDTSASTAYMELSSLRSEDTAVYYCARSPYGHYAMDYWGQGTLVTVSS

SEQ ID NO: 65 - protein, 107 aa, synthetic construct
  source 1..107: note = Light chain variable domain VL_1
DIQMTQSPSSLSASVGDRVTITCKASQDVSTAVDWYQQKPGKAPKLLIYWASTRHTGVPSRFSGSGSGTDFTLTISSLQPEDFATYYCRQHYSTPFTFGQGTKLEIK

SEQ ID NO: 66 - protein, 449 aa, synthetic construct
  source 1..449: note = heavy chain of AH02875_HC
QIQLVQSGAELKKPGASVKVSCKASGYSFTDYNMYWVRQAPGQRLEWMGYIDPYNGDTTYNQKFKGRATITRDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 449 aa, synthetic construct
  source 1..449: note = heavy chain of AH02877_HC
QVQLVQSGAEVVKPGASVKVSCKASGYSFTDYNMYWVKQAPGQRLEWMGYIDPYNGDTTYNQKFKGRATLTVDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 449 aa, synthetic construct
  source 1..449: note = heavy chain of AH02879_HC
QIQLVQSGAEVVKPGASVKVSCKASGYSFTDYNMYWVKQAPGQRLEWIGYIDPYNGDTTYNQKFKGRATLTRDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 449 aa, synthetic construct
  source 1..449: note = heavy chain of AH02886_HC
QVQLVQSGAELKKPGASVKVSCKASGYSFTDYNMYWVKQAPGQRLEWMGYIDPYNGDTTYNQKFKGRVTLTVDTSASTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 449 aa, synthetic construct
  source 1..449: note = heavy chain of AH02895_HC
QIQLVQSGAEVVKPGASVKVSCKASGYSFTDYNMYWVKQAPGQRLEWMGYIDPYNGDTTYNQKFKGRATITRDTSASTAYIELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, synthetic construct
  source 1..214: note = light chain of AH02875_LC
DIQMTQSPSSLSASVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 214 aa, synthetic construct
  source 1..214: note = light chain of AH02877_LC
DIQMTQSPSSLSASVGDRVTITCKASQDVSTAVDWYQQKPGKSPKLLINWASTRHTGVPSRFSGSGSGTDYTLTISSLQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 214 aa, synthetic construct
  source 1..214: note = light chain of AH02879_LC
DIQMTQSPSSLSTSVGDRVTITCKASQDVSTAVDWYQQKPGKSPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, synthetic construct
  source 1..214: note = light chain of AH02886_LC
DIQMTQSPSSLSTSVGDRVTITCKASQDVSTAVDWYQQKPGKAPKLLINWASTRHTGVPSRFSGSGSGTDYTLTISSLQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 214 aa, synthetic construct
  source 1..214: note = light chain of AH02895_LC
DIQMTQSPSSLSASVGDRVTITCKASQDVSTAVDWYQQKPGKAPKLLINWASTRHTGVPSRFSGSGSGTDYTLTISSMQPEDFATYYCRQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, synthetic construct; identical to SEQ ID NO: 75
  source 1..214: note = light chain of AH02871_LC

SEQ ID NO: 77 - protein, 307 aa, synthetic construct
  source 1..307: note = PRO 4077_evi-50 RMD (GlymaxX protein)
AAAMTQRLFVTGLSGFVGKHLQAYLAAAHTPWALLPVPHRYDLLEPDSLGDLWPELPDAVIHLAGQTYVPEAFRDPARTLQINLLGTLNLLQALKARGFSGTFLYISSGDVYGQVAEAALPIHEELIPHPRNPYAVSKLAAESLCLQWGITEGWRVLVARPFNHIGPGQKDSFVIASAARQIARMKQGLQANRLEVGDIDVSRDFLDVQDVLSAYLRLLSHGEAGAVYNVCSGQEQKIRELIELLADIAQVELEIVQDPARMRRAEQRRVRGSHARLHDTTGWKPEITIKQSLRAILSDWESRVREE

SEQ ID NO: 78 - protein, 448 aa, synthetic construct
  source 1..448: note = Heavy chain DuoBody 1
EVQLVESGGGLVQPGGSLRLSCAASGFSLSYNAMNWVRQAPGKGLEWVSIIFASGRTDYASWAKGRFTISRDNSKNTLYLQMNSLRAEDTAVYYCAREGSTWGDALDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHGALHNHYTQKSLSLSPG

SEQ ID NO: 79 - protein, 217 aa, synthetic construct
  source 1..217: note = Light chain DuoBody 1
YDMTQSPSTLSASVGDRVTITCQASQNIIDYLAWYQQKPGKAPKLLIHKASTTLASGVPSRFKGSGSGTEFTLTISSLQPDDFATYYCQQGYSNSNIDNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 449 aa, synthetic construct
  source 1..449: note = Heavy chain DuoBody 6
EVQLVESGGGLVQPGGSLRLSCAASGFSLSNYNMGWVRQAPGKGLEWVSVIDASGTTYYATWAKGRFTISRDNSKNTLYLQMNSLRAEDTATYYCARELLYFGSSYYDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHGALHNHYTQKSLSLSPG

SEQ ID NO: 81 - protein, 217 aa, synthetic construct
  source 1..217: note = Light chain DuoBody 6
DVVMTQSPSTLSASVGDRVTITCQASQNIDSNLAWYQQKPGKAPKFLIYYASNLPFGVPSRFKGSGSGTEFTLTISSLQPDDFATYYCQSADVGSTYVAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 19 aa, synthetic construct
  source 1..19: note = Expression leader peptide
MGWSWILLFLLSVTAGVHS

SEQ ID NO: 83 - protein, 112 aa, synthetic construct; identical to SEQ ID NO: 8
  source 1..112: note = light chain variable domain (VL) [AH02871_LC]

SEQ ID NO: 84 - DNA, 1350 bp, synthetic construct
  source 1..1350: mol_type = other DNA; note = DNA encoding AH02871_HC
caggtccagctggtgcagtcaggagcagaagtggtcaaacccggcgcatccgtgaaggtgtcttgtaaggcatcaggctactcatttaccgactacaacatgtattgggtgaagcaggctccaggacagcgactggagtggatggggtacatcgacccttataacggagataccacatacaatcagaagttcaaaggccgggtgactattaccagagacacatctgccagtactgcttatatggagctgagctccctgaggagcgaagatacagcagtctactattgcgcccgctctccttacgggcactatgctatggactattgggggcagggcactctggtcactgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - DNA, 1350 bp, synthetic construct
  source 1..1350: mol_type = other DNA; note = DNA encoding AH02875_HC
cagattcagctggtgcagagcggggcagaactgaagaagcctggggcaagcgtcaaggtgtcatgtaaagcaagtggatactcattcaccgactacaacatgtattgggtgaggcaggctccaggacagcgcctggagtggatgggatacatcgacccttataacggagataccacatacaatcagaagttcaaaggccgggccactatcaccagagacacatctgccagtactgcttatattgagctgagctccctgcgaagcgaagatacagcagtctactattgcgccagatctccttatggacattacgctatggattattggggacagggaactctggtcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - DNA, 1350 bp, synthetic construct
  source 1..1350: mol_type = other DNA; note = DNA encoding AH02877_HC
caggtgcagctggtccagagcggagcagaagtcgtcaagcccggagcatcagtcaaggtgtcatgtaaagcatcagggtattcatttaccgactacaacatgtattgggtgaagcaggctccaggacagaggctggagtggatggggtacatcgacccttataacggagataccacatacaatcagaagttcaaaggccgggccactctgaccgtggacacatctgccagtactgcttatattgagctgagctccctgcggagcgaagatacagcagtctactattgcgccagatctccttatggacattacgctatggattattggggacagggcactctggtcacagtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 87 - DNA, 1350 bp, synthetic construct
  source 1..1350: mol_type = other DNA; note = DNA encoding AH02879_HC
cagattcagctggtgcagagcggggcagaagtcgtcaaacctggggcaagtgtcaaagtcagttgtaaagcaagcggatactccttcaccgactacaacatgtattgggtgaagcaggctccaggacagcgactggagtggatcgggtacattgacccttataacggagataccacatacaatcagaagttcaaaggccgggccactctgaccagagacacatctgccagtactgcttatatggagctgagctccctgaggagcgaagatacagcagtctactattgcgcccgctctccttatggacattatgctatggactactgggggcagggcacactggtgaccgtgagttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 88 - DNA, 1350 bp, synthetic construct
  source 1..1350: mol_type = other DNA; note = DNA encoding AH02886_ HC
caggtgcagctggtccagagcggggcagaactgaaaaagccaggggcaagcgtcaaagtctcatgtaaagcaagcgggtattccttcaccgactacaacatgtattgggtgaagcaggctccaggacagaggctggagtggatggggtacatcgacccttataacggagataccacatacaatcagaagttcaaaggccgggtgactctgaccgtcgacacatctgccagtactgcttatatggagctgagctccctgcggagcgaagatacagcagtctactattgcgccagatctccttatggacattatgctatggattattgggggcagggcactctggtgaccgtctcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - DNA, 1350 bp, synthetic construct
  source 1..1350: mol_type = other DNA; note = DNA encoding AH02895_HC
cagattcagctggtgcagagcggagcagaggtggtcaagcctggggcaagcgtcaaagtcagttgtaaagcaagcggatactcatttaccgactacaacatgtattgggtgaagcaggctccaggacagcgactggagtggatggggtacatcgacccttataacggagataccacatacaatcagaagttcaaaggccgggccactatcaccagagacacatctgccagtactgcttatattgagctgagctccctgaggagcgaagatacagcagtctactattgcgcccgctctccttacgggcactacgcaatggattactgggggcaggggactctggtcactgtctcatcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 90 - DNA, 645 bp, synthetic construct
  source 1..645: mol_type = other DNA; note = DNA encoding AH02871_LC
gatattcagatgacccagtctcccagtagtctgtcagcttcagtcggcgatagagtcaccatcacttgtaaggcttcccaggacgtgagtaccgcagtggactggtaccagcagaagcccggcaaagcccctaagctgctgatcaactgggctagcactcgacacaccggagtcccatccagattctctggcagtgggtcaggaacagactataccctgacaattagctccatgcagcccgaggattttgccacttactattgtagacagcattactcaaccccattcaccttcggacagggaactaaactggaaatcaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 91 - DNA, 645 bp, synthetic construct
  source 1..645: mol_type = other DNA; note = DNA sequence encoding AH02875_LC
gatattcagatgacccagagccctagttcactgtcagccagcgtgggagatagagtcacaatcacatgcaaagccagccaggacgtgtctaccgccgtggactggtaccagcagaagcccggcaaaagccctaagctgctgatcaactgggctagcactcgacacaccggagtcccatccagattctctggcagtgggtcaggaacagactataccctgacaattagctccatgcagcccgaggattttgccacttactattgcaggcagcactattctaccccattcacattcgggcagggaactaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 92 - DNA, 645 bp, synthetic construct
  source 1..645: mol_type = other DNA; note = DNA encoding AH02877_LC
gatattcagatgacccagagcccttccagcctgtccgccagcgtcggagaccgcgtgaccattacttgcaaagcttcccaggatgtgtctaccgccgtggactggtaccagcagaagcccggcaaaagccctaagctgctgatcaactgggctagcactcgacacaccggagtcccatccagattctctggcagtgggtcaggaacagactataccctgacaattagctccctgcagcccgaggattttgccacttactattgtaggcagcattactcaaccccattcacattcggacagggcaccaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 93 - DNA, 645 bp, synthetic construct
  source 1..645: mol_type = other DNA; note = DNA encoding AH02879_LC
gatattcagatgacccagtccccctcaagcctgtccacaagcgtcggcgatagagtgaccattacctgtaaagcaagccaggatgtctctaccgccgtggactggtaccagcagaagcccggcaaaagccctaagctgctgatcaactgggctagcactcgacacaccggagtcccatccagattctctggcagtgggtcaggaacagactataccctgacaattagctccatgcagcccgaggattttgccacttactattgtagacagcattactcaacaccattcacattcgggcagggaaccaaactggaaatcaagcgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 94 - DNA, 645 bp, synthetic construct
  source 1..645: mol_type = other DNA; note = DNA encoding AH02886_LC
gatattcagatgacccagagcccttcttccctgtcaacttcagtcggagatagagtcacaatcacctgtaaagccagccaggacgtgagtaccgcagtggactggtaccagcagaagcccggcaaagcccctaagctgctgatcaactgggctagcactcgacacaccggagtcccatccagattctctggcagtgggtcaggaacagactataccctgacaattagctccctgcagcccgaggattttgccacttactattgtagacagcactattcaacaccttttaccttcggacagggcaccaaactggaaatcaaacgaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 95 - DNA, 645 bp, synthetic construct; identical to SEQ ID NO: 90
  source 1..645: mol_type = other DNA; note = DNA encoding AH02895_LC

SEQ ID NO: 96 - DNA, 57 bp, synthetic construct
  source 1..57: mol_type = other DNA; note = DNA encoding leader sequence
atgggctggagctggatcctgctgttcctcctgagcgtgacagcaggagtgcacagc

(19) United States
(12) Patent Application Publication
(10) Pub. No.: US 2025/0333531 A1
(43) Pub. Date: Oct. 30, 2025

(54) HUMANIZED HH1

(71) Applicant: Nordic Nanovector ASA, Oslo (NO)

(72) Inventors: Roman Generalov, Oslo (NO); Veronique Martine Marie Pascal, Vanløse (DK); Helen Heyerdahl, Forneblu (NO); Ada Helena Vilma Repetto Llamazares, Oslo (NO); Jan Terje Andersen, Oslo (NO); Stian Foss, Oslo (NO); Jostein Dahle, Bekkastua (NO)

(21) Appl. No.: 18/698,732

(22) PCT Filed: Oct. 6, 2022
(86) PCT No.: PCT/EP2022/077836

§ 371 Date: Apr. 4, 2024

(30) Foreign Application Priority Data
Oct. 6, 2021 (EP) 21201292.6

(51) Int. Cl. C07K 16/28; A61K 39/00; A61K 51/10; A61P 35/00

(52) U.S. Cl. CPC C07K 16/2896; A61K 51/1093; A61P 35/00; A61K 2039/505; A61K 2039/507; C07K 2317/24; C07K 2317/565; C07K 2317/732; C07K 2317/734; C07K 2317/92; C07K 2317/94

(57) ABSTRACT

The present disclosure relates to antibodies, antibody fragments and antibody derivates thereof and conjugates thereof and their use in immunotherapy and immunoconjugate therapy, including radioimmunotherapy of cancer with a humanized antibody with a high cytotoxicity as well as various applications of the antibodies.

### CROSS REFERENCE TO RELATED APPLICATIONS

This application is a U.S. National Phase Application of PCT International Application Number PCT/EP2022/077836, filed on Oct. 6, 2022, designating the United States of America and published in the English language, which is an International Application of and claims the benefit of priority to European Patent Application No. 21201292.6, filed on Oct. 6, 2021. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SeqList-AERA002-006APC, created Apr. 4, 2024, which is approximately 150,841 bytes in size. The information in the electronic format of the Sequence Listing is expressly incorporated herein by reference in its entirety.

### FIELD

The present disclosure relates to antibodies, antibody fragments and antibody derivates thereof and conjugates thereof and their use in immunotherapy and immunoconjugate therapy, including radioimmunotherapy of cancer with a humanized antibody with a high cytotoxicity as well as various applications of the antibodies.

### BACKGROUND

The present disclosure relates to anti-CD37 molecules, conjugates thereof and use thereof in the treatment of cancers and autoimmune diseases.

Immunotherapy using monoclonal antibodies (mAbs) has been emerging as a safe and selective method for the treatment of cancer and other diseases.

In particular, the role of monoclonal antibodies in therapies that are based on B-cell depletion, e.g. in the treatment of B-cell malignancies, has expanded since the introduction of rituximab, an antibody that is directed against the CD20 antigen on the B-cell surface.

The CD37 antigen is a cell surface antigen that has not been considered as a target for B cell malignancies to the same extent as the B-cell antigen CD20.

CD37, a member of the tetraspanin superfamily, is a heavily glycosylated cell surface molecule with four transmembrane domains and two extracellular loops.

CD37 expression is observed in normal B-cells, non-Hodgkin's lymphoma (NHL), including mantle cell lymphoma (MCL), Burkitts Lymphoma (BL), small lymphocytic lymphoma (SLL) and follicular lymphoma (FL), marginal zone lymphoma (MZL), Diffuse large B-cell lymphoma (DLBCL), lymphoblastic lymphoma (LL), and chronic lymphoid leukemia (CLL).

This expression pattern makes CD37 an attractive target for antibody-mediated cancer therapy.

CD37 was first described in 1986 and characterized by the murine monoclonal antibody MB-1 (Link et al, 1986).

CD37 controls both humoral i.e., the aspect of immunity that is mediated by macromolecules found in extracellular fluids such as secreted antibodies, complement proteins, and certain antimicrobial peptides, and cellular immune responses.

CD37-deficiency in mice leads to spontaneous development of B cell lymphoma, and patients with CD37-negative lymphomas have a worse clinical outcome.

Binding of a CD37-specific mAb to cancer cells may trigger various mechanisms of action: after the antibody binds to the extracellular domain of the CD37 antigen, it may activate the complement cascade and lyse the targeted cell.

Also, an anti-CD37 antibody may mediate antibody-dependent cell-mediated cytotoxicity (ADCC) to the target cell, which occurs after the Fc portion of the bound antibody is recognized by appropriate receptors on cytotoxic cells of the immune system.

In addition, the antibody may alter the ability of B-cells to respond to antigen or other stimuli, and the anti-CD37 antibody may initiate programmed cell death (apoptosis).

Anti-CD37 mAb MB-1 was evaluated in two radio-immunotherapy trials in B-NHL patients (B-cell non-Hodgkin's lymphoma; Press et al., 1989; Kaminski et al., 1992).

Others have also disclosed anti-CD37 mABs that show potential (e.g. WO 2009/019312 by Heider et al., WO2012/007576 by Stilgenbauer et. al., and WO 2011/092295 by the present inventors) but there is still a long way to go before CD37 is proven the ideal alternative to CD20 for treating B-cell malignancies.

Thus, it has been shown that the CD37 antigen is frequently expressed on tumor cells in several human B-cell malignancies and on mature normal B-lymphocytes and that anti-CD37-based therapy may be a promising approach for treating B cell malignancies.

Although the anti-CD37 antibodies or antibody-like molecules described above (e.g. MB-1) have shown anti-tumor efficacy in B-cell malignancies and the potential to target CD37, there is a need for alternate anti-CD37 molecules to improve the therapeutic applicability of anti-CD37 molecules.

Hence, improved anti-CD37 molecules would be advantageous in the pursuit against new treatments against B-cell malignancies.

### SUMMARY

One or more aspect(s) of the present disclosure relates to an antibody, antibody fragment or antibody derivative thereof, which comprises,
a) a heavy chain variable domain (VH) comprising VH-CDR1, VH-CDR2 and VH-CDR3, and
b) a light chain variable domain (VL) comprising VL-CDR1, VL-CDR2 and VL-CDR3, wherein,
c) the heavy chain variable domain (VH) comprises the amino acid sequence of any one of SEQ ID NO: 1 [heavy chain of H02871],
   wherein, according to SEQ ID NO: 1, position 2, or position 11 is I or V, position 12 is V or K, position 38 is K or R, position 48 is M or I, position 68 is A or V, position 70 is I or L, position 72 is R or V, position 81 is I or M, and wherein
i. the heavy chain VH-CDR1 comprises the amino acid sequence GYSFTD,
ii. the heavy chain VH-CDR2 comprises the amino acid sequence PYN,
iii. the heavy chain VH-CDR3 comprises the amino acid sequence PYGHYAM,
d) the light chain variable domain (VL) comprises the amino acid sequence of any one of SEQ ID NO: 8 [light chain of H02871],
   wherein, according to SEQ ID NO: 8, position 13 is A or T, position 43 is A or S, position 49 is Y or N, position 71 is F or Y, position 78 is M or L, position 106 is I, M, or V, position 110 is V or D, and wherein
i. the light chain VL-CDR1 comprises the amino acid sequence ASQDVST,
ii. the light chain VL-CDR2 comprises the amino acid sequence WA,
iii. the light chain VL-CDR3 comprises the amino acid sequence HYSTP.

In one embodiment, the antibody, antibody fragment or antibody derivative thereof is an anti-CD37 antibody, antibody fragment or antibody derivative thereof.

In another embodiment, the antibody, antibody fragment or antibody derivative thereof is a monoclonal antibody.

In a further embodiment, the antibody, antibody fragment or antibody derivative thereof is a fragment selected from the group consisting of a Fab, Fab′, scFV, F(ab′)₂, F(ab)₂, F(ab)₃ and scFv-Fc fragment.

In yet another embodiment, the antibody, antibody fragment or antibody derivative thereof the antibody fragment is a minibody, diabody, triabody, or tetrabody.

In yet a further embodiment, the antibody, antibody fragment or antibody derivative thereof have a heavy chain variable domain (VH) that comprises the amino acid sequence of any one of SEQ ID NOs: 1-7 [VH sequence of AH02871, AH02875, AH02877, AH02879, AH02886 and AH02895] and a light chain variable domain (VL) comprises the amino acid sequence of any one of SEQ ID NOs: 8-18, 83 [VL sequences of AH02871, AH02875, AH02877, AH02879, AH02886, AH02895, AH02877_I106M, AH02877_1106V, AH02877_V110D, AH02877_I106M_V110D and AH02877_1106V_V110D].

In a preferred embodiment, the antibody, antibody fragment or antibody derivative thereof have a heavy chain variable domain (VH) that comprises the amino acid of SEQ ID NO: 2 [VH sequence of AH02871] and a light chain variable domain (VL) that comprises the amino acid sequence of any one of SEQ ID NO: 10, 14-18 [VL sequences of AH02877, AH02877_I106M, AH02877_I106V, AH02877_V110D, AH02877_I106M_V110D and AH02877_1106V_V110D].

In a more preferred embodiment, the antibody, antibody fragment or antibody derivative thereof have a heavy chain variable domain (VH) that comprises the amino acid of SEQ ID NO: 2 [VH sequence of AH02871] and a light chain variable domain (VL) that comprises the amino acid sequence of SEQ ID NO: 16 [VL sequences of AH02877_V110D].

In one embodiment, the antibody, antibody fragment or antibody derivative thereof have a predicted immunogenicity risk score (IRS) of the VH domain according to any one of SEQ ID NOs: 1-7 that is lower than the predicted IRS of SEQ ID NO: 19 [VH of Lilotomab].

In another embodiment, the antibody, antibody fragment or antibody derivative thereof have a predicted immunogenicity risk score (IRS) of the VL domain according to any one of SEQ ID NOs: 8-18 that is lower than the predicted IRS of SEQ ID NO: 20 [VL of Lilotomab].

In a preferred embodiment, the amino acid sequence of said antibody, antibody fragment or antibody derivative thereof is a combination of heavy chain and light chain fragments, where said antibody, antibody fragment or antibody derivative comprises,
a) a light chain having an amino acid sequence which is SEQ ID NO: 24 [AH02877_V110D] and a heavy chain having an amino acid sequence which is SEQ ID NO: 29 [NNV023 heavy chain],
b) a light chain having an amino acid sequence which is SEQ ID NO: 24 and a heavy chain having an amino acid sequence which is SEQ ID NO: 30 [NNV030 heavy chain, AH2871+ delCT Lys].

In one or more embodiments, the antibody, antibody fragment or antibody derivative is glycosylated.

In a preferred embodiment, said glycosylation of said antibody, antibody fragment or antibody derivative thereof is fucose deficient.

In a further embodiment said fucose deficient antibody, antibody fragment or antibody derivative thereof have an enhanced and/or improved induction of antibody-dependent cell-mediated cytotoxicity (ADCC), compared to a non-fucose deficient antibody, antibody fragment or antibody derivative thereof.

In one embodiment, the antibody, antibody fragment or antibody derivative thereof is a human or humanized antibody.

In a further embodiment, said human or humanized antibody have an enhanced and/or improved induction of antibody-dependent cell-mediated cytotoxicity (ADCC), antibody dependent cellular phagocytosis (ADCP) and/or complement-dependent cytotoxicity (CDC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37.

In a further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37, optionally in such as but not limited to Daudi and/or Ramos cells.

In a yet further embodiment, said human or humanized antibody have an enhanced and/or improved induction of antibody-dependent cell-mediated cytotoxicity (ADCC), antibody dependent cellular phagocytosis (ADCP) and/or complement-dependent cytotoxicity (CDC), compared to a non-humanized antibody comprising the light chain of SEQ ID NO: 22 [NNV003] and the heavy chain of SEQ ID NO: 23 and/or the therapeutic antibody Rituximab.

In a yet further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody Rituximab optionally in such as but not limited to Daudi and/or Ramos cells.

In one embodiment, the antibody, antibody fragment or antibody derivative thereof has an affinity for human CD37 expressing cells below 10 nM, such as below 9 nM, 8 nM, 7 nM, 6 nM, 5 nM, 4 nM, 3 nM, 2 nM, or 1 nM and/or such as below 1 nM, such as below 900 pM, 800 pM, 700 pM, 600 pM, 500 pM, 400 pM or 300 pM.

One or more aspect(s) of the present disclosure relates to a nucleic acid sequence encoding an antibody, antibody fragment or antibody derivative thereof according to the present disclosure.

In one embodiment, the nucleic acid sequence encodes an antibody, antibody fragment or antibody derivative thereof that is a combination of heavy chain and light chain fragments, where said antibody, antibody fragment or antibody derivative comprises,
a) a light chain having an amino acid sequence which is SEQ ID NO: 24 [AH02877_V110D] and a heavy chain having an amino acid sequence which is SEQ ID NO: 29 [NNV023 heavy chain].

In one embodiment, the nucleic acid sequence encodes an antibody, antibody fragment or antibody derivative thereof with a variable light chain and/or variable heavy chain of any one of SEQ ID NOs: 1-18.

Examples 14 and 15 show an immunoglobulin, such as an antibody, with V110D mutation extends the serum half-life as compared to the without the V110D mutation.

Thus, in a further embodiment, the immunoglobulin, such as an antibody, with V110D mutation extends the serum half-life as compared to the without the V110D mutation.

One or more aspect(s) of the present disclosure relates to a nucleic acid construct comprising one or more nucleic acid sequence(s) according to the present disclosure.

One or more aspect(s) of the disclosure relates to a host cell comprising one or more nucleic acid sequence(s) according to the present disclosure and/or nucleic acid construct(s) in the present disclosure.

In one embodiment, the host cell is a mammalian cell selected from the group consisting of chinese hamster ovary (CHO) cells, CHO-K1, CHO-DG44, mouse myeloma (NS0) cells, baby hamster kidney (BHK) cells, and human embryonic kidney lines (HEK293) cells, or an insect cell.

In one embodiment, the host cell is capable of producing an antibody, antibody fragment or antibody derivative thereof according to the present disclosure, wherein the cellular fucose glycosylation pathway of said host cell is modulated, such that the host cell produces a fucose deficient antibody, antibody fragment or antibody derivative thereof.

One or more aspect(s) of the present disclosure relates to an antibody, antibody fragment or antibody derivative thereof according to the present disclosure, produced in a host cell according to the present disclosure.

One or more aspect(s) of the present disclosure relates to an antibody, antibody fragment or antibody derivative thereof, drug conjugate that binds to human CD37 comprising:
a) an antibody, antibody fragment or antibody derivative thereof according to the present disclosure,
b) a linker, and
c) a drug selected from the group consisting of a toxin, a radioisotope, an anticancer drug, a cytotoxic drug and a cytostatic drug.

In one embodiment, said linker is a chelating linker.

In a preferred embodiment, said linker is a chelating linker selected from the group consisting of p-SCN-bn-DOTA, DOTA-NHS-ester and p-SCN-Bn-TCMC.

In one embodiment, said drug is a radionuclide, selected from the group consisting of ²¹¹At, ²¹³Bi, ²¹²Bi ²¹²Pb, ²²⁵Ac, ²²⁷Th, ⁹⁰Y, ¹⁸⁶Re, ¹⁸⁸Re, ¹⁹⁹Au, ¹⁹⁴Ir, ¹⁶⁶Ho, ¹⁵⁹Gd, ¹⁵³Sm, ¹⁶¹Tb, ¹⁴⁹Pm ¹⁴²Pr, ¹¹¹Ag, ¹⁰⁹Pd, ⁷⁷As, ⁶⁷Cu, ⁶⁴Cu, ⁴⁷Sc, and ¹⁷⁷Lu.

In another embodiment said drug is an anticancer drug.

One or more aspect(s) of the present disclosure relates to a pharmaceutical composition comprising, as the active ingredient, one or more antibody/antibodies, antibody fragment(s) or antibody derivative(s) thereof and/or an antibody, antibody fragment or antibody derivative thereof drug conjugate according to the present disclosure, and a pharmaceutically acceptable carrier.

In one embodiment, said composition further comprises an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

One or more aspect(s) of the present disclosure relates to a method for producing an antibody, antibody fragment or antibody derivative thereof according to the present disclosure, the method comprising,
a) introducing into a mammalian host cell one or more nucleic acid construct(s) of the present disclosure,
b) culturing said host cell in a suitable media,
c) recovering said antibody, antibody fragment or antibody derivative thereof from the culturing broth, and
d) purifying the antibody, antibody fragment or antibody derivative thereof.

In one embodiment of said method, the host cell is a mammalian cell selected from the group consisting of chinese hamster ovary (CHO) cells, CHO-K1, CHO-DG44, mouse myeloma (NS0) cells, baby hamster kidney (BHK) cells, and human embryonic kidney lines (HEK293) cells, or an insect cell.

In one embodiment of said method, the cellular fucose glycosylation pathway of said host cell is modulated, such that the host cell produces a fucose deficient antibody, antibody fragment or antibody derivative thereof.

One or more aspect(s) of the present disclosure relates to a method of depleting CD37 expressing B-cells from a population of cells, comprising administering to said population of cells, an antibody, antibody fragment or antibody derivative thereof, an antibody, antibody fragment or antibody derivative thereof, drug conjugate, or a pharmaceutical composition according to the present disclosure.

One or more aspect(s) of the present disclosure relates to a method of treating disease, wherein targeting of CD37 expressing B-cells can provide an inhibition and/or amelioration of said disease, comprising administering a therapeutically effective amount of an antibody, antibody fragment or antibody derivative thereof, an antibody, antibody fragment or antibody derivative thereof, drug conjugate, or a pharmaceutical composition according to the present disclosure.

One or more aspect(s) of the present disclosure relates to a method of treating cancer and/or inflammatory disease(s) and/or autoimmune disease(s) comprising administering a therapeutically effective amount of an antibody, antibody fragment or antibody derivative thereof, and/or a drug conjugate thereof, or a pharmaceutical composition according to the present disclosure.

One or more aspect(s) of the present disclosure relates to a method of treating cancer comprising administering a therapeutically effective amount of an antibody, antibody fragment or antibody derivative thereof, and/or a drug conjugate thereof, or a pharmaceutical composition according to the present disclosure.

One or more aspect(s) the present disclosure relates to the use of an antibody, antibody fragment or antibody derivative thereof, and/or a drug conjugate thereof, or a pharmaceutical composition according to the present disclosure, in inhibiting cancer and/or inflammatory disease(s) and/or autoimmune diseases.

One or more aspect(s) of the present disclosure relates to the use of an antibody, antibody fragment or antibody derivative thereof, and/or a drug conjugate thereof or a pharmaceutical composition according to the present disclosure, in ameliorating cancer and/or inflammatory disease(s) and/or autoimmune diseases.

One or more aspect(s) of the present disclosure relates to an antibody, antibody fragment or antibody derivative thereof, and/or a drug conjugate thereof, or a pharmaceutical composition according to the present disclosure, for use as a medicament.

In one embodiment said medicament is for use in the treatment of cancer.

In a preferred embodiment said medicament is for use in the treatment of B-cell malignancies.

In a more preferred embodiment, said medicament is for treating of a B-cell malignancy selected from the group consisting of B-cell non-Hodgkin's lymphoma, B-cell chronic lymphocytic leukemia, hairy cell leukemia, lymphoplasmacytic lymphoma and multiple myeloma, comprising administering to the individual in need thereof, an effective amount of an antibody, antibody fragment or antibody derivative thereof, and/or a drug conjugate thereof, or a pharmaceutical composition according to the present disclosure.

In one embodiment said medicament is for treating of inflammatory and autoimmune diseases wherein CD37-positive B cells are enriched.

In one embodiment said medicament is administered once or sequential.

One or more aspect(s) of the present disclosure relates to a formulation of an antibody, antibody fragment or antibody derivative thereof, and/or a drug conjugate thereof, or a pharmaceutical composition according to the present disclosure, for use in pre-treatment, wherein human CD37 is blocked in normal tissues before treatment with immunotoxic anti-CD37 or immunotoxic antibody-drug conjugate.

In one embodiment said formulation is suitable for administration by one or more administration routes selected from the group consisting of oral, topical, intravenous, intramuscular, and subcutaneous administration.

In one embodiment, the amount of the antibody fragment or antibody derivative thereof, and/or a drug conjugate thereof according to the present disclosure is at least 0.1 mg and not more than 1 g.

One or more aspect(s) of the present disclosure relates to a kit for the production of an antibody fragment or antibody derivative thereof, and/or a drug conjugate thereof according to the present disclosure comprising,
a) two or more vials, wherein one vial contains a conjugate comprising a drug linked to a linker, and one vial comprising an antibody fragment or antibody derivative thereof according to the present disclosure, and
b) optionally instructions for preparing said antibody-drug conjugate.

One or more aspect(s) of the present disclosure relates A kit for the production of an antibody fragment or antibody derivative thereof, drug conjugate according to the present disclosure comprising,
a) two or more vials, wherein one vial contains a conjugate comprising a chelator linked to an antibody fragment or antibody derivative thereof according to the present disclosure, a second vial containing a radionuclide, and
b) optionally, instructions for preparing said antibody-radionuclide conjugate.

One or more aspect(s) of the present disclosure relates to an antibody, antibody fragment or antibody derivative thereof, and/or conjugates thereof that binds to human CD37 comprising:
a) an antibody, antibody fragment or antibody derivative thereof according to the present disclosure,
b) a linker, and
c) a compound enriched in one or more isotopes selected from the group consisting of ¹¹C, ¹³N, 15O, ¹⁸F, ⁶⁴Cu and ⁸⁹Zr.

One or more aspect(s) of the present disclosure relates to an antibody, antibody fragment or antibody derivative thereof and/or conjugates thereof according to the present disclosure, for use in positron emission tomography imaging.

In one embodiment, said imaging is for providing diagnosis, staging, and monitoring treatment of cancers.

In another embodiment said cancer is B-cell non-Hodgkin's lymphoma, B-cell chronic lymphocytic leukemia, hairy cell leukemia, lymphoplasmacytic lymphoma and multiple myeloma One or more aspect(s) of the present disclosure relates to a pharmaceutical composition, comprising an antibody fragment or antibody derivative thereof, or an antibody fragment or antibody derivative thereof, drug conjugate according to the present disclosure, further comprising one or more further molecule(s), wherein the further molecules is selected from the group consisting of one or more antibodies, small molecule(s), peptide(s) and toxin(s).

### BRIEF DESCRIPTION OF FIGURES

FIG. 1 [Residues in inner core alignment]

Residues in inner core alignment: all framework residues in the inner core were highlighted in grey (responsible for the inner hydrophobic interaction between core aa); canonical FR residues (underlined); VH-VL interface residues (bold and italic). The residues, that were selected for priority back mutation are shown in boxes.

FIG. 2 [Indicated back mutations]

Indicated back mutations are assumed to contribute to the retention of the binding ability of the antibody. Back mutations can potentially increase the immunogenicity potential of the antibody. Core Back Mutations (CBM) in the boxes and CDR in bold.

FIG. 3 [Phage display figure]

General scheme of clone selection using Phage display and affinity maturation techniques.

FIG. 4 [position-specific immunogenicity]

Self-adjusted position-specific immunogenicity risk scores for lilotomab sequences.

Illustration of the self-adjusted position specific risk scores for the light (top) and heavy (bottom) chain sequences of lilotomab. The figure depicts the self-adjusted position-specific IRS calculated for the world population. Dotted lines highlight the position of the CRDs.

FIG. 5 [In-silico immunogenicity risk score]

T cell neo-epitope spanning NNV003 mV_{L}-hC_{L} in position 102-116.

Graphic representation of the in silico MHC class II-binding peptide mapping of lilotomab and NNV003 light chain sequence spanning VL-CL using the self-adjusted position-specific immunogenicity risk score for the world average population. The aa differences between lilotomab and NNV003 are highlighted in light grey. Dotted lines identified the CDRs.

FIG. 6 [position-specific immunogenicity risk scores for NNV020 heavy chain candidates]

Self-adjusted position-specific immunogenicity risk scores for NNV020 candidates (heavy chain). Illustration of the self-adjusted position specific risk scores for the light chain sequences of NNV020 drug candidates, lilotomab, NNV003 and rituximab. The figure depicts the self-adjusted position-specific IRS calculated for the world population. Dotted lines highlight the position of the CRDs.

FIG. 7 [self-adjusted position specific IRS for NNV020 heavy chain candidates]

NNV020 drug candidates self-adjusted position specific IRS heat map (heavy chain). Graphic representation of the in silico MHC class II-binding peptide mapping of NNV020 heavy chain sequences using the self-adjusted position-specific immunogenicity risk score for the world average population. Lilotomab, NNV003 and rituximab are included for benchmarking. Dotted lines identified the CDRs. Grey bars represent the gaps between aligned sequences.

FIG. 8 [immunogenicity risk scores for NNV020 light chain candidates]

Self-adjusted position-specific immunogenicity risk scores for NNV020 candidates (light chain) Illustration of the self-adjusted position specific risk scores for the light chain sequences of NNV020 drug candidates, lilotomab, NNV003 and rituximab. The figure depicts the self-adjusted position-specific IRS calculated for the world population. Dotted lines highlight the position of the CRDs. Replacing lilotomab C_{L} with hIgG1 C_{L} generates a new promiscuous overlapping T cell neo-epitope spanning NNV003, NNV020 drug candidates and rituximab mV_{L}-hC_{L} in position 102-116.

FIG. 9 [self-adjusted position IRS for NNV020 light chain candidates]

NNV020 drug candidates self-adjusted position specific IRS heat map (light chain).

Graphic representation of the in silico MHC class II-binding peptide mapping of NNV020 light chain sequences using the self-adjusted position-specific immunogenicity risk score for the world average population. Lilotomab, NNV003 and rituximab are included for benchmarking. Dotted lines identified the CDRs. Grey bars represent the gaps between aligned sequences.

FIG. 10 [T-cell neo epitope]

T cell neo-epitope spanning NNV020 drug candidates mV_{L}-hC_{L} in position 102-116. Graphic representation of the in silico MHC class II-binding peptide mapping of lilotomab, NNV003 and NNV020 drug candidates light chain sequence spanning V_{L}-C_{L} using the self-adjusted position-specific immunogenicity risk score for the world average population. The aa differences between lilotomab, NNV003 and NNV020 candidates are highlighted in light grey. Dotted lines identified the CDRs.

FIG. 11 [Conservation score for AH02877 light chain]

Conservation score vs. change in overall IRS for AH02877 light chain sequence spanning position 100-115

FIG. 12 [IRS heat map]

Self-adjusted position specific IRS heat map (light chain).

Graphic representation of the in-silico MHC class II-binding peptide mapping of lilotomab, NNV003, AH02877 and variants light chain sequences using the self-adjusted position-specific immunogenicity risk score for the world average population. Dotted lines identified the CDRs.

FIG. 13 [SDS-PAGE analysis of antibodies]

SDS-PAGE analysis of antibodies. SDS-PAGE analysis of IgG antibodies under native (left) and reducing conditions (right).

FIG. 14 [Coating levels of IgG variants in ELISA]

Coating levels of IgG variants in ELISA. ELISA showing detection of IgG variants (1000-0.45 ng/ml) coated directly in wells using (a) an AP-conjugated anti-human Fc specific Ab or (b) an AP-conjugated anti-human kappa LC antibody. Data shown as mean±s.d of duplicates from a representative experiment.

FIG. 15 [pH dependent binding of IgG variants to human FcRn]

pH dependent binding of IgG variants to human FcRn. ELISA showing binding of IgG variants (1000-0.45 ng/ml) to biotinylated human FcRn at (a) pH 6.0 and (b) pH 7.4. Data shown as mean±s.d of duplicates from a representative experiment. Arrow point to the O-Obin curve.

FIG. 16 [Binding of IgG variants to human FcγRs]

Binding of IgG variants to human FcγRs. ELISA showing binding of IgG variants (10.000-4.5 ng/ml) to biotinylated human (a) FcγRI, (b) FcγRIIa-H131, (c) FcγRIIa-R131, (d) FcγRIIb, (e) FcγRIIIa-V158, (f) FcγRIIIa-F158 and (g) FcγRIIIb. Data shown as mean±s.d of duplicates from a representative experiment. Arrows point to the O-Obin curves.

FIG. 17 [Binding of IgG variants to human C1q]

Binding of IgG variants to human C1q. ELISA showing binding of IgG variants (20.000-156.25 ng/ml) to human complement factor C1q. Data shown as mean±s.d of duplicates from a representative experiment. Arrow point to the O-Obin curve.

FIG. 18 [Repeated binding of IgG variants to human C1q]

Repeated binding of IgG variants to human C1q. Second ELISA showing binding of IgG variants (20.000-156.25 ng/ml) to human complement factor C1 q. Data shown as mean±s.d of duplicates from a representative experiment. Arrow point to the O-Obin curve.

FIG. 19 [Results of ADCC pilot experiment]

Results of ADCC pilot experiment (Exp #1). The upper row is for the data set obtained in Daudi cell line, the lower row is for Ramos cells. The dataset is presented in form of a clustered column plot to highlight the difference of each Ab at each concentration and line & scatter plot for better visualization of dose-response.

FIG. 20 [Results of ADCC repeat]

Results of ADCC repeat (Exp #2). The upper row is for the data set obtained in Daudi cell line, the lower row is for Ramos cells. The dataset is presented in form of a clustered column plot to highlight the difference of each Ab at each concentration and line & scatter plot for better visualization of dose-response.

FIG. 21 [Mean body weights Dauid mice 49 days]

Mean body weights including Standard error of the mean (SEM) of CB-17-Scid mice (n=5), with intravenously injected Daudi lymphoma cells (10 million cells per mouse) on Day −1. The mice were randomized into treatment groups according to body weight on Day −1. Treatment was initiated on Day 0, one day after inoculation. The mice received 7 different treatments from day 0 to day 19. The figure shows weight changes from 8 days prior to treatment start, to day 49 post treatment start in the obinutuzumab and Control subgroups. The “A” subgroups of the obinutuzumab groups were treated with the New and Old batches and subgroups “B” of obinutuzumab were treated with the old batch according to Table 22. Arrows on the x-axis indicate treatment days.

FIG. 22 [Mean body weight Daudi mice 139 days]

Mean body weights including Standard error of the mean (SEM) of CB-17-Scid mice (n=10), with intravenously injected Daudi-lymphoma cells (10 million cells per mouse) on day Day-1. The mice were randomized into treatment groups according to body weight on Day −1. Treatment was initiated one day after inoculation (Day 0). The mice received 7 different treatments from day 0 to day 19. The figure shows weight changes from 8 days prior to treatment start to day 139 post treatment start. Each group received 1-6 treatments of the relevant drug. Number of treatments per group is indicated with an asterix (*) and a number behind the asterix refers to number of times the group received the treatment in question.

FIG. 23 [Survival of Daudi mice]

Survival of 7 groups of CB-17-Scid mice with intravenously injected Daudi-lymphoma cells (10 million cells per mouse). The mice were randomized into treatment groups according to body weight on Day −1. Treatment was initiated one day after inoculation (Day 0). The mice received 7 different treatments from day 0 to day 19 according to Table 22. The figure shows survival portions up until 139 days from treatment start. Each group received 1-6 treatments of the relevant drug. Number of treatments per group is indicated with an asterix (*) and the number behind the asterix refers to number of times the group received the treatment in question. A humane end point was reached when one or more of the following clinical findings were present: Hind leg paralysis, Weight loss of 15% plus signs of discomfort, Weight loss of 20% if no abdominal tumors are palpable, hind leg paralysis is not present or other signs of substantial discomfort. All treatment groups (A-F) had statistically significant better survival than the control group (G), p<0.0001. There was not a statistically significant survival between treatment groups A-F (p>0.05). Kaplan Meier: Log-rank (Mantel-Cox Test).

FIG. 24 [Survival analysis]

Survival analysis. Mice were treated with 10 or 50 mg of either NNV024 or obinutuzumab. The survival of the mice treated with 50 mg and 10 mg NNV024 was significantly higher than the survival of mice treated with 50 mg and 10 mg obinutuzumab, respectively (log rank test p=0.049 and 0.043, respectively).

FIG. 25 [A DCC NNV023, NNV024]

Activation of ADCC induced by NNV023, NNV024, obinutuzumab and duohexabody-CD37 (DXBD37). The induction of ADCC was significantly higher for NNV024 than for the other antibodies (Holm Sidak test, p<0.05).

FIG. 26 [CDC Effects]

CDC Effects on cell viability induced by NNV Abs, obinutuzumab, and Duohexabody-37 on Raji target cells. A. Clustered bar plot representing CDC effects in presence of 12.5% Human Serum Complement (CTS-006, Creative Biolabs). B. Clustered bar plot representing CDC effects in 12.5% C3&C5 Removed Human Serum (CTS-054, Creative Biolabs). On the x axis, antibody concentration: CTR (0 pg/mL), 0.016, 0.4, 10 μg/mL; on the y axis, relative cell viability, expressed as % of cell viability compared to control cells not treated with any antibody. Results from two independent biological replicates.

FIG. 27 [CDC effects Daudi]

CDC Effects on cell viability induced by NNV Abs, obinutuzumab, and Duohexabody-37 on Daudi target cells. A. Clustered bar plot representing CDC effects in presence of 12.5% Human Serum Complement (CTS-006, Creative Biolabs). B. Clustered bar plot representing CDC effects in 12.5% C3&C5 Removed Human Serum (CTS-054, Creative Biolabs). On the x axis, antibody concentration: CTR (0 pg/mL), 0.016, 0.4, 10 μg/mL; on the y axis, relative cell viability, expressed as % of cell viability compared to control cells not treated with any antibody. Results from two independent biological replicates.

FIG. 28 [Survival analysis]

Survival analysis of mice were treated with 10 or 50 μg of either NNV024 or Obinutuzumab.

FIG. 29 [Mean body weight]

Mean body weights (error bars=standard error) of mice in the different treatment groups. The figure shows weight changes from inoculation (day 0) to study end at day 118 post treatment start.

FIG. 30 [ADCC induction in Burkitt's Lymphoma cell lines]

Normalized ADCC induction assessed with ADCC FcγRIIIa-158V reporter assay in Burkitt's Lymphoma cell lines (Ramos, Raji and Daudi cell lines). Bioluminescence is induced through FcγRIIIa/NFAT-associated luciferase activation. The result is a median of three independently prepared replicates normalized to the untreated within-the-plate control (Target+Effector cells without antibody). The error bars are SD. The spline lines are the fit of the data to a sigmoidal 4PL curve

FIG. 31 [ADCC induction in DLBCL cell lines]

Normalized ADCC induction assessed with ADCC FcγRIIIa-158V reporter assay in Diffused Large B-Cell Lymphoma (DLBCL) cell lines (DOHH-2, U2932 or WSU-DCLC-2 cells). Bioluminescence is induced through FcγRIIIa/NFAT-associated luciferase activation. The result is a median of three independently prepared replicates normalized to the untreated within-the-plate control (Target+Effector cells without Ab). The error bars are SD. The spline lines are the fit of the data to a sigmoidal 4PL curve.

FIG. 32 [ADCC induction in MCL or ALL cell lines]

Normalized ADCC induction assessed with ADCC FcγRIIIa-158V reporter assay in Mantle Cell (MCL) cell lines (Granta-519 or Rec-1 cells) or Acute Lymphoblastic Leukemia (ALL) non-T non-B (CD20-/CD37-) cell line (REH cells). Bioluminescence is induced through FcγRIIIa/NFAT-associated luciferase activation. The result is a median of three independently prepared replicates normalized to the untreated within-the-plate control (Target+Effector cells without Ab). The error bars are SD. The spline lines are the fit of the data to a sigmoidal 4PL curve.

FIG. 33 [ADCC concentration-response parameters]

Main parameters (Eₘₐₓ, EC₅₀, and AUC) for the ADCC concentration-response (conc.-resp.) curves for each Ab tested in the ten B-NHL cell lines. Plot-A—Effect (Eₘₐₓ) describes the range of the response. This parameter expresses a difference between the upper and lower asymptote of a conc.-resp. curve (sometimes termed the efficacy). The parameter is a function of the receptor occupancy and the ability to induce ADCC. The higher the Eₘₐₓ value, the stronger effect achieved at any equal EC₅₀. Plot-B—Half Maximal Effective Concentration (EC₅₀) is the potency of a drug. The lower EC₅₀ value, the better potency is. Plot-C—Area Under the Curve (AUC) is a function of Eₘₐₓ and EC₅₀ combined. The grey dash in each group of scatter represents the mean of the group, the numeric value of the mean is displayed next to it. The results for REH cell line are not shown since the cell line does not express CD20 and CD37 to a sufficient level to generate a concentration-response curve. Abbreviations: Ritux—rituximab; Obinutuzumab.

FIG. 34 [Plasma clearance of antibodies in mice]

Plasma clearance (% antibody remaining in plasma over time) of antibodies in Tg32 hemizygous mice. N=5 for NNV023, Obinutuzumab and DuoHexabody-CD37 and N=4 for NNV025 and NNV024. Data shown as mean±SD; The lines and the dashed lines are the fitting to Two Phase Exponential Decay model. ***p<0.001 by two-way ANOVA. Abbreviations: Obinutuzumab, DHXBD37-DuoHexaBody-CD37.

FIG. 35 [Plasma half-life of antibodies]

Plasma half-life of antibodies] in Tg32 hemizygous IVIg pre-loaded mice. Percent antibody remaining in plasma over time. NNV024 (n=5) is shown compared to NNV023 (n=5) and Obinutuzumab (n=4). Shown as mean+/−SD. The results from all the time points were fitted to a two-phase decay regression curve. The mean plasma half-life+/−SD for each antibody is indicated next to the legend. Significancy results by two-way ANOVA are reported as *=p<0.05 and ns=not significant.

FIG. 36 [Plasma concentration of antibodies]

Plasma concentration of antibodies in Tg32 hemizygous IVIg pre-loaded mice. Concentration of antibodies remaining in plasma over time. NNV024 (n=5) is shown compared to NNV023 (n=5) and Obinutuzumab (n=4). Shown as mean+/−SEM with lines connecting all the mean values from each time point.

FIG. 37 [Survival of female SCID FcRn−/− hFcRn (32) Tg mice]

Survival of female SCID FcRn−/− hFcRn (32) Tg mice with i.v. injected Daudi-lymphoma cells (10 million cells) treated with 2.69 mg/kg of NNV024, Obinutuzumab or recombinant DuoHexabody-CD37 or 100 ml NaCl.

FIG. 38 [Mean body weight]

Mean body weights of SCID FcRn−/− hFcRn (32) Tg mice (n=8-9), with intravenously injected Daudi-lymphoma cells (10 million cells per mouse) on day 0. The mice were randomized into treatment groups according to body weight from day −1. Treatment was initiated one day after inoculation (Day 1). The mice received 2.69 mg/kg of NNV024, Obinutuzumab or recombinant DuoHexabody-CD37 or 100 pl NaCl.

FIG. 39 [Activation of ADCC signaling]

Activation of ADCC signaling in treatment naive CLL patient samples. Signaling is activated through FcγIIa (V158) receptor on the Jurkat effector cells in response to binding to the test antibodies (NNV023, NNV024, rituximab, Obinutuzumab and recombinant version of DuoHexabody-CD37) on the target CLL patient cells. The effector-to-target cells ratio was 1:3. The spline curves are the regression of the datasets to four-parameter logistic (4PL) functions.

FIG. 40 [Average median fluorescence intensity (MFI)]

Average median fluorescence intensity (MFI) of the CLL patient samples labelled with either FITC-rituximab (anti-CD20) or Alexa648-Lilotomab (anti-CD37). All values are background corrected. The bars represent standard deviation (SD).

### DETAILED DESCRIPTION

The present disclosure relates to humanized antibodies, antibody fragments or antibody derivatives thereof from the mouse monoclonal antibody HH1 (lilotomab) and the chimeric monoclonal antibody chHH1 (NNV003).

### An Antibody, Antibody Fragment or Antibody Derivative Thereof

Humanized antibodies, antibody fragments or antibody derivatives thereof are antibodies from non-human species whose protein sequences have been modified to increase their similarity to antibody variants produced naturally in humans.

The process of “humanization” is usually applied to monoclonal antibodies developed for administration to humans.

Humanization can be necessary when the process of developing a specific antibody involves generation in a non-human immune system (such as that in mice).

The protein sequences of antibodies produced in this way are partially distinct from homologous antibodies occurring naturally in humans and are therefore, potentially immunogenic when administered to human patients.

Not all monoclonal antibodies designed for human administration need be humanized since many therapies are short-term interventions.

Humanization is usually seen as a distinct from the creation of a mouse-human antibody chimera, such as but not limited to chHH1.

So, although the creation of an antibody chimera is normally undertaken to achieve a more human-like antibody (by substituting the mouse Fc region of the antibody with that from human) simple chimeras of this type are not usually referred to as humanized.

Rather, the protein sequence of a humanized antibody is essentially identical to that of a human variant, despite the non-human origin of some of its complementarity determining region (CDR) segments responsible for the ability of the antibody to bind to its target antigen, as exemplified herein in examples 1-3, which exemplifies the development, deimmunization and manufacturing of a series of humanized antibodies.

The immunoglobulin heavy chain (Ig-HC) is the large polypeptide subunit of an antibody (immunoglobulin).

A typical antibody is composed of two immunoglobulin (Ig) heavy chains and two Ig light chains.

Several different types of heavy chain exist that define the class or isotype of an antibody.

These heavy chain types vary between different animals.

The immunoglobulin light chain is the small polypeptide subunit of an antibody (immunoglobulin).

There are two types of light chain in humans (as in other mammals), kappa (κ) chain, encoded by the immunoglobulin kappa locus on chromosome 2 and the lambda (λ) chain, encoded by the immunoglobulin lambda locus on chromosome 22.

Antibodies are produced by B lymphocytes, each expressing only one class of light chain.

Once set, light chain class remains fixed for the life of the B lymphocyte.

In a healthy individual, the total kappa to lambda ratio is roughly 2:1 in serum (measuring intact whole antibodies) or 1:1.5 if measuring free light chains, with a highly divergent ratio indicative of neoplasm.

The exact normal ratio of kappa to lambda ranges from 0.26 to 1.65.

Both the kappa and the lambda chains can increase proportionately, maintaining a normal ratio.

Both variable and constant domains in a humanized antibody fragments or antibody derivatives thereof derived from the mouse monoclonal antibody HH1 and/or the chimeric chHH1 can differ from known sequences.

Examples of such variations are clear from the present disclosure and include selection of constant domains, genetic variation of variable chains and variations of the Fc domain in order to modulate effector functions.

The present inventors have genetically engineered, humanized antibody fragments or antibody derivatives thereof, derived from the mouse monoclonal antibody HH1, lilotomab (NNV001) or the chimeric monoclonal antibody chHH1 (NNV003).

These antibodies show a promising effect in the search for optimal treatment of B-cell related malignancies and/or inflammatory disease(s) and/or autoimmune disease(s).

These antibodies show a promising effect in the search for optimal treatment of B-cell related malignancies.

These effects are shown in the experiments of the present disclosure.

Thus, one or more aspect(s) of the present disclosure relates to an antibody, antibody fragment or antibody derivative thereof, which comprises,
e) a heavy chain variable domain (VH) comprising VH-CDR1, VH-CDR2 and VH-CDR3, and
f) a light chain variable domain (VL) comprising VL-CDR1, VL-CDR2 and VL-CDR3, wherein,
g) the heavy chain variable domain (VH) comprises the amino acid sequence of any one of SEQ ID NO: 1 [heavy chain of H02871],
   wherein, according to SEQ ID NO: 1, position 2, or position 11 is I or V, position 12 is V or K, position 38 is K or R, position 48 is M or I, position 68 is A or V, position 70 is I or L, position 72 is R or V, position 81 is I or M, and wherein
iv. the heavy chain VH-CDR1 comprises the amino acid sequence GYSFTD,
v. the heavy chain VH-CDR2 comprises the amino acid sequence PYN,
vi. the heavy chain VH-CDR3 comprises the amino acid sequence PYGHYAM,
h) the light chain variable domain (VL) comprises the amino acid sequence of any one of SEQ ID NO: 8 [light chain of H02871],
   wherein, according to SEQ ID NO: 8, position 13 is A or T, position 43 is A or S, position 49 is Y or N, position 71 is F or Y, position 78 is M or L, position 106 is I, M, or V, position 110 is V or D,
iv. the light chain VL-CDR1 comprises the amino acid sequence ASQDVST,
v. the light chain VL-CDR2 comprises the amino acid sequence WA,
vi. the light chain VL-CDR3 comprises the amino acid sequence HYSTP.

In one or more embodiment(s) the heavy chain variable domain (VH) comprises the amino acid sequence of any one of SEQ ID NO: 1, or a functional homologue thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1.

In one or more embodiment(s) the heavy chain variable domain (VH) comprises the amino acid sequence of any one of SEQ ID NO: 2, or a functional homologue thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 2.

A functional homologue of an amino acid/nucleic acid sequence as described herein is a amino acid/nucleic acid sequence with alterations in the sequence, which retain its original functionality. A functional homologue may be obtained by mutagenesis. The functional homologue should have a remaining functionality of at least 70%, such as 80%, 90% or 100% compared to the functionality of the amino acid/nucleic acid sequence.

A functional homologue of any one of the disclosed amino acid or nucleic acid sequences can also have a higher functionality. A functional homologue of any one of the proposed antibodies, antibody fragments or antibody derivates thereof, comprising any one or more of SEQ ID NOs: 1-18, should ideally retain a high affinity binding to a human CD37 protein, and may induce antibody-dependent cell-mediated cytotoxicity (ADCC) in Ramos or Daudi cells or other beneficial effectors according to the present disclosure, furthermore a reduction in consumables, resulting in a lowered production cost or a prolonged shelf life is also a favourable feature.

In one or more exemplified embodiment(s) the heavy chain variable domain (VH) comprises the amino acid sequence of any one of SEQ ID NO: 1.

In one or more embodiment(s), the light chain variable domain (VL) comprises the amino acid sequence of any one of SEQ ID NOs: 8 [light chain of H02871], or a functional homologue thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 8.

In one or more embodiment(s), the light chain variable domain (VL) comprises the amino acid sequence of any one of SEQ ID NOs: 83 [light chain of H02871], or a functional homologue thereof having an amino acid sequence which is at least 80% identical, such as at least 81%, 82%, 83 %, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 83.

The term “sequence identity of [a certain]%” in the context of two or more nucleic acid or amino acid sequences means that the two or more sequences have nucleic acids or amino acid residues in common in the given percent, when compared and aligned for maximum correspondence over a comparison window or designated sequences of nucleic acids or amino acids (e.g., the sequences have at least 90 percent (%) identity). Percent identity of nucleic acid or amino acid sequences can be measured using a BLAST 2.0 sequence comparison algorithm with default parameters, or by manual alignment and visual inspection (see e.g. http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.20+ algorithm, which is described in Altschul et al. Nucl. Acids Res. 25, 3389 (1997). BLAST 2.2.20+ is used to determine percent sequence identity for the nucleic acids and proteins of the disclosure. Software for performing BLAST analyses is publicly available through the National Center for Biotechnology Information (http://www.ncbi.nlm.nih.gov/). Examples of commonly used sequence alignment algorithms are
CLUSTAL Omega (http://www.ebi.ac.uk/Tools/msa/clustalo/),
EMBOSS Needle (http://www.ebi.ac.uk/Tools/psa/emboss_needle/),
MAFFT (http://mafft.cbrc.jp/alignment/server/), or
MUSCLE (http://www.ebi.ac.uk/Tools/msa/muscle/).

In that regard, in one or more embodiment(s) the sequence identity of a sequence is at least 80% identical compared to a reference sequence. In one or more embodiment(s) the sequence identity of a sequence is at least 81% identical compared to a reference sequence. In one or more embodiment(s) the sequence identity of a sequence is at least 82% identical compared to a reference sequence. In one or more embodiment(s) the sequence identity of a sequence is at least 83% identical compared to a reference sequence. In one or more embodiment(s) the sequence identity of a sequence is at least 84% identical compared to a reference sequence. In one or more embodiment(s) the sequence identity of a sequence is at least 85% identical compared to a reference sequence. In one or more embodiment(s) the sequence identity of a sequence is at least 86% identical compared to a reference sequence. In one or more embodiment(s) the sequence identity of a sequence is at least 87% identical compared to a reference sequence. In one or more embodiment(s) the sequence identity of a sequence is at least 88% identical compared to a reference sequence. In one or more embodiment(s) the sequence identity of a sequence is at least 89% identical compared to a reference sequence. In one or more embodiment(s) the sequence identity of a sequence is at least 90% identical compared to a reference sequence. In one or more embodiment(s) the sequence identity of a sequence is at least 91% identical compared to a reference sequence. In one or more embodiment(s) the sequence identity of a sequence is at least 92% identical compared to a reference sequence. In one or more embodiment(s) the sequence identity of a sequence is at least 93% identical compared to a reference sequence. In one or more embodiment(s) the sequence identity of a sequence is at least 94% identical compared to a reference sequence. In one or more embodiment(s) the sequence identity of a sequence is at least 95% identical compared to a reference sequence. In one or more embodiment(s) the sequence identity of a sequence is at least 96% identical compared to a reference sequence. In one or more embodiment(s) the sequence identity of a sequence is at least 97% identical compared to a reference sequence. In one or more embodiment(s) the sequence identity of a sequence is at least 98% identical compared to a reference sequence. In one or more embodiment(s) the sequence identity of a sequence is at least 99% identical compared to a reference sequence. In one or more embodiment(s) the sequence identity of a sequence is at least 99.9% identical compared to a reference sequence.

In one or more embodiment(s) the sequence identity of a sequence is 100% identical to a reference sequence.

In one or more embodiment(s), the heavy chain variable domain (VH) comprises any one of the amino acid sequence variants of SEQ ID NO: 1 [heavy chain of H02871 w variants] and the light chain variable domain (VL) comprises any one of the amino acid sequence variants of SEQ ID NO: 8 [light chain of H02871 w variants].

In one or more embodiment(s), the heavy chain variable domain (VH) comprises the amino acid sequence of SEQ ID NO: 2 [heavy chain of H02871] and the light chain variable domain (VL) comprises the amino acid sequence variants of SEQ ID NO: 83 [light chain of H02871].

In one or more embodiments the antibody, antibody fragment or antibody derivative thereof, comprises,
a) a heavy chain variable domain (VH) comprising VH-CDR1, VH-CDR2 and VH-CDR3, and
b) a light chain variable domain (VL) comprising VL-CDR1, VL-CDR2 and VL-CDR3, wherein,
c) the heavy chain variable domain (VH) comprises the amino acid sequence of any one of SEQ ID NO: 1 [heavy chain of H02871],
   wherein, according to SEQ ID NO: 1, position 2, or position 11 is I or V, position 12 is V or K, position 38 is K or R, position 48 is M or I, position 68 is A or V, position 70 is I or L, position 72 is R or V, position 81 is I or M, and wherein the heavy chain optionally comprises,
      i. VH-CDR1 of the amino acid sequence GYSFTD, and/or
      ii. VH-CDR2 of the amino acid sequence PYN, and/or
      iii. VH-CDR3 of the amino acid sequence PYGHYAM,
d) the light chain variable domain (VL) comprises the amino acid sequence of any one of SEQ ID NOs: 8 [light chain of H02871],
   wherein, according to SEQ ID NO: 8, position 13 is A or T, position 43 is A or S, position 49 is Y or N, position 71 is F or Y, position 78 is M or L, position 106 is I, M, or V, position 110 is V or D and wherein the heavy chain optionally comprises,
      vii. VL-CDR1 of the amino acid sequence ASQDVST and/or,
      viii. VL-CDR2 of the amino acid sequence WA,
      ix. VL-CDR3 of the amino acid sequence HYSTP.

A light chain variable domain (VL) and/or a heavy chain variable domain (VH) An antibody, antibody fragment or antibody derivative thereof, may comprise a light chain variable domain (VL) and/or a heavy chain variable domain (VH). In some cases, an antibody, antibody fragment or antibody derivative thereof comprises a light chain variable domain (VL) and no heavy chain variable domain (VH). In other cases, an antibody, antibody fragment or antibody derivative thereof comprises a heavy chain variable domain (VH) and no light chain variable domain (VL) Thus, in one or more embodiment(s), the antibody, antibody fragment or antibody derivative thereof, comprises a light chain variable domain (VL) and/or a heavy chain variable domain (VH).

### VH and VL Variants

In the examples of the present disclosure the CDRs of lilotomab were grafted into the human acceptors to obtain five humanized light chains and five humanized heavy chains for each antibody. Twenty-five humanized antibodies were expressed in HEK293 cells and the supernatants were assessed by fluorescence-activated single cell sorting (FACS), as described in example 1. In addition, in order to retain a conformational structure and conserved amino acids of the initial NNV001 Ab (lilotomab), the two sequences HC1, LC1 was subjected to back mutations. Increasing the number of exemplified antibodies to six humanized light chains and six humanized heavy chains for each antibody, of which 26 different variants are exemplified in Example 1 and 4.

Thus, in one or more exemplified embodiment(s), the antibody, antibody fragment or antibody derivative thereof have a heavy chain variable domain (VH) that comprises the amino acid sequence of any one of SEQ ID NOs: 1-7 [VH sequence of AH02871, AH02875, AH02877, AH02879, AH02886 and AH02895] and a light chain variable domain (VL) comprises the amino acid sequence of any one of SEQ ID NOs: 8-18 [VL sequences of AH02871, AH02875, AH02877, AH02879, AH02886, AH02895, AH02877_I106M, AH02877_1106V, AH02877_V110D, AH02877_I106M_V110D and AH02877_1106V_V110D].

In a preferred embodiment, the antibody, antibody fragment or antibody derivative thereof have a heavy chain variable domain (VH) that comprises the amino acid of SEQ ID NO: 2 [VH sequence of AH02871] and a light chain variable domain (VL) that comprises the amino acid sequence of any one of SEQ ID NO: 10, 14-18 [VL sequences of AH02877, AH02877_I106M, AH02877_I106V, AH02877_V110D, AH02877_I106M_V110D and AH02877_1106V_V110D].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 18 [AH02877_I106V_V110D_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 18 [AH02877_I106V_V110D_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 18 [AH02877_I106V_V110D_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 18 [AH02877_1106V_V110D_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 18 [AH02877_I106V_V110D_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 83 [AH02871_LC_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 9 [AH02875_LC_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 10 [H02877_LC_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 11 [H02879_LC_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 12 [H02886_LC_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 13 [H02895_LC_VL].

In one or more embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 14 [H02877_I106M_VL].

In one or more embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 15[AH02877_I106V_VL].

In one or more embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 17 [AH02877_I106M_V110D_VL].

In one or more embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 18 [AH02877_I106V_V110D_VL].

### V110D Variants

Example 2 discloses a combination of epitope mapping and overall self-adjusted immunogenicity risk score-based ranking and predicts SEQ ID NO: 16 as one of the optimal tested combinations to reduce light chain predicted immunogenicity potential.

Thus, in a preferred embodiment, the light chain variable domain (VL) comprises the amino acid sequence variants of SEQ ID NO: 16 [AH02877_V110D_VL], or a functional homologue thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 16.

In one or more preferred embodiment(s), the heavy chain variable domain (VH) comprises the amino acid sequence of SEQ ID NO: 2[AH02871_HC_VH] and the light chain variable domain (VL) comprises the amino acid sequence variants of SEQ ID NO: 16 [AH02877_V110D_VL].

In one or more preferred embodiment(s), the heavy chain variable domain (VH) comprises the amino acid sequence of SEQ ID NO: 3 [AH02875_HC_VH] and the light chain variable domain (VL) comprises the amino acid sequence variants of SEQ ID NO: 16 [AH02877_V110D_VL].

In one or more exemplified embodiment(s), the heavy chain variable domain (VH) comprises the amino acid sequence of SEQ ID NO: 4 [AH02877_HC_VH] and the light chain variable domain (VL) comprises the amino acid sequence variants of SEQ ID NO: 16 [AH02877_V110D_VL].

In one or more preferred embodiment(s), the heavy chain variable domain (VH) comprises the amino acid sequence of SEQ ID NO: 5 [AH02879_HC_VH] and the light chain variable domain (VL) comprises the amino acid sequence variants of SEQ ID NO: 16 [AH02877_V110D_VL].

In one or more preferred embodiment(s), the heavy chain variable domain (VH) comprises the amino acid sequence of SEQ ID NO: 6 [AH02886_HC_VH] and the light chain variable domain (VL) comprises the amino acid sequence variants of SEQ ID NO: 16 [AH02877_V110D_VL].

In one or more exemplified embodiment(s), the heavy chain variable domain (VH) comprises the amino acid sequence of SEQ ID NO: 7 [AH02895_HC_VH] and the light chain variable domain (VL) comprises the amino acid sequence variants of SEQ ID NO: 16 [AH02877_V110D_VL].

### A Typical Antibody

A typical antibody is composed of two immunoglobulin (Ig) heavy chains and two Ig light chain domains.

### Lambda or Kappa Light Chain Constant Domain

In the present disclosure humanization of the light chain domain was done by grafting the CDR regions onto human light chain acceptor sequences. In example 1, the human kappa light chain was used as the constant light chain domain, followed by back mutations onto the sequence with the highest homology to lilotomab.

Thus, in one embodiment, the antibody, antibody fragment or antibody derivative thereof comprises a lambda or kappa light chain constant domain.

In one embodiment, the antibody, antibody fragment or antibody derivative thereof is comprises a kappa light chain constant domain having an amino acid sequence of SEQ ID NO: 31, or a functional homologue thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 31.

In one or more embodiment(s) the light chain constant domain is of human origin.

### Heavy Chain Constant Domain

Similarly, to the light chain constant domain, humanization of the heavy chain domain was done by by grafting the CDR regions onto human heavy chain acceptor sequences. In example 1, the human IgG1 chain was used as the constant light chain domain, followed by back mutations onto the sequence with the highest homology to lilotomab.

Thus, in one or more embodiment(s) of the present disclosure the antibody, antibody fragment or antibody derivative thereof is defined by i) a constant heavy chain is selected from the group consisting of IgG1, IgG2, IgG3 and IgG4 chain, and ii) a constant light chain is a kappa or a lambda chain, wherein heavy chain variable regions according to the present disclosure are grafted onto the constant heavy chain, and light chain variable regions according to the present disclosure are grafted onto the constant light chain.

### IgG Subtypes

In a further embodiment, the antibody, antibody fragment or antibody derivative thereof is an antibody that comprises an IgG1, IgG2, IgG3 or IgG4, IgM, IgA, IgE and/or IgD heavy chain constant domain of human origin.

In another embodiment, the antibody, antibody fragment or antibody derivative thereof comprises a lambda and/or kappa light chain constant domain of human origin and/or an IgG1, IgG2, IgG3 or IgG4, IgM, IgA, IgE or IgD heavy chain constant domain of human origin.

In another embodiment, the antibody, antibody fragment or antibody derivative thereof comprises a lambda or kappa light chain constant domain of human origin and/or an IgG1, IgG2, IgG3 or IgG4, IgM, IgA, IgE or IgD heavy chain constant domain of human origin.

In another embodiment, the antibody, antibody fragment or antibody derivative thereof is an antibody that comprises a lambda or kappa light chain constant domain of human origin and an IgG1, IgG2, IgG3 or IgG4, IgM, IgA, IgE or IgD heavy chain constant domain of human origin.

In a further embodiment, the antibody, antibody fragment or antibody derivative thereof is an antibody that comprises a kappa light chain constant domain of human origin and an IgG1, IgG2, IgG3 or IgG4 heavy chain constant domain of human origin.

In a further embodiment, the antibody, antibody fragment or antibody derivative thereof is an antibody that comprises a kappa light chain constant domain of human origin and an IgG1 or IgG3 heavy chain constant domain of human origin.

In a preferred embodiment, the antibody, antibody fragment or antibody derivative thereof is an antibody that comprises a kappa light chain constant domain of human origin and an IgG1 heavy chain constant domain of human origin.

### Heavy Chain and Light Chain Constant Domain

In a preferred embodiment, the amino acid sequence of said antibody, antibody fragment or antibody derivative thereof is a combination of heavy chain and light chain fragments, where said antibody, antibody fragment or antibody derivative comprises,
a) a light chain having an amino acid sequence which is SEQ ID NO: 24 [AH02877_V110D] and a heavy chain having an amino acid sequence which is SEQ ID NO: 29 [NNV023 heavy chain],

Example 2 relates to the predicted immunogenicity of the different heavy chain, and light chain back mutations, as a consequence, the heavy chain of SEQ ID NO: 29 [AH02871_HC/NNV023_HC] and the light chain of SEQ ID NO: 24 [AH02877_LC], comprising the back mutation V110D were selected to generate an optimized candidate as the model suggested that this had the lowest immunogenicity risk score (IRS).

Thus, in a preferred embodiment, the amino acid sequence of said antibody, antibody fragment or antibody derivative thereof is a combination of heavy chain and light chain fragments, comprising, a light chain having an amino acid sequence which is SEQ ID NO: 24 [AH02877_V110D] and a heavy chain having an amino acid sequence which is SEQ ID NO: 29 [NNV023 heavy chain].

### Monoclonal Antibody

Immunotherapy using monoclonal antibodies (mAbs) has been emerging as a safe and selective method for the treatment of cancer and other diseases.

In particular, the role of monoclonal antibodies in therapies that are based on B-cell depletion, e.g. in the treatment of B-cell malignancies, has expanded since the introduction of rituximab, an antibody that is directed against the CD20 antigen on the B-cell surface.

Thus, in one or more preferred exemplified embodiment(s), the antibody, antibody fragment or antibody derivative thereof is a monoclonal antibody.

In one or more embodiment(s), the monoclonal antibody comprises the heavy chain domain of SEQ ID NO: 29 and the light chain domain of SEQ ID NO: 25.

In one or more embodiment(s), the monoclonal antibody comprises the heavy chain domain of SEQ ID NO: 29 and the light chain domain of SEQ ID NO: 26.

In one or more embodiment(s), the monoclonal antibody comprises the heavy chain domain of SEQ ID NO: 29 and the light chain domain of SEQ ID NO: 27.

In one or more embodiment(s), the monoclonal antibody comprises the heavy chain domain of SEQ ID NO: 29 and the light chain domain of SEQ ID NO: 28.

In one or more embodiment(s), the monoclonal antibody comprises the heavy chain domain of SEQ ID NO: 30 and the light chain domain of SEQ ID NO: 25.

In one or more embodiment(s), the monoclonal antibody comprises the heavy chain domain of SEQ ID NO: 30 and the light chain domain of SEQ ID NO: 26.

In one or more embodiment(s), the monoclonal antibody comprises the heavy chain domain of SEQ ID NO: 30 and the light chain domain of SEQ ID NO: 27.

In one or more embodiment(s), the monoclonal antibody comprises the heavy chain domain of SEQ ID NO: 30 and the light chain domain of SEQ ID NO: 28.

In one or more preferred embodiment(s), the monoclonal antibody comprises the heavy chain domain of SEQ ID NO: 29 [NNV023_HC/AH02871_HC] and the light chain domain of SEQ ID NO: 24 [NNV023 full light chain AH02877_V110D].

In one or more preferred embodiment(s), the monoclonal antibody comprises the heavy chain domain of SEQ ID NO: 30 and the light chain domain of SEQ ID NO: 24 [NNV023 full light chain AH02877_V110D], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 30 and/or SEQ ID NO: 24.

In one or more preferred embodiment(s), the monoclonal antibody comprises the heavy chain domain of SEQ ID NO: 29 [NNV023_HC/AH02871_HC] and the light chain domain of SEQ ID NO: 24 [NNV023 full light chain AH02877_V110D].

In one or more preferred embodiment(s), the monoclonal antibody comprises the heavy chain domain of SEQ ID NO: 30 and the light chain domain of SEQ ID NO: 24 [NNV023 full light chain AH02877_V110D].

### Traditional Antibody

A traditional antibody comprises two disulphide bridge linked heavy chains and two light chains, linked to the heavy chain via disulphide bridges. The heavy chain, in general comprise a variable domain (VH) and potentially three constant domains CH₁-CH₃, wherein CH₁ and CH₂ is linked via a hinge region comprising one or more cysteines partially responsible for heavy chain dimerization.

CH₂ and CH₃ also comprises cysteines also partially responsible for heavy chain dimerization. The light chain comprises a variable domain (VL) and a constant domain (CL). The heavy chain CH₁ and light chain CL are linked via one or more cysteine residues forming one or more disulphide bridge(s).

Due to the many different domains and chain linkage options several different antibody fragment variants have emerged.

Fab, Fab′, scFV, F(ab′)₂, F(ab)₂, F(ab)₃ and scFv-Fc Fragment.

Modifying antibody features such as molecular size, valency, binding affinity, and pharmacokinetics allows for the development of antibody fragments with tailor-made properties for a variety of clinical applications. Variation in molecular size and binding properties among antibody fragments and antibody derivates thereof is considered to possess a central role in the tissue distribution of targeting molecules.

Thus, in one or more embodiment(s), the antibody, antibody fragment or antibody derivative thereof is a fragment selected from the group consisting of a Fab, Fab′, scFV, F(ab′)₂, F(ab)₂, F(ab)₃ and scFv-Fc fragment.

### Fab

The antigen-binding fragment (Fab), according to the present disclosure, is a region on an antibody that binds to antigens. It is composed of one constant and one variable domain of each of the heavy and the light chain, thus a Fab fragment does not contain an Fc fragment. The variable domains comprises the antigen-binding site, comprising the CDRs.

Fab fragments may be prepared from an IgG like antibody by enzymatic degradation targeting the hinge region of said antibody. Alternatively, a Fab fragment is produced in a host cell, comprising a nucleotide encoding the Fab fragment, thus, only producing the portion of the antibody fragment that is the Fab fragment.

Thus, in one or more embodiments, the antibody, antibody fragment or antibody derivative thereof is a Fab fragment, comprising a heavy chain variable domain (VH) that comprises any one of the amino acid sequence variants of SEQ ID NO: 1 [heavy chain of H02871 w variants] and the light chain variable domain (VL) comprises any one of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8.

Thus, in one or more further embodiments, the antibody, antibody fragment or antibody derivative thereof is a Fab fragment, comprising a heavy chain variable domain (VH) that comprises the amino acid sequence of SEQ ID NO: 2 [heavy chain of H02871 w variants] and the light chain variable domain (VL) comprises the amino acid sequence of SEQ ID NO: 83 [light chain of H02871 w variants].

### Fab′

A Fab′ fragment, according to the present disclosure, is a Fab fragment, further comprising at least a portion of the hinge region of a traditional antibody, but which does not comprise a disulphide bridge responsible for dimerization of the individual fragments.

Fab′ fragments may be prepared from an IgG like antibody by enzymatic degradation targeting the hinge region of said antibody. Alternatively, a Fab′ fragment is produced in a host cell, comprising a nucleotide encoding the Fab′ fragment, thus, only producing the portion of the antibody fragment that is the Fab′ fragment, wherein the Fab′ fragment is postprocessed in order to reduce disulphide bridges formed between individual Fab′ fragments.

Thus in one or more embodiments, the antibody, antibody fragment or antibody derivative thereof is a Fab′ fragment, comprising a heavy chain variable domain (VH) that comprises any one of the amino acid sequence variants of SEQ ID NO: 1 [heavy chain of H02871 w variants] and the light chain variable domain (VL) comprises any one of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8.

Thus, in one or more further embodiments, the antibody, antibody fragment or antibody derivative thereof is a Fab′ fragment, comprising a heavy chain variable domain (VH) that comprises the amino acid sequence of SEQ ID NO: 2 [heavy chain of H02871 w variants] and the light chain variable domain (VL) comprises the amino acid sequence of SEQ ID NO: 83 [light chain of H02871 w variants].

### F(ab)₂,

A Fab′ fragment, according to the present disclosure, is a Fab fragment, further comprising at least a portion of the hinge region of a traditional antibody i.e., a Fab′ fragment, wherein, the disulphide bridges linking individual Fab′ fragments is not reduced, thus making a dimeric antibody Fab′ fragment, denoted as F(ab′)₂.

Thus, in one or more embodiment(s), the disulphides of the antibody fragment hinge region is reduced, resulting in a Fab′ antibody fragment. In one or more embodiment(s) the disulphides are oxidized resulting in a F(ab′)₂ antibody fragment.

In that regard, in one or more embodiments, the antibody, antibody fragment or antibody derivative thereof is a F(ab′)₂ fragment, comprising one or more heavy chain variable domain(s) (VH) that comprises any one or more of the amino acid sequence variants of SEQ ID NO: 1 [heavy chain of H02871 w variants] and one or more light chain variable domain(s) (VL) comprises any one or more of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8.

Thus, in one or more further embodiments, the antibody, antibody fragment or antibody derivative thereof is a F(ab′)₂ fragment, comprising a heavy chain variable domain (VH) that comprises the amino acid sequence of SEQ ID NO: 2 [heavy chain of H02871 w variants] and a light chain variable domain (VL) comprises the amino acid sequence of SEQ ID NO: 83 [light chain of H02871 w variants].

Alternatively, the Fab or Fab′ fragments are chemically linked, by a chemical linker, such as but not limited to a disuccinimidyl suberate (DSS) linker, N-Hydroxysuccinimide-Polyethyleneglycol (NHS-PEG) linker or similar chemical linker, resulting in a chemically linked F(ab)₂ or F(ab′)₂ fragment.

In that regard the F(ab)₂ or F(ab′)₂ may be a monospecific or bi-specific antibody fragment.

### F(ab)₃

Fab, Fab′, F(ab)₂ or F(ab′)₂ may also be combined into a F(ab)₃ fragment comprising three individual Fab or Fab′, or a F(ab)₂, a F(ab′)₂ and a Fab or Fab′ fragment, thus, comprising three light chains and three heavy chain fragments, linked into a tripart fragment.

The F(ab)₃ fragment may be assembled by disulphide linkage or chemical linkage as disclosed herein.

In that regard, in one or more embodiments, the antibody, antibody fragment or antibody derivative thereof is a F(ab)₃ fragment, comprising one or more heavy chain variable domain (VH) that comprises any one or more of the amino acid sequence variants of SEQ ID NO: 1 [heavy chain of H02871 w variants] and one or more light chain variable domain (VL) comprises any one or more of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8.

Thus, in one or more further embodiments, the antibody, antibody fragment or antibody derivative thereof is a F(ab)₃ fragment, comprising a heavy chain variable domain (VH) that comprises the amino acid sequence of SEQ ID NO: 2 [heavy chain of H02871 w variants] and a light chain variable domain (VL) comprises the amino acid sequence of SEQ ID NO: 83 [light chain of H02871 w variants].

In that regard the F(ab)₃ may be a monospecific, bi-specific or trispecific antibody fragment.

scFV

An scFV fragment, according to the present disclosure, is an antibody fragment, comprising a heavy chain fragment comprising a variable domain (VH) and optionally a constant domain (CH) and a light chain fragment comprising a variable domain (VL) and optionally a constant light chain domain (CL), wherein the light chain and the heavy chain fragments are linked by a linker, thus making a single fragment. Such as linker may be a chemical linker as described in the present disclosure, an amino acid linker, such as but not limited to a poly-Gly-Ser linker or it may be a combination of a chemical and amino acid linker.

In that regard, in one or more embodiments, the antibody, antibody fragment or antibody derivative thereof is a scFV fragment, comprising a heavy chain variable domain (VH) that comprises any one of the amino acid sequence variants of SEQ ID NO: 1 [heavy chain of H02871 w variants] and the light chain variable domain (VL) which comprises any one of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8, wherein the fragment further comprises a linker linking the heavy chain and the light chain.

Thus, in one or more further embodiments, the antibody, antibody fragment or antibody derivative thereof is an scFV fragment, comprising a heavy chain variable domain (VH) that comprises the amino acid sequence of SEQ ID NO: 2 [heavy chain of H02871 w variants] and a light chain variable domain (VL) comprises the amino acid sequence of SEQ ID NO: 83 [light chain of H02871 w variants].

Individual scFV fragment may also be linked to form a dimeric, trimeric or tetrameric scFV construct i.e., scFV2, scFV3, scFV4. Such a link may also be a disulphide link. scFV2, scFV3, scFV4 are also referred to as diabodies, tribadies or tetrabodies.

### Diabody, Tribody or Tetrabody

Thus, in yet another embodiment, the antibody, antibody fragment or antibody derivative thereof the antibody fragment is a diabody, triabody, or tetrabody.

scFv-Fc

An scFV fragment, according to the present disclosure, is an antibody fragment, comprising a heavy chain fragment comprising a variable domain (VH) and more than one constant domain (CH), such as but not limited to CH₁ and CH₂ or CH₁, CH₂ and CH₃, and a light chain fragment comprising a variable domain (VL) and optionally a constant light chain domain (CL), wherein the light chain and the heavy chain fragments are linked by a linker, thus making a single fragment. Such as linker may be a chemical linker as described in the present disclosure, an amino acid linker, such as but not limited to a poly-Gly-Ser linker or it may be a combination of a chemical and amino acid linker.

In that regard, in one or more embodiments, the antibody, antibody fragment or antibody derivative thereof is a scFV-Fc fragment, comprising a heavy chain variable domain (VH) that comprises any one of the amino acid sequence variants of SEQ ID NO: 1 [heavy chain of H02871 w variants] and the light chain variable domain (VL) which comprises any one of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8, wherein the fragment further comprises a linker linking the heavy chain and the light chain.

Thus, in one or more further embodiments, the antibody, antibody fragment or antibody derivative thereof is an scFV-Fc fragment, comprising a heavy chain variable domain (VH) that comprises the amino acid sequence of SEQ ID NO: 2 [heavy chain of H02871 w variants] and a light chain variable domain (VL) comprises the amino acid sequence of SEQ ID NO: 83 [light chain of H02871 w variants].

### Minibody

A scFV-Fc fragment comprising a CH₁ and CH₂ domain may also be referred to as a minibody.

Thus, in one or more embodiment(s), the antibody, antibody fragment or antibody derivative thereof the antibody fragment is a minibody.

### An Anti-CD37 Antibody

The present disclosure relates to an antibody, antibody fragment or antibody derivative thereof, wherein the antibody, antibody fragment or antibody derivative thereof is optimized for binding to CD37. Binding of a CD37-specific antibody, antibody fragment or antibody derivative thereof, to cancer cells may trigger various mechanisms of action:
After the antibody, antibody fragment or antibody derivative thereof, binds to the extracellular domain of the CD37 antigen, it may activate the complement cascade and lyse the targeted cell.
An anti-CD37 antibody, antibody fragment or antibody derivative thereof, may mediate antibody-dependent cell-mediated cytotoxicity (ADCC), antibody dependent cellular phagocytosis (ADCP) and/or complement-dependent cytotoxicity (CDC), to the target cell, which occurs after the Fc portion of the bound antibody is recognized by appropriate receptors on cytotoxic cells of the immune system.
The antibody may alter the ability of B-cells to respond to antigen or other stimuli. Finally, anti-CD37 antibody, antibody fragment or antibody derivative thereof, may initiate programmed cell death (apoptosis).

Thus, in one embodiment, the antibody, antibody fragment or antibody derivative thereof is a CD37 targeting molecule.

In one embodiment, the antibody, antibody fragment or antibody derivative thereof is an optimized CD37 targeting molecule.

In one embodiment, the antibody, antibody fragment or antibody derivative thereof comprises an optimized CD37 targeting light chain.

Example 1 and 2 provides non-exhaustive examples of optimized CD37 targeting light chains.

In one embodiment, the antibody, antibody fragment or antibody derivative thereof is an anti-CD37 antibody, antibody fragment or antibody derivative thereof according to the present disclosure.

In another embodiment, the antibody, antibody fragment or antibody derivative thereof is a polyclonal anti-CD37 antibody.

In one or more exemplified embodiment(s), the antibody, antibody fragment or antibody derivative thereof is a monoclonal anti-CD37 antibody.

In one or more embodiment(s) the antibody, antibody fragment or antibody derivative thereof, forms IgG complexes in the presence of CD37.

### A Derivate Thereof

In the present disclosure an antibody derivate relates to antibody derivatives that make use of selected parts of an antibody resulting in molecules with novel biological activity and rationally designed mechanisms of action.

In that regard, in one or more embodiment(s) of the present disclosure relates to an antibody derivate.

### Human or Humanized Antibody

In one embodiment, the antibody, antibody fragment or antibody derivative thereof is a human or humanized antibody.

### Glycosylation

Antibody glycosylation defines the functional potential of the antibody by delineating the structure of the antibody Fc region and determining which Fc receptors it can bind to in order to recruit effector cells.

The effector functions that antibodies mediate, including cytotoxicity and phagocytosis, are critical for protection against and prevention of many diseases.

Antibody glycosylation has been harnessed to improve the efficacy of monoclonal therapeutics. Antibody glycosylation can be modulated by vaccination, indicating that rational immunogen design could seek to elicit a specific antibody glycosylation response.

Thus, in one or more exemplified embodiments, the antibody, antibody fragment or antibody derivative thereof is glycosylated.

### Fucose Deficient

Glycoengineered therapeutic antibodies lacking core fucose residue from the Fc N-glycans exhibit strong ADCC at lower concentrations with much higher efficacy compared to fucosylated counterparts and can evade the inhibitory effect of serum immunoglobulin G (IgG) on ADCC through its high binding to gamma receptor IIIa (Fc FcγRIIIa).

The antibodies tested (NNV023 and NNV024) showed the ability to induce ADCC in both Ramos and Daudi cell lines. In both cell lines NNV024 demonstrated superior ADCC activation which maximum was 6-fold (for 0.01 μg/mL in Ramos) to 9.5-fold (for 0.1 μg/mL in Daudi) times higher than that of rituximab for the same doses. The main comparator, obinutuzumab, was just 4.5-fold times (0.025 μg/mL in Ramos) to 8.3-fold times (0.1 μg/mL in Daudi) more potent than rituximab. The NNV023 Ab had also a good ability for ADCC induction in both cell lines: 2.1-fold time at 0.04 μg/mL in Ramos and 5-fold times at 0.1 μg/mL in Daudi compared to rituximab.

In example 7, an afucosylated antibody NNV024, showed the ability to induce ADCC in both Ramos and Daudi cell lines. In both cell lines NNV024 demonstrated superior ADCC activation which maximum was 6-fold to 9.5-fold times higher than that of rituximab, a fucosylated antibody, for the same doses. Another afucosylated clinical antibody obinutuzumab, was 4.5-fold times to 8.3-fold times more potent than rituximab and in that regard the combination of features comprised in NNV024 is superior to obinutuzumab. The fucosylated variant, NNV023 also has the ability for ADCC induction, 2.1-fold time to 5-fold times compared to rituximab, thus the afucosylated variant is a stronger inducer of ADCC.

Thus, in a preferred embodiment, said glycosylation of said antibody, antibody fragment or antibody derivative thereof is fucose deficient.

In one or more embodiment(s) said fucose deficient antibody, antibody fragment or antibody derivative thereof have an enhanced and/or improved induction of antibody-dependent cell-mediated cytotoxicity (ADCC), compared to a non-fucose deficient antibody, antibody fragment or antibody derivative thereof.

In that regard an enhanced and/or improved induction of antibody-dependent cell-mediated cytotoxicity (ADCC), may relate an at least 1.01-fold increase, such as at least 1.1, 1.2, 1.3, 1.4, 1.5, 1.6, 1.7, 2, or 5-fold increase, in induction of antibody-dependent cell-mediated cytotoxicity (ADCC), compared to a non-fucose deficient antibody, antibody fragment or antibody derivative thereof.

In another exemplified embodiment said fucose deficient antibody, antibody fragment or antibody derivative thereof have an enhanced and/or improved induction of antibody-dependent cell-mediated cytotoxicity (ADCC), compared to a non-fucose deficient antibody, antibody fragment or antibody derivative thereof.

Said fucose deficiency may be obtained in several ways, such as but not limited to introduction of a GDP-4-keto-6-deoxy mannose reductase of SEQ ID NO: 77 in the production host cell. This protein is a bacterial GDP-4-keto-6-deoxy mannose reductase (RMD) that depletes the cytosolic pool of GDP-4-keto-6-deoxy mannose, which is a precursor for the synthesis of fucose. This precursor is being transformed to GDP-D-Rhamnose—an important for bacteria, but inactive sugar in mammalian cell. An alternative may also be the introduction of 4-b-N-acetylglucosaminyltransferase (GnT-III) and Golgi a-mannosidase II (aManII), which also inhibits fucosylation, thus producing a fucose deficient product.

Thus, in one or more embodiment(s), the production host cell is engineered such that it expresses and/or overexpresses a GDP-4-keto-6-deoxy mannose reductase of SEQ ID NO: 77 and/or a 4-b-N-acetylglucosaminyltransferase (GnT-III) and/or a Golgi a-mannosidase II (aManII).

### Improved Cytotoxicity

As mentioned in the present disclosure, a selection of the humanized antibodies showed the ability to induce ADCC or complement-dependent cytotoxicity (CDC) in both Ramos and Daudi cell lines, this is exemplified in examples 7, 10 or 11 of the present disclosure. The ability of the humanized antibodies to induce ADCC was greater than the non-humanized antibodies of the present disclosure.

In a further embodiment, said human or humanized antibody have an enhanced and/or improved induction of antibody-dependent cell-mediated cytotoxicity (ADCC), antibody dependent cellular phagocytosis (ADCP) and/or complement-dependent cytotoxicity (CDC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37.

In a further embodiment said antibody, antibody fragment or antibody derivative thereof have an enhanced and/or improved induction of antibody dependent cellular phagocytosis (ADCP), compared to Obinutuzumab.

In a further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37.

In a further embodiment, said human or humanized antibody have an enhanced and/or improved induction of antibody dependent cellular phagocytosis (ADCP), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37.

In a further embodiment, said human or humanized antibody have an enhanced and/or improved induction of complement-dependent cytotoxicity (CDC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37.

In a further embodiment, said human or humanized antibody have an enhanced and/or improved induction of complement-dependent cytotoxicity (CDC), compared to a non-humanized antibody comprising the light chain of SEQ ID NO: 22 [NNV003] and the heavy chain of SEQ ID NO: 23.

In a further embodiment, said human or humanized antibody have an enhanced and/or improved induction of complement-dependent cytotoxicity (CDC), compared to Rituximab.

In a further embodiment, said human or humanized antibody have an enhanced and/or improved induction of complement-dependent cytotoxicity (CDC), compared Obinutuzumab.

Example 11 shows that the performance of the NNV antibodies achieve higher CDC induction than Obinutuzumab in Daudi cells.

Thus, in an embodiment, said human or humanized antibody have an enhanced and/or improved induction of complement-dependent cytotoxicity (CDC), compared to Obinutuzumab in Daudi cells.

In another embodiment, said human or humanized antibody have an enhanced and/or improved induction of complement-dependent cytotoxicity (CDC), compared to Obinutuzumab in Daudi cells, wherein the human or humanized antibody comprises an optimized CD37 targeting light chain, such as but not limited to SEQ ID NO: 24 and the heavy chain of SEQ ID NO: 29 [NNV023], and wherein said human or humanized antibody is fucose deficient [NNV024].

In a further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37, in mammalian cells.

Thus, in one or more embodiment(s), the human or humanized antibody of the present disclosure have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody selected from the group consisting of Rituximab, Obinutuzumab and duohexabody-CD37, optionally in Daudi and/or Ramos cells.

In a yet further embodiment, said human or humanized antibody have an enhanced and/or improved induction of antibody-dependent cell-mediated cytotoxicity (ADCC), antibody dependent cellular phagocytosis (ADCP) and/or complement-dependent cytotoxicity (CDC), compared to a non-humanized antibody comprising the light chain of SEQ ID NO: 22 [NNV003] and the heavy chain of SEQ ID NO: 23 and/or the therapeutic antibody Rituximab.

In example 17, it is shown how an afucosylated variant of an antibody of the present invention induces higher ADCC activation in patient derived chronic lymphocytic leukemia (CLL) cells, than a non-fucosylated variant, NNV023 or the known antibodies Obinutuzumab and Duohexabody-CD37.

Accordingly, in embodiments, an afucosylated antibody, antibody fragment or antibody derivative thereof, according to the present invention results in an enhanced ADCC activation compared to a fucosylated antibody, antibody fragment or antibody derivative thereof according to the present invention or compared to Obinutuzumab, Duohexabody-CD37.

In embodiments, an afucosylated antibody, antibody fragment or antibody derivative thereof antibody, antibody fragment or antibody derivative thereof comprising a light chain of SEQ ID NO: 24 [NNV023] and a heavy chain of SEQ ID NO: 29 results in an enhanced ADCC activation compared to a fucosylated antibody, antibody fragment or antibody derivative thereof antibody, antibody fragment or antibody derivative thereof comprising a light chain of SEQ ID NO: 24 [NNV023] and a heavy chain of SEQ ID NO: 29 or compared to Obinutuzumab, Duohexabody-CD37.

This is also shown in example 13 where the potency of the ADCC response of the afucosylated antibody variant NNV024, was shown to be higher than the fucosylated variant NNV023 and the known antibodies rituximab, Obinutuzumab and Duohexabody-CD37. The potency of NNV024 was on average found to be 0.6 nM, while NNV023 had an average potency of 3 nM, and rituximab, Obinutuzumab and Duohexabody-CD37 had a potency of 21.3 nM, 1.1 nM and 0.9 nM respectively.

Accordingly, in embodiments, an afucosylated antibody, antibody fragment or antibody derivative thereof, according to the present invention has an ADCC activation potency of less than 3 nM, such as less than 2 nM, such as less than 1 nM or such as least 0.9, 0.8 or 0.7 or 0.6 nM, or such as between 0.6 nM and 3 nM.

In embodiments, an afucosylated antibody, antibody fragment or antibody derivative thereof antibody, antibody fragment or antibody derivative thereof comprising a light chain of SEQ ID NO: 24 [NNV023] and a heavy chain of SEQ ID NO: 29 has an ADCC activation potency of less than 3 nM, such as less than 2 nM, such as less than 1 nM or such as least 0.9, 0.8 or 0.7 or 0.6 nM, or such as between 0.6 nM and 3 nM.

In further embodiments, an antibody, antibody fragment or antibody derivative thereof, according to the present invention has an ADCC activation potency which is at least 5-fold higher, such as at least 10-fold, 15-fold, 20-fold, 25-fold, or such as at least 30-fold higher than rituximab.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, has an ADCC activation potency which is at least 5-fold higher, such as at least 10-fold, 15-fold, 20-fold, 25-fold, or such as at least 30-fold higher than rituximab.

In embodiments, an afucosylated antibody, antibody fragment or antibody derivative thereof, according to the present invention has an ADCC activation potency which is at least 30-fold, such as at least 35-fold higher than rituximab.

In embodiments, an afucosylated antibody, antibody fragment or antibody derivative thereof comprising a light chain of SEQ ID NO: 24 [NNV023] and a heavy chain of SEQ ID NO: 29 has an ADCC activation potency which is at least 30-fold, such as at least 35-fold higher than rituximab.

In a yet further embodiment, said human or humanized antibody have an enhanced and/or improved induction of antibody dependent cellular phagocytosis (ADCP), compared to a non-humanized antibody comprising the light chain of SEQ ID NO: 22 [NNV003] and the heavy chain of SEQ ID NO: 23 and/or the therapeutic antibody Rituximab.

In a yet further embodiment, said human or humanized antibody have an enhanced and/or improved induction of complement-dependent cytotoxicity (CDC), compared to a non-humanized antibody comprising the light chain of SEQ ID NO: 22 [NNV003] and the heavy chain of SEQ ID NO: 23 and/or the therapeutic antibody Rituximab.

In a yet further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody Rituximab in mammalian cells.

In a further embodiment, said human or humanized antibody have an enhanced and/or improved induction of antibody-dependent cell-mediated cytotoxicity (ADCC), compared to a non-humanized antibody comprising the light chain of SEQ ID NO: 22 [NNV003] and the heavy chain of SEQ ID NO: 23 and/or the therapeutic antibody Rituximab, optionally in Daudi and/or Ramos cells.

An example of such an antibody is exemplified in example 7 of the present disclosure.

### Rituximab

Rituximab is a chimeric monoclonal antibody targeting CD20 i.e., an anti-CD20 monoclonal chimeric antibody.

CD 20 is primarily found on the surface of immune system B cells.

Rituximab, sold under the brand name Rituxan amongst others, is a medication used to treat certain autoimmune diseases and types of cancer.

Rituximab is used for non-Hodgkin lymphoma, chronic lymphocytic leukemia, rheumatoid arthritis, granulomatosis with polyangiitis, idiopathic thrombocytopenic purpura, pemphigus vulgaris, myasthenia gravis and Epstein-Barr virus-positive mucocutaneous ulcers.

Rituximab is given by slow injection into a vein.

Rituximab is used as a comparator for several of the humanized antibodies of the present disclosure.

### Obinutuzumab

Obinutuzumab (also known as afutuzumab) is a humanized anti-CD20 monoclonal antibody.

It is used as a first-line treatment for chronic lymphocytic leukemia in combination with chemotherapy or with venetoclax, as a first-line treatment for follicular lymphoma in combination with chemotherapy, and as treatment for relapsed or refractory follicular lymphoma in combination with bendamustine chemotherapy.

Obinutuzumab is used in combination with chlorambucil as a first-line treatment for chronic lymphocytic leukemia.

Obinutuzumab is used as a comparator for several of the humanized antibodies of the present disclosure.

duohexabody-CD37

Duohexabody-CD37 (Genmab) is a biparatopic anti-CD37 antibody, targeting two distinct epitopes on CD37.

Furthermore, duohexabody-CD37 comprises a E430G hexamerization-enhancing mutation.

Duohexabody-CD37 have shown great potential as a therapeutic biparatopic antibody, with high ADCC and complement-dependent cytotoxicity (CDC) activity.

### Affinity for Human CD37

In the present disclosure, the antibody, antibody fragment or antibody derivative thereof targeting human CD37 are high affinity molecules as disclosed in example 6 of the present disclosure. In general, high affinity molecules are highly preferable in the development of novel therapeutics, as a high affinity may limit off-target side effects, may enhance the on-target effects and may reduce the dosage needed in order to obtain the desired effect.

Thus, in one or more embodiment(s), the antibody, antibody fragment or antibody derivative thereof has an affinity for human CD37 expressing cells below 10 nM, such as below 9 nM, 8 nM, 7 nM, 6 nM, 5 nM, 4 nM, 3 nM, 2 nM, or 1 nM and/or such as below 1 nM, such as below 900 pM, 800 pM, 700 pM, 600 pM, 500 pM, 400 pM or 331 pM.

In one or more preferred embodiment(s), the antibody, antibody fragment or antibody derivative thereof comprises the heavy chain variable domain (VH) comprises the amino acid sequence of SEQ ID NO: 2 [AH02871_HC_VH] and the light chain variable domain (VL) comprises the amino acid sequence variants of SEQ ID NO: 16 [AH02877_V110D_VL] and wherein the antibody, antibody fragment or antibody derivative thereof have an affinity for human CD37 expressing cells below 2 nM such as such as below 1 nM, such as below 900 pM, 800 pM, 700 pM, or 600 pM.

### Immunogenicity Risk Score (IRS)

An immunogenicity score for each aa residue within the binding core was calculated as the population frequency of the restricting HLA molecules overlapping the residue position. The scores are here referred to as an immunogenicity risk score (IRS). It reflects the number of HLA molecules peptides overlapping a given position they are predicted to bin to, but not the clinical immunogenicity in general. A part of the process of antibody humanization may comprise an evaluation of the IRS score of the variable domains of said antibodies compared to the IRS of the starting point, in this case HH1 or chHH1 as described in the present disclosure. A lower IRS score is preferable.

In that regard, in one embodiment, the antibody, antibody fragment or antibody derivative thereof have a predicted immunogenicity risk score (IRS) of the VH domain according to any one of SEQ ID NOs: 1-7 that is lower than the predicted IRS of SEQ ID NO: 19 [VH of Lilotomab].

In another embodiment, the antibody, antibody fragment or antibody derivative thereof have a predicted immunogenicity risk score (IRS) of the VL domain according to any one of SEQ ID NOs: 8-18 that is lower than the predicted IRS of SEQ ID NO: 20 [VL of Lilotomab].

### Improved Plasma Stability

Surprisingly, inclusion of the variant V110D which had the possible function of reducing the immunogenicity, into the light chain variable domain of NNV025, thereby generating NNV023, also increased the plasma and/or serum stability of the construct, this was shown in both example 14 and 15. In example 14, it is shown how the plasma half-life of both NNV023 and NNV024, both comprising the V110D variant was extended compared to NNV025, with a plasma half-life of 12 days, 8.9 days and 7.5 days respectively. In addition, it was shown that both NNV023 and NNV024 exhibit a longer plasma half-life than the known antibodies Obinutuzumab and duohexabody-CD37, which exhibited a plasma half-life of 4.4 days and 4.1 days respectively. In example 15 it is shown how the serum half-life of both NNV023 and NNV024 is longer than Obinutuzumab at 8.6 days, 7.4 days and 3.9 days respectively.

In embodiments an antibody, antibody fragment or antibody derivative thereof of the present invention has a plasma half-life of at least 4.5 days, such as at least 5 days, 6 days, 7 days, 8 days, 9 days, 10 days, or such as at least 11 days.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, has a plasma half-life of at least 4.5 days, such as at least 5 days, 6 days, 7 days, 8 days, 9 days, 10 days, or such as at least 11 days.

In embodiments an antibody, antibody fragment or antibody derivative thereof of the present invention has a serum half-life of at least 4 days, such as at least 5 days, 6 days, 7 days, or such as at least 8 days.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, has a plasma half-life of at least 4.5 days, such as at least 5 days, 6 days, 7 days, 8 days, 9 days, 10 days, or such as at least 11 days.

In embodiments an antibody, antibody fragment or antibody derivative thereof comprising a light chain of SEQ ID NO: 24 [NNV023], has a serum half-life of at least 4 days, such as at least 5 days, 6 days, 7 days, or such as at least 8 days.

In embodiments an antibody, antibody fragment or antibody derivative thereof comprising a light chain of SEQ ID NO: 24 [NNV023], have a plasma half-life of at least 4 days, such as at least 5 days, 6 days, 7 days, or such as at least 8 days.

In one or more embodiment(s) the antibody, antibody fragment or antibody derivative thereof comprising a light chain of SEQ ID NO: 24 [NNV023], have an increased plasma half-life, such as but not limited to more than 1.01-fold, 1.1-fold, 1.4-fold, 1.5-fold, or more than 1.6-fold increased plasma half-life, compared to an antibody comprising a light chain of SEQ ID NO: 72 [NNV025].

In embodiments an antibody, antibody fragment or antibody derivative thereof comprising a light chain of SEQ ID NO: 24 [NNV023], have an increased plasma half-life, such as but not limited to more than 1.01-fold, 1.1-fold, 1.4-fold, 1.5-fold, or more than 1.6-fold increased plasma half-life, compared to Obinutuzumab.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, have an increased plasma half-life, such as but not limited to more than 1.01-fold, 1.1-fold, 1.4-fold, 1.5-fold, or more than 1.6-fold increased plasma half-life, compared to Obinutuzumab.

In embodiments the antibody, antibody fragment or antibody derivative thereof comprising a light chain of SEQ ID NO: 24 [NNV023], have an increased plasma half-life, such as but not limited to more than 1.01-fold, 1.1-fold, 1.4-fold, 1.5-fold, 1.6-fold, or more than 1.7-fold increased plasma half-life, compared to duohexabody-CD37.

In another embodiment the antibody, antibody fragment or antibody derivative thereof comprising a light chain of SEQ ID NO: 24 [NNV023] and the heavy chain of SEQ ID NO: 29 have an increased plasma half-life, such as but not limited to more than 1.01-fold, 1.1-fold, 1.4-fold, 1.5-fold, or more than 1.6-fold increased plasma half-life, compared to an antibody comprising a light chain of SEQ ID NO: 72 [NNV025] and the heavy chain of SEQ ID NO: 29.

In embodiments an antibody, antibody fragment or antibody derivative thereof comprising a light chain of SEQ ID NO: 24 [NNV023], have an increased serum half-life, such as but not limited to more than 1.01-fold, 1.4-fold, 1.5-fold, 1.7-fold, or more than 1.8-fold increased serum half-life, compared to Obinutuzumab.

In embodiments an antibody, antibody fragment or antibody derivative thereof comprising a light chain of SEQ ID NO: 24 [NNV023] and a heavy chain of SEQ ID NO: 29, have an increased serum half-life, such as but not limited to more than 1.01-fold, 1.4-fold, 1.5-fold, 1.7-fold, or more than 1.8-fold increased serum half-life, compared to Obinutuzumab.

### A Nucleic Acid Sequence

One or more aspect(s) of the present disclosure relates to a nucleic acid sequence encoding an antibody, antibody fragment or antibody derivative thereof according to the present disclosure.

In one or more embodiments(s) of the present disclosure a nucleic acid sequence encodes one or more amino acid sequences according to the present disclosure.

In one embodiment, the nucleic acid sequence encodes an antibody, antibody fragment or antibody derivative thereof that is a combination of heavy chain and light chain fragments, where said antibody, antibody fragment or antibody derivative comprises,
a) a light chain having an amino acid sequence which is SEQ ID NO: 24 [AH02877_V110D] and a heavy chain having an amino acid sequence which is SEQ ID NO: 29 [NNV023 heavy chain], or
b) a light chain having an amino acid sequence which is SEQ ID NO: 72 [NNV025 light chain] and a heavy chain having an amino acid sequence which is SEQ ID NO: 29 [NNV025 heavy chain].

In one embodiment, a nucleic acid sequence encoding the light chain of the antibody, antibody fragment or antibody derivative thereof according to the present disclosure, encodes an amino acid sequence according to any one of SEQ ID NOs: 24-28 [amino acid sequences of light chain variants of AH02877], or a functional homologue thereof having amino acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 24-28 [amino acid sequences of light chain variants of AH02877].

In one embodiment, the nucleic acid sequence encoding the heavy chain of the antibody, antibody fragment or antibody derivative thereof according to the present disclosure is a nucleic acid sequence of any one of SEQ ID NO: 32-37 [DNA encoding the heavy chains AH2871-AH2895] or a functional homologue thereof having nucleic acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 32-37 [DNA encoding the heavy chains AH2871-AH2895].

In a preferred embodiment, the nucleic acid sequence encoding the heavy chain of the antibody, antibody fragment or antibody derivative thereof according to the present disclosure is a nucleic acid sequence of SEQ ID NO: 32.

In one embodiment, the nucleic acid sequence encoding the heavy chain of the antibody, antibody fragment or antibody derivative thereof according to the present disclosure is a nucleic acid sequence of any one of SEQ ID NO: 38-43 [DNA encoding the light chains AH2871-AH2895], or a functional homologue thereof having nucleic acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 38-43 [DNA encoding the light chains AH2871-AH2895].

In one embodiment, a nucleic acid sequence encoding the heavy chain of the antibody, antibody fragment or antibody derivative thereof according to the present disclosure, encodes an amino acid sequence according to any one of SEQ ID NOs: 29 or 30 [amino acid sequences of heavy chain variants of AH02871], or a functional homologue thereof having amino acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 29 or 30 [amino acid sequences of heavy chain variants of AH02871].

In one embodiment, the nucleic acid sequence encoding the heavy chain of the antibody, antibody fragment or antibody derivative thereof according to the present disclosure is a nucleic acid sequence of any one of SEQ ID NO: 84-89 [DNA encoding the heavy chains AH2871-AH2895 we Leader seq] or a functional homologue thereof having nucleic acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 84-89 [DNA encoding the heavy chains AH2871-AH2895 we leader seq].

In a preferred embodiment, the nucleic acid sequence encoding the heavy chain of the antibody, antibody fragment or antibody derivative thereof according to the present disclosure is a nucleic acid sequence of SEQ ID NO: 84 [DNA encoding AH02871 HC].

In one embodiment, the nucleic acid sequence encoding the heavy chain of the antibody, antibody fragment or antibody derivative thereof according to the present disclosure is a nucleic acid sequence of any one of SEQ ID NO: 90-95 [DNA encoding the light chains AH2871-AH2895 we Leader seq], or a functional homologue thereof having nucleic acid sequence which is at least 70% identical, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 90-95 [DNA encoding the light chains AH2871-AH2895 we Leader seq].

In one embodiment, the nucleic acid sequence encoding the heavy chain of the antibody, antibody fragment or antibody derivative thereof according to the present disclosure is a nucleic acid sequence of SEQ ID NO: [DNA encoding the light chains AH2877_V110D].

In one embodiment, the nucleic acid sequence encodes an antibody, antibody fragment or antibody derivative thereof with a variable light chain and/or variable heavy chain of any one or more of SEQ ID NOs: 1-18.

The nucleic acid sequence of the present disclosure may also comprise additional elements than the antibody, antibody fragment or antibody derivative thereof coding region. Such elements are in example, regulatory elements.

Furthermore, the host cell according to the present disclosure may comprise regulatory elements enabling the controlled over-expression of endogenous or heterologous and/or synthetic nucleic acid sequences.

The term “regulatory element”, comprises promoter sequences, signal sequence, and/or arrays of transcription factor binding sites that affect transcription and/or translation of a nucleic acid sequence operably linked to the regulatory element.

Regulatory elements are found at transcriptional and post-transcriptional levels and further enable molecular networks at those levels. For example, at the post-transcriptional level, the biochemical signals controlling mRNA stability, translation and subcellular localization are processed by regulatory elements. RNA binding proteins are another class of post-transcriptional regulatory elements and are further classified as sequence elements or structural elements. Specific sequence motifs that may serve as regulatory elements are also associated with mRNA modifications. A variety of DNA regulatory elements are involved in the regulation of gene expression and rely on the biochemical interactions involving DNA, the cellular proteins that make up chromatin, gene activators and repressors, and transcription factors.

In general, the transcriptional and translational regulatory sequences include, but are not limited to, promoter sequences, ribosomal binding sites, transcriptional start and stop sequences, translational start and stop sequences, binding sites for gene regulators and enhancer sequences.

Promoters and enhancers are the primary genomic regulatory components of gene expression. Promoters are DNA regions within 1-2 kilobases (kb) of a gene's transcription start site (TSS); they contain short regulatory elements (DNA motifs) necessary to assemble RNA polymerase transcriptional machinery. However, transcription is often minimal without the contribution of DNA regulatory elements located more distal to the TSS. Such regions, often termed enhancers, are position-independent DNA regulatory elements that interact with site-specific transcription factors to establish cell type identity and regulate gene expression. Enhancers may act independently of their sequence context and at distances of several to many hundreds of kb from their target genes through a process known as looping. Because of these features, it is difficult to identify suitable enhancers and link them to their target genes based on DNA sequence alone.

The promoter, together with other transcriptional and translational regulatory nucleic acid sequences (also termed “control sequences”) is necessary to express a given gene or group of genes (an operon).

Identification of suitable promoter sequences that promotes expression of the specific gene of interest is a tedious task, which in many cases require laborious efforts. In relation to the present disclosure regulatory elements may or may not be post-translational regulators or it may or may not be translational regulators.

Thus, in one embodiment of the present disclosure the regulatory element comprises one or more elements capable of enhancing the expression, i.e. over-expression of the one or more nucleic acid sequence(s) according to the present disclosure.

The regulatory elements and the nucleic acid sequence encoding an antibody, antibody fragment or antibody derivative thereof may be combined into a single nucleic acid construct.

Nucleic acid construct One or more aspect(s) of the present disclosure relates to a nucleic acid construct comprising one or more nucleic acid sequence(s) according to the present disclosure.

### A Nucleic Acid Construct

One or more aspect(s) of the disclosure relates to a host cell comprising one or more nucleic acid sequence(s) according the present disclosure and/or nucleic acid construct(s) the present disclosure.

The nucleic acid construct may comprise at least one regulatory element that facilitates the expression of the antibody, antibody fragment or antibody derivative thereof.

The nucleic acid construct can be a recombinant nucleic acid sequence. By the term “recombinant nucleic acid sequence”, “recombinant gene/nucleic acid/DNA encoding” or “coding nucleic acid sequence” used interchangeably is meant an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

### A Recombinant Nucleic Acid Sequence

The recombinant nucleic sequence may be a coding DNA sequence e.g., a gene, or non-coding DNA sequence e.g., a regulatory DNA, such as a promoter sequence.

Accordingly, in one exemplified embodiment the disclosure relates to a nucleic acid construct comprising a coding nucleic sequence, i.e. a recombinant DNA sequence encoding an antibody, antibody fragment or derivate thereof, combined with a non-coding regulatory DNA sequence, e.g. a recombinant promoter DNA sequence, or a synthetic promoter sequence, wherein the coding and promoter sequences are operably linked.

The term “operably linked” refers to a functional relationship between two or more nucleic acid (e.g., DNA) segments. Operably linked refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the disclosure may be a part of the vector DNA, in another embodiment the construct it is an expression cassette/cartridge that is integrated in the genome of a host cell.

Accordingly, the term “nucleic acid construct” means an artificially constructed segment of nucleic acid, in particular a DNA segment, which is intended to be ‘transplanted’ into a target cell, e.g. a mammalian cell, express or to modify expression of a gene/coding DNA sequence that may be included in the construct.

Integration of the nucleic acid construct of interest comprised in the construct (expression cassette) into the genome of the host cell can be achieved by conventional methods known to the skilled person.

### A Host Cell

In general, mammalian cells are preferred for the production of therapeutic antibodies, as they produce antibodies with mammalian glycosylation patterns and generally mammalian cells are better for the production of correctly folded antibody, antibody fragment or antibody derivative thereof.

In one embodiment, the host cell is a mammalian cell selected from the group consisting of Chinese hamster ovary (CHO) cells, CHO-K1, CHO-DG44, mouse myeloma (NS0) cells, baby hamster kidney (BHK) cells, and human embryonic kidney lines (HEK293) cells, or an insect cell.

In a preferred embodiment the host cell is a Chinese hamster ovary (CHO) cell, such as but not limited to CHO-K1 and CHO-DG44.

In another embodiment, the host cell is a mouse myeloma (NS0) cell.

In yet another embodiment baby hamster kidney (BHK) cells.

In a yet further the host cell is a human embryonic kidney lines (HEK293) cell.

In a yet further the host cell is an Insect cell.

Furthermore, as described in the present disclosure, a antibody, antibody fragment or antibody derivative thereof with a humanized glycosylation pattern may be preferred, i.e., glycosylated antibody, antibody fragment or antibody derivative thereof that is fucose deficient.

In that regard, one or more embodiments relates to a host cell, wherein the cellular fucose glycosylation pathway is modified to reduce the amount of fucose in the glycosylation of said antibody, antibody fragment or antibody derivative thereof. A modified fucose glycosylation pathway may be obtained as disclosed herein, by inclusion of one or more enzymes that modulates the fucose pathway, so that the glycosylation is fucose deficient.

Accordingly, in one embodiment, the host cell is capable of producing an antibody, antibody fragment or antibody derivative thereof according to the present disclosure, wherein the cellular fucose glycosylation pathway of said host cell is modulated, such that the host cell produces a fucose deficient antibody, antibody fragment or antibody derivative thereof.

In one or more embodiment(s), the host cell according to the present disclosure, comprises one or more nucleic acid sequence(s) encoding an antibody, antibody fragment or antibody derivative thereof according to the present disclosure.

In one or more embodiment(s), the host cell according to the present disclosure, comprises one or more nucleic acid constructs comprising at least one nucleic acid sequence encoding an antibody, antibody fragment or antibody derivative thereof according to the present disclosure.

In that regard, one or more aspect(s) of the present disclosure relates to an antibody, antibody fragment or antibody derivative thereof according to the present disclosure, produced in a host cell according to the present disclosure.

In one or more embodiment(s) the antibody, antibody fragment or antibody derivative thereof according to the present disclosure is produced in a host cell, wherein the fucose glycosylation pathway is modulated in order to produce a fucose deficient antibody, antibody fragment or antibody derivative thereof.

### Leader Sequence

In the production of the antibodies of the present disclosure, the production is optimized using a leader sequence inserted at the N-terminal of the amino acid sequence of the heavy chain and/or the light chain.

In one or more embodiments of the present disclosure, the leader peptide has the amino acid sequence of SEQ ID NO: 82.

In one or more embodiments of the present disclosure, the leader peptide is encoded by the nucleic acid sequence of SEQ ID NO: 96.

In that regard, in one or more embodiment(s) the antibody, antibody fragment or antibody derivative thereof according to the present disclosure comprise a heavy chain of any one of SEQ ID NOs: 44-49 and/or a light chain according to any of SEQ ID NOs: 50-55.

In one or more further embodiment(s), the antibody, antibody fragment or antibody derivative thereof, comprises a heavy chain variable domain (VH) that comprises any one of the amino acid sequence variants of SEQ ID NO: 1 [heavy chain of H02871 w variants] and the light chain variable domain (VL) comprises any of the amino acid sequence variants of SEQ ID NO: 8 [light chain of H02871 w variants], or functional homologues thereof having an amino acid sequence which is at least 80% identical, such as at least 81%, 82%, 83%, 84%, 85%, 86%, 87%, 88%, 89%, 90%, 91%, 92%, 93%, 94%, 95%, 95%, 96%, 97%, 98%, 99% or 99.9% identical to any of the variants of SEQ ID NO: 1 and/or SEQ ID NO: 8, and wherein the antibody, antibody fragment or antibody derivative thereof, comprises further comprises an N-terminal leader sequence of SEQ ID NO: 82.

### An Immunoconjugate

Conjugates, and specifically, Immunoconjugates are antibody, antibody fragment or antibody derivative thereof conjugated (joined) to a second molecule, usually a toxin, radioisotope or label.

Such conjugates of the antibody, antibody fragment or antibody derivative thereof are all aspects of the present disclosure.

Thus, one or more aspect(s) of the present disclosure relates to an antibody, antibody fragment or antibody derivative thereof, drug conjugate that binds to human CD37 comprising:
a) an antibody, antibody fragment or antibody derivative thereof according to the present disclosure,
b) a linker, and
c) a drug selected from the group consisting of a toxin, a radioisotope, an anticancer drug, a cytotoxic drug and a cytostatic drug.

Chelating linkers are discussed in the below section regarding radioimmunoconjugates and the chelating linkers described therein are therefore all considered useful for conjugates comprising an antibody, antibody fragment or antibody derivative thereof of the present disclosure connected to or associated with a chelating linker.

Thus, in one or more embodiment(s), said linker is a chelating linker.

In a preferred embodiment, said linker is a chelating linker selected from the group consisting of p-SCN-bn-DOTA, DOTA-NHS-ester and p-SCN-Bn-TCMC.

### A Toxin

An immunotoxin is a human-made protein that consists of a targeting portion such as an antibody, linked to a toxin. When the protein binds to that cell, it is taken in through endocytosis or similar pathway, and the toxin kills the cell.

These immunotoxins are usually used for the treatment of some kinds of cancer and a few viral infections.

These proteins are usually made of a modified antibody or antibody fragment, attached to a fragment of a toxin.

The targeting portion is composed of the Fv portion of an antibody that targets a specific cell type.

In that sense the targeting portion may be an antibody, antibody fragment or antibody derivative thereof according to the present disclosure.

The toxin is usually a cytotoxic protein or compound derived from a bacterial or plant protein or of synthetic origin, from which the natural binding domain has been removed so that the Fv directs the toxin to the antigen on the target cell.

In one or more embodiment(s) the toxin is a chemotherapeutic molecule, including, but not limited to alkylating agents (cisplatin, carboplatin, oxaliplatin, mechlorethamine, cyclophosphamide, chlorambucil, ifosfamide), anti-metabolites (azathioprine, mercaptopurine, pyrimidines), alkaloids (vincristine, vinblastine, cinorelbine, vindesine, paclitaxel, docetaxel, etoposide, teniposide), topoisomerase inhibitors (irinotecan, topotecan, amascrine, etoposide, teniposide) and cytotoxic antibiotics (actinomycin, doxorubicin, daunorubicin, calrubicin, idarubicin, epirubicin, bleomycin, plicamycin, mitomycin).

In one embodiment doxorubicin is conjugated to the antibody, antibody fragment or antibody derivative thereof via the cross-linker SMCC-hydrazide (4-[N-maleimidomethyl]cyclohexane-1-carboxylhydrazide).

The immunotoxin works by the antibody (or other targeting moiety) binding to an antigen on the target cell followed by toxin that enters and kills the cell.

Thus, an aspect of the present disclosure relates to an immunotoxin that comprises antibody, antibody fragment or antibody derivative thereof according to the present disclosure.

An aspect of the present disclosure relates to an antibody, antibody fragment or antibody derivative thereof, drug conjugate that binds to human CD37 according to present invention, coupled or linked to an anticancer drug, a cytotoxic drug, or a cytostatic drug.

### Drug is a Radionuclide

An aspect of the present disclosure relates to a radioimmunoconjugate that binds human CD37 comprising an antibody, antibody fragment or antibody derivative thereof according to the present disclosure, a linker, and a radionuclide selected from the group consisting of ²¹¹At, ²¹³Bi, ²¹²Bi, ²¹²Pb, ²²⁵Ac, ²²⁷Th, ⁹⁰Y, ¹⁶¹Tb, ¹⁸⁶Re, ¹⁸⁸Re, ¹⁹⁹Au, ¹⁹⁴Ir ¹⁶⁶Ho, ¹⁵⁹Gd, ¹⁵³Sm, ¹⁴⁹Pm ¹⁴²Pr ¹¹¹Ag, ¹⁰⁹Pd, ⁷⁷As, ⁶⁷Cu, ⁶⁴Cu, ⁴⁷Sc, and ¹⁷⁷Lu.

In a preferred embodiment, said drug is a radionuclide, selected from the group consisting of ¹⁷⁷Lu, ²²⁵Ac, ²²⁷Th, ²¹²Pb and ⁹⁰Y.

In another embodiment of the present disclosure the radionuclide is ¹⁷⁷Lu.

In another embodiment of the present disclosure the radionuclide is ²¹²Pb.

In yet another embodiment the radionuclide is another beta-emitter or an alpha-emitter.

The radionuclide may be attached to the antibody by first reacting a bifunctional chelator, e.g., p-SCN-bn-DOTA (Macrocyclics, Tx, USA), with the antibody, followed by purification to remove unconjugated chelator, and then reaction of the chelator antibody conjugate with the radionuclide, followed by purification to remove any unconjugated radionuclide.

Alternatively, the chelator and the radionuclide can be combined firstly and subsequently conjugated to the antibody.

Chelating linkers like, e.g., p-SCN-bn-DOTA, can be used for conjugating other metal radionuclides to the antibody, antibody fragment or antibody derivative thereof according to the present disclosure.

Any type of linker with sufficient complexing ability and a functional group allowing direct or indirect conjugation to a protein or a peptide could be used.

Examples of such linkers are described in the literature (e.g. Brechbiel, 2008; Liu, 2008). Some useful examples are bifunctional cyclic chelators like p-SCN-bn-DOTA, DOTA-NHS-ester, p-SCN-Bn-TCMC; bifunctional linear chelators like p-SCN-Bn-DTPA and CHX-A″-DTPA.

The radionuclides in the present disclosure will preferably be conjugated to a targeting molecule by using bifunctional chelators.

These could be cyclic, linear or branched chelators. Particular reference may be made to the polyaminopolyacid chelators which comprise a linear, cyclic or branched polyazaalkane backbone with acidic (e.g. carboxyalkyl) groups attached at backbone nitrogens.

Examples of suitable chelators include DOTA derivatives such as p-isothiocyanatobenzyl-1,4,7,10-tetraazacyclododecane-1,4,7,10-tetraacetic acid (p-SCN-Bz-DOTA) or S-2-(4-Isothiocyanatobenzyl)-1,4,7,10-tetra(2-carbamoylmethyl)cyclododecane and DTPA derivatives such as p-isothiocyanatobenzyl-diethylenetriaminepentaacetic acid (p-SCN-Bz-DTPA), the first being cyclic chelators, the latter linear chelators.

Metallation of the complexing moiety may be performed before or after conjugation of the complexing moiety to the targeting moiety.

The radiolabeling procedure will in general be more convenient in terms of time used etc if the chelator is conjugated to the antibody before the radiolabeling takes place.

The principles of preparing radiolabeled conjugates using chelators attached to antibodies are described broader in e.g. Liu, 2008.

Thus, an antibody, antibody fragment or antibody derivative thereof according to the present disclosure can be used to prepare radioimmunoconjugates with differences in radiation properties and effective half-lives.

For example anti-CD37 radioimmunoconjugate consisting of a an antibody comprising a light chain according to SEQ ID NO: 24 and a heavy chain according to SEQ ID NO: 29, a chelating linker and a beta or alpha emitting radionuclide including, but not limited to ¹⁷⁷Lu, ²¹¹At, ²¹³Bi, ²¹²Bi, ²¹²Pb, ²²⁵Ac, ²²⁷Th, ⁹⁰Y ¹⁶¹Tb, ¹⁸⁶Re, ¹⁸⁸Re, ¹⁹⁹Au, ¹⁹⁴Ir ¹⁶⁶Ho, ¹⁵⁹Gd, ¹⁵³Sm, ¹⁴⁹Pm ¹⁴²Pr ¹¹¹Ag, ¹⁰⁹Pd, ⁷⁷As, ⁶⁴Cu, ⁶⁷Cu, ⁴⁷Sc can be prepared and used for preparing pharmaceutical preparations and used in therapeutic applications.

### A Compound Enriched in One or More Isotopes

An aspect of the present disclosure relates to a positron emitting immunoconjugate that binds human CD37 comprising an antibody, antibody fragment or antibody derivative thereof according to the present disclosure, a linker, and a positron emitting nuclide selected from the group consisting of ¹¹C, ¹³N, ¹⁵O, ¹⁸F, ⁶⁴Cu, and ⁸⁹Zr.

Immunoconjugates may also be used in positron emission tomography.

In that sense the immunoconjugates for positron imaging are enriched in positron emitting nuclides.

The position emitting conjugates are usually prepared just prior to the imaging due to the relatively short half-life of the positron emitting nuclides.

Position emitting conjugates, usually comprises of a targeting molecule conjugated to a compound that is enriched in a positron emitting isotope.

The positron emitting nuclide may be attached to the antibody by first reacting a bifunctional chelator, e.g., p-SCN-bn-Deferoxamine, (macrocycles, US) with the antibody, followed by purification to remove unconjugated chelator, and then reaction of the chelator antibody conjugate with the positron emitting nuclide, followed by purification to remove any unconjugated positron emitting nuclide.

Alternatively, the chelator and the positron emitting nuclide can be combined firstly and subsequently conjugated to the antibody.

Furthermore, the compound conjugated to the targeting molecule may be a compound enriched in positron emitting nuclide may be enriched in ¹¹C, ¹³N, ¹⁵O or ¹⁸F.

Chelating linkers like, e.g., p-SCN-Bn-NOTA, can be used for conjugating other metal positron emitting nuclide to an antibody, antibody fragment or antibody derivative thereof in similar fashion to that described for ⁸⁹Zr and ⁶⁴Cu.

Any type of linker with sufficient complexing ability towards the positron emitting nuclide and a functional group allowing direct or indirect conjugation to a protein or a peptide could be used.

The positron emitting nuclides of the present disclosure will preferably be conjugated to a targeting molecule by using bifunctional chelators.

These could be cyclic, linear or branched chelators. Particular reference may be made to the polyaminopolyacid chelators which comprise a linear, cyclic or branched polyazaalkane backbone with acidic (e.g. carboxyalkyl) groups attached at backbone nitrogens.

A pharmaceutical composition Antibodies, fragments and derivates thereof are usually applied in the treatment of diseases formulated in pharmaceutical compositions.

Such compositions are optimized for parameters such as physiological tolerance and shelf-life.

Thus one or more aspect(s) of the present disclosure relates to a pharmaceutical composition comprising, as the active ingredient, one or more antibody/antibodies, antibody fragment(s) or antibody derivative(s) thereof and/or an antibody, antibody fragment or antibody derivative thereof drug conjugate according to the present disclosure, and a pharmaceutically acceptable carrier.

An embodiment of the present disclosure relates to a pharmaceutical composition as described above, further comprising one or more additional therapeutic agents.

In one embodiment of the present disclosure are said one or more additional therapeutic agents are selected from agents that target a B-cell antigen other than CD37.

Such antigen may be the B-cell antigen CD20.

In another embodiment of the present disclosure are said one or more additional therapeutic agents selected from agents that induce apoptosis.

An immunotherapeutic molecule, such as an antibody, antibody fragment or antibody derivative thereof and/or conjugate thereof as described in the present disclosure, would typically be provided as a pharmaceutical composition potentially consisting of a radionuclide, according to the description above, linked via a chelator to the antibody, antibody fragment or antibody derivative thereof dissolved in a buffer solution, which to a substantial degree maintain the chemical integrity of the immunotherapeutic and/or conjugate thereof and is physiologically acceptable for infusion into patients.

Thus, an aspect of the present disclosure relates to a pharmaceutical composition comprising a antibody, antibody fragment or antibody derivative thereof according to the present disclosure, and an pharmaceutically acceptable carrier and/or excipient.

In one or more embodiment(s) of present disclosure relates to a pharmaceutical composition comprising a drug-immunoconjugate of the present disclosure, and a pharmaceutically acceptable carrier and/or excipient.

In one or more embodiment(s) of present disclosure relates to a pharmaceutical composition comprising a radioimmunoconjugate of the present disclosure, and a pharmaceutically acceptable carrier and/or excipient.

Acceptable pharmaceutical carriers include but are not limited to non-toxic buffers, fillers, isotonic solutions, etc. More specifically, the pharmaceutical carrier can be but are not limited to normal saline (0.9%), half-normal saline, Ringer's lactate, 5% Dextrose, 3.3% Dextrose/0.3% Saline.

The physiologically acceptable carrier can contain a radiolytic stabilizer, e.g., ascorbic acid, which protect the integrity of the pharmaceutical during storage and shipment.

One embodiment of the present disclosure comprises the pharmaceutical composition of the present disclosure and one or more additional antibodies or immunoconjugates.

Antibodies include but are not limited to Rituximab, Epratuzumab, L19, F8, F16, Galiximab, Obinutuzumab Toralizumab, Alemtuzumab, Ofatumumab, Veltuzumab, Afutuzumab, DuoHexabody 37, Tositumomab, Reditux, Ibritumomab, K7153A, 37.1 and HH1.

Radioimmunoconjugates include but are not limited to Zevalin, Bexxar and Betalutin.

In another embodiment of the present disclosure one or more additional antibodies or radioimmunoconjugates targeting CD20. Antibodies include but are not limited to Rituximab, Veltuzumab, Ofatumumab, Afutuzumab, Tositumomab, Reditux and Ibritumomab.

Radioimmunoconjugates include but are not limited to Zevalin and Bexxar.

In one embodiment, said composition further comprises an additional therapeutic agent, preferably selected in the group consisting of alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogues, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

One or more aspect(s) of the present disclosure relates to a pharmaceutical composition, comprising an antibody fragment or antibody derivative thereof, or an antibody fragment or antibody derivative thereof, drug conjugate according to the present disclosure, further comprising one or more further molecule(s), wherein the further molecules is selected from the group consisting of one or more antibodies, small molecule(s), peptide(s) and toxin(s).

One or more embodiment(s) of the present disclosure relates to a pharmaceutical composition of the present disclosure for treating B-cell malignant cells expressing the CD37 antigen.

One or more further embodiment(s) of the present disclosure relates to a pharmaceutical composition of the present disclosure for treating inflammatory disease(s) and/or autoimmune disease.

In an embodiment of the present disclosure the pharmaceutical composition is for treatment of a B-cell malignancy selected from the group consisting of B-cell non-Hodgkins lymphoma, B-cell chronic lymphocytic leukemia, hairy cell leukemia, small lymphoblastic lymphoma and multiple myeloma.

### A Method for Producing

One or more aspect(s) of the present disclosure relates to a method for producing an antibody, antibody fragment or antibody derivative thereof according to the present disclosure, the method comprising,
a) introducing into a mammalian host cell one or more nucleic acid construct(s) of the present disclosure,
b) culturing said host cell in a suitable media,
c) recovering said antibody, antibody fragment or antibody derivative thereof from the culturing broth, and
d) purifying the antibody, antibody fragment or antibody derivative thereof.
   Introducing into a Mammalian Host Cell

In one embodiment of said method, the host cell is a mammalian cell selected from the group consisting of Chinese hamster ovary (CHO) cells, CHO-K1, CHO-DG44, mouse myeloma (NS0) cells, baby hamster kidney (BHK) cells, and human embryonic kidney lines (HEK293) cells, or an insect cell.

In one embodiment of said method, the cellular fucose glycosylation pathway of said host cell is modulated, such that the host cell produces a fucose deficient antibody, antibody fragment or antibody derivative thereof.

In the present disclosure, culturing refers to the process by which cells are grown under controlled conditions, generally outside their natural environment, thus a method used to cultivate, propagate, and grow a large number of cells.

After culturing of the host cells and expression of the gene product, the purification of the protein is required; but since the vector is introduced to a host cell, the protein of interest should be recovered and/or purified from the proteins of the host cell.

Therefore, to make the purification process easy, the cloned gene could have a tag. This tag could be histidine (His) tag or any other marker peptide or protein such as but not limited to the Albumin-binding protein. As some embodiment(s) of the present disclosure relates to antibodies, antibody fragments and antibody derivates thereof, such a tag could also be the Fc domain of said antibodies, antibody fragments and antibody derivates thereof. The Fc fragment may be used as a purification tag where an interaction partner such as but not limited to immobilized protein A is used for the purification.

### A Method of Depleting CD37 Expressing B-Cells

One or more aspect(s) of the present disclosure relates to a method of depleting CD37 expressing B-cells from a population of cells, comprising administering to said population of cells, an antibody, antibody fragment or antibody derivative thereof, an antibody, antibody fragment or antibody derivative thereof, drug conjugate, or a pharmaceutical composition according to the present disclosure.

### A Method of Treating Disease

Therapeutic use of a pharmaceutical composition or solution according to the present disclosure may be for treatment against malignant cells expressing the CD37 antigen, including but not limited to a B-cell malignancy selected from the group consisting of B-cell non-Hodgkins lymphoma, B-cell chronic lymphocytic leukemia, hairy cell leukemia, lymphoplasmacytic lymphoma and multiple myeloma.

Example 9 describes a study with single injections of a pharmaceutical composition or solution comprising an an antibody, antibody fragment or antibody derivative thereof according to the present disclosure or Obinutuzumab in an animal model. The therapeutic efficacy of the pharmaceutical composition comprising an an antibody, antibody fragment or antibody derivative thereof according to the present disclosure was statistically significantly higher than comprising Obinutuzumab when compared at the same amount of antibody injected, for both dosages in an intravenous Daudi lymphoma model in SCID mice.

Thus, in one or more embodiments a pharmaceutical composition or solution comprising an an antibody, antibody fragment or antibody derivative thereof according to the present disclosure improves the survival of SCID mice in an intravenous Daudi lymphoma model when compared to Obinutuzumab.

In a further embodiment a pharmaceutical composition or solution comprising an an antibody, antibody fragment or antibody derivative thereof according to the present disclosure improves the survival of SCID mice in an intravenous Daudi lymphoma model with more than 1.01-fold, 2-fold, 3-fold or more than 4-fold, when compared to Obinutuzumab.

In a further embodiment administration of a pharmaceutical composition or solution comprising an an antibody, antibody fragment or antibody derivative thereof according to the present disclosure, provides a survival of SCID mice in an intravenous Daudi lymphoma model that is higher than 30%, such as more than 40%, 50% or 59%, or such as between 30-60%, when evaluated 11-weeks after initiation of treatment.

In a further embodiment a pharmaceutical composition or solution comprising an an antibody, antibody fragment or antibody derivative thereof comprising the light chain of SEQ ID NO: 24 and the heavy chain of SEQ ID NO: 29 [NNV024], provides a survival of SCID mice in an intravenous Daudi lymphoma model that is higher than 30%, such as more than 40%, 50% or 59%, or such as between 30-60%, when evaluated 11-weeks after initiation of treatment.

One or more aspect(s) of the present disclosure relates to a method of treating disease, wherein targeting of CD37 expressing B-cells can provide an inhibition and/or amelioration of said disease, comprising administering a therapeutically effective amount of an antibody, antibody fragment or antibody derivative thereof, an antibody, antibody fragment or antibody derivative thereof, drug conjugate, or a pharmaceutical composition according to the present disclosure.

Other uses could be treatment of autoimmune diseases and treatment of transplantation related effects.

One or more aspect(s) of the present disclosure relates to a method of treating cancer and/or inflammatory disease(s) and/or autoimmune disease(s) comprising administering a therapeutically effective amount of an antibody, antibody fragment or antibody derivative thereof, an antibody, antibody fragment or antibody derivative thereof, drug conjugate, or a pharmaceutical composition according to the present disclosure.

One or more aspect(s) of the present disclosure relates to a method of treating cancer comprising administering a therapeutically effective amount of an antibody, antibody fragment or antibody derivative thereof, an antibody, antibody fragment or antibody derivative thereof, drug conjugate, or a pharmaceutical composition according to the present disclosure.

One or more aspect(s) the present disclosure relates to the use of an antibody, antibody fragment or antibody derivative thereof, an antibody, antibody fragment or antibody derivative thereof, drug conjugate, or a pharmaceutical composition according to the present disclosure, in inhibiting cancer and/or inflammatory disease(s) and/or autoimmune diseases.

One or more aspect(s) of the present disclosure relates to the use of an antibody, antibody fragment or antibody derivative thereof, an antibody, antibody fragment or antibody derivative thereof drug conjugate or a pharmaceutical composition according to the present disclosure, in ameliorating cancer and/or inflammatory disease(s) and/or autoimmune diseases.

The therapy could be based on, but are not limited to, immunotherapy, beta-particle-radiation or alpha-particle-radiation or a combination of these.

The therapy could be administered either as a monotherapy or in combination with other therapies, preferentially standard treatments. Such other therapies may be pretreatment, surgery, chemotherapy (including doxorubicin, vinblastin and gemcitabine), immunotherapy, photodynamic therapy, proteasome inhibitor (including bortezomib), histone deacetylase inhibitors (including vorinostat and suberoylanilide hydroxamic acid), vitamin D3 and vitamin D3 analogs, cell cycle checkpoint inhibitors (including UCN-01 and 2-(4-(4-Chlorophenoxy)phenyl)-1H-benzimidazole-5-carboxamide), hypoxic cell radiosensitizers (including metronidazole and misonidazole), apoptosis inducers (including withaferin A) radiosensitizers, radioimmunotherapy or a combination of two or more of these.

By administered is meant intravenous infusion or intravenous injection. More specifically, the pharmaceutical composition of the present disclosure can be administered directly in a vein by a peripheral cannula connected to a drip chamber that prevents air embolism and allows an estimate of flow rate into the patient.

In one embodiment the antibody, antibody fragment or antibody derivate thereof or conjugates thereof can be administered in a repeated fashion.

In another embodiment of the present disclosure the antibody, antibody fragment or antibody derivate thereof or conjugates thereof, according to the present disclosure could be administered in a repeated fashion but with different conjugates, such as but not limited to radionuclides, e.g., beta-radioimmunotherapy could be followed by alpha-radioimmunotherapy or chemo-immunoconjugates or vice versa.

An aspect of the present disclosure relates to the use of the antibody, antibody fragment or antibody derivate thereof or conjugates thereof, of the present disclosure for the treatment of B-cell malignancies.

In example 16 of the present disclosure, the antibody variant NNV024 is shown to increase the percent survival of the animals with B-cell malignancies, compared to treatment naive animals.

This highlights the suitability of antibodies of the present disclosure for treatment of B-cell malignancies. This indication is also strengthened by the data presented in example 13, which shows that two antibodies of the present invention, NNV023 and NNV024 induces ADCC in cell lines mimicking both diffuse large B-cell lymphoma, Burkitt's Lymphoma and Mantle Cell lymphoma.

Accordingly, in embodiments, an antibody, antibody fragment or antibody derivate thereof or conjugates thereof, according to the present disclosure is used in the treatment of B-cell malignancies such as but not limited to chronic lymphocytic leukemia (CLL) and non-Hodgkin lymphomas (NHL) such as diffuse large B-cell lymphoma, Burkitt's Lymphoma and Mantle Cell lymphoma.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of B-cell malignancies.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of lymphocytic leukemia (CLL).

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of non-Hodgkin lymphomas (NHL).

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of diffuse large B-cell lymphoma.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of Burkitt's Lymphoma.

In embodiments, an antibody, antibody fragment or antibody derivative thereof antibody, antibody fragment or antibody derivative thereof comprising a light chain of SEQ ID NO: 24 [NNV023] and a heavy chain of SEQ ID NO: 29, is for use in the treatment of Mantle Cell lymphoma.

In addition, it is shown in example 12, that the antibody variant NNV024 outperforms Obinutuzumab in relation to both survival and body weight in an animal model of non-Hodgkin lymphoma, supporting the ability of NNV024 to induce CDC/ADCC in vitro as indicated in examples 7, 10, 11 and 13.

Accordingly, in embodiments, treatment of a subject suffering from B-cell malignancies with an effective amount of an antibody, antibody fragment or antibody derivate thereof or conjugates thereof, according to the present disclosure enhances the chances of survival for said subject, compared to treatment naive subjects suffering from B-cell malignancies.

In additional embodiments, treatment of a subject suffering from B-cell malignancies with an effective amount of an antibody, antibody fragment or antibody derivate thereof or conjugates thereof, according to the present disclosure extends the lifetime prognosis of said subject, compared to an untreated subject suffering from B-cell malignancies.

In embodiments, an afucosylated antibody, antibody fragment or antibody derivative thereof antibody, antibody fragment or antibody derivative thereof comprising a light chain of SEQ ID NO: 24 [NNV023] and a heavy chain of SEQ ID NO: 29, extends the lifetime prognosis of said subject, compared to an untreated subject suffering from B-cell malignancies.

In embodiments, treatment of a subject suffering from B-cell malignancies with an effective amount of an antibody, antibody fragment or antibody derivate thereof or conjugates thereof, according to the present disclosure extends the lifetime prognosis of said subject, compared to a subject suffering from B-cell malignancies treated with Obinutuzumab.

A general problem in treatment of cancers is the loss of body mass in subjects being treated. The reasons that patients are losing weight during treatment are many, and only in some cases related to the disease itself, while the method of treatment also plays a critical role in cancer treatment associated weight loss. In the present disclosure it shown, in example 12, that one of the antibody variants, NNV024, has a reduced tendency towards weight reduction in animals undergoing treatment with NNV024 compared to animals treated with Obinutuzumab.

Accordingly, in embodiments, treatment of a subject suffering from B-cell malignancies with an effective amount of an antibody, antibody fragment or antibody derivate thereof or conjugates thereof, according to the present disclosure reduces the weight loss of said subject, compared to a treatment naive subject suffering from B-cell malignancies.

In embodiments, an afucosylated antibody, antibody fragment or antibody derivative thereof antibody, antibody fragment or antibody derivative thereof comprising a light chain of SEQ ID NO: 24 [NNV023] and a heavy chain of SEQ ID NO: 29, reduces the weight loss of said subject, compared to a treatment naive subject suffering from B-cell malignancies.

In additional embodiments, treatment of a subject suffering from B-cell malignancies with an effective amount of an antibody, antibody fragment or antibody derivate thereof or conjugates thereof, according to the present disclosure reduces the weight loss of said subject, compared to a subject suffering from B-cell malignancies treated with Obinutuzumab.

In embodiments, an afucosylated antibody, antibody fragment or antibody derivative thereof antibody, antibody fragment or antibody derivative thereof comprising a light chain of SEQ ID NO: 24 [NNV023] and a heavy chain of SEQ ID NO: 29, reduces the weight loss of said subject, compared to a subject suffering from B-cell malignancies treated with Obinutuzumab.

An embodiment of the present disclosure relates to the use of the antibody, antibody fragment or antibody derivate thereof or conjugates thereof, of the present disclosure administered in combination with or in addition to other therapy.

In an embodiment of the present disclosure the other therapies are selected from pretreatment, chemotherapy, monoclonal antibody therapy, surgery, radiotherapy, radioimmunotherapy, and/or photodynamic therapy.

In another embodiment of the present disclosure the other therapies are bone marrow transplantation or stem cell transplantation and/or therapy.

Another embodiment of the present disclosure comprises therapeutic pre-treatment using anti-CD20 and/or anti-CD37 monoclonal antibody prior to the treatment with the antibody, antibody fragment or antibody derivate thereof or conjugates thereof, of the present disclosure.

In an embodiment of the present disclosure is the pretreatment done by administering the antibody, antibody fragment or antibody derivate thereof or conjugates thereof, according to the present disclosure followed by treatment by radioimmunoconjugates of the radioimmunoconjugates of the antibody, antibody fragment or antibody derivate thereof.

An aspect of the present disclosure relates to a method for treatment of a B-cell malignancy selected from the group consisting of B-cell non-Hodgkins lymphoma, B-cell chronic lymphocytic leukemia, hairy cell leukemia, lymphoplasmacytic lymphoma and multiple myeloma, comprising administration of an effective amount of the pharmaceutical composition of the present disclosure.

In one embodiment of the present disclosure are the uses and methods of treatment of the present disclosure performed in vitro or ex vivo.

In one embodiment said formulation is suitable for administration by one or more administration routes selected from the group consisting of oral, topical, intravenous, intramuscular, and subcutaneous administration.

In one embodiment, the amount of the antibody fragment or antibody derivative thereof, or the antibody fragment or antibody derivative thereof, drug conjugate according to the present disclosure is at least 0.1 mg and not more than 1 g.

In an embodiment of the present disclosure the antibody, antibody fragment or antibody derivate thereof or conjugate thereof dosing is 1-1000 mg per patient, more preferably 5-50 mg per patient.

In an embodiment of the present disclosure the radioimmunoconjugate dosing is 1-1000 mg per patient, more preferably 5-50 mg per patient, and ¹⁷⁷Lu amounting to 1-200 MBq/kg, more preferably 10-100 MBq/kg of bodyweight.

The pharmaceutical compositions of the present disclosure comprising antibody, antibody fragment or antibody derivate thereof or conjugate thereof of the present disclosure can be used in depleting B cells that express CD37 on their surface.

In one or more aspect(s) of the present disclosure relates to an antibody, antibody fragment or antibody derivative thereof, an antibody, antibody fragment or antibody derivative thereof drug conjugate, or a pharmaceutical composition according to the present disclosure, for use as a medicament.

In one embodiment said medicament is for use in the treatment of cancer.

In a preferred embodiment said medicament is for use in the treatment of B-cell malignancies.

In a preferred embodiment, said medicament is for treating of a B-cell malignancy selected from the group consisting of B-cell non-Hodgkin's lymphoma, B-cell chronic lymphocytic leukemia, hairy cell leukemia, lymphoplasmacytic lymphoma and multiple myeloma, comprising administering to the individual in need thereof, an effective amount of an antibody, antibody fragment or antibody derivative thereof, an antibody, antibody fragment or antibody derivative thereof, drug conjugate, or a pharmaceutical composition according to the present disclosure.

In one embodiment said medicament is for treating of inflammatory and autoimmune diseases wherein CD37-positive B cells are enriched.

Example 8 of the present disclosure relates to the in vivo function of the antibody, antibody fragment or antibody derivative thereof, evaluated in mice, which suggests that a pharmaceutical composition according to the present disclosure may be given by single or multiple administration.

In one embodiment said medicament is administered once or sequential.

One or more aspect(s) of the present disclosure relates to a formulation of an antibody, antibody fragment or antibody derivative thereof, an antibody fragment or antibody derivative thereof drug conjugate, or a pharmaceutical composition according to the present disclosure, for use in pre-treatment, wherein human CD37 is blocked in normal tissues before treatment with an immunotoxic anti-CD37 molecule or antibody-drug conjugate according to the present disclosure.

### Use in Positron Emission Tomography Imaging

One or more aspect(s) of the present disclosure relates to an antibody, antibody fragment or antibody derivative thereof, conjugate that binds to human CD37 comprising:
a) an antibody, antibody fragment or antibody derivative thereof according to the present disclosure,
b) a linker, and
c) an compound enriched in one or more isotopes selected from the group consisting of ¹¹C, ¹³N, ¹⁵O, ¹⁸F, and ⁸⁹Zr.

One or more aspect(s) of the present disclosure relates to an antibody, antibody fragment or antibody derivative thereof conjugate according to the present disclosure, for use in positron emission tomography imaging.

In one embodiment, said imaging is for providing diagnosis, staging, and monitoring treatment of cancers.

In another embodiment said cancer is B-cell non-Hodgkin's lymphoma, B-cell chronic lymphocytic leukemia, hairy cell leukemia, lymphoplasmacytic lymphoma and multiple myeloma.

### Kits

### A Kit for the Production of a Drug Conjugate

One or more aspect(s) of the present disclosure relates to a kit for the production of an antibody fragment or antibody derivative thereof, drug conjugate according to the present disclosure comprising,
a) two or more vials, wherein one vial contains a conjugate comprising a drug linked to a linker, and one vial comprising an antibody, antibody fragment or antibody derivative thereof according to the present disclosure, and
b) optionally instructions for preparing said antibody-drug conjugate.

### A Kit for the Production of a Radionuclide Conjugate

In the present disclosure “radioimmunoconjugate” and “radionuclide conjugate” are used interchangeably.

One or more aspect(s) of the present disclosure relates a kit for the production of an antibody fragment or antibody derivative thereof, radionuclide or positron emitting nuclide conjugate, according to the present disclosure comprising,
a) two or more vials, wherein one vial contains a conjugate comprising a chelator linked to an antibody fragment or antibody derivative thereof according to the present disclosure, a second vial containing a radionuclide or positron emitting nuclide, and
b) optionally, instructions for preparing said radioimmunoconjugate, or positron emitting nuclide-immunoconjugate.

One or more aspect(s) of the present disclosure relates a kit for the production of an antibody fragment or antibody derivative thereof, radioimmunoconjugate according to the present disclosure comprising,
a) two or more vials, wherein one vial contains a conjugate comprising a chelator linked to an antibody fragment or antibody derivative thereof according to the present disclosure, a second vial containing a radionuclide, and
b) optionally, instructions for preparing said radioimmunoconjugate conjugate.

A kit, according to the present disclosure may require some procedures to be performed, e.g., radiolabeling and/or purification to take place before infusion.

An embodiment of the present disclosure relates to a kit of the present disclosure, wherein the content of one or several of the vials are either lyophilized or in a solution.

By mixing the contents of the two vials to generate the drug-immunoconjugate or radioimmunoconjugate the final product will appear. Thus, in another embodiment of the present disclosure the radioimmunoconjugate is generated by mixing the content of the two vials.

This product may need purification prior to use.

It should be noted that embodiments and features described in the context of one of the aspects of the present disclosure also apply to the other aspects of the disclosure.

All patent and non-patent references cited in the present application, are hereby incorporated by reference in their entirety.

The disclosure will now be described in further details in the following non-limiting examples.

### EXAMPLES

In the present examples, references are made to different antibodies, which are listed in Table 1.

TABLE 1
antibody identifiers.

| | Heavy | LC | |
|---|---|---|---|
| | chain (HC) | sequence (LC) | |
| Identifier | SEQ ID NO: | SEQ ID NO: | Modification |
| NNV003 | 23 | 22 | — |
| NNV009 | 23 | 22 | Radioconjugate |
| NNV010 | 23 | 22 | DOTA conjugate |
| NNV020* | 29, 66-70 | | — |
| NNV023 | 29 | 24 | — |
| NNV024 | 29 | 24 | afucosylation |
| NNV025 | 29 | 72 | — |
| NNV026** | 29 | 76 | — |
| NNV027*** | 71 | 74 | — |
| NNV030 | 30 | 24 | — |
| DuoHexaBody- | 78, 80 | 79, 81 | — |
| CD37**** | | | |

*NNV020 refer to six different antibodies, with identifiers AH02871, AH02875, AH02877, AH02879 AH02886, AH02895, as exemplified in below examples.
**NNV026 comprise the AH02871_HC_LC, and is also denoted AH02871 in the examples
***NNV027 comprise the AH02886_HC_LC, and is also denoted AH02886 in the exampels
****recombined construct of two different antibody fragments, thus comprises two heavy chain sequences and two light chain sequences.

### Example 1—Development of Humanized Anti-CD37 Monoclonal Antibodies

The aim of this project is to humanize the lilotomab (also named HH1 or NNV001) antibody using the complementarity-determining region (CDR) grafting method. In the present examples the terms “lilotomab”, “HH1” and “NNV001” are used interchangeably.

### Methods

Briefly, The CDRs of lilotomab were grafted into the human acceptors to obtain five humanized light chains and five humanized heavy chains for each antibody. Twenty-five humanized antibodies were expressed in HEK293 cells and the supernatants were assessed by fluorescence-activated single cell sorting (FACS).

### Antibody Humanization by CDR Grafting: Selection of Acceptor Frameworks

Murine constant regions of lilotomab were substituted with human regions and the variable regions were humanized using CDR grafting. The variable domain sequences of lilotomab were searched in the database of human germline using NCBI Ig-Blast (http://www.ncbi.nlm.nih.gov/projects/igblast/). Five diverse human acceptors (i.e. human variable domains with high homology to the parental antibody) for each heavy chain and light chain were chosen. The CDRs of human acceptors were replaced with their mouse counterparts, resulting in the humanized variable domain sequences. The humanized variable domains of heavy chains were named VH1, VH2, VH3, VH4 and VH5; while the humanized variable domains of light chains were named VL1, VL2, VL3, VL4 and VL5. Pairwise combination of these light and heavy chains gave 25 variants of humanized constructs, as is also described in Example 4.

The DNA sequences encoding the parental antibody and humanized IgG heavy and light chains were optimized and inserted into pTT5 vector to construct expression plasmids of full-length IgGs. All complementarity-determining regions of lilotomab (VL-CDR1, VL-CDR2, VL-CDR3 and VH-CDR1 . . . VH-CDR3) were grafted into the chosen human acceptors after the sequence alignment.

The five heavy human chains (HC1, HC2, HC3, HC4, HC5) and five light human chains (LC1, LC2, LC3, LC4, LC5) containing murine CDRs were generated. The number in the chain's name (e.g. HC1 or LC4) indicates the degree of the homology (1 being the highest, 5 is the lowest) of the variable part to the murine sequence. Pairwise combinations of these light and heavy chains gave 25 different constructs of humanized IgG1. Binding of these antibodies to Ramos cells are presented in Example 4.

In addition, in order to retain a conformational structure and conserved amino acids of the initial NNV001 Ab (lilotomab), the two sequences HC1, LC1 was subjected to back mutations. The resulting HC1 and LC1 with back mutations were named CVH and CHL correspondingly. With this antibody consisting of CVH and CVL the total number of constructs is 26. The CVH+CHL Ab was also named CBM for core back mutations.

### Optimization of the HC1+LC1 Sequences

In the next step of humanization only one IgG1 construct was considered, HC1+LC1, since this had the highest degree of homology. The aim of this step was to identify the putative sites in the sequences of human acceptor that should be mutated back to those of the murine sequence in order to regain the binding strength of the humanized antibody.

The CBM sites may include, but not limited to, those framework region residues that are believed to be important for the binding activity, such as canonical and inner core FR residues and VH-VL interface residues to retain the inner hydrophobic interaction.

The HC1+LC1 construct became a basis for further optimization because of two reasons:
1) Introduction of CBMs to HC1+LC1 improves binding so that it becomes not worse than the one of NNV003 (see Example 4).
2) The variable part of HC1+LC1 shares the highest degree of similarity of the amino acid sequence to the parental NNV001. Should necessary back mutations be introduced to restore the binding, the minimum of these CBMs are expected to be in the sequence of the highest homology to the parental one. In other words, HC2+LC2 exhibiting better binding to Ramos would require more CBMs, which might increase immunogenicity.

The initial sequences to be humanized:

> Lilotomab _VH
(SEQ ID NO: 19)
EIQLQQSGPELVKPGASVKVSCKASGYSFTDYNMYWVKQSHGKSL
EWIGYIDPYNGDTTYNQKFKGKATLTVDKSSSTAFIHLNSLTSED
SAVYYCARSPYGHYAMDYWGQGTSVTVSS
> Lilotomab_VL
(SEQ ID NO: 20)
DIVMTQSHKLLSTSVGDRVSITCKASQDVSTAVDWYQQKPGQSPK
LLINWASTRHTGVPDRFTGSGSGTDYTLTISSMQAEDLALYYCRQ
HYSTPFTFGSGTKLEIK

The CDR regions are in bold font in the above sequences.

The human germline sequences that are closely resemble the lilotomab VH and VL sequences were selected from NCBI database of human germlines using Ig-BLAST algorithm (http://www.ncbi.nlm.nih.gov/projects/igblast/).

### Human Germline Sequences:

VH
> IGHV1-3*01,
SEQ ID NO: 60
QVQLVQSGAEVKKPGASVKVSCKASGYTFTSYAMHWVRQAPGQRL
EWMGWINAGNGNTKYSQKFQGRVTITRDTSASTAYMELSSLRSED
TAVYYCARX
VL
> IGKV1-39*01,
SEQ ID NO: 61
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTPP

### Human Acceptor Sequences:

VH
> AEB00269.1 immunoglobulin mu heavy chain,
partial [Homo sapiens],
SEQ ID NO: 62
LVAAATGAHSQVQLVQSGAEVKKPGASVKVSCKASGYTFTSYAMH
WVRQAPGQRLEWMGWINAGNGNTKYSQKFQGRVTITRDTSASTAY
MELSSLRSEDTAVYYCAREQWLVRENFDYWGQGTLVTVSS
VL
> BAH04867.1 immunoglobulin kappa light chain,
partial [Homo sapiens],
SEQ ID NO: 63
DIQMTQSPSSLSASVGDRVTITCRASQSISSYLNWYQQKPGKAPK
LLIYAASSLQSGVPSRFSGSGSGTDFTLTISSLQPEDFATYYCQQ
SYSTPYTFGQGTKLEIK

The Human Acceptor Sequences with Grafted CDRs:

Then, the aa sequences VH_1 and VL_1 of the sizes equal to the sizes of the germline sequences were derived from the human acceptor protein sequences. CDRs were grafted into those fragments:

VH
> VH_1,
SEQ ID NO: 64
QVQLVQSGAEVKKPGASVKVSCKASGYSFTDYNMYWVRQAPGQRL
EWMGYIDPYNGDTTYNQKFKGRVTITRDTSASTAYMELSSLRSED
TAVYYCARSPYGHYAMDYWGQGTLVTVSS
VL
> VL_1,
SEQ ID NO: 65
DIQMTQSPSSLSASVGDRVTITCKASQDVSTAVDWYQQKPGKAPK
LLIYWASTRHTGVPSRFSGSGSGTDFTLTISSLQPEDFATYYCRQ
HYSTPFTFGQGTKLEIK

### Backmutation Design Method

The sites of the domain interface (these sites are involved in the IgG₁ interdomain interaction) should be left unchanged in the humanized sequences. If these sites are back mutated to the residues of NNV001, the domains of the given sequence will retain inner hydrophobic interaction (domain-to-domain interaction). In order to identify these sites in the sequences of NNV001, 3D homology modeling of NNV001 antibody Fv fragments was carried out. The sequences of NNV001 were aligned against PDB_Antibody database (Protein Data Base https://www.rcsb.org/) using BLAST algorithm for identifying the best templates for Fv fragments and especially for building the domain interface. A well described structural template of PDB identifier 1H8N (3D structure of anti-ampicillin single chain Fv fragment from phage-displayed murine antibody libraries), being closely similar (identity=73%) to NNV001, was selected.

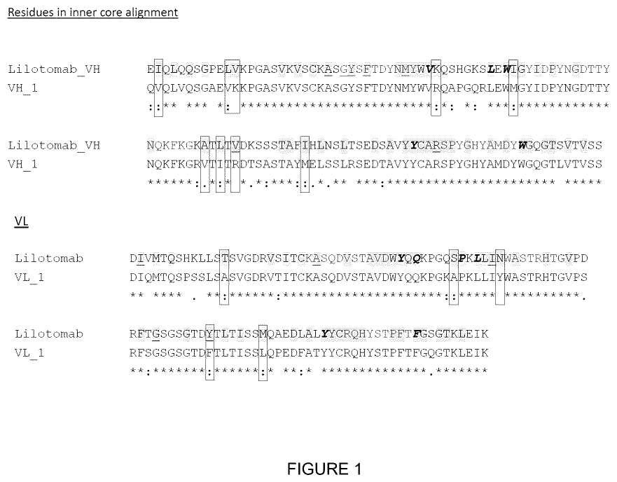

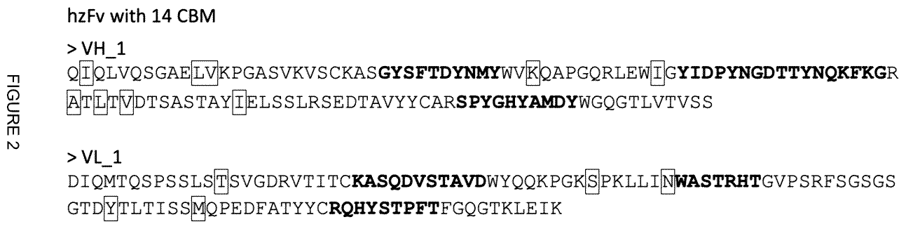

Homology models and disulfide bridges were specified and linked. Based on the homology model of 1H8N, all framework residues in inner core were highlighted in grey in FIG. 1. All framework region (FR) residues that are believed to be important for the binding activity were determined: canonical FR residues and VH-VL interface residues (see FIG. 1). FR residues of the humanized antibody with grafted CDRs were selected for priority back mutation (replacement with NNV001 equivalent residues) according to the following guideline:
1) FR residues, which do not conform to the canonical structure set;
2) FR residues in the inner core;
3) VH-VL interface residues;
4) The residues that are similar or with same FR group in the grafted antibody should be selected for less priority back mutation.

### Identification of Necessary CBM Sites

Residues in the grafted antibody that fall in all categories and are different from those of NNV001 were selected for replacement with NNV001 residues (shown in boxes in FIG. 1). The humanized variable part with grafted CDRs and 14 priority core back mutations (CBM in the boxes) are shown in FIG. 2.

Phage Display, Selection of Best Clones with Minimal Number of Necessary CBMs.

In order to reduce the number of back mutations in the variable domains, a library of sequences for variable heavy and variable light domains (VH and VL) was developed using a combinatory design exploiting all 14 putative back mutations. The theoretical diversity of the library was 2¹⁴ sequences. Each sequence of the library contained one or more back mutated sites in various combinations. Multiple phagemid vectors containing random pairwise combination of VH and VL sequences were constructed and used to transfect E. coli TG1 cells. When cultivated, E. coli produce a vast amount of bacteriophage particles that display various Fab (one Fab per a phage particle) at their surface. The mix of these phages was isolated from bacterial cells and pre-incubated with HEK293 6e to exclude a nonspecific binding to this producing cell line. The supernatant (the mix of phages that did not bind HEK293 6e cells) was incubated, “panned”, with Ramos cells followed by a washing with PBS to remove non-binding Fab. Prior eluting bound phages from Ramos cells, the system was co-incubated with NNV003 to exclude those Fab variants affinities of which were lower than that of NNV003 (exclusion through a competitive binding). Some of the retained bacteriophage clones were randomly picked (384 individual clones in total), cultivated in 96 well plates, and the binding to Ramos was validated using FACS. The panning was repeated one more time (2 cycles in total). The output collection (40 clones) contained a good percentage of phages binding to Ramos, but not to HEK293 6e cells.

These 40 clones were used to infect exponentially growing E. coli TG1 cells. After cultivation of the transfected bacterial cells, the DNAs encoding Fab fragments were extracted and sequenced to learn the composition of Fabs.

As much as 37 clones were successfully sequenced. Out of this amount, six clones containing the least number of back mutations were selected to be lead clones. The selected clones possess the lowest non-specific binding to HEK293 6e cells, good expression level in this cell line and the affinity to Ramos cells of the same magnitude as the one of NNV003.

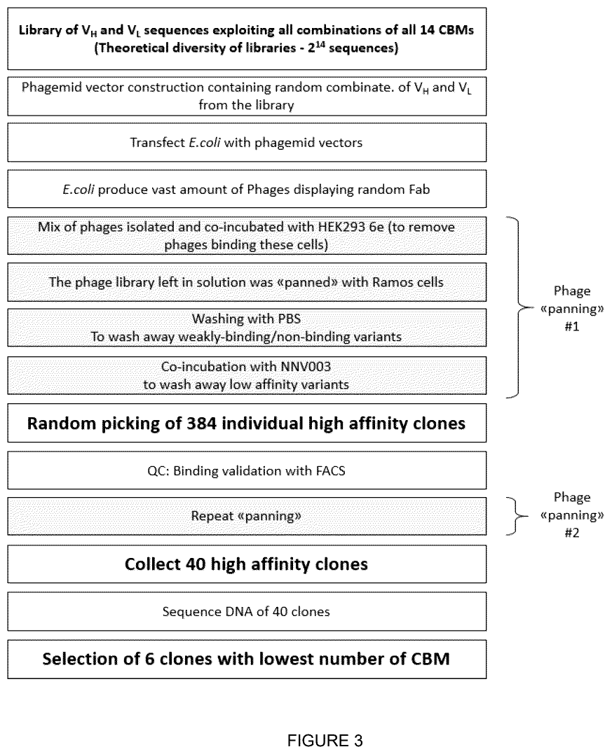

FIG. 3 represents a graphical overview of the steps and processes involved in the selection of the best Fab clones using Phage display and the affinity selection techniques.

### Generation of Six Full Length IgG₁ Out of the Six Selected Fab Clones

When the selection of the best binding Fab clones using the Phage display was accomplished, these Fab-clones were transformed to full-length IgG₁.

For this, to each of the selected VH or VL sequences, a corresponding constant part (human) was added:
1) VH domain was spliced with human IgG₁ heavy constant domain (hIgG1HC) consisting of C_{H}1, C_{H}2, C_{H}3 domains.
2) VL domain was spliced with human IgG₁ kappa light constant domain (hIgKLC) consisting of only C_{L} domain

The source of constant domains was the sequence of the chimeric Ab (NNV003), light chain constant domain of SEQ ID NO: 22 and heavy chain constant domain of SEQ ID NO: 23.

The pairs of complimentary sequences (heavy and light chains) were inserted into pTT5 vector to construct IgG-encoding plasmids. These plasmids were used for transient co-transfection of HEK 293 6e cells. After a week of cell culturing, the six IgG₁ clones were harvested from the supernatants of the cell suspension, filtered using 0.22 μm filter membrane, characterized for purity using SDS-PAGE, Western-blot, and for the binding to CD37 using flow cytometry. Aliquots of these 6 Abs were tested for binding to CD37 positive RAMOS cells, see Example 4.

### Results

All six generated humanized Abs (NNV020) belong to immunoglobulin isotype G, subclass 1.

The heavy chain of NNV020 is compounded of μ heavy chain variable domain and the constant region of y heavy chain of the allotype G1 m17.1.2. This allotype is formed of the following alleles: The G1 m(z) allele also known as G1 m17, corresponds to Lys (K) at position 214 in the HC1 domain (EU numbering); the allele G1 m(a) also known as G1 ml, corresponds to Asp (D) and Leu (L) at positions 356 and 358 in the HC3 domain; the allele nG1 m(x), also known as nG1 m2, corresponds to Ala (A) at position 431 in the HC3 domain. The light chain belongs to K type of the allotype Km1,2. This allotype of the kappa light chain is formed of the following alleles: the Km1 corresponds to Ala (A) at position 153; the Km2 corresponds to Val (V) at position 191.

### List of the Humanized NNV020 Variants

This section contains an overview of names, codes and sequences of humanized antibodies and their parts (heavy and light chains).

Table 2 lists the clones (hits) that were selected in the Phage display and affinity maturation stages. DNA sequences and amino acid sequences of the variants are described in the table 3, which also describes the variable domain sequences of each of the variants.

TABLE 2
The list of the selected NNV020 variants. The CBMs
were defined for heavy and light chain of each Ab.

| | Name or | | SEQ |
|---|---|---|---|
| | initial | | ID |
| Identifier | Code | Core Back Mutations (CBMs) | NOs |
| NNV020 | AH02871 | >AH02871_HC: K12V, R38K, | 2 |
| | | >AH02871_LC: Y49N, F71Y, L78M | 8 |
| NNV020 | AH02875 | >AH02875_HC: V2I, V11L, V68A, | 3 |
| | | M81I | |
| | | >AH02875_LC: A43S, Y49N, F71Y, | 9 |
| | | L78M | |
| NNV020 | AH02877 | >AH02877_HC: K12V, R38K, V68A, | 4 |
| | | I70L, R72V, M81I | |
| | | >AH02877_LC: A43S, Y49N, F71Y | 10 |
| NNV020 | AH02879 | >AH02879_HC: V2I, K12V, R38K, | 5 |
| | | M48I, V68A, I70L | |
| | | >AH02879_LC: A13T, A43S, Y49N, | 11 |
| | | F71Y, L78M | |
| NNV020 | AH02886 | >AH02886_HC: V11L, R38K, I70L, | 6 |
| | | R72V | |
| | | >AH02886_LC: A13T, Y49N, F71Y | 12 |
| NNV020 | AH02895 | >AH02895_HC: V2I, K12V, R38K, | 7 |
| | | V68A, M81I | |
| | | >AH02895_LC: Y49N, F71Y, L78M | 13 |

Dna and Amino Acid Sequences of the Six Heavy and Light Chains with the Least Back Mutations

TABLE 3

| | | | DNA Sequence | Amino acid | |
|---|---|---|---|---|---|
| | | | (Leader sequence- | sequence (Leader | Variable |
| | | | VH/VL- IgG1_HC/ | sequence-VH/VL- | domain |
| | NNV | | IgKLC- Stop codon) | hIgG1CH/hIgKCL) | (VH or VL) |
| Identifier | identifier | Chain | SEQ ID NO: | SEQ ID NO: | SEQ ID NO: |
| — | NNV020 | HC | — | — | 1 |
| | | LC | — | — | 8 |
| AH02871 | NNV020 | HC | 32 | 44 | 2 |
| | | LC | 38 | 50 | 8 |
| AH02875 | NNV020 | HC | 33 | 45 | 3 |
| | | LC | 39 | 51 | 9 |
| AH02877 | NNV020 | HC | 34 | 46 | 4 |
| | | LC | 40 | 52 | 10 |
| AH02879 | NNV020 | HC | 35 | 47 | 5 |
| | | LC | 41 | 53 | 11 |
| AH02886 | NNV020 | HC | 36 | 48 | 6 |
| | | LC | 42 | 54 | 12 |
| AH02895 | NNV020 | HC | 37 | 49 | 7 |
| | | LC | 43 | 55 | 13 |

### Discussion

The lilotomab antibody was humanized by grafting the CDR regions onto human acceptor sequences followed by back mutations onto the sequence with the highest homology to lilotomab. To improve binding to the target antigen 14 core back mutations were introduced in the framework region important for binding. Phage display was then used to select the clones with the best binding and minimal number of back mutations resulting in antibodies with 5 to 11 back mutations. Binding of these antibodies to CD37 expressing Ramos cells is described in Example 4. The back mutations can be immunogenic. Potential immunogenicity of the humanized antibodies was therefore analyzed and discussed in Example 2.

The lilotomab antibody was humanized. Six humanized IgG1 clones were selected for further characterization and development. The clones retained the murine CDR regions of lilotomab and chimeric HH1/chHH1 (NNV003) and the rest of the sequence have a high degree of homology to the lilotomab sequence.

### Example 2 Selection of Anti-CD37 Humanized Antibodies Using in Silico Prediction Tools

The Aim of this Example was
1) to assess the predicted immunogenicity potential of six NNV020 drug candidates generated during the humanization of lilotomab (AH02871, AH02875, AH02877, AH02879, AH02886 and AH02895),
2) to rank the humanized antibodies and
3) to suggest how to decrease the immunogenicity potential further.

### Methods

### Antibody Sequences

The sequences of humanized antibodies included in this example are reported in example 1, table 1, 2 and 3. The sequences of lilotomab, chHH1 (NNV003) and rituximab are SEQ ID NOs 56-57 (lilotomab), SEQ ID NOs: 22-23 (NNV003) and SEQ ID NOs: 58-59, respectively.

### Sequence Alignment

Alignment of the tested sequences was performed using the Bioinformatics Software for Sequence Data Analysis Geneious (www.geneious.com) version 11.0.2.

### In Silico MHC Class II-Peptide Binding Prediction Analysis Parameters

For MHC II binding predictions, the NetMHCIIpan-3.1 algorithm was used. Each protein sequence was analysed for sub-15mer MHC II binding peptides restricted to each of the HLA alleles in Appendix C, by decomposing the protein to overlapping 15-mer peptides. A predicted binding affinity (nM) to MHC class II alleles is calculated based on in vitro generated data points compiled into the IEDB database (http://www.iedb.org). Only peptides predicted with a rank score of less than 10 where considered as potential MHC II binders. For different peptides predicted to bind to the same HLA allele and having identical predicted binding cores, only the peptide with the strongest predicted binding affinity was considered.

### Calculation of Alleles Frequencies

Allele frequencies for the HLA alleles encoding MHC class II proteins in humans can be found in various databases for a diverse number of populations. Frequencies for an average world population and 11 geographical regions have been used in the present example and were calculated from the allelefrequencies.net database (http://www.allelefrequencies.net/). The allele frequency database consists of several thousand individual studies from various geographic locations and ethnic populations. The size and location of the cohort analysed varies greatly between studies. The region-specific frequencies were calculated as an average of allele frequencies from the individual studies in each region weighted by the number tested subjects. To avoid bias towards large studies, the number of test subjects in studies with more than 5000 was set to 5000. This ensures a lower bias in the region-specific population. However, as most available studies have been conducted in North America and Europe, the World Averaged is biased towards these populations. No frequencies for HLA-DRB3, HLA-DRB4, HLA-DRB5, HLA-DQ and HLA-DP alleles could be obtained from the database, thus alleles from these subclasses were selected based on their availability for prediction and the frequency set to 0.025. A frequency of 0.025 is comparable to sub-dominant HLA-DRB1 alleles. Of note, only the world population was considered for the analysis of the sequences generated after in silico de-immunisation.

### Immunogenicity Risk Score

An immunogenicity score for each aa residue within the binding core was calculated as the population frequency of the restricting HLA molecules overlapping the residue position. The scores are here referred to as an immunogenicity risk score (IRS). It reflects the number of HLA molecules peptides overlapping a given position they are predicted to bin to, but not the clinical immunogenicity in general.

Immunogenicity risk score is calculated according to formula 1:

$${s}_{p} = {\sum }_{a - HLA} {\sum }_{p - 14}^{p} {r}_{p} \leq {T}_{ept} : {f}_{a} \qquad Formula 1$$

In formula 1, rp is the predicted rank score of the peptide starting at position p binding to allele a. fa is the allele frequency for allele a; Tepi is the binding threshold for considering a peptide a potential epitope (i.e. 10% rank) and n is the length.

The maximum value of the IRS depends on the coverage of HLA alleles of the investigated population. However, the theoretical maximum is 1, which is achieved when all overlapping 15-mers for a given position are predicted to be binders. In reality, this maximum is never achieved.

As less peptide are overlapping positions at the N and C-terminal, Glycines are added to the sequence before calculating the position specific IRS. The overall protein IRS is calculated as the sum of position-specific risk scores.

Immunogenicity risk-scores (IRS) were calculated based on HLA binding profiles of protein derived 15-mer peptides, to a “world average” population of HLA-alleles consisting of more than 300 different MHC Class II alleles and 11 geographical defined populations. The IRS reflects the number of HLA binding sub-peptides weighted by the frequency in the investigated population. Note that, due to unevenly efforts in HLA typing in different parts of the world, some populations are defined from very little data and the world average population is biased against the North American and the European populations.

### Identification of the CDR

A trained hidden markov model (HMM) was used to identify complementary determining regions (CDR) of the test articles and align the light and heavy chain sequences to the same template for easy comparison and visualization.

### T Cell Epitope Heat Map

T cell epitope heat map allows the visualisation of the core sequence of the test article sequences that bind to the various HLA tested with their corresponding binding affinity for a given population.

### In Silico De-Immunisation

De-immunization was performed in silico by introducing all natural aa at the positions 100-115 of the AH02877 light chain. The IRS was calculated as described below for the average world population. The effect of the mutations on the overall IRS is reported as change in IRS score e.g., mutated IRS subtracted to the IRS for the native sequence.

For the de-immunization, conservation scores were calculated using the psiblast algorithm. To narrow the search for antibody only, a database of 2287 antibody light chain sequences were used to calculate the conservation scores, downloaded from the PDB database.

### Results

In silico MHC class II-binding prediction analysis of peptide derived from lilotomab and derivatives hereof was performed to,
i) predict potential neo-epitopes potentially able to trigger an immune response, and
ii) rank the NNV020 drug candidates based on their immunogenicity potential to select a new anti-CD37 mAb with a lower predicted immunogenicity potential than lilotomab.

### Sequence Alignment of the Test Articles

The sequences of lilotomab and derivative hereof are listed above, and in the sequence listing. Identification of residues involved in antigen binding was performed using a trained HMM model. The CDR sequences and their start-end positions in the Kabatt system are listed in Table 4. Of note, the sequence of CDRs identified previously using the DIGIT (database of ImmunoGlobulins with Integrated Tools) database are also reported (http://biocomputing.it/digit)

TABLE 4
Complementary determining regions (CDR) for the test articles

| | | | CDR | Start | End |
|---|---|---|---|---|---|
| | | | sequence | posi- | posi- |
| Test | | CDR sequence | (HMM | tion | tion |
| article | CDR | (www.biocomputing.it.digit) | model) | (Kabatt) | (Kabatt) |
| Lilotomab | H1 | DYNMY | GYSFTD | 26 | 32 |
| and | H2 | YIDPYNGDTTYNQKFKG | PYN | 52A | 55 |
| derivatives | H3 | SPYGHYAMDY | PYGHYAM | 96 | 101 |
| hereof | L1 | KASQDVSTAVD | ASQDVST | 24 | 42 |
| | L2 | WASTRHT | WA | 59 | 70 |
| | L3 | RQHYSTPFT | HYSTP | 116 | 130 |
| Rituximab | H1 | SYNMH | GYTFTS | 26 | 32 |
| | H2 | AIYPGNGDTSYNQKFKG | PGN | 52A | 55 |
| | H3 | STYYGGDWYFNV | VYYSNSYWYF | 96 | 101 |
| | L1 | RASSSVSYIH | ASSSVS | 24 | 42 |
| | L2 | ATSNLAS | AT | 59 | 70 |
| | L3 | QQWTSNPPT | WTSNP | 116 | 130 |

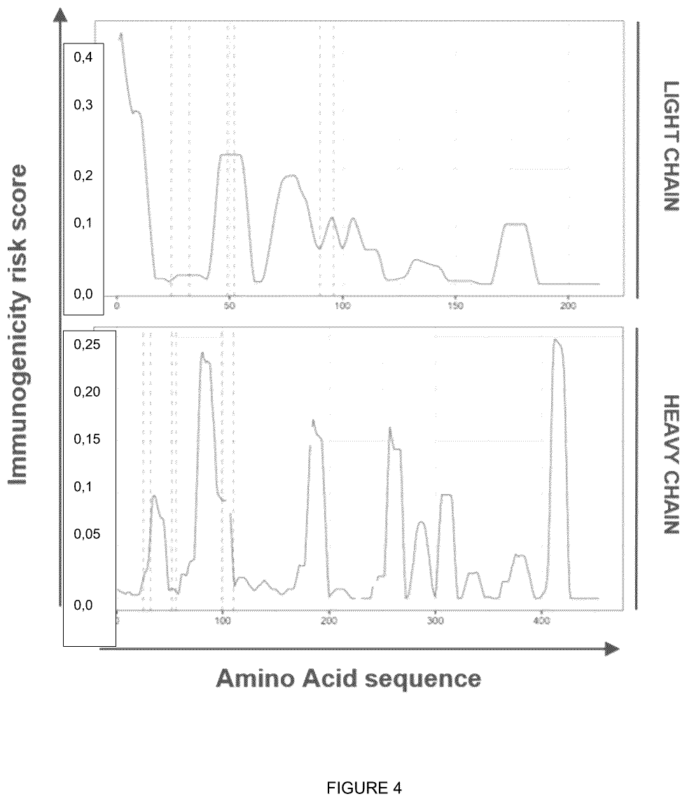

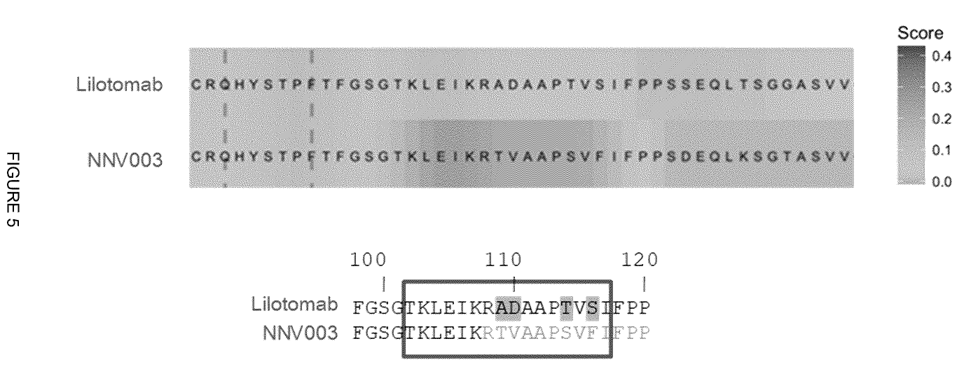

In this example, the CDR in the aligned sequences have the following same start and end position: H1: 25-39, H2: 59-70, and H3: 113-141 for the heavy chain, and L1: 24-42, L2: 59-70, and L3:116-130 for the light chain. These alignments will be used in the FIGS. 4-12 of the present disclosure.

### Identification of Germline Sequences

Immune tolerance is an essential process design to prevent responses to self-antigens. During thymic selection, T cells bearing alfa/beta TCR recognize antigens in the contest of self MHC molecules. In the case of high affinity, TCR binding to self-antigens peptide/MHC complexes leading to T cell removal occurs through a deletional mechanism. Consequently, this process leads to the development of functional immune repertoire fit for response to a diverse array of potential foreign antigens but unable to respond to self-antigens.

V and J germline genes were identified by blasting the heavy and light chain of the tested test articles against a database of V and J germline aa sequences obtained from the IMGT database. The best hit measures by BLAST e-value were selected as the germline gene for each test article. Results as displayed in Table 5.

TABLE 5
Identification of germline sequences for the test articles

| Test article | Germline heavy chain | Germline light chain |
|---|---|---|
| Lilotomab | IGHV1-3*01, IGHJ4*03 | IGKV1D-39*01, IGKJ2*01 |
| NNV003 | IGHV1-3*01, IGHJ4*03 | IGKV1D-39*01, IGKJ2*01 |
| AH02871 | IGHV1-3*01, IGHJ4*03 | IGKV1D-39*01, IGKJ2*01 |
| AH02875 | IGHV1-3*01, IGHJ4*03 | IGKV1D-39*01, IGKJ2*01 |
| AH02877 | IGHV1-3*01, IGHJ4*03 | IGKV1D-39*01, IGKJ2*01 |
| AH02877 | IGHV1-3*01, IGHJ4*03 | IGKV1D-39*01, IGKJ2*01 |
| AH02879 | IGHV1-3*01, IGHJ6*02 | IGKV4-1*01, IGKJ2*01 |
| AH02886 | IGHV1-3*01, IGHJ6*02 | IGKV4-1*01, IGKJ2*01 |
| AH02895 | IGHV1-3*01, IGHJ2*01 | IGKV3-20*01, IGKJ2*02 |
| Rituximab | IGHV4-4*08, IGHJ1*01 | IGKV1-NL1*01, IGKJ4*02 |

The most common alleles among the antibodies included in this example are IGHV1-3*01, IGHJ4*03 and IGKV1 D-39*01, IGKJ2*01 for the heavy and light chain respectively. These were used to create the germline reference sequences for each antibody.

### Identification of T Cell Neo-Epitopes for Lilotomab

In silico MHC class II-binding peptide mapping identified sequences in lilotomab light and heavy chain binding to HLA-DR, -DP, and -DQ. For the mouse IgG1 mAb lilotomab, the repertoire of T cell epitopes and T cell neo-epitopes overlapped, as expected. MHC class II-peptide binding mapping identifies promiscuous overlapping T cell neo-epitopes for both the light chain and the heavy chain. The IRS score plot illustrates the distribution of the T cell neo-epitopes across the sequence of lilotomab light and heavy chain by plotting the self-adjusted position specific IRS (FIG. 4).

### Identification of T Cell Neo-Epitopes for NNV003, m/h IgG1 Chimeric Analogue of Lilotomab

Generation of the m/h IgG1 chimeric analogue of lilotomab, NNV003, was generated by replacing the constant part of the light (C_{L}) and heavy (C_{H}) chain of lilotomab by that of human IgG1. In silico MHC class II-binding peptide mapping identified sequences in NNV003 light and heavy chain binding to HLA-DR, -DP, and -DQ. When subtracting the MHC class II-binding epitopes counting only germline sequences, T cell neo-epitopes predicted to bind to HLA-DR, -DP, and -DQ were identified in the variable regions of NNV003 light (VL) and heavy (HL) chain.

In addition of those identified for lilotomab LC and HC, MHC class II-peptide binding mapping predicts that replacing lilotomab LC with hIgg1 LC generates a new promiscuous overlapping T cell neo-epitope spanning NNV003 mVL-hLC in position 102-116 (FIG. 5).

### Identification of T Cell Neo-Epitopes for NNV020 Drug Candidates

NNV020 drug candidates were generated from NNV003 sequence by combining in silico CDR-grafting and structure-based back mutation as described in Example 1. The six NNV020 drug candidates included in this example were selected from a pool of 26 variants for their binding affinity to the CD37-expressing human cell line RAMOS (Example 4).

### Heavy Chains

The mutations inserted into NNV003 heavy chain sequence during the humanisation process are reported in Table 6.

TABLE 6
Mutations inserted in NNV020 drug candidates heavy
chain sequence during the humanisation process

| Test | | |
|---|---|---|
| articles | Mutations inserted in the variable region of the heavy chain | IRS |
| AH02871 | E1Q I2V Q5V P9A L11V S40A H41P K43Q S44R I48M K67R A68V L70I | 4.5 |
| | V72R K74T S76A F80Y I81M H82E N84S T87R S91T S114L | |
| AH02875 | E1Q Q5V P9A V12K K38R S40A H41P K43Q S44R I48M K67R L70I | 8.0 |
| | V72R K74T S76A F80Y H82E N84S T87R S91T S114L | |
| AH02877 | E1Q I2V Q5V P9A L11V S40A H41P K43Q S44R I48M K67R K74T | 7.2 |
| | S76A F80Y H82E N84S T87R S91T S114L | |
| AH02879 | E1Q Q5V P9A L11V S40A H41P K43Q S44R K67R V72R K74T S76A | 5.3 |
| | F80Y I81M H82E N84S T87R S91T S114L | |
| AH02886 | E1Q I2V Q5V P9A V12K S40A H41P K43Q S44R I48M K67R A68V | 4.8 |
| | K74T S76A F80Y I81M H82E N84S T87R S91T S114L | |
| AH02895 | E1Q Q5V P9A L11V S40A H41P K43Q S44R I48M K67R L70I V72R | 7.5 |
| | K74T S76A F80Y H82E N84S T87R S91T S114L | |

Note:
The mutations inserted in all NNV020 variant sequences are highlighted in bold.
IRS: overall self-adjusted immunogenicity risk score for the world average population.

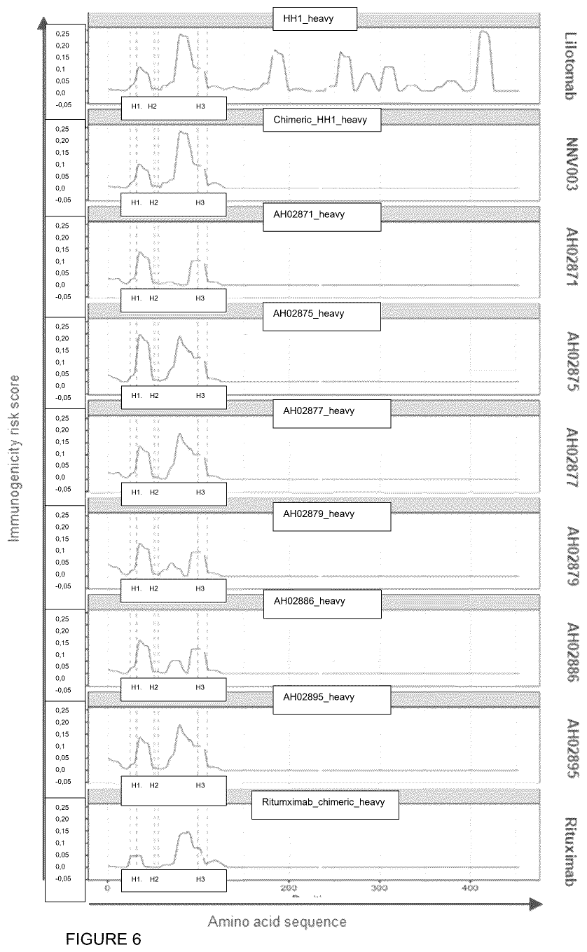

In silico MHC class II-binding peptide mapping identified sequences in NNV020 drug candidates heavy chain binding to HLA-DR, -DP, and -DQ. When subtracting the MHC class II-binding epitopes counting only germline sequences, T cell neo-epitopes predicted to bind to HLA-DR, -DP, and -DQ were identified in the variable regions of the heavy (VH) chain. The IRS score plot illustrates the distribution of the T cell neo-epitopes across the sequence of NNV020 drug candidates' heavy chain by plotting the self-adjusted position specific IRS (FIG. 6).

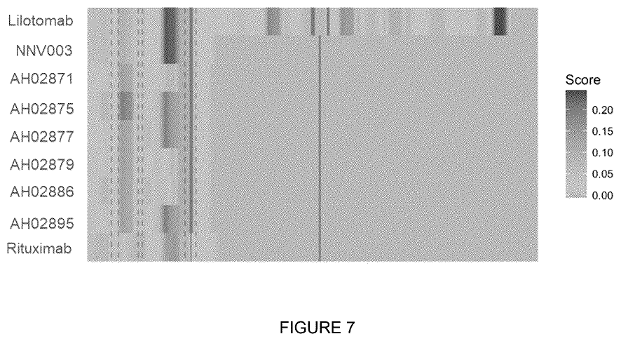

Alignment of the self-adjusted position specific IRS calculated for the average world population for all heavy chain of the test articles included in this example is illustrated in FIG. 7.

Data suggest that the humanisation process applied to NNV003 heavy chain sequence removed T cell neo-epitopes and/or decreased their promiscuity. Calculation of the overall self-adjusted IRS for the world average population allows the following IRS-based ranking of the NNV020 drug candidate heavy chains:
AH02871<AH02886<AH02879<AH02877<NNV003<AH02895<AH02875<lilotomab with AH02871 heavy chain predicted to have the lowest immunogenicity potential.

The mutations E1Q 12V Q5V P9A L11 V S40A H41 P K43Q S44R 148M K67R A68V L701 V72R K74T S76A F80Y 181 M H82E N84S T87R S91T S114L introduced into NNV003 heavy chain sequence to generate AH02871 would thus appear to be the optimal tested combination to reduce overall the number of T cell neo-epitopes and/or their promiscuity. Nevertheless, mutations E1Q 12V and S40A H41P K43Q S44R 148M are predicted to create few new T cell neo-epitopes and slightly increases the promiscuity of an existing T cell neo-epitope, respectively. Further de-immunisation or back mutation at these positions could further decrease the predicted immunogenicity potential of AH02871 heavy chain.

In conclusion, combination of epitope mapping and overall self-adjusted IRS-based ranking predicts AH02871 heavy chain to have the lowest immunogenicity potential. Further de-immunisation or back mutation in position 1-2, 40, 41, 43 and 44 could further decrease the predicted immunogenicity potential of AH02871 heavy chain.

### Light Chains

The mutations inserted into NNV003 light chain sequence during the humanisation process are reported in the Table 7.

TABLE 7
Mutations inserted in NNV020 drug candidates light
chain sequence during the humanisation process

| Test | | |
|---|---|---|
| articles | Mutations inserted in the variable region of the light chain | IRS |
| AH02871 | V3Q H8P K9S L10S T13A S20T Q42K S43A D60S T63S A80P L83F | 9.2 |
| | L85T | |
| AH02875 | V3Q H8P K9S L10S T13A S20T Q42K D60S T63S A80P L83F L85T | 9.2 |
| AH02877 | V3Q H8P K9S L10S T13A S20T Q42K D60S T63S M78L A80P L83F | 7.9 |
| | L85T | |
| AH02879 | V3Q H8P K9S L10S S20T Q42K D60S T63S A80P L83F L85T | 9.8 |
| AH02886 | V3Q H8P K9S L10S S20T Q42K S43A D60S T63S M78L A80P L83F | 8.5 |
| | L85T | |
| AH02895 | V3Q H8P K9S L10S T13A S20T Q42K S43A D60S T63S A80P L83F | 9.2 |
| | L85T | |

Note:
The mutations inserted in all NNV020 variant sequences are highlighted in bold.
IRS: overall self-adjusted immunogenicity risk score for the world average population.

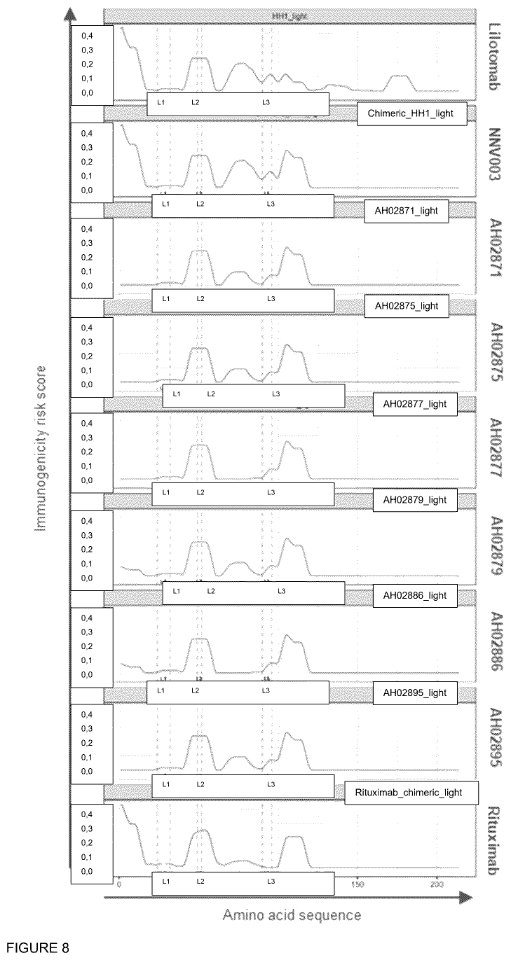

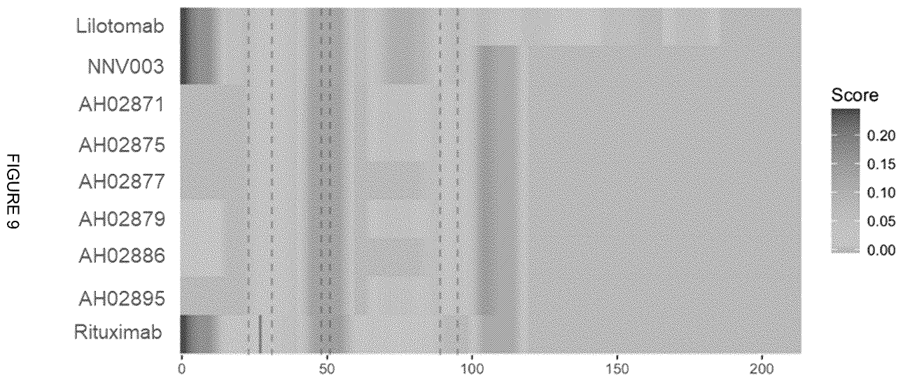

In silico MHC class II-binding peptide mapping identified sequences in NNV020 drug candidates' light chain binding to HLA-DR, -DP, and -DQ. When subtracting the MHC class II-binding epitopes counting only germline sequences, T cell neo-epitopes predicted to bind to HLA-DR, -DP, and -DQ were identified in the variable regions of the light (VL) chain. The IRS score plot illustrates the distribution of the T cell neo-epitopes across the sequence of NNV020 drug candidates' light chain by plotting the self-adjusted position specific IRS (FIG. 8).

Alignment of the self-adjusted position specific IRS calculated for the average world population for all light chain of the test articles included in this example is illustrated in FIG. 9.

Data suggest that the humanisation process applied to NNV003 light chain removed T cell neo-epitopes and/or decreased their promiscuity.

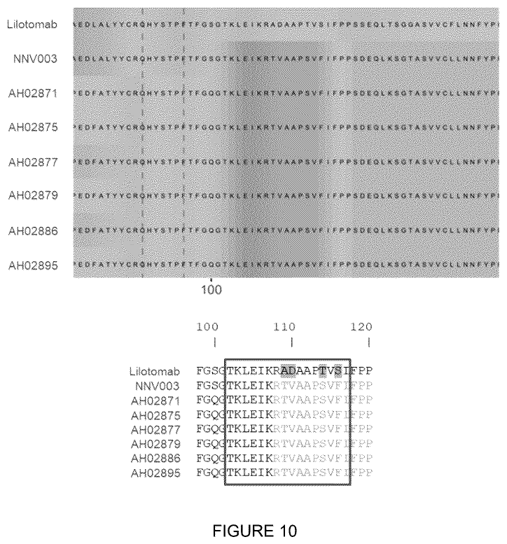

The promiscuous overlapping T cell neo-epitope spanning NNV003 mV_{L}-hLC in position 102-116 was also identified for all NNV020 candidates and the other m/h IgG1 chimeric mAb included in this example, rituximab (FIG. 10).

Calculation of the overall self-adjusted IRS for the world average population allows the following IRS-based ranking of the NNV020 drug candidate light chains:
AH02877<AH02886<AH02871 #AH02875 #AH02895<AH02879<NNV003 #lilotomab with AH02877 light chain predicted to have the lowest immunogenicity potential.

The mutations V3Q H8P K9S L10S T13A S20T Q42K D60S T63S M78L A80P L83F L85T introduced into NNV003 light chain sequence to generate AH02877 would thus appear to be the optimal tested combination to reduce overall the number of T cell neo-epitopes and/or their promiscuity.

In conclusion, combination of epitope mapping and overall self-adjusted IRS-based ranking predicts AH02877 light chain to have the lowest immunogenicity potential. Further de-immunisation of the T cell neo-epitope spanning AH02877 mV_{L}-hLC in position 102-116 could further decrease its predicted immunogenicity potential.

### De-Immunisation of AH02877Light Chain

The promiscuous overlapping T cell neo-epitope spanning AH02877 mV_{L}-hC_{L} in position 102-116 was also identified for the m/h IgG1 chimeric anti-CD20 mAb rituximab. Data obtained in an in vitro MHC-Associated Peptide Proteomics (MAPP) assay identified rituximab-derived peptides spanning mVL-hLC, increasing the likelihood for this predicted T cell neo-epitope to trigger an ADA response in the clinic. Therefore, de-immunisation was performed in silico by introducing all natural aa at the positions 100-115 of the AH02877 light chain.

Conservation scores for each aa substitution were calculated using the psiblast algorithm using a database of 2287 antibody light chain sequences downloaded from the PDB database. The self-adjusted IRS or mutated IRS was calculated for the average world population for each aa substitution. The effect of the mutations on overall IRS was reported as change in IRS by subtracting the mutated IRS to the IRS for the native sequence. Calculation of change in AH02877 overall risk score after single mutation in the sequence spanning mV_{L}-hLC supports de-immunisation in position 106-114.

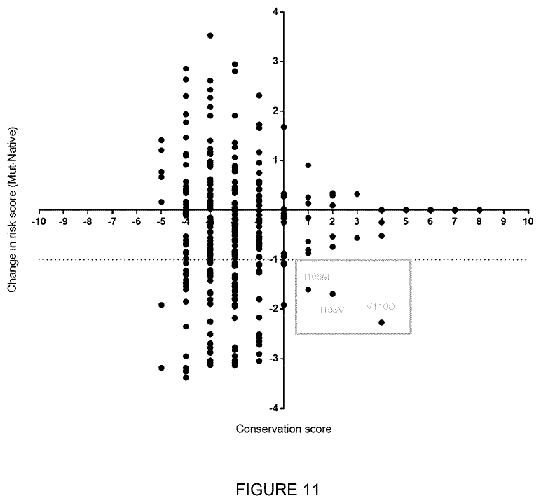

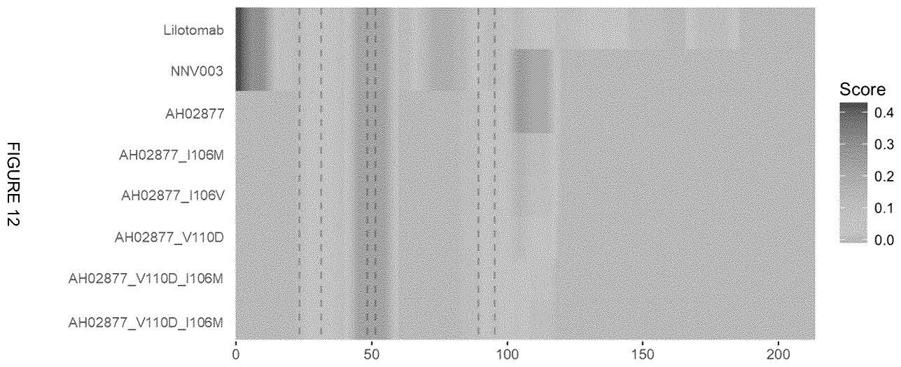

Plotting of the conservation scores vs. the change in IRS allowed to identify 2 single mutations 1106M and 1106V as well as one back mutation to lilotomab sequence V110 D combining a high conservation score and a high change in IRS (FIG. 11).

The overall IRS for the world average population was calculated for the 5 variants of AH02877 light chain sequences generated in silico by introducing one or two of the aa substitutions predicted to decrease its immunogenicity potential. The sequence spanning the position 100-115 of the light chain of the different AH2877 variants are reported in Table 8. Their predicted overall IRS for the average world population is indicated. Lilotomab, NNV003 and rituximab are included for benchmarking.

TABLE 8
AH02877 light chain variants overall
IRS for the average population

| | Sequence spanning | Light |
|---|---|---|
| Test article | position 100-115 | chain IRS |
| Lilotomab | — | 15.8 |
| NNV003 | A109T D110V T114S S116F | 15.8 |
| AH02871 | A109T D110V T114S S116F | 9.2 |
| AH02875 | A109T D110V T114S S116F | 9.2 |
| AH02877 | A109T D110V T114S S116F | 7.9 |
| AH02879 | A109T D110V T114S S116F | 9.8 |
| AH02886 | A109T D110V T114S S116F | 8.5 |
| AH02895 | A109T D110V T114S S116F | 9.2 |
| AH02877_I106M | A109T I106M D110V T114S | 6.25 |
| | S116F | |
| AH02877_I106V | A109T I106V D110V T114S | 6.16 |
| | S116F | |
| AH02877_V110D | A109T V110D T114S S116F | 5.58 |
| AH02877_I106M_V110D | A109T I106M V110D T114S | 4.91 |
| | S116F | |
| AH02877_I106V_V110D | A109T I106V V110D T114S | 4.85 |
| | S116F | |
| Rituximab | — | 12.6 |

The IRS score plot illustrates the distribution of the T cell neo-epitopes across the sequence of AH02877 and its variants' light chain by plotting the self-adjusted position specific IRS (FIG. 12).

Calculation of the overall self-adjusted IRS for the world average population allows the following IRS-based ranking of the AH02877 variants light chains:
V110D_I106V <V110D_I106M <V110D <1106M <AH02877<NNV003/lilotomab
   with AH02877_V110D_I106V light chain predicted to have the lowest immunogenicity potential.

In conclusion, in silico de-immunisation of AH02877 light chain sequence spanning mV_{L}-hC_{L} in position 102-116 allowed to decrease its predicted immunogenicity potential. Mutation 1106M and 1106V and back mutation V110D were identified as key substitutions. Combination of epitope mapping and overall self-adjusted IRS-based ranking predicts AH02877_V110D_I106V the most optimal tested combination to reduce AH02877 light chain predicted immunogenicity potential.

Sequences of AH02877_V110D_I106V, AH02877_V110D_I106M, AH02877_V110D, AH02877_I106M, AH02877_1106V light chain variable region are shown as SEQ ID NOs: 14-18, and the full-length light chain domains are shown in SEQ ID NOs: 24-28.

Selection of a new anti-CD37 humanised mAb Using in silico immunogenicity prediction tools and in silico de-immunisation allowed to select a new anti-CD37 humanised mAb with a predicted immunogenicity potential lower than that for lilotomab, the m/h IgG1 chimeric analogue of lilotomab, NNV003, and rituximab. The combination of the heavy chain from AH02871 and the light chain of AH02877 after introducing mutation 1106V and back mutation V110D. As mutation in position 106 would be introduced close to the CDR3, potential impact on the binding capacity of the anti-CD37 mAb must be considered. Therefore, the heavy chain of AH02871 and the light chain of AH02877 with the back mutation V110D were selected to generate a new NNV020 drug candidate. This new anti-CD37 humanised antibody was designated as NNV023.

Combined light and heavy chain IRS are reported in Table 9. The IRS-based ranking of all the test articles included in this example for the world average population is then the following: NNV023<AH02886<AH02871>AH02877<AH02879<AH02895<AH02875<rituximab <NNV003<Lilotomab

TABLE 9
Self-adjusted immunogenicity risk
scores - World average population.

| Test articles | Heavy chain IRS | Light chain IRS | Combined IRS |
|---|---|---|---|
| NNV023 | 4.5 | 5.6 | 10.0 |
| AH02886 | 4.8 | 8.5 | 13.3 |
| AH02871 | 4.5 | 9.2 | 13.6 |
| AH02877 | 7.2 | 7.9 | 15.1 |
| AH02879 | 5.3 | 9.8 | 15.2 |
| AH02895 | 7.5 | 9.2 | 16.7 |
| AH02875 | 8.0 | 9.2 | 17.2 |
| Rituximab | 4.7 | 12.6 | 17.4 |
| NNV003 | 7.3 | 15.8 | 23.1 |
| Lilotomab | 20.5 | 15.8 | 36.4 |

IRS: Immunogenicity Risk Score

### Discussion

Any of the promiscuous non-germline MHC class II-binding peptides identified in this in silico binding predictive has the potential to be a neo-epitope recognized by an active CD4⁺ T cell.

Provided that such T cells are present together with B cells capable of binding to the protein, an ADA response may develop.

Nevertheless, there are certain caveats that need to be considered when analysing sequences for MHC class II-binding peptides as stand-alone analysis. Although the current in silico tools offer a reasonable level of accuracy in predicting MHC class II-binding peptides against a large number of HLA alleles, it must be noted that this method over-predicts the number of T cell epitopes since it does not take into consideration other important biological aspects limiting the number of true functional neo-epitopes such as protein and peptide processing in the APC, T cell receptor repertoire capable of recognizing the MHC class II/peptide complex, as well as induction of T cell tolerance to foreign peptides.

In this example, we report the in silico MHC class II binding prediction analysis of peptides derived from variant of a novel anti-CD37 humanized mAb to,
i) predict potential neo-epitopes potentially able to trigger an immune response, and
ii) rank the drug candidates at the pre-clinical stage of drug development based on their IRS to support the lead candidate selection by reducing the risk for immunogenicity in the clinic.

The drug candidates tested included six NNV020 drug candidates generated during the humanisation of lilotomab i.e. AH02871, AH02875, AH02877, AH02879, AH02886, and AH02895. Lilotomab, the m/h IgG1 chimeric analogue of lilotomab (NNV003), and the m/h IgG1 chimeric anti-CD20 mAb rituximab were included in the example for benchmarking.

In silico MHC class II-binding peptide mapping performed with the NetMHCIIpan-3.1 algorithm identified potential T cell epitopes for lilotomab and its derivatives hereof binding to HLA-DR, -DP and -DQ. When subtracting the MHC class II-binding peptides containing germline sequence of hIgG1, a variable number of T cell neo-epitopes predicted to bind to HLA-DR, -DP and -DQ were identified for each of the test articles.

Data suggest that lilotomab predicted T cell neo-epitopes are either of murine origin or spanning the CDR.

The replacement of the murine LC of lilotomab with a human IgG1 C_{L} to generate the m/h IgG1 chimeric analogue of lilotomab, NNV003, was predicted to generate a new neo-epitope spanning NNV003 and all NNV020 drug candidates mVL-hLC in position 102-116. This neo-epitope is shared with the m/h IgG1 chimeric rituximab.

Combination of T cell epitope mapping and overall self-adjusted IRS-based ranking predicted AH02871 heavy chain and AH02877 light chain to have the lowest immunogenicity potential. Additional de-immunisation of the AH02877 light chain in position 100-115 allowed to reduce further its immunogenicity potential when introducing mutation in position 106 and the back mutation V110 D. The combination of mutation 1106V and back mutation V110 D was predicted to be the optimal tested combination to reduce AH02877 light chain immunogenicity potential. As mutation in position 106 would be introduced close to the CDR3 and per extension could impact the binding capacity of the mAb, the heavy chain of AH02871 and the light chain of AH02877 with the back mutation V110 D were selected to generate a new NNV020 drug candidate. This new anti-CD37 humanised antibody was designated as NNV023.

The IRS-based ranking of all the test articles included in this example for the world average population is then:
NNV023<AH02886<AH02871>AH02877<AH02879<AH02895<AH02875<rituximab <NNV003<Lilotomab

By exploring the peptide-MHC class II interactions, the in silico binding predictive analysis of the NNV020 variants allowed the selection of a new anti-CD37 humanized mAb predicted to have a lower predicted immunogenicity potential in the clinic than lilotomab, NNV003 and rituximab.

### Example 3—Manufacturing of Humanized Antibodies

The aim of this example was to manufacture five different humanized antibodies that was selected after studies described example 1 and 2. The selected antibodies were AH02871 (NNV026), AH02886 (NNV027), AH02871HC_2877LC (AH02871HC+AH028771LC, NNV025), AH02871 HC_2877LC-110 D (AH02871 HC+AH028771 LC with V110 D mutation, NNV023) NONA2871 HC_2877LC-110D GlymaxX (Afucosylated AH02871 HC+AH028771 LC with V110 D mutation, NNV024).

### Methods

### Design, Expression, and Purification of Antibodies

There were 6 samples of humanized Abs manufactured in 4 sub-projects, as shown in table 10.

TABLE 10

| | | | Project | Date of | Stored |
|---|---|---|---|---|---|
| Antibody | NNV ID | Amount | number | completion | at NaNo |
| AH02871 | NNV026 | 40-mg scale | 21751 | May 2018 | 5° C., −80° C. |
| AH02886 | NNV027 | 40-mg scale | 21751 | May 2018 | 5° C., −80° C. |
| AH2871HC_2877LC | NNV025 | 40-mg scale | 21751 | May 2018 | 5° C., −80° C. |
| AH02871HC_2877LC-110D | NNV023 | 40-mg scale | 22405 | July 2018, | Stored 5° C. |
| AH02871HC_2877LC-110D | NNV023 | 50 mg scale | 31709 | May 2019, | 5° C., −80° C. |
| AH02871HC_2877LC-110D | NNV024 | 50 mg scale | 31708 | May 2019, | 5° C., −80° C. |
| GlymaxX | | | | | |

Each project included two work packages:
Initial example (section I)
Larger scale expression and purification (section II)

During the initial example the following activities were performed:
1. Gene design for the variable domain (cDNA optimization) and synthesis
2. Cloning into vector system
3. Generation of low-endotoxin plasmid DNA
4. Pilot 10 mL transfection
5. Analytics: DNA sequencing (provided by 3rd party; max. two sequencing reactions) and titer quantification

The larger scale expression and purification included the steps:
1. Preparation of transfection-quality plasmid DNA
2. Transfection of CHO cells (supernatant generation)
3. Purification (with MabSelect SuRe alkali-tolerant protein A-derived affinity matrix)
4. Final buffer: PBS with 100 mmol/1 L-arginine (pH 6.5-6.7)
5. Sterile filtration
6. Analytics included determination of
   quantity (mg),
   volume (ml),
   concentration (mg/ml; A280 nm reading incl. extinction coefficient),
   purity (%; HPLC-SEC),
   endotoxin level (EU/mg)
      Preparation of cDNA Vector for Transfection

The cDNAs were cloned into the vector system using conventional (non-PCR based) cloning techniques and the plasmids were synthesized. Plasmid DNA was prepared under low-endotoxin conditions based on anion exchange chromatography. DNA concentration was determined by measuring the absorption at a wavelength of 260 nm. Correctness of the sequences was verified with Sanger sequencing (with up to two sequencing reactions per plasmid depending on the size of the cDNA.)

### Cell Transfection

Suspension-adapted CHO K1 cells were used for production. The seed was grown in eviGrow medium, a chemically defined, animal-component free, serum-free medium. Cells were transfected with eviFect, and cells were grown after transfection in eviMake2, an animal-component-free, serum-free medium.

Purification of IgG1s from Supernatant

Supernatant was harvested by centrifugation and subsequent filtration (0.2 μm filter). The antibody was purified using regenerated (by making a low pH wash as well as a 0.1 M NaOH wash, for 1 hour in total) FPLC column filled with MabSelect™ SuRe™ resin. One can normally recover appr. 50% of the titer measured for recombinant human IgG1 antibodies.

### Analytical Procedures

The concentration of Ab was determined by measuring absorption at a wavelength of 280 nm. The extinction coefficient was calculated using a proprietary algorithm at Evitria. Purity was determined by analytical size exclusion chromatography with an Agilent AdvanceBio SEC column (300A 2.7 um 7.8×300 mm) and DPBS as running buffer at 0.8 ml/min with detection at 280 nm.

Endotoxin content was measured with the Charles River Endosafe PTS system. Titers were measured with ForteBio Protein A biosensors (using kinetic assay method) and calculated based on a human IgG1 standard.

Sequences of humanized IgG1 are shown in the previous examples. In the present example, combination of NNV020 variants was also prepared, combining AH02871_HC with AH02877_LC, and combining AH02871_HC with the AH02877_LC_V110 D variant, thus creating two additional antibodies, NNV025 (SEQ ID NOs: 29, 72) and NNV023 (SEQ ID NOs: 29, 24) respectively.

In order to produce an afucosylated variant of the antibody, cells also comprised a protein of SEQ ID NO: 77.

### Results

### Analytical Summary

Table 11 below displays the analytical summary of all products manufactured.

| NNV Code | NNV026 | NNV027 | NNV025 | NNV023 | NNV023 | NNV024 |
|---|---|---|---|---|---|---|
| Old NAME | AH02871 | AH02886 | AH02871_HC_{—} | AH02871_HC_{—} | AH02871_HC_{—} | AH02871_HC_{—} |
| | | | 02877_LC | 02877_LC- | 02877_LC- | 02877_LC- |
| | | | | V110D | V110D | 110D |
| | | | | | | GlymaxX |
| Date of | 14 May 2018 | 14 May 2018 | 14 May 2018 | 9 Jul. 2018 | 11 Sep. 2019 | 11 Sep. 2019 |
| completion | | | | | | |
| Project | 21751 | 21751 | 21751 | 22405 | 31709 | 31708 |
| Quantity | 256 | 256 | 263 | 297 | 248 | 221 |
| (mg) | | | | | | |
| Conc. | 7.77 | 8 | 8.2 | 7.62 | 7.5 | 7.38 |
| (mg/ml) | | | | | | |
| Volume (ml) | 33 | 32 | 32 | 39 | 33 | 30 |
| Buffer | PBS 100 mM L-arginine | | | | | |
| Endotoxin | <1 EU/mg | | | | | |
| (EU/mg) | | | | | | |
| Monomericity | 98.90% | 99.00% | 98.90% | 99.30% | 99.20% | 99.10% |
| (%) | | | | | | |
| OD_{280 nm} | 11.8 | 12.1 | 12.4 | 11.5 | 11.4 | 11.2 |
| Ext. coeff. | 1.51 | 1.51 | 1.51 | 1.51 | 1.51 | 1.51 |

### Discussion

Six humanized IgG1 antibodies were manufactured over 4 projects. All manufactured humanized antibodies are of good quality (monomer %, endotoxin level, concentration). The good quality of the manufactured hzAbs mean that they can be used for various tests for further identification of the best lead and for both in vitro and in vivo PoC studies for Fc-modified hzIgG1.

Manufacturing of NNV024 required co-transfection of CHO cell line with an additional protein, GDP-4-keto-6-deoxy mannose reductase (SEQ ID NO: 77). This protein is a bacterial GDP-4-keto-6-deoxy mannose reductase (RMD) that depletes the cytosolic pool of GDP-4-keto-6-deoxy mannose, which is a precursor for the synthesis of fucose. This precursor is being transformed to GDP-D-Rhamnose—an important for bacteria, but inactive sugar in mammalian cell.

The choice of the buffer (containing L-arginine), however, is suboptimal—there might be certain difficulties in conjugation with bifunctional groups like chelating agents or various cross-linkers (e.g. for Antibody Drug conjugates).

The L-arginine which is present in the buffer contains a reactive amine which might be an obstacle in the reactions where the primary amines of, primarily Lysines in the antibody are used as sites for conjugation. An example of such reactions could be, but not limited to, a conjugation with bifunctional reagents containing isothiocyanate groups (e.g. p-SCN-Bn-EDTA, p-SCN-Bn-DOTA, p-SCN-Bn-TCMS, etc). This obstacle can be overcome by adding an extra step for buffer exchange and washing of the protein before the conjugation.

### Example 4—Binding of Humanized Anti-CD37 Antibodies to Ramos Cells

The aim of the example is to compare ability of 26 humanized antibodies to bind to the CD37 expressing Ramos cell line. The binding of each antibody will be compared with the binding of the chimeric HH1 (NNV003). Non-specific binding will be evaluated with an isotype control antibody. The ability of the antibodies to bind to Ramos cells was tested in two separate labs, and the results were compared and combined to rank the antibodies.

### Methods

### Cells

The Ramos cell line were grown in RPMI 1640 medium supplemented with Glutamax (Gibco, Paisley, UK), 10% heat-inactivated FCS (Gibco) and 1% penicillin-streptomycin (Gibco) in a humidified atmosphere with 5% CO₂.

The suspension is routinely split with pre-warmed fresh cell growth medium in proportion 1:5 every 3-4 days. The cells were washed 3 times with 20-30 ml of 0.5% BSAPBS, and then 2 ml of the cell suspension (3×10⁶ cells/ml) was prepared for the analysis.

### Preparation of Samples

The samples were aliquots of HEK293 cell growth medium supernatants containing only one type of humanized test antibody (see Example 1).

All samples (25) were split in 3 groups: 5, 10, and 10 samples. Each group was stained and data acquired at a separate day.

The samples were added to Ramos cells distributed to wells of 96 well plates. Either 15 μl or 150 μl of the HEK293 supernatant were added to the wells with cells. Each supernatant aliquoted by 15 μl and 150 μl was tested in triplicates. The cells were kept on ice.

The samples (5 or 10 at a time) were prepared by incubating 3×10⁵ cells (approximately 100 μl) with either 15 μl or 150 μl of supernatant containing 0.15-0.2 μg/ml of the humanized antibodies. Cell growth medium was used to equalize volume in a well for 15 μl samples and 150 μl samples. The content of the wells was mixed and incubated for 1 hour on ice in a refrigerator. After each incubation, the cells were washed 3 times. At the each washing step, the cell pellets were mixed with 200 μl of 0.5% BSA_{PBS} using 8-channel pipettor with disposable tips. The cell suspension was then centrifuged on 96 well plates at 1200 RPM and 5° C. for 5 min. Before acquisition on a flow cytometer, the cells were stained with Alexa Fluor 647 goat anti-human IgG (H+L) [Molecular Probes, Ref.: A21445, Lot. 1711471]. Concentration 2.0 μg/ml was prepared by diluting the original solution 1000-fold times with PBS.

To each cell pellet, 150 μl of anti-human Ab (2.0 μg/ml) was added, mixed with 8-channel pipette as described above and incubated for 30 min on ice in a refrigerator. The 96 well plate with cells was gently shaken in the middle and at the end of incubation.

After incubation, the cells were washed 3 times as described above. The cells pellet was resuspended in 200 μl of ice-cold growth medium as a final step of the sample preparation.

### Flow Cytometry

An acquisition of each sample on the flow cytometer takes 1.5-3 min; therefore, special measures were taken to obtain results independent on the time lag between the samples acquired in the beginning and at the end of the 96 well plate. For this, the first replicas (out of three) of each sample were acquired first, followed by the second replicas and then by the third replicas of each sample.

The data acquisition of the stained cells was performed using the Guava EasyCyte 12HT flow cytometer. Medium aspiration rate was used and the capillary was flushed with cleaning solution after each 12 samples. The stopping gate was the gate for single cells SSC/FSC. The number of events to acquire in the stopping gate was set to 5×10³ events for each sample. The Median Fluorescence Intensity (MFI) of Alexa Fluor 647 of each sample was used as a raw data in the data analysis.

A similar flow cytometry method (FACSCalibur, BD Bioscience, San Jose, CA) and the same Ramos cell lines was used for measurement of binding of the construct to CD37.

### Data Analysis

The averaged value (MFI of AlexaFluor 647) of triplicates for each sample was used for analysis. In order to diminish an influence of the laser intensity variations from different days of the experiment, the values of each sample (X) were normalized relatively to the ratio of _{MFI}NSB (denoted as Xₘᵢₙ) to _{MFL}chHH1 (denoted as Xₘₐₓ):

$${X}_{norm} = ( X - {X}_{min} ) / ( {X}_{max} - {X}_{min} )$$

Then, the signals were scaled between minimum (Xₘᵢₙ=28.3%, i.e. an average of MFI of NSB in all experiments) and maximum values (Xₘₐₓ=112.2%, i.e. MFI of chHH1, as it showed the highest binding among the samples).

$${X}_{s c a led} = {X}_{norm} * ( {X}_{max} - {X}_{min} ) + {X}_{min}$$

chHH1-DOTA was used as a positive control and binding of this antibody conjugate was normalized to 100%. chHH1-DOTA is a conjugate of chHH1/NNV003 with p-SCN-Bn-DOTA, and is also referred to as NNV009.

### Results

Each sample had values for 15 μl and 150 μl aliquot at both labs. Table 12 shows average normalized binding data merged for both aliquots and both labs. The core back mutated construct showed the highest binding with 112% of the positive control. The other constructs showed lower binding, indicating that core back mutations are necessary to get a high binding affinity. The highest binders, not taking the CBM (CVH+CHL Ab was as defined in Example 1) into account was HC2+LC2 with 66.6%.

TABLE 12
Normalized average binding of the different constructs.

| Construct | Normalized average binding (%) |
|---|---|
| chHH1-DOTA | 100 |
| HC1 + LC1 with 14 CBM | 112 |
| HC2 + LC2 | 66.6 |
| HC3 + LC2 | 60.4 |
| HC1 + LC2 | 59.5 |
| HC2 + LC5 | 57.7 |
| HC2 + LC1 | 56.2 |
| HC5 + LC2 | 55.4 |
| HC2 + LC3 | 54.6 |
| HC3 + LC1 | 52.4 |
| HC3 + LC5 | 52.3 |
| HC5 + LC5 | 51.7 |
| HC5 + LC3 | 50.5 |
| HC5 + LC4 | 50.2 |
| HC5 + LC1 | 49.3 |
| HC3 + LC3 | 49.0 |
| HC3 + LC4 | 48.3 |
| HC2 + LC4 | 46.3 |
| HC1 + LC3 | 45.9 |
| HC1 + LC4 | 43.9 |
| HC4 + LC4 | 42.6 |
| HC4 + LC3 | 42.5 |
| HC1 + LC5 | 42.0 |
| HC1 + LC1 | 40.0 |
| HC4 + LC2 | 39.9 |
| HC4 + LC1 | 38.5 |
| HC4 + LC5 | 36.8 |
| Non-specific binding | 29.5 |
| Blank HEK293 supernatant | 28.3 |

### Discussion

It was clear that the core back mutated construct was the best binder, even better than the positive control chHH1l-DOTA.

A core back mutated construct should be used for further work since this gives the best binding to CD37.

### Example 5—Binding of Humanized Anti-CD37 Antibodies to FcRn, FcgR and C1q

The aim of the current example was to determine the binding capacity of the afucosylated humanized NNV024 antibody to a panel of recombinant human effector molecules and to compare it with the NNV003 (chHH1) antibody, conjugated NNV003 (TCMC-NNV003) and the anti-CD20 antibody obinutuzumab (0-Obin).

### Methods

### Measurement of Antibody Concentration

The concentration of the IgG variants was measured using a Denovix spectrophotometer (Denovix) using the built in IgG function with an extinction coefficient of 210,000 cm⁻¹M⁻¹.

### SDS-PAGE

SDS-PAGE was performed using the Bolt MiniTank electrophoresis system (ThermoFisher) with precast 12% Bolt Bis-Tris protein gels (ThermoFisher). For native SDS-PAGE, 2 g of each IgG variant was diluted in a total volume of 10 μl dH₂O (Table 13) before addition of 2 μl 4× Bolt LDS Sample Buffer (ThermoFisher). For reducing SDS-PAGE, 2 g of each IgG variant was diluted in a final volume of 10 μl dH₂O before addition of 2 μl 4× Bolt LDS Sample buffer and 4 μl 10× Bolt Sample Reducing Agent (ThermoFisher). Reduced samples were boiled at 95° C. for 5 min. The gel was run at 200V for 22 min in 1× Bolt MES SDS running buffer (ThermoFisher) before staining with Coomassie Blue (BioRad) and repeated washing with dH₂O.

TABLE 14
Dilution of antibody samples for SDS-PAGE

| | Initial | | |
|---|---|---|---|
| | dilution | Volume of 1:10 | |
| Antibody | factor | dilutions added | Concentration |
| NNV003 | 1:10 | 3.18 μl in 6.82 μl dH2O | 5.0 mg/ml |
| NNV024 | 1:10 | 2.40 μl in 6.82 μl dH2O | 7.4 mg/ml |
| TCMC- | 1:10 | 1.61 μl in 8.39 μl dH2O | 9.4 mg/ml |
| NNV003 | | | |
| O-Obin | 1:10 | 7.49 μl in 2.51 μl dH2O | 25 mg/ml |

### Quantification of Coated IgG Variants

96 well EIA/RIA plates (CorningCostar) were coated with 100 μl titrated amounts of IgG variants (1000-0.45 ng/ml) diluted in phosphate buffered saline (PBS) and incubated overnight (0/N) at 4° C. Remaining surface area were blocked with PBS containing 0.05% Tween20 (T) and 4% skimmed milk powder (S) for 1 hour (h) at room temperature (RT). The plates were washed four times with PBS/T using a Hydrospeed™ plate washer (Tecan). Two different detection antibodies, alkaline phosphatase (AP) conjugated anti-human IgG (Fc-specific) from goat (Sigma Aldrich) and AP conjugated anti-human kappa light chains (Sigma Aldrich) (both diluted 1:5000 in PBS/T/S) were added to separate plates and incubated for 1 h at RT. Following washing as above bound detection antibodies were visualized by addition of phosphatase substrate (1 mg/ml in diethanolamine buffer). The plates were developed for 20-30 min before the 405 nm absorption values were recorded using a TECAN Sunrise spectrophotometer (Tecan).

### Human FcRn ELISA

96 well EIA/RIA plates (CorningCostar) were coated with 100 μl titrated amounts of IgG variants (1000-0.45 ng/ml) diluted in PBS and incubated O/N at 4° C. Remaining surface area were blocked with PBS containing 0.05% T and 4% S for 1 h at RT. The plates were washed four times with PBS/T using a Hydrospeed™ plate washer (Tecan). Preformed complexes of 250 ng/ml biotinylated soluble human FcRn (Immunitrack) and AP conjugated streptavidin (GE Healthcare) (1:1 molar ratio) were then added and incubated for 1 h at RT. Following washing as above with PBS/T or FcRn binding buffer (67 nM phosphate, 0.1 M NaCl, 0.05% T, pH 6.0), bound receptor was visualized by addition of phosphatase substrate (1 mg/ml in diethanolamine buffer) (Sigma Aldrich). The plates were developed for 20-30 min before the 405 nm absorption values were recorded using a TECAN Sunrise spectrophotometer (Tecan).

### Human FcγR ELISA's

96 well EIA/RIA plates (CorningCostar) were coated with 100 μl titrated amounts of IgG variants (10.000-4.5 ng/ml) diluted in PBS and incubated O/N at 4° C. Remaining surface area were blocked with PBS containing 0.05% T and 4% S for 1 h at RT. The plates were washed four times with PBS/T using a Hydrospeed™ plate washer (Tecan). Then 250 ng/ml biotinylated truncated soluble human FcγRI, FcγRIIa-H131, FcγRIIa-R131, FcγRIIb, FcγRIIIa-V158, FcγRIIIa-F158 and FcγRIIIb (Sino Biological) in preformed complex with AP conjugated streptavidin (GE Healthcare) (1:1 molar ratio) were added and incubated for 1 h at RT. Following washing as above, bound receptor was visualized by addition of phosphatase substrate (1 mg/ml in diethanolamine buffer) (Sigma Aldrich). The plates were developed for 15-20 min before the 405 nm absorption values were recorded using a TECAN Sunrise spectrophotometer (Tecan).

### C1q ELISA

96 well EIA/RIA plates (CorningCostar) were coated with 100 μl titrated amounts of IgG variants (20.000-156.25 ng/ml) diluted in PBS and incubated O/N at 4° C. Remaining surface area were blocked with PBS containing 0.05% T and 4% S for 1 h at RT. The plates were then washed four times with PBS/T using a Hydrospeed™ plate washer (Tecan). Human C1q (Complement Technologies) diluted to 360 ng/ml in veronal buffer (Complement Technologies) were added and incubated for 30 min at 37° C. Detection of bound C1q was performed using a primary anti-human C1q antibody from rabbit (DAKO) diluted 1:10.000 in PBS/T/S and a secondary horseradish peroxidase (HRP) conjugated anti-rabbit IgG antibody diluted 1:5000 in PBS/T/S (GE Healthcare). Binding was visualized by addition of 100 μl TMB substrate (Calbiochem) for 15-20 min before the enzymatic reaction was stopped by addition of 50 μl 1 M HC1. The 450 nm absorption values were recorded using a Sunrise TECAN spectrophotometer (Tecan).

### Results

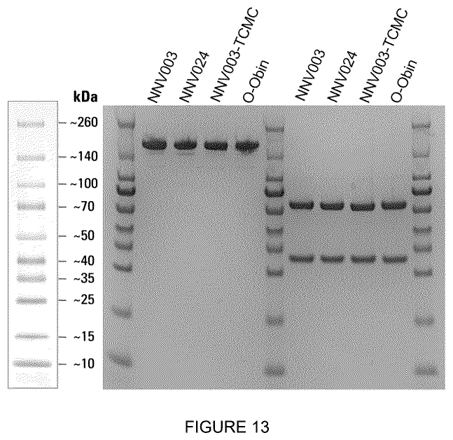

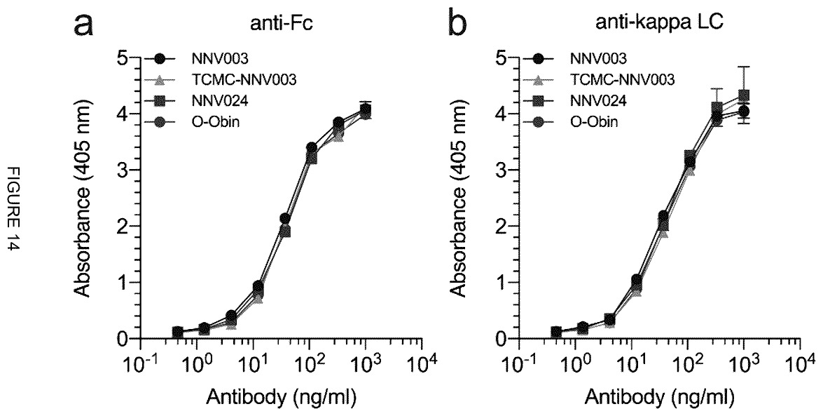

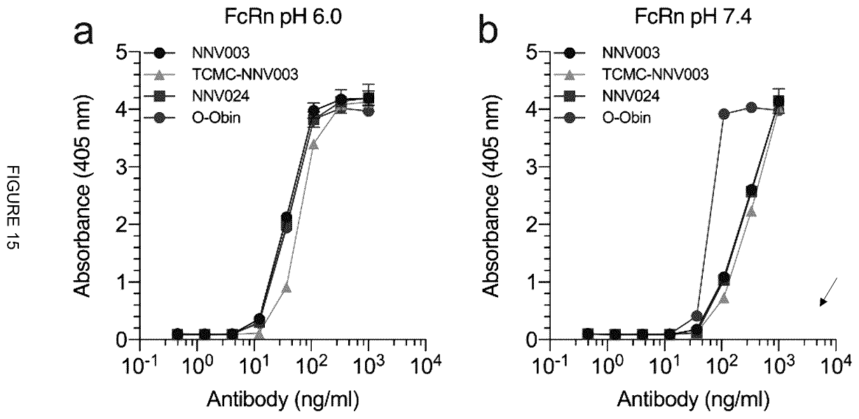

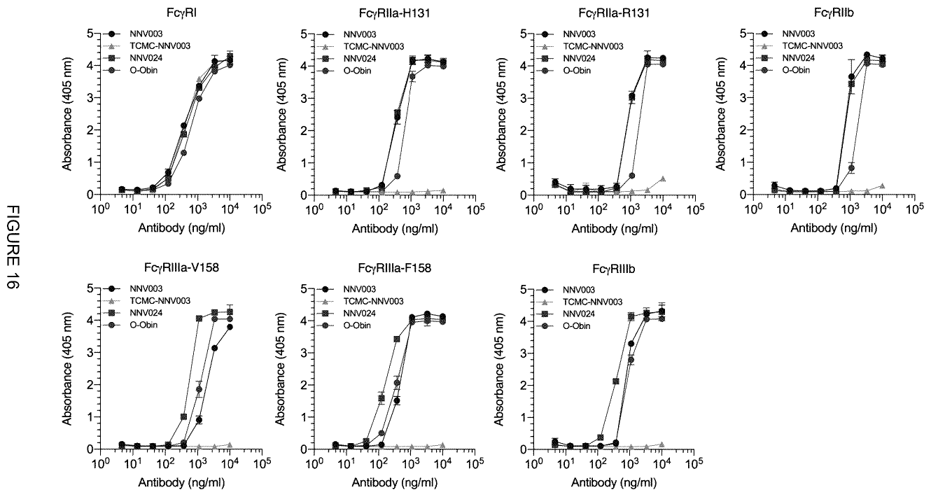

The concentrations of the antibody samples were measured using a Denovix spectrophotometer and analyzed by non-reducing and reducing SDS-PAGE. All four IgG samples migrated according to their expected molecular weights (FIG. 13). The analysis revealed highly comparable bands under both non-reducing and reducing conditions, confirming that the protein concentrations were accurately determined (Table 14). These concentrations were used instead of the concentrations in Table 14.

TABLE Y5.2
Measured protein concentrations

| Antibody | Concentration | |
|---|---|---|
| NNV003 | 6.28 | mg/ml |
| NNV024 | 8.31 | mg/ml |
| TCMC-NNV003 | 12.39 | mg/ml |
| O-Obin | 26.60 | mg/ml |

To ensure that receptor binding is not affected by different coating efficacy in the ELISA set-ups, detection antibodies specific for the IgG Fc or the IgG kappa light chain were used to control the coating levels. As shown in FIG. 14 (a-b), the anti-Fc and anti-LC antibodies bound equally well to all four test antibodies confirming uniform coating levels.

Human FcRn binding was performed at both acidic and neutral pH to mimic endosomal and extracellular conditions. At acidic pH, NNV003, NNV024 and obniutuzumab bound equally well to FcRn while NNV003-TCMC showed slightly reduced binding (FIG. 15a). At neutral pH, NNV003, NNV024 and NNV003-TCMC bound equally well while obniutuzumab bound FcRn somewhat stronger (FIG. 15b, arrow shows the O-Obin curve).

The NNV003 variant bound to all human FcγRs while NNV003-TCMC did not, except for binding to FcγRI (FIG. 16). Binding of NNV024 to FcγRI, FcγRIIa-H131, FcγRIIa-R131 and FcγRIIb was comparable to that of NNV003 while obniutuzumab showed reduced binding activity. Increased binding of obniutuzumab and NNV024 was observed for FcγRIIIa-V158, FcγRIIIa-F158 and FcγRIIIb compared to NNV003.

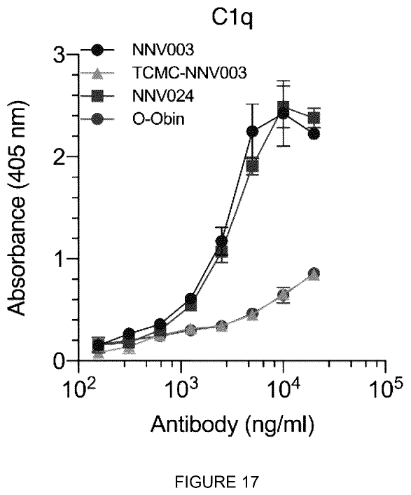

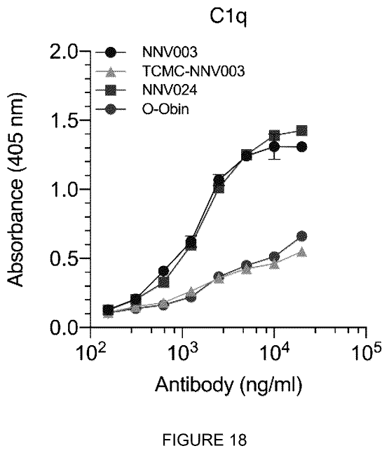

Efficient binding to complement factor C1 q was measured for NNV003 and NNV024 while NNV003-TCMC and obniutuzumab showed strongly reduced binding activity. Due to the overlapping binding responses of obniutuzumab and NNV003 (FIG. 17), the experiment was repeated with a near identical result (FIG. 18).

### Discussion

Human FcRn binding was performed at both acidic and neutral pH to mimic endosomal and extracellular conditions. At acidic pH, NNV003, NNV024 and obniutuzumab bound equally well to FcRn while NNV003-GGG-TCMC (NNV003-TCMC) showed slightly reduced binding. Surface plasmon resonance (SPR) could be used to accurately compare the binding kinetics of NNV003 and NNV003-TCMC. At neutral pH, NNV003, NNV024 and NNV003-TCMC bound equally well while obniutuzumab bound FcRn somewhat stronger. The relatively high binding responses obtained at neutral pH-conditions are a result of direct coating of the antibodies on the plastic and a highly sensitive ELISA assay. As different pH buffers were used, the data cannot be directly compared. Despite this, pH dependent binding appears to be maintained for NNV003, NNV024 and NNV003-TCMC. In contrast, the stronger binding of obniutuzumab at neutral pH may affect its ability to be efficiently released from FcRn during exocytosis and may consequently result in shorter half-life. However, this is highly speculative based on the ELISA results. Surface plasmon resonance (SPR) may be used to accurately compare binding at the two pH conditions and to derive binding kinetics.

At neutral pH, mimicking extracellular conditions, NNV003, NNV024 and NNV003-TCMC bound equally well to FcRn, while obinutuzumab bound somewhat stronger.

Binding of NNV024 to FcγRI, FcγRIIa-H131, FcγRIIa-R131 and FcγRIIb was comparable to that of NNV003 while obinutuzumab showed reduced binding activity. Increased binding of obinutuzumab and NNV024 was observed for FcγRIIIa-V158, FcγRIIIa-F158 and FcγRIIIb compared to NNV003. Efficient binding to complement factor C1 q was measured for NNV003 and NNV024 while NNV003-TCMC and obniutuzumab showed strongly reduced binding activity.

### Example 6—Competitive Binding of Humanized Anti-CD37 Ab Variants Vs DOTA Conjugated NNV003 (NNV009)

The aim of the example is to 1) validate the specificity of NNV020 Abs to bind to CD37, 2) rank the 8 different NNV020 Ab candidates with respect to binding affinity, 3) compare affinities of NNV020 variants to NNV003/009.

### Methods

### Antibodies

Standard ligand (NNV009)
Competitors (variants of NNV020)

TABLE 15
List of antibodies used in the experiment.

| Test items (NNV020) | Manufactured by |
|---|---|
| #1 (AH02871) | Evitria |
| #2 (AH02875) | Genscript |
| #3 (AH02877) | Genscript |
| #4 (AH02879) | Genscript |
| #5 (AH02886) | Evitria |
| #6 (AH02895) | Genscript |
| #7 (AH02871HC + AH02877LC) | Evitria |
| #8 (AH02871HC + AH02877LC + V110D) | Evitria |
| NNV010 (Ab-DOTA) G-384-1601-FDS | NaNo |
| NNV003 (Ab) | NaNo |

### Analysing Concentration of NNV020 Variants

Knowing precise concentration of tested articles (NNV020 variants) is essential for correct determination of affinity parameter (Ki) and comparison of IC₅₀ of all NNV020 variants.

As it is seen from the Table 15, the tested antibodies have been manufactured and received from different companies. The concentration of those Abs that were received from Evitria (AH02871, AH02886, AH02871 HC+AH02877LC, AH02871 HC+AH02877LC+V110D) as well as manufactured at NaNo (NNV003 and NNV010) were determined with A280.

The concentrations of those Ab variants that were received from GenScript were determined using a graphical method to compare bands of non-reduced protein versus 3 concentration standards on a Western blot membrane.

As a part of this example, we therefore determined the concentration of all Abs more precisely with bio-layer interferometry with Protein A as an IgG binder (BLITz instrument) in addition to repeated measurements with A280 with background correction at 320 nm using UV-Vis spectrophotometer Evolution 60S v4.006 (Instrument ID GL056) vs. a standard curve of Bovine Gamma Globulin.

### Preparation of NNV009 (Radiolabeling of NNV010)

A 0.15 mg aliquot of NNV010 (chHH1-DOTA) was radiolabeled with 78.2 MBq of ¹⁷⁷Lu³⁺ by incubation for 30 minutes yielding a radioconjugate (NNV009) with a specific activity (SA) equal to 521.33 MBq/mg. A high SA reduces the counting error.

The activity concentration after radiolabeling was 78.2 MBq/mL and the radiochemical purity was 99.4%.

### Preparation of Cells for Competitive Binding Assay

The competitive binding assay was performed using Ramos cells. 100 million of Ramos cells were harvested, washed twice with 0.5% BSAPBS as described in SOP-LB-006 and up-concentrated into 25 mL of 0.5% BSA in PBS so that the concentration becomes around 4.0 million cells/mL. The cell suspension was left on ice (at 4° C.) for approx. 30 min prior to addition of any Abs.

### Preparation of the Test System and Running the Assay

The Eppendorf LoBind tubes 1.5 mL were used to prepare dilutions (working concentrations) of Abs. As much as 0.2 mL of the cell suspension (800 000 cells/tube), after it has been cooled down for 30 min on ice, was aliquoted to glass 12×75 mm counting tubes. Each tube was pre-labeled with a code specifically determining each sample. The tubes with cells were maintained on ice for whole time course of the experiment except for the time when counted on the gamma counter. The working solution of NNV020 variants (competitors) were added to the cells to make final concentrations as shown in the Table 16. The tubes were agitated on a planetary shaker at 350 RPM, 4° C. (on ice) for 15 min. Then, the radiolabeled standard ligand (NNV009) was added to all tubes to a final concentration 0.4 μg/mL. The tubes were placed on the planetary shaker (GL 064), on ice, and shaken for 4 hours at 350 RPM. After 3.5 hours past from the beginning of incubation, the samples were assayed on Wizard2 gamma counter using protocol “Lu-177 General” (all detectors except for the detector #3 were used). Upon completion the counting, the cells were washed with ice-cold 0.5 mL 0.5% BSAPBS three times. All the tubes were assayed on the Wizard2 gamma counter again using the same sequence and parameters as before the washing.

TABLE 16
The final concentrations of the test substances in the sample tubes

| | | |
|---|---|---|
| | Conc. of | Conc. of |
| Tube | Radiolabeled NNV010 | Competitor, NNV020 |

| nr. | (NNV009) (μg/mL) | (μg/mL) | nM |
|---|---|---|---|
| 01 | 0.4 (=2.7 nM) | 150 | 1000 |
| 02 | 0.4 | 75 | 500 |
| 03 | 0.4 | 15 | 100 |
| 04 | 0.4 | 7.5 | 50 |
| 05 | 0.4 | 1.5 | 10 |
| 06 | 0.4 | 0.75 | 5 |
| 07 | 0.4 | 0.15 | 1 |
| 08 | 0.4 | 0.075 | 0.5 |
| 09 | 0.4 | 0.015 | 0.1 |
| 10 | 0.4 | 0.0075 | 0.05 |
| 11 | 0.4 | 0.0015 | 0.01 |
| 12 | 0.4 | 0.00075 | 0.005 |
| 13 | 0.4 | 0.00015 | 0.001 |

### Data Analysis

The data sets, as CSV files from Wizard2 gamma counter, were first imported, sorted, and normalized in the Excel.

All CPM values of the sample set (13 tubes) were normalized between the highest and the smallest CPM values, according to Formula 2:

$$C P {M}_{n o r m} = \frac{C P M - C P {M}_{min}}{C P {M}_{max} - C P {M}_{min}} \times 100 \qquad Formula 2$$

The normalized CPM values vs. concentration (in nM) were transferred to SigmaPlot 12.0 to calculate IC₅₀ and Ki.

First, a 50% inhibitory concentration of NNV020 (log(IC₅₀), and IC₅₀) were determined using the nonlinear regression function of SigmaPlot to fit the competitive binding curve to the formula according to Formula 3:

$$Y = Nonspecific + \frac{( Total - Nonspecific )}{1 + {10}^{log [ D ] - log ( IC 50 )}} \qquad Formula 3$$

Where the Log[D] is the logarithm of the concentration of the competitor plotted on X axis. Y is the binding of the radioligand (CPM) measured in the various concentrations of NNV020. Total is the binding (CPM) of radiolabeled NNV010 (NNV009) in the absence of NNV020 (i.e. Tube #1). Total, Y, and Nonspecific are all expressed in CPM.

Then, the affinity parameter Ki was calculated using Formula 4:

$${K}_{i}^{N N V 020} = \frac{{IC}_{50}}{1 + \frac{[ N N V 0 \; 09 ]}{{K}_{d}}} \qquad Formula 4$$

Where the [NNV009] is equal to 2.7 nM (see Table 16). Kd is the Kd for NNV009, but not having this value for NNV009, we assumed it was the same as for NNV003 and used that instead. The value of this parameter had been determined earlier as 2.3.

A pre-made macro “Simple Ligand Binding” from the “Tool Box” menu of SigmaPlot 12.0 was used for the calculation of Ki.

### Results

The antibody concentrations were determined using BLITZ and A280 (Table 17).

TABLE 17
Concentrations of the Ab-solutions used in this example.

| | Concentration, |
|---|---|
| Test items (NNV020) | μg/mL |
| #1 (AH02871) | 7770_{A280} |
| #2 (AH02875) | 9.65_{BLITz} |
| #3 (AH02877) | 10.06_{BLITz} |
| #4 (AH02879) | 11.77_{BLITz} |
| #5 (AH02886) | 8000_{A280} |
| | (7870_{BLITz}) |
| #6 (AH02895) | 9.90_{BLITz} |
| #7 (AH02871HC + AH02877LC) | 8200_{A280} |
| #8 (AH02871HC + AH02877LC + V110D) | 7600_{A280} |
| NNV010 (Ab-DOTA) G-384-1601-FDS | 10 000_{A280} |
| NNV003 (Ab) | 5000_{A280} |

The results of the competitive binding were very similar for all eight NNV020 variants (Table 18):

TABLE 18
Values of EC50, affinities, and increase of affinities
relatively to NNV009 for all NNV020 variants.

| # of | NNV020 | | Affinity | Kᵢ(NNV009) |
|---|---|---|---|---|
| NNV020 | variant | EC₅₀ | (Kᵢ)* | Kᵢ(NNV020 var.) |
| #1 | AH02871 | 1.1710 | 0.5387 | 4.27 |
| #2 | AH02875 | 1.1470 | 0.5276 | 4.36 |
| #3 | AH02877 | 0.9175 | 0.4221 | 5.45 |
| #4 | AH02879 | 0.9586 | 0.4410 | 5.22 |
| #5 | AH02886 | 1.1630 | 0.5350 | 4.30 |
| #6 | AH02895 | 0.7178 | 0.3302 | 6.97 |
| #7 | AH02871HC + | 1.1590 | 0.5331 | 4.31 |
| | AH02877LC | | | |
| #8 | AH02871HC + | 1.2420 | 0.5713 | 4.03 |
| | AH02877LC + V110D | | | |

*Ki is the equilibrium dissociation constant for the binding of the unlabeled drug (i.e. competitor, NNV020 in our case). It reflects the concentration of the unlabeled drug that will bind to half the binding sites at equilibrium in the absence of radioligand or other competitors.

### Discussion

The affinity of NNV003 was assumed to be equal to the affinity of the radioimmunoconjugate/conjugate NNV009/NNV010. It is unlikely that the affinity is dramatically different between the antibody and the conjugate. However, there is a possibility to introduce a DOTA moiety into a variable part of immunoglobulin since NNV010 is manufactured using a random DOTA-conjugation technique. The more DOTA moieties are introduced to the antibody-binding part of variable chains or if these moieties stay close to CDR sequences, the more the affinity of the immunoglobulin is affected. However, with an average of 2 DOTAs per Ab it is unlikely that the affinity is affected much.

The affinities of the humanized NNV020 variants were 4-7 times higher than that of the radiolabelled chimeric antibody (NNV009). The best binder was AH02895, with the heavy chain sequence of SEQ ID NO: 70 and the light chain sequence of SEQ ID NO: 75.

### Example 7—Comparison of Antibody-Dependent Cell Mediated Cytotoxicity (ADCC) of Single Treatments of Rituximab, Obinutuzumab, NNV003, NNV023, NNV024 on Daudi and Ramos Cells

The aim of this example was to measure the ability of NNV023 and NNV024 to induce ADCC in Daudi and Ramos cells and compare these effects with the ones of Rituximab, Obinutuzumab, NNV003 tested in the same conditions.

### Methods

### Definition of the NNV023 and NNV023

NNV023—is humanized IgG1 comprising the heavy chain AH02871 of SEQ ID NO: 29 and the light chain AH02877_V110D of SEQ ID NO: 24.

NNV024—is afucosylated NNV023 manufactured using GlymaxX technology.

### Antibodies and Test Articles

Table 19 shows the antibodies tested in this example.

TABLE 19
List of Abs used in this example.

| Test antibody | Concentration | Batch |
|---|---|---|
| Name as in Protocol | [mg/mL] | number |
| NNV003 | 5 | PD-382-BRX005- |
| | | CON-1503 |
| Rituximab | 10 | MabThera, (Lot. |
| | | N7114) |
| Obinituzumab | 25 | Gazyvaro, Roche |
| | | (Lot. H0047) |
| NNV023 | 7.5 | 31709.1-9300 |
| NNV024 | 7.4 | 23708.1-9301 |

### Culturing and Preparation of Target Cells

The cell lines are cultured in RPMI1640 supplemented with GlutamaxX (Gibco, Paisley, UK), 10% heat-inactivated FCS (Gibco) and 1% penicillin-streptomycin mix (Gibco). The incubation takes place in a humidified atmosphere with 5% CO₂ at 37° C. Cell suspensions are diluted 1:5 with pre-warmed medium twice a week (unless otherwise stated based on cell viability). To ensure an exponential growth at the beginning of the experiment, the cells were diluted 2 days before.

### Experiment Procedure in Short

The target cells (Ramos and Daudi) were diluted two days prior the day of the experiment. They were harvested and plated into white flat-bottom 96 plates at concentration of approx. 25 000 cells/well in the 25 μL of the assay buffer/well.

The tested Abs were prepared in triplicates at four different concentrations: 0.001, 0.01, 0.1, and 1.0 μg/mL. The dilutions were prepared in 1.5 mL Eppendorf tubes (10-fold dilution) and then transferred to inner 60 wells of v-bottom transparent 96 well plate following the layout described in the protocol. The solutions from this plate were added to both plates containing the target cells.

The cells and target Abs were co-incubated on the bench (LAF-bench) for 30 min.

After 20 min, the effector cells of Promega ADCC Reporter Bioassay Core kit (Ref.G701A) were thawed, diluted and added to the assay plates with the target cells. The volume of the suspension of the effector cells added to each well was 25 μL.

In the 2-nd experiment the wells H2-H5 were used as “No Abs control”. The cells in these wells were incubated without any Abs.

After addition of the effector cells, the assay plates were incubated for 5 hours at 37° C. in a humidified incubator.

After 5 hours of incubation, the cells were placed on the bench for 15 min to equilibrate the temperature to RT. Bio-Glo luciferase assay reagent was added to the plates (all wells containing cells plus into B1, C1, D1, E1 for background control). Bio-Glo luciferase was added to these wells at the same time as to the other wells and incubated in the dark at RT for 15 or 25 min in the first experiment and for 10 min in the 2^{nd} experiment. The luminescence of the luciferin was measured using Tecan plate reader (integration time of the acquisition in the ₁St experiment was 0.5 and 1.0 sec/well; in the 2^{nd} experiment 0.5 sec/well).

### Data Analysis

The value of the background control (the mean of RLU for B1, C1, D1, E1) was withdrawn from the signals of all wells of the same plate.

The mean RLU and standard deviation of triplicates for each concentration of the test antibody were calculated.

The resulting data set was plotted using dot-line plot and clustered bar plot in the axes RLU vs. [Ab]μg/mL.

A degree of ADCC induction was also calculated relatively to Rituximab for all other test antibodies.

### Results

First, the result of two different incubation times (either 15 or 25 min) with Bio-Glo luciferase assay reagent and two different acquisition time (0.5 and 1.0 sec/well) were evaluated by comparing all four datasets separately for NNV024 and Obinituzumab in Ramos and Daudi cells. The different acquisition parameters tested did not have a great influence the bioluminescence of the samples.

A minor improvement in signal to noise ratio was observed for Ramos cells at 30 min incubation and for Daudi cells at 15 min incubation with luciferase.

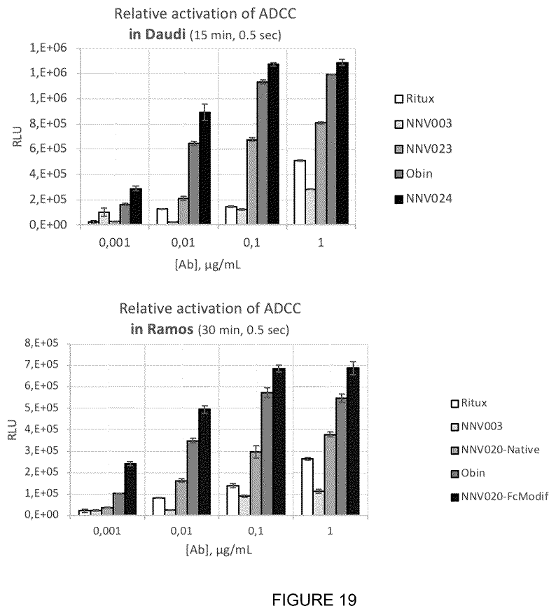

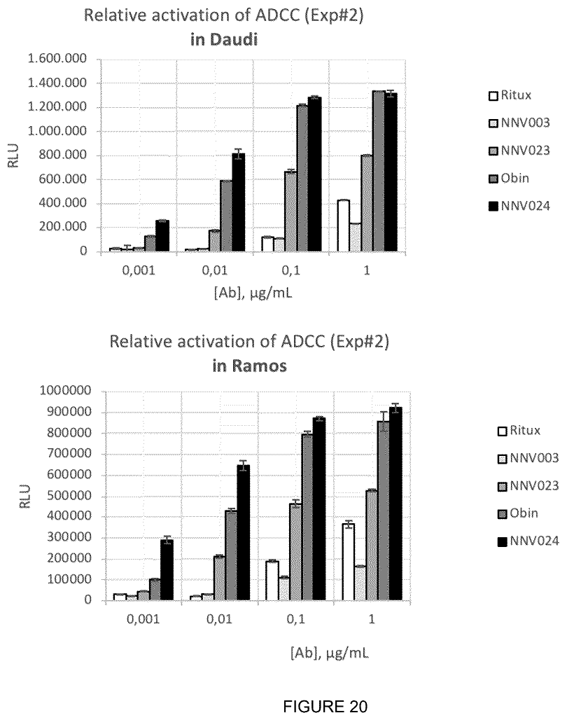

In both cell lines, NNV024 induced the strongest ADCC of all test Abs followed by Obinutuzumab, NNV023, Rituximab, and finally NNV003 in descending order (FIGS. 19 and 20).

### Discussion

The luminescence signal for Daudi cells was almost two times higher than for Ramos cells. Despite this difference, the dose-response relationships were the same for all antibodies (see FIG. 20, the column chart).

The reason for the two times stronger ADCC induction in Daudi cells than in Ramos cells for the anti-CD37 antibodies (NNV003, NNV023, NNV024) might be attributed to a 2.6 times higher number of CD37 antigens in Daudi than in Ramos (Table 20). This same difference in ADCC induction was also apparent for the anti-CD20 antibodies, but here the number of CD20 antigens was similar for the two cell lines. Therefore, it is unlikely that the difference in antigen expression can explain the difference in ADCC induction.

TABLE 20
CD37 and CD20 expression of the target cell lines.

| Cell | CD37 | CD20 |
|---|---|---|
| line | antigens/cell | antigens/cell |
| Ramos | 128 000 ± 40 000 | 279 000 ± 51 000 |
| Daudi | 340 000 ± 5 000 | 307 000 ± 12 000 |

In the second experiment, ADCC induced by NNV024 at 1.0 μg/mL was very similar to the result obtained for Obinutuzumab for that dose. An explanation could be a simple variation of prepared concentration in experiment #1 and #2 even though the protocol was strictly followed.

It is noticeable that the humanized de-immunized antibodies (NNV023 and NNV024) displayed not only better binding to CD37-expressing cells (Example 6), but also greatly improved ability to induce ADCC, which is superior to such a market standard as rituximab and NNV024 displayed superiority even to obinituzumab in majority of the tests.

NNV023 showed stronger ADCC activation than NNV003. The amino acid composition of the Fc region of NNV023 (AH02871HC+AH02877LC V110D), is similar to NNV003. Neither C_{H}2 nor C_{H}3 domains were modified during humanization/de-immunization frameworks (the amino acid structures for both Abs are given in Example 1). During humanization of NNV003 several amino acids were, however, substituted in the framework (FR1, FR2, FR3) regions of the variable region of both heavy and light chains (in VL1 and VH1 domains), see Example 1. These substitutions resulted in a 4.03 higher affinity to CD37 for NNV023 than for NNV003, see example 6. That means that a higher amount of NNV023 than NV003 will be bound to CD37 molecules at the same concentration and this could potentially explain the difference in ADCC induction.

Another reason for the difference in ADCC induction could be that these two antibodies were manufactured at different companies and in different CHO cultures.

When comparing the cytotoxic effect of afucosylated antibodies (obinutuzumab and NNV024) it is important to remember that these Abs could have some different residual amount of fucose. Obinutuzumab is IgG1 with low fucose content (<30%) produced by CHO cell line overexpressing 4-b-N-acetylglucosaminyltransferase (GnT-III) and Golgi a-mannosidase II (aManII). NNV024 was produced in CHO cells co-transfected with bacterial GDP-4-keto-6-deoxy mannose reductase (RMD). There might be a possibility to enhance ADCC of NNV024 even more by using some other than RMD-based approaches manufacturing afucosylated Abs.

For better understanding of the observed differences in ADCC between obinutuzumab and NNV024, a qualitative and quantitative analysis of the glycan content for each of these mAbs should be performed.

Rituximab is a mouse-human chimera IgG1 with human Fc part. The antibody contains natural unmodified glycan and the ADCC effect originates solely from the point mutations introduced to the Fc part during development of the Ab.

The antibodies tested (NNV023 and NNV024) showed the ability to induce ADCC in both Ramos and Daudi cell lines. In both cell lines NNV024 demonstrated superior ADCC activation which maximum was 6-fold (for 0.01 μg/mL in Ramos) to 9.5-fold (for 0.1 μg/mL in Daudi) times higher than that of rituximab for the same doses. The main comparator, obinutuzumab, was just 4.5-fold times (0.025 μg/mL in Ramos) to 8.3-fold times (0.1 μg/mL in Daudi) more potent than rituximab.

The NNV023 Ab had also a good ability for ADCC induction in both cell lines: 2.1-fold time at 0.04 μg/mL in Ramos and 5-fold times at 0.1 μg/mL in Daudi compared to rituximab.

### Example 8—In Vivo Therapy

### Methods

### Test Articles

NNV024 humanized monoclonal antibody and obinutuzumab antibody (Gazyvaro, Roche, batch numbers H0047 and H0115) diluted in 0.9% NaCl to the correct injection concentration (1 mg/ml). Injection solutions were sterile filtered. The injectates were stored at 3-8° C. Protected from light.

### Animals and Housing

70 female CB17-SCID mice, 6-7 weeks of age, were ordered from Envigo, France, and allowed for one week of acclimation prior to study start. The mice were weighed and earmarked during the acclimation period. The mice were housed in a mouse IVC-rack (individually ventilated cages). Five mice were housed per cage in Green line Sealsafe cages from Tecniplast, with a Floor Area: 501 cm². Overall dimensions (W×D×H): 391×199×160 mm. The mice were kept on Alpha Dry irradiated paper bedding (from Brogarden, Denmark) and in irradiated disposal cages GM 500DPSB (purchased from Tecniplast, Italy). Cages were changed once a week. The nesting material, hideaways, drinking bottles, cage lids and the cage barlids were autoclaved prior to use. The mice were provided with filtered drinking water (Danmil filter, Product code DA25CSSS02 19252077, Rating 0.2 MIC http://danmil.dana5.dk/). Water bottles were changed 3 times a week. The mice were fed ad libitum with irradiated rodent diet (Altromin NIH #31 M—from Brogarden, Denmark). The animals were maintained with a 12 hours lighting cycle at a room temperature of 21-24° C. and air relative humidity of ˜40%.

### Experimental Design

The current example included 7 study groups, each with 10 animals per group. The treatment groups were according to the Table 21. This example was a “blinded” study, so the study director and the pathologist did not know what treatment each group was receiving.

TABLE 21
Study groups. Treatment regimen, dose levels, dosing
frequencies and number of mice per group.

| | | Dose | | | Dosage |
|---|---|---|---|---|---|
| Group ID | Treatment | μg/mouse/inj | # mice | # injections | regimen |
| GA and GB | Control - 0.9% NaCl | (100 μL) | 10 | x3 | Twice a week |
| | | | | | for 1.5 weeks |
| AA and AB | NNV024 | 100 | 10 | x1 | Once |
| DA and DB | NNV024 | 100 | 10 | x3 | Twice a week |
| | | | | | for 1.5 weeks |
| EA and EB | NNV024 | 100 | 10 | x6 | Twice a week |
| | | | | | for 3 weeks |
| FA | Obinutuzumab (N-Obin) | 100 | 5 | x1 | Once |
| FB | Obinutuzumab (O-Obin) | 100 | 5 | x1 | Once |
| BA | Obinutuzumab (N-Obin) | 100 | 5 | x3 | Twice a week |
| | | | | | for 1.5 weeks |
| BB | Obinutuzumab (O-Obin) | 100 | 5 | x3 | Twice a week |
| | | | | | for 1.5 weeks |
| CA | Obinutuzumab (N-Obin) | 100 | 5 | x6 | Twice a week |
| | | | | | for 3 weeks |
| CB | Obinutuzumab (O-Obin) | 100 | 5 | x6 | Twice a week |
| | | | | | for 3 weeks |

Due to the need to use two different batches of obinutuzumab (N-Obin within expiry date and 0-Obin expired, but found still to have efficacy in vitro), subgroups A and B were introduced for all treatment groups, to mitigate the risk of having just one obinutuzumab group with a different batch number and to ensure that the study director would not know which group was receiving what treatment.

Mice were injected intravenously with 10 million Daudi cells on day −1. The mice were randomized according to body weight into 14 sub-groups (AA, AB, BA, BB and so on to GA, GB) so that each group had the same average body weight at study start and the next day (day 0) treatment was initiated.

Animals were euthanized at one or more of the following humane endpoints: Hind leg paralysis, weight loss of ≥20% if the mice did not have palpable tumors over a period of one week, kidney tumors >7 and <10 mm in diameter or signs of substantial discomfort.

At termination all animals were necropsied and signs of tumors were investigated.

The Control group was expected to survive for 24-26 days from inoculation of the tumor cells.

The study was scheduled to run for about 5 months (150 days) from inoculation with the possibility of extension if needed.

At certain time points, a statistical analysis of the survival of the mice was performed. The experiment was scheduled to continue until a statistical difference was found between corresponding groups of the two drugs or until it is clear that no statistical difference will be attained by prolonging the experiment.

### Cell Culturing and Preparation for Inoculation

The lymphoma cell line (Daudi) was grown in T75 flasks (Thermo) 25 ml medium/per flask. In 37° C., 5% CO2 humidified incubator. Harvested when 1-1.5 millions cells per ml, viability minimum 85% at harvest (determined by counting live cells vs. dead cells).

Cell culture medium RPMI-1640—GlutaMAX (Life Technologies Cat no 72400-021) supplemented with 10% inactivated FBS (Life Technologies cat no 10270), 1% Pen Strep (Life Technologies cat no 15140-122) and 1 mM Sodium Pyruvate (Sigma cat no p5280).

The mice were scheduled to be injected with the tumor cells suspended in RPMI medium from a fresh bottle without any supplements within 1.5-2 hours after they were placed in transport vials.

The cells were kept on ice once placed in the transport vial until injected into the mice.

The mice were injected in the lateral tail vein with 10 million cells pr. animal in a volume of 100 μl per mouse. The animals were specifically observed for 15-20 minutes with regards to registering any treatment related adverse effect after the injection.

### Injection of Test Articles

The mice were 8 weeks of age when treatment was initiated and according to Envigo's website female mice in that age will weigh on average 18.4 gr. Dosing volume was set to 100 μl per mouse. The dose was fixed at 100 μg (100 μl) independent of body weight.

### Necropsy Protocol

At termination, all mice were necropsied and at least the following organs inspected for signs of tumors: Skull, eyes, brown adipose tissue (located between shoulders), ovaries and uterus (broad ligament), kidneys, cervical lymph nodes (superficial and deep), abdominal lymph nodes located along the aorta and kidneys, tissue along columna in thorax and abdomen (brown adipose tissue and muscle), on the surface of the ventriculus, lungs, spleen and liver.

Length, width and weight of tumors were measured when possible (applied specially to kidney tumors). If an animal was euthanized with signs of substantial discomfort but necropsy did not reveal any clear signs of tumors, organs were collected for histopathology analysis of the organs listed below. Organs were not collected from animals with hindleg paralysis even though they did not have visible tumors during necropsy because previous experience with this model showed that all mice with hindleg paralysis had tumor infiltration in the spinal canal.

At study end, when symptom free animals were euthanized, the following organs were collected for histopathological evaluation to investigate microscopic tumor infiltration: femur (bone marrow), brain, skull, lung, liver, spleen, easily accessible lymph nodes, ovaries and uterus, kidneys, the vertebral column (spine), muscle, eye, brown adipose tissue, tumors. The organs were fixed in 10% buffered formalin and further processed for histopathological.

### Results

After inoculation with Daudi tumor cells on Day −1, the mice were randomized into 7 different treatment groups (n=10) and each treatment group were divided into two subgroups according to body weight, so that each group had similar average body weight at treatment start (Table 22). Each treatment group was divided into two subgroups. The obinutuzumab subgroups received different batches of the drug referred to as New (within expiration date) and Old (expired but found still to have efficacy in vitro). Each group received 1-6 treatments of the relevant drug. Treatment was initiated 1 day post inoculation of the tumor cells on Day 0.

TABLE 22
Randomization into 7 treatment groups.

| | | Treatment and number | Average | SD |
|---|---|---|---|---|
| Group ID | | of treatments (*x) | weight | (weight) |
| A | AA | NNV024 *1 | 19.5 | 0.8 |
| | AB | | 19.5 | 1.4 |
| B | BA | Obinutuzumab: New*2, Old *1 | 19.6 | 0.7 |
| | BB | Obinutuzumab: Old*3 | 19.6 | 1.4 |
| C | CA | Obinutuzumab: New*2, Old *4 | 19.5 | 1.5 |
| | CB | Obinutuzumab: Old*6 | 19.5 | 1.5 |
| D | DA | NNV024 *3 | 19.5 | 1.3 |
| | DB | | 19.5 | 1.5 |
| E | EA | NNV024*6 | 19.5 | 1.2 |
| | EB | | 19.5 | 1.4 |
| F | FA | Obinutuzumab: New*1 | 19.5 | 1.4 |
| | FB | Obinutuzumab: Old*1 | 19.6 | 2.0 |
| G | GA | Control*3 | 19.5 | 1.4 |
| | GB | | 19.6 | 2.0 |

Randomization into 7 treatment groups according to average body weight (also including standard deviation (SD)) at the time of i.v. inoculation of the tumor cells. Number of treatments per group is indicated with an asterix (*) and a number behind the asterix refers to number of times the group received the treatment in question.

Treatment groups were divided into two subgroups because of the two different Obinutuzumab batches so that it was possible to investigate differences in effect between the two subgroups. Table 23 shows that at the subgroup level, the median survival of each subgroup from the control and obinutuzumab*6 treatments were identical, 23 days and 76 days, respectively. The subgroups of the Obinutuzumab that received 1 treatment and 3 treatments did not have identical survival times, but the difference is not statistically significant (p>0.05, Kaplan Meier: Log-rank (Mantel-Cox Test)).

TABLE 23
Median survival of Obinutuzumab- and control subgroups.

| | | | Median | P- value |
|---|---|---|---|---|
| | | Treatment | Survival | summary |
| | | and number | in days from | subgroup |
| Group ID | | of treatments (*x) | treatment start | A vs B |
| B | BA | Obinutuzumab: New*2, Old *1 | 83 | — |
| | BB | Obinutuzumab: Old *3 | 101 | 0.8800 |
| C | CA | Obinutuzumab: New *2, Old *4 | 76 | — |
| | CB | Obinutuzumab: Old *6 | 76 | 0.7105 |
| F | FA | Obinutuzumab: New *1 | 101 | — |
| | FB | Obinutuzumab: Old *1 | 80 | 0.7887 |
| G | GA | Control*3 (0.9% NaCl) | 23 | — |
| | GB | | 23 | 0.7245 |

The obinutuzumab subgroups received different batches of the drug referred to as New (within expiration date) and Old (expired but found still to have efficacy in vitro). Each group received 1-6 treatments of the relevant drug. Number of treatments per group is indicated with an asterix (*) and a number behind the asterix refers to number of times the group received the treatment. The difference in survival times between subgroups A and B was not statistically significant (p > 0.05), Kaplan Meier: Log-rank (Mantel-Cox Test).

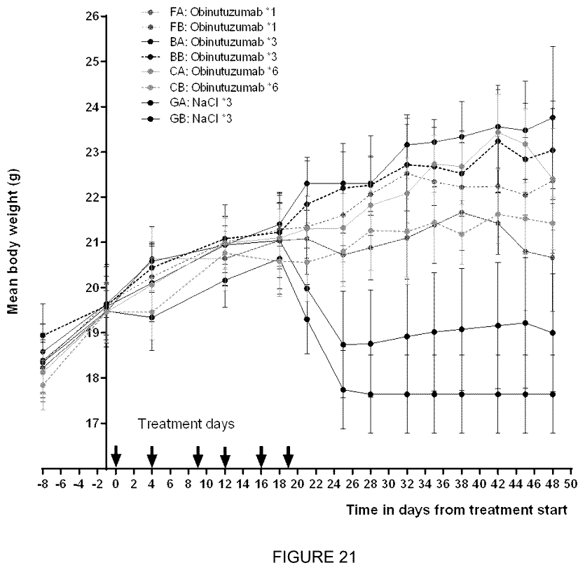

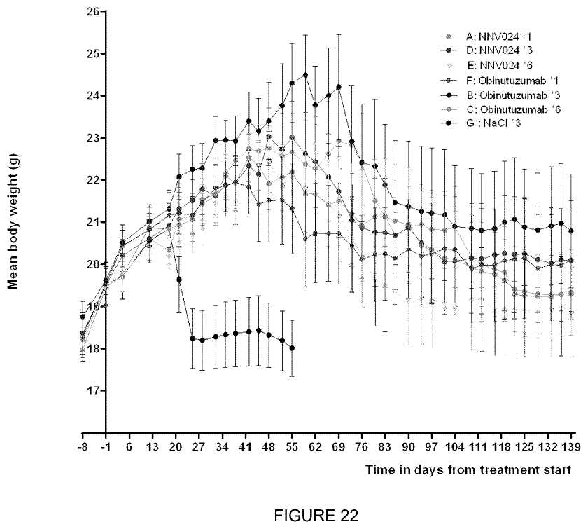

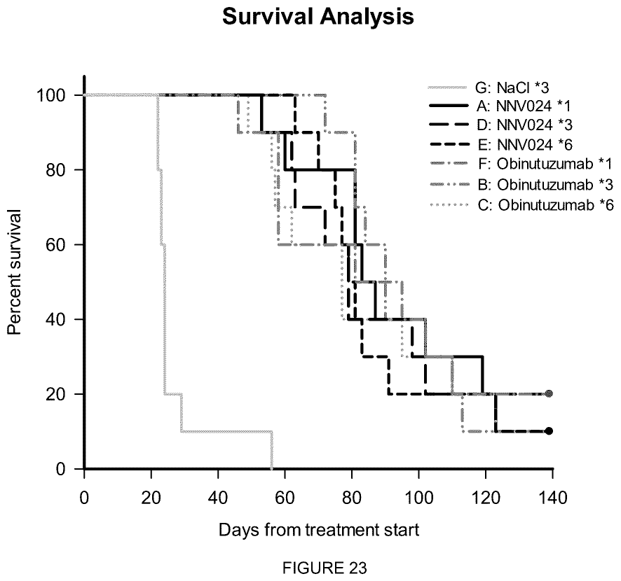

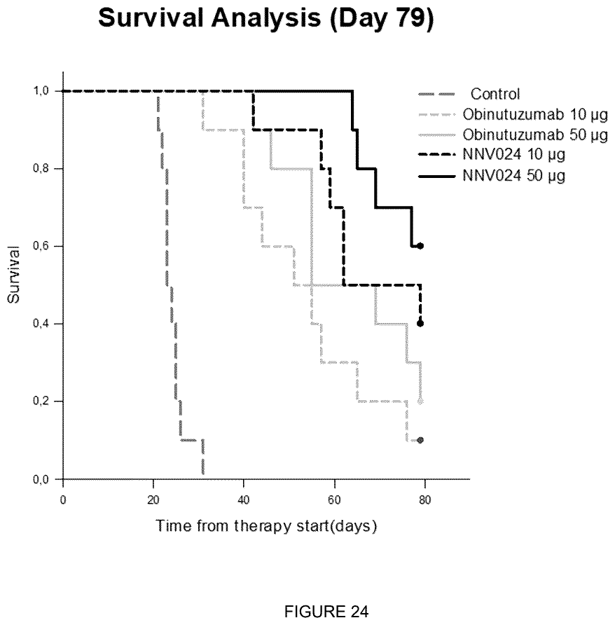

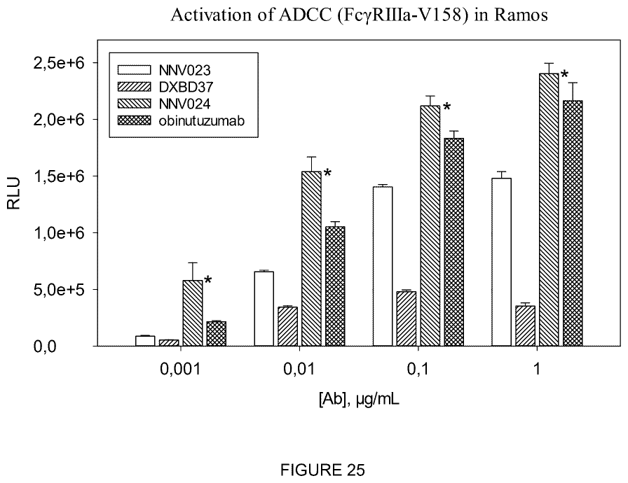

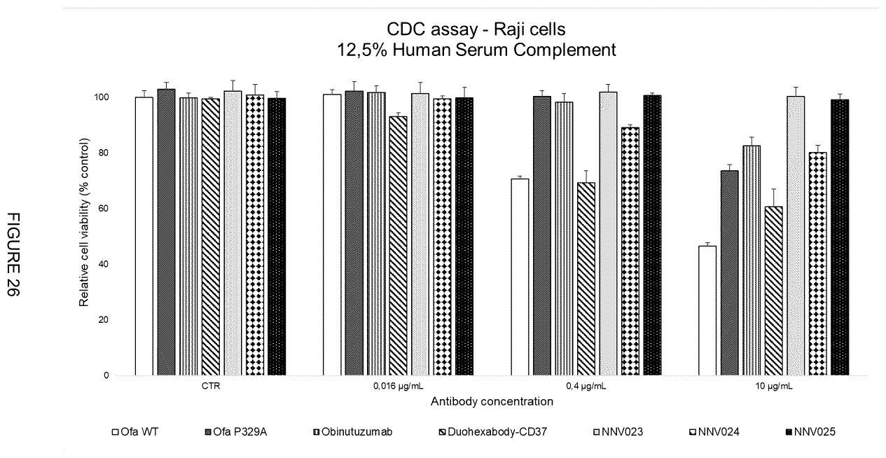

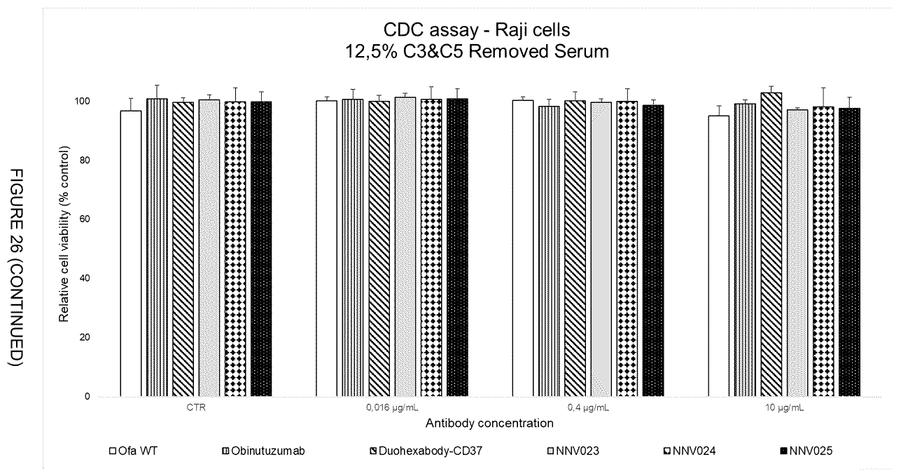

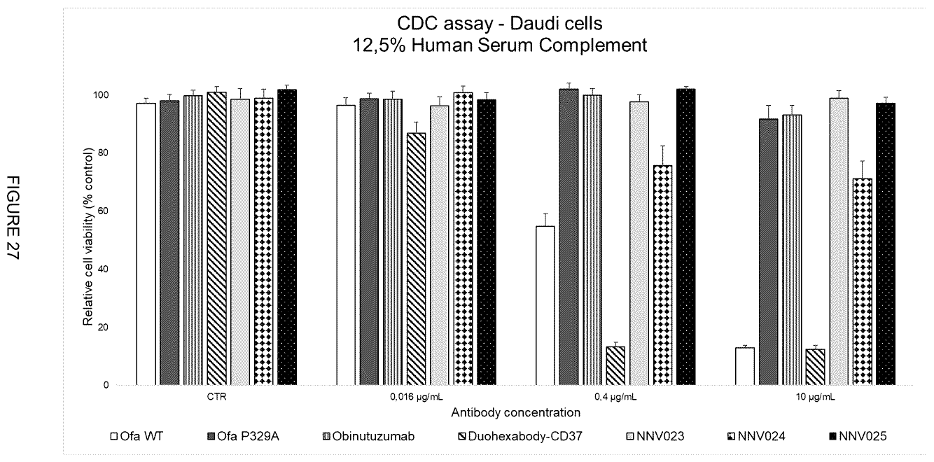

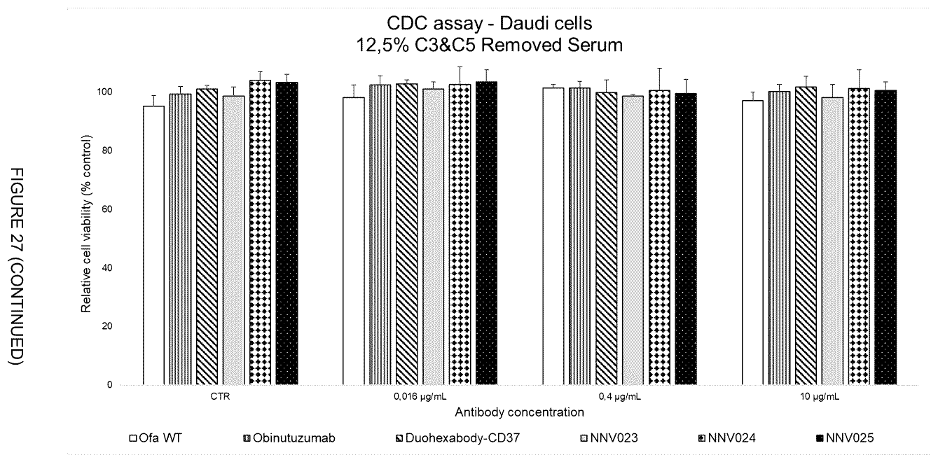

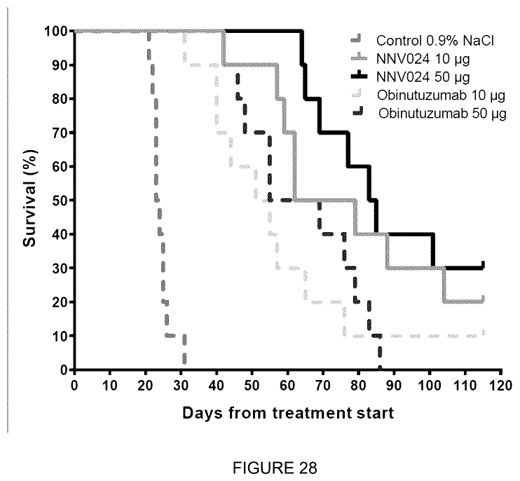

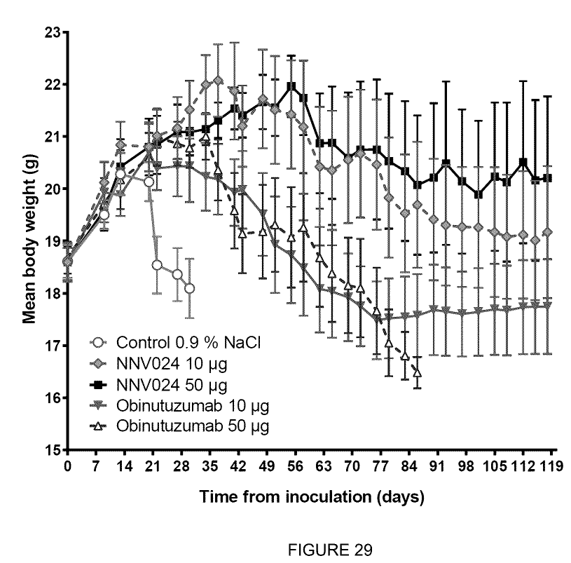

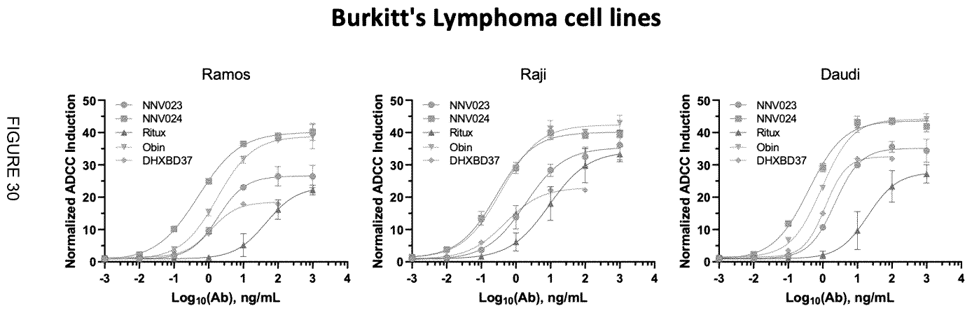

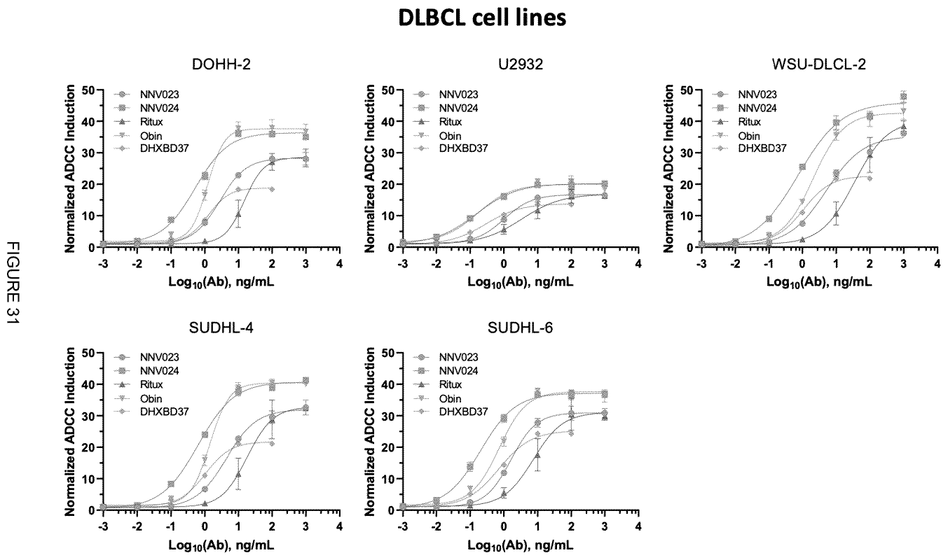

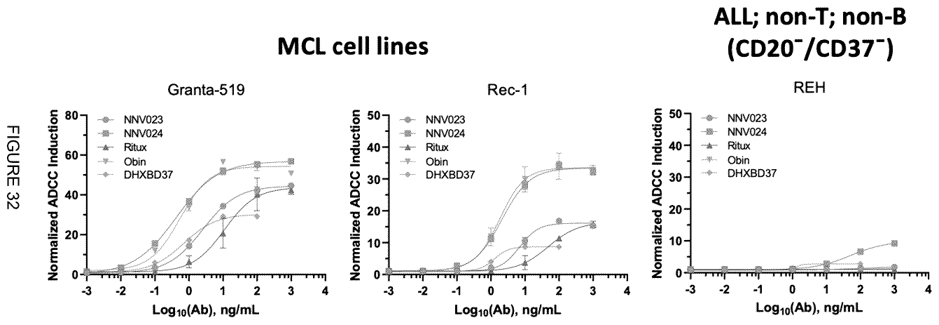

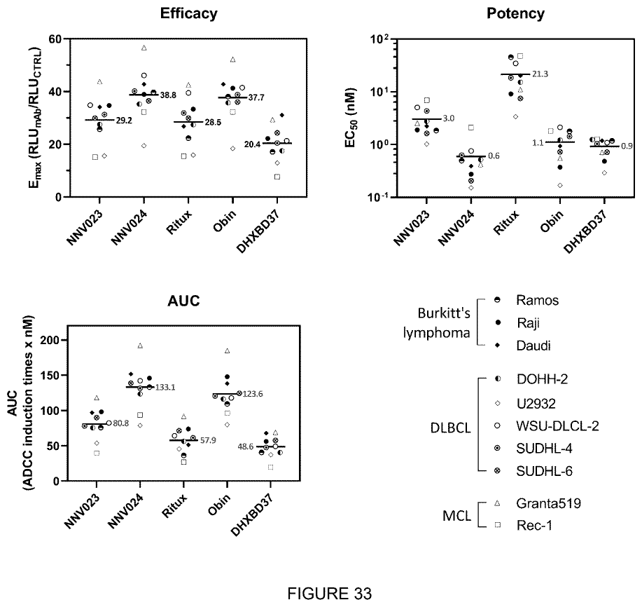

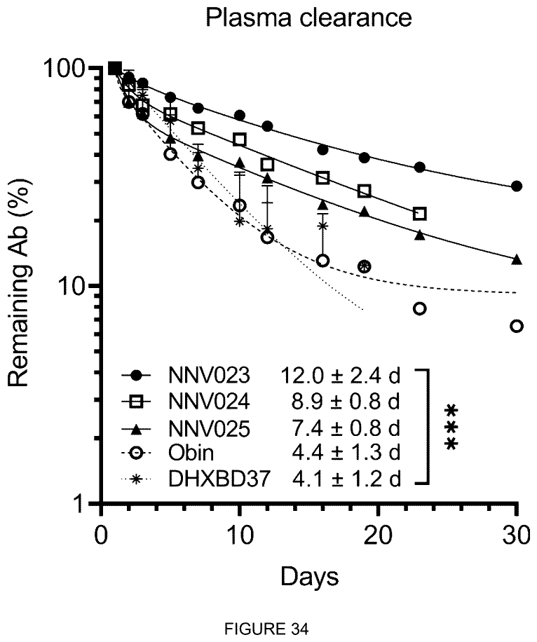

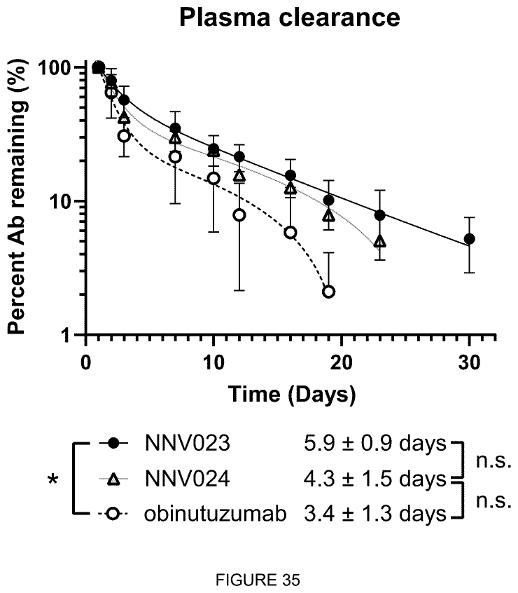

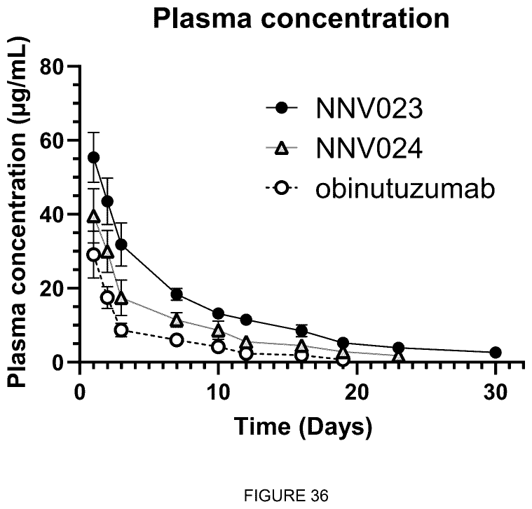

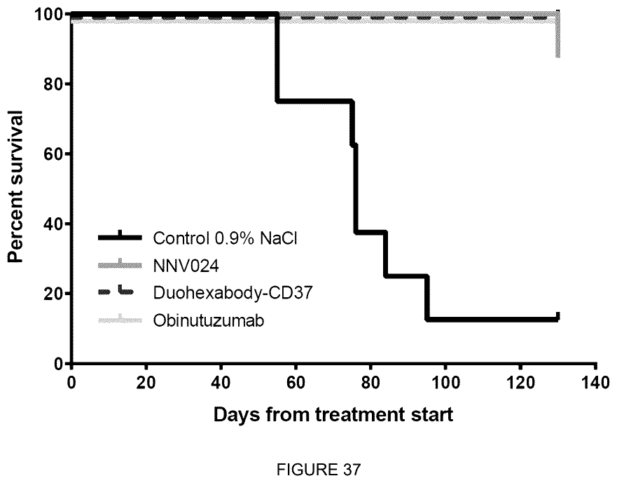

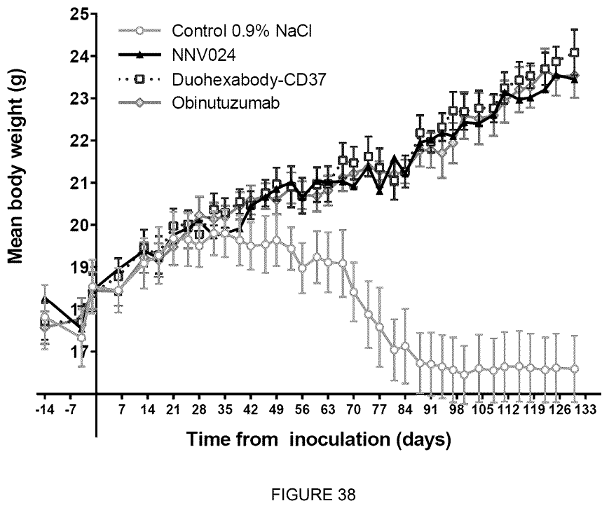

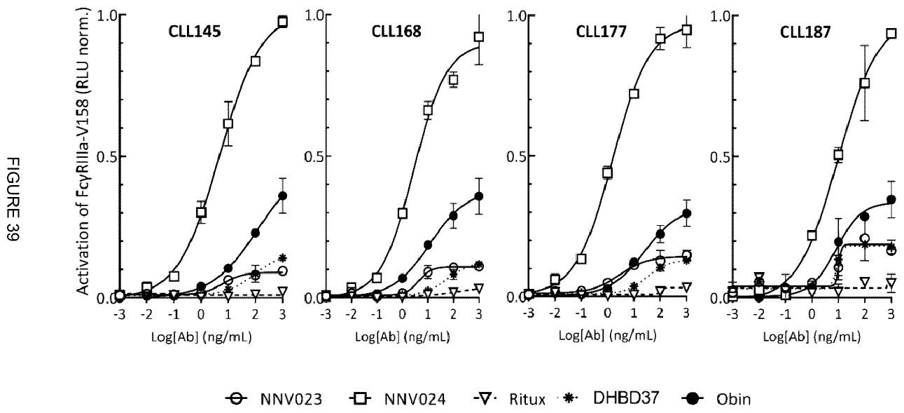

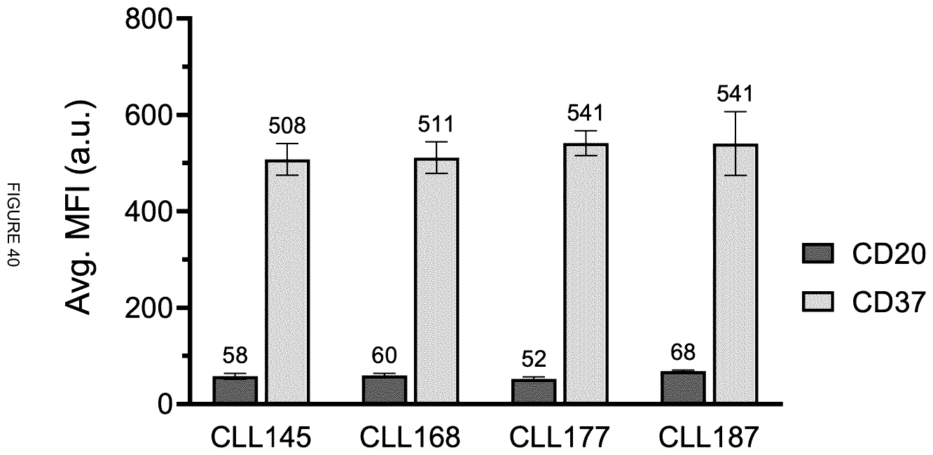

FIG. 21 presents mean body weights of each obinutuzumab and Control subgroup from 8 days prior to treatment start until 49 days post treatment start. During this period, all subgroups had small differences. However, the difference in the obinutuzumab subgroups did not appear to be greater than in the saline subgroups. It was therefore decided to combine the subgroups and stop following body weight changes at the subgroup levels.

FIG. 22 presents mean body weights of each treatment group from 8 days prior to treatment start until study end (Day 139). The mice were weighed weekly the first two weeks after treatment was initiated and at least twice a week after that. 18 days from treatment start the Control group (Group G) started losing weight rapidly and all mice except one had been euthanized 26 days after treatment was initiated. The last mouse in this group was euthanized on day 55. The other treatment groups all gained weight until day 38, when the first of the treatment groups started to lose weight (Group F). Next group to start losing weight was group C (after day 45) and groups and D followed closely around day 55. Groups A and B appeared be more resilient and started losing weight around day 70. Group B appeared to gain more rapidly weight than the remaining treatment groups up until day 69 when the groups mean started to decline. Two mice in group B had large abdominal tumors when euthanized on days 109 and 112. These two mice did however not account for this difference alone between Group B and the other treatment groups and no explanation is obvious.

Table 24 summarizes clinical findings at the time of euthanasia in all 7 treatment groups. The most prevalent clinical findings at the time of euthanasia was different degrees of paresis or paralysis 20% weight loss and reduced activity.

TABLE 24
Summary of clinical symptoms.

| Symptoms present at the | Number of mice per group (n = 10) with symptoms present | | | | | | |
|---|---|---|---|---|---|---|---|
| time of euthanzia | A: NN*1 | B: Obi*3 | C: Obi*6 | D: NN*3 | E: NN*6 | F: Obi*1 | G: Control*3 |
| Front leg paralysis (grade 3) | | | | | | 1 | |
| Front leg paresis (grade 2) | 1 | | | | | | |
| Hind leg paralysis (grade 3) | 2 | 2 | 3 | 3 | 4 | | 7 |
| Hind leg paralysis (grade 2) | | 1* | 1* | 1* | 1* | | 3 |
| Hind leg paresis (grade 2) | 2 | 2(+1*) | 2(+1*) | 3 (+1*) | 2(+1*) | 3 | |
| | | (+2**) | | | | | |
| Hind leg paresis (grade1) | 1 | 1 (+2**) | 1 | | 1 | 1 | |
| Tail paralized | 2 | 2 | 2 | 1 | 2 | 3 | |
| Weight loss (grade 1) <10% | | | 1 | | | | 2 |
| Weight loss (grade 2) 10-20% | 1 | 3 | 3 | 3 | 5 | 1 | 6 |
| Weight loss (grade 3): >20% | 8 | 5 | 6 | 5 | 4 | 6 | 2 |
| Reduced activity | 6 | 6 | 6 | 7 | 8 | 4 | 9 |
| Increased activity | | | | | | 1 | |
| Labored respriation | 1 | 2 | 2 | | 1 | 1 | |
| Timed | 3 | 1 | | 3 | 1 | | 1 |
| Rough hair coat | 2 | 3 | 3 | 3 | 4 | 4 | 1 |
| Piloerection | 1 | | 1 | | | | 5 |
| Hunced | 2 | 2 | 2 | | 2 | | |
| Protruding scull | 1 | 1 | | | | 1 | |
| Tumor on os. Incisivium | 1 | | 2 | | 1 | 1 | |
| Visable tumor on hind leg | | | | 1 | 1 | 1 | |
| Visual tumor over m. | | 1 | 1 | | | | |
| Erectus | | | | | | | |
| Ocular tumor | | | 1 | 1 | | | |
| Tail with swellings | | 1 | | | | | |
| ulcerations on tail | | 1 | | | | 2 | |
| Tumor between | | | 1 | | | | |
| shoulderblades | | | | | | | |
| Tumor (>2000 mm3) | | | | | | 1 | |
| Palpable abdominal tumor | | 2 | | | | | |
| 20*20 mm | | | | | | | |
| Ulceration on thigh | | | | 1 | | | |
| Number of mice survivin >140 days | 1 | 1 | 0 | 2 | 1 | 1 | 0 |

Summary of clinical symptoms present at the time of euthanasia in each treatment group (A-G). Numbers indicate how many animals in each group had a given clinical sign at the time of euthanasia. In some cases, one animal would have intermittently signs of paralysis and paresis or different grades of paresis. In these cases, there is an asterisk or two above the number and that indicates the same animal in each group with two different types of symptoms interchanging.
NN = NNV024, Obi = obinutuzumab, Control = NaCl.
Asterix and numbers behind drug name indicate number of treatments.

Table 25 presents a summary of visible tumors in each treatment group and median survival times from treatment start. All mice in the control group had tumors in the same location (kidneys and ovaries); one mouse in this group that survived longer than most of the other mice in this group also had tumors in abdominal lymph nodes. Only three mice in 3 different treatment groups had visible tumors on kidneys. All treatment groups except Group C (obinutuzumab*6 treatments) had 1-3 mice with tumors in ovaries. The most common location for tumors were ovaries (19), cervical Inn (17), muscles (in. Gluteus superficialis and m. Erector spinae) (17), abdominal lymph nodes (renal (11) and iliac (14)). Tumors in other location were less common (<10 mice).

TABLE 25
Summary of tumors found during necropsy in each group (A-G).

| | Number of mice per group (A-G) with tumors in specific location | | | | | | | |
|---|---|---|---|---|---|---|---|---|
| Location of tumors | A: NN*1 | B: Obi*3 | C: Obi*6 | D: NN*3 | E: NN*6 | F: Obi*1 | G: NaCl*3 | Sum |
| Kidneys | | 1 | 1 | | | 2 | 10 | 13 |
| Ovaries | 1 | 2 | | 1 | 3 | 2 | 10 | 19 |
| Uterus | | | | | 1 | | | 1 |
| Brown adipose tissue | 1 | 1 | 1 | | | 1 | | 4 |
| between shoulders | | | | | | | | |
| Brown adipose tissue | | | 1 | 1 | | 1 | | 3 |
| by ovaries | | | | | | | | |
| Cervical Inn | 2 | 2 | 5 | 3 | 2 | 3 | | 17 |
| Renal Inn or brown | 1 | 3 | 1 | 1 | 2 | 2 | 1 | 11 |
| adipose tissue | | | | | | | | |
| Iliaca Inn or brown | 1 | 4 | 2 | 2 | 2 | 2 | 1 | 14 |
| adipose tissue | | | | | | | | |
| m.gluteus superficialis | 2 | 3 | 2 | 5 | 2 | 3 | | 17 |
| and m.erector spinae | | | | | | | | |
| m.lumbosacrocaudalis | | 2 | | 1 | 1 | | | 4 |
| ventralis lateralis | | | | | | | | |
| Pancreas | | | | | 1 | 1 | | 2 |
| Ventriculus/ | 1 | 1 | 1 | 1 | 2 | | | 6 |
| duodenum | | | | | | | | |
| Scull | 1 | 1 | | 1 | 1 | 3 | | 7 |
| Eyes | | | 2 | 2 | | | | 4 |
| Along columna in | | 1 | 1 | | 1 | | | 3 |
| abdomen | | | | | | | | |
| Along columna in | | 3 | 1 | 2 | 1 | | | 7 |
| Thorax | | | | | | | | |
| Tail | | 1 | | | | 1 | | 2 |
| At base of tail | | 1 | | | | | | 1 |
| On Os. Incisivum | 1 | 1 | 3 | | 1 | 2 | | 8 |
| Mediastinal tumor | | | | | | 1 | | 1 |
| Tumor >2000 mm3 | | | | | | 1 | | 1 |
| Abdominal tumor | | 2 | | | | | | 2 |
| 20*20 mm | | | | | | | | |
| Median survival in | 84 | 89 | 76 | 78 | 79 | 87 | 23 | |
| days from treatment | | | | | | | | |
| start | | | | | | | | |

Numbers indicate how many animals in each group had tumors in each location.
NN = NNV024, Obi = obinutuzumab, Control = NaCl.
Asterix and numbers behind drug name indicate number of treatments.

Histology slides were analyzed from 15 mice. Results are presented in Table 26. Tumor infiltration was noted in 10/15 investigated individuals. Tumor infiltrations were consistent with lymphoma and were composed of poorly demarcated sheets of medium sized round cells (lymphoblasts) with moderate cytoplasm, a central round nucleus with coarse chromatin and 1-5 nucleoli. Mitotic figures were moderate to abundant.

Tumor infiltration was detected in vertebrae in 7 individuals from groups A (3/5 individuals), C (1/1 individuals), D (1/3 individuals) and E (2/2 individuals). In two of these individuals (from groups A and E), there was tumor infiltration in thoracic vertebrae. In two individuals from group B and F, only tail was investigated. In these individuals, there was tumor infiltration in vertebrae of the tail.

Tumor infiltration was commonly noted in surrounding skeletal muscle, spinal cord meninges and spinal cord.

In addition, tumor infiltration was noted in brain and/or brain meninges (4 individuals), skeletal muscle (3 individuals), skull and skull periosteum (3 individuals) and femur (2 individuals).

Histopathological changes not related to tumor infiltration was only noted in one individual, where there was a cyst in the spinal cord. This was regarded as an incidental lesion.

TABLE 26
Summary of histology findings

| Location | Groups and animal numbers | | | | | | | | | | | | | | |
|---|---|---|---|---|---|---|---|---|---|---|---|---|---|---|---|
| of | A: NN*1 | | | | | B: Obi*3 | C: Obi*6 | D: NN*3 | | | E: NN*6 | | F: Obi*1 | | |
| tumors | A30 | A29 | A15 | A40 | A52 | B3 | C28 | D70 | D32 | D44 | E25 | E4 | F1 | F39 | F61 |

| | | | | | | | | | | |
|---|---|---|---|---|---|---|---|---|---|---|
| Muscle | | | x | | | | x | x | | |
| Skull | | | | | | x | x | x | | |
| Brain | | x | | | | | x | x | x | |
| Femur | | | | | | x | x | | | |
| Throracic | | | | x | | | | x | | |
| vertebrae | | | | | | | | | | |
| Total | x | | x | x | | x | x | x | x | |
| vertebrae | | | | | | | | | | |
| Tail | | | | | x | | | | | x |

X indicates that tumor was present in the organ in question.
NN = NNV024, Obi = obinutuzumab, asterix and numbers behind drug name indicate number of treatments.
In mice B3 and F1 the tail was the only organ collected.
Grey shading indicates animals that survived to the end of the study period without signs of a humane endpoint and did not have visible signs of tumors during necropsy.

The data were analyzed for survival (Humane endpoint was Hind leg paralysis, Weight loss of 15% plus signs of discomfort or Weight loss of 20% without signs of substantial discomfort) using Kaplan Meier: Log-rank (Mantel-Cox Test (GraphPad Prism 8.3.0)). The data are presented in FIG. 23 and in Table 27.

The survival analysis revealed that all treatment groups had significantly better survival than the control group, p<0.0001 (Kaplan Meier: Log-rank (Mantel-Cox Test). All treatment regimens tested in this study gave statistically significantly better survival as compared to the control group (P<0.0001 Kaplan Meier: Log-rank (Mantel-Cox Test). Treatments with NNV024 and obinutuzumab did not differ significantly (p>0.05, Kaplan Meier: Log-rank (Mantel-Cox Test) and median survival of the antibody treatment groups ranged from 76-89 days, whereas the control group had a median survival of 23 days.

The obinutuzumab treatment group that received 3 treatments had the longest median survival (89 days) and the obinutuzumab group that received the 6 treatments had the shortest median survival (76 days). These two groups differed by 13 days; the difference is however not statistically significant (P>0.05) (Kaplan Meier: Log-rank (Mantel-Cox Test). Furthermore, there was not a statistically significant difference in survival between any of the treatment groups (A-F).

The NNV024 treatment group that received 1 treatment had the longest median survival (84 days) when compared to the other NNV024 groups with one mouse still surviving at the end of the study period without any signs of tumors. The NNV024 group that received 3 treatments had a median survival of 78 days and 2 mice still surviving at the end of the study period with no signs of tumors.

The NNV024 group that received 6 treatments had a median survival of 79 days, with one mouse still surviving at the end of the study period but histopathology revealed signs of tumors.

TABLE 27
Median survival of 7 groups of CB-17-Scid mice with
i.v. injected Daudi-lymphoma cells million cells).

| | | Median | | % animals |
|---|---|---|---|---|
| | | Survival | | surviving at |
| | Treatment | (days from | % animals | day 139 |
| | (*number of | treatment | surviving | without signs |
| Group ID | treatments) | start) | at day 139 | of tumor |
| G | 0.9% NaCl *3 | 23 | 0 | 0 |
| A | NNV024 *1 | 84 | 10 | 10 |
| D | NNV024 *3 | 78 | 20 | 20 |
| E | NNV024 *6 | 79 | 10 | 0 |
| F | Obinutuzumab*1 | 87 | 20 | 20 |
| B | Obinutuzumab*3 | 89 | 10 | 0 |
| C | Obinutuzumab*6 | 76 | 10 | 0 |

One day after inoculation the mice received 7 different treatments (10 mice per group). A humane end point was reached when one or more of the following clinical findings were present: Hind leg paralysis, Weight loss of ≥15% plus signs of substantial discomfort, Weight loss of ≥20%. All treatment groups had significantly better survival than the control group (Group G), p < 0.0001. Kaplan Meier: Log-rank (Mantel-Cox Test)

### Discussion

The aim of the present example was to investigate the efficacy potential of the humanized CD37-targeting antibody NNV024 and compare it to the well-established CD20-targeting antibody obinutuzumab in an animal model mimicking disseminated B-cell malignancy.

Two batches of obinutuzumab were used in this example (N-Obin within expiry date and O-Obin expired but found still to have efficacy in vitro). Subgroups a and b were therefore introduced to mitigate the risk of having just one obinutuzumab group with a different batch number. Body weights did not differ more between the control subgroups and the obinutuzumab subgroups.

Furthermore, the survival times of the obinutuzumab subgroups did not differ significantly in any of the treatment groups (B, C and F). It is therefore concluded that the two batches of obinutuzumab appeared to be equally effective as treatment regimen.

The mice were followed for up to 140 days after start of therapy and there was a significant therapeutic effect of all treatments as compared with control. Treatment with NNV024 appears to be equally effective as obinutuzumab in treating disseminating B-cell malignancy in this mouse model.

There were no significant differences between one, three and six injections of either NNV024 or obintuzumab, which may indicate that the dose of 100 mg/mouse per injection was too high. With lower dose of antibody per injection it should be possible to observe differences between single and multiple injections and maybe also between NNV024 and Obinutuzumab since NNV024 was shown to be more effective than obinituzumab in inducing ADCC at the lower concentrations (see example 7).

Both NNV024 and obinituzumab were effective for treatment of mice with intravenous Daudi cells, but no differences were seen between single and multiple injections and between the two antibodies.

### Example 9 Therapeutic Effect of NNV024 and Obinutuzumab in SCID Mice Injected with Intravenous Daudi Cells

### Aim

The aim of this study is to compare the therapeutic effect of a single injection of NNV024 with obinutuzumab in mice with intravenous NHL Daudi model.

### Materials and Methods

Test articles: Vials containing the necessary amounts of NNV024 humanized monoclonal antibody (Nordic Nanovector) (7.4 mg/ml), obinutuzumab antibody (Gazyvaro, Roche) (5 mg/ml diluted in 0.9% NaCl from 25 mg/ml stock), and 0.9% NaCl were provided by Nordic Nanovector. All the solutions in the shipped vials were sterile filtered and stored at 3-8° C., protected from light. Animals and housing: 50 female CB17-SCID mice, 6-7 weeks of age were ordered from Envigo, France and allowed to acclimate for 5 days prior to study start. The mice will be weighed and earmarked in the acclimation week. The mice were housed in an individually ventilated cages, 5 mice per cage. The nesting material, hideaways, drinking bottles, cage bars lids and lids were autoclaved prior to use. The mice were fed ad libidum with irradiated rodent diet. The animals were maintained with a 12-hour lighting cycle at a room temperature of 21-23° C. and air relative humidity of 40%.

Cell culturing: Daudi cells in suspension cells were grown in T75 flasks (Thermo) 25 ml medium/per flask in 37° C., 5% CO₂ humidified incubator. Cells were harvested when the cell concentration was 1-1.5 million cells per ml, viability was minimum 85% at harvest. Cell culture medium RPMI-1640—GlutaMAX (Life Technologies Cat no 72400-021) supplemented with 10% inactivated FBS (Life Technologies cat no 10270), 1% PenStrep (Life Technologies cat no 15140-122) and 1 mM Sodium Pyruvate (Sigma cat no p5280).

Injection of cells: The mice were injected with tumor cells suspended in RPMI medium from a fresh bottle without any supplements within 1.5-2 hours after they have been placed in transport vials. The cells were kept on ice once placed in the transport vial until injected into the mice. The mice were injected in the lateral tail vein with 10 million cells pr. animal in a volume of 100 μl per mouse. The animals were specifically observed for 15-20 minutes in regard to register any treatment related adverse effect after the injection.

Experimental design: The current study includes 5 study groups, each with 10 animals per group (Table 28). Each mouse will be injected once with 100 ml of the indicated injection solution. This study was a “blinded” study, so the study director and the pathologist did not know what treatment each group received. On day 0 the mice were inoculated intravenously with 10 million Daudi cells, weighed and randomized according to body weight into 5 groups so that each group had similar average body weight at study start. The next day (day 1) treatment was initiated. Animals were euthanized at one or more of the following humane endpoinds: Hind leg paralysis, overall weight loss of 20% or weight loss of 15% over a period of one week if signs of discomfort, kidney tumors 7-10 mm in diameter or larger, or signs of substantial discomfort.

TABLE 28
Study groups.

| Treatment | Dose μg/mouse/injection |
|---|---|
| Control - Formulation Buffer (0.9% NaCl) | 100 μL |
| NNV024 | 10 mg |
| NNV024 | 50 mg |
| Obinutuzumab | 10 mg |
| Obinutuzumab | 50 mg |

### Results

Four weeks after start of treatment all mice in the control group were euthanized while all the mice in the treatment groups were alive. At 11 weeks after start of treatment the survival of mice treated with 50 mg or 10 mg NNV024 was 60% and 40%, respectively, while the survival was 20% and 10% for mice treated with 50 or 10 mg obinutuzumab (Table 29).

TABLE 29
Surviving fraction 11 weeks after start of treatment.

| Treatment | Survival |
|---|---|
| Control - Formulation Buffer (0.9% NaCl) | 0% |
| 10 mg NNV024 | 40% |
| 50 mg NNV024 | 60% |
| 10 mg obinutuzumab | 10% |
| 50 mg obinutuzumab | 20% |

FIG. 23 shows the survival of the mice in the different treatment groups as a function of time after injection of the treatments. The survival in all groups of ab-treated mice was significantly longer than the survival of the mice in the control group (log rank test, p<0.01). The survival of the mice treated with 50 mg and 10 mg NNV024 was significantly higher than the survival of mice treated with 50 mg and 10 mg obinutuzumab, respectively (log rank test p=0.049 and 0.043, respectively).

The trend seen for the surviving fraction was also seen for body weight of the mice (Table 30). The average weight of mice treated with NNV024 increased with time, while the average weight of mice treated with obinutuzumab started to drop approximately 40 days after start of treatment. 79 days after start of treatment there was 2.3-3.4 g higher average weight of mice in the NNV024 groups than the Obinutuzumab groups.

TABLE 30
Average bodyweight of mice after start of treatment.

| | Days from start of treatment | | | | | | | | | | | |
|---|---|---|---|---|---|---|---|---|---|---|---|---|
| Group | 0 | 9 | 13 | 22 | 30 | 37 | 43 | 51 | 58 | 65 | 72 | 79 |
| Control | 18.6 | 19.5 | 20.3 | 18.5 | 18.1 | | | | | | | |
| 10 mg | 18.6 | 20.1 | 20.8 | 21.0 | 21.5 | 22.1 | 21.1 | 21.5 | 21.2 | 20.4 | 20.7 | 19.8 |
| NNV024 | | | | | | | | | | | | |
| 50 mg | 18.6 | 19.6 | 20.4 | 20.8 | 21.1 | 21.3 | 21.4 | 21.6 | 21.7 | 20.9 | 20.8 | 20.5 |
| NNV024 | | | | | | | | | | | | |
| 10 mg | 18.6 | 19.9 | 19.9 | 20.4 | 20.4 | 20.2 | 20.0 | 18.9 | 18.5 | 18.0 | 17.8 | 17.5 |
| obi | | | | | | | | | | | | |
| 50 mg | 18.7 | 19.7 | 20.2 | 21.0 | 20.8 | 20.4 | 19.1 | 19.3 | 19.3 | 18.4 | 18.1 | 17.1 |
| obi | | | | | | | | | | | | |

### Discussion

In example 8 no difference in survival was observed for mice injected intravenously with Daudi lymphoma cells and treated with 1-4 injections of 100 mg NNV024 or obinutuzumab. The ADCC experiments in example 10 show that the differences between NNV024 and obinutuzumab was highest for the lowest antibody concentrations tested. Therefore, the therapy experiment of example 8 was repeated in the current study with single injections of 10 or 50 mg of NNV024 or obinutuzumab. With the lower antibody doses the superior ADCC results seen in example 10 for NNV024 was confirmed in vivo, indicating that mechanism of action for NNV024 in vivo is induction of ADCC and/or CDC in the tumor cells since the ADCC and CDC effect of NNV024 is higher than of obinutuzumab while the ADCP effect is lower than for obinutuzumab.

### Conclusion

The therapeutic efficacy of the NNV024 ab was statistically significantly higher than of Obinutuzumab when compared at the same amount of antibody injected for both 10 and 50 mg of antibody dose in an intravenous Daudi lymphoma model in SCID mice.

### Example 10 Comparison of ADCC Induced by NNV023, NNV024, Duohexabody-CD37 and Obinutuzumab in Ramos Cells

The aim of this study is to compare induction of ADCC by NNV023, NNV024, duohexabody-CD37 and obinutuzumab in Ramos cells

### Materials and Methods

### Culturing and Preparation of Target Cells

The Ramos cell line was cultured in RPM11640 supplemented with GlutamaxX (Gibco, Paisley, UK), 10% heat-inactivated FCS (Gibco) and 1% penicillin-streptomycin mix (Gibco). The incubation takes place in a humidified atmosphere with 5% CO₂ at 37° C. Cell suspensions are diluted 1:5 with pre-warmed medium twice a week (unless otherwise stated based on cell viability). To ensure an exponential growth at the beginning of the experiment, the cells were diluted 2 days before.

### Experiment Procedure

The target cells (Ramos) were diluted two days prior the day of the experiment. They were harvested and plated into white flat-bottom 96 well plates at concentration of approx. 25 000 cells/well in the 25 μL of the assay buffer/well.

The dilutions of the test items were prepared in triplicates at four different concentrations: 0.001, 0.01, 0.1, and 1.0 μg/mL. The dilutions were prepared in 1.5 mL Eppendorf tubes (10-fold dilution) and then transferred into a v-bottom transparent 96 well plate. The solutions from this plate were added to both plates containing the target cells.

The cells and target Abs were co-incubated on the bench (LAF-bench) for 30 min.

The effector cells from Promega ADCC Reporter Bioassay Core kit (Ref.G701A) were thawed, diluted and added to the assay plates with the target cells. The ratio of the Effector to Target cells became 2.65:1. After addition of the effector cells, the assay plates were incubated for 5 hours at 37° C. in a humidified incubator.

After 5 hours of incubation, the cells were placed on the bench for 15 min to equilibrate the temperature to RT. Bio-Glo luciferase assay reagent was added to the plates (all wells containing cells plus the cells for background control that does not contain any Abs). Bio-Glo luciferase was added to these wells at the same time as to the other wells and incubated in the dark at RT for 10 or 15 min. The luminescence of the luciferin was measured using Tecan plate reader (integration time of the acquisition was 0.5 sec/well).

### Data Analysis

The value of the background control (the mean of RLU for cells without Abs) was withdrawn from the signals of all wells of the same plate. The mean RLU and standard deviation of triplicates for each concentration of the test antibody were calculated. The resulting data set was plotted using dot-line plot and clustered bar plot in the axes: RLU vs. [Ab]μg/mL. A degree of ADCC induction was also calculated relatively to Obinutuzumab for all other test items of the same concentration levels.

### Results

The result obtained from a single experiment testing ADCC activation in Ramos cells is presented in FIG. 25.

The NNV023 and NNV024 showed a prominent induction of ADCC in Ramos cells. As in the previous study (Example 7), NNV024 demonstrated a superior and statistically different ADCC activation compared to the other Abs (Holm Sidak test, p<0.05). The smaller the concentration of antibodies, the greater difference in ADCC of NNV024 compared to the effect of Obinutuzumab: 1.0 μg/mL-111%, for 0.01 μg/mL-146%, for 0.001 μg/mL-269% of Obinutuzumab. Duohexabody-CD37 induced the lowest ADCC in Ramos cells, from 16.3% at 0.001 μg/mL to maximum of 32.7% of Obinutuzumab at 0.01 μg/mL. The further increase in concentration of duohexabody-CD37 reduces induction of ADCC to only 16.3% at 1.0 μg/mL. This effect may presumably be attributed to a spontaneous hexamerisation of IgG1 at higher concentrations.

### Discussion

Absence of fucose in the core of the N-glycan is a key factor in enhancing ADCC induction. Antibodies manufactured with different technology of afucosylation may have different composition of the N-glycan or different fraction of afucosylated N-glycan. The afucosylated Abs tested in the study were manufactured at different dates and by different manufacturers. Therefore, qualitative and quantitative analysis of the glycan content of NNV024 and obinutuzumab is warranted in order to understand the observed differences in ADCC for these Abs.

### Conclusion

NNV024 was superior in inducing ADCC as compared with the other antibodies tested.

### Example 11 Comparison of CDC Induced by NNV023, NNV024, NNV025, Duohexabody-CD37 and Obinutuzumab in Raji and Daudi Cells

The goal of this study was to measure the ability of the anti-CD37 antibodies NNV023, NNV024 and NNV025, to induce complement dependent cytotoxicity on the target cells Raji and Daudi. The CDC performance of these antibodies was evaluated through the quantification of cell viability upon treatment and compared to the CDC activity displayed by the anti-CD20 antibody Ofatumumab (positive control) and the C1q-binding impaired Ofatumumab P329A (negative control). Moreover, this study aimed to compare the CDC potential of NNV antibodies against the approved anti-CD20 therapeutic antibody obinutuzumab (Gazyvaro, Roche) and against Duohexabody-CD37 (HexaBody®-CD37 or GEN-3009, Genmab), a biparatopic anti-CD37 antibody currently in Phase 1/11 of clinical testing.

### Materials and Methods

Table 30 presents an overview of the test items.

TABLE 30
Antibodies used in the study.

| Test items | Concentration | |
|---|---|---|
| Ofatumumab WT (positive CTRL) | 22.25 | mg/mL |
| Ofatumumab P329A (negative CTRL) | 13.5 | mg/mL |
| Obinutuzumab (Gazyvaro) | 25 | mg/mL |
| DuoHexabody-CD37 (Biparatopic anti-CD37 mAb) | 9.35 | mg/mL |
| NNV025 (Humanized NNV003 (IgG1-WT)) | 8.2 | mg/mL |
| NNV023 (Reduced Immunogenicity risk (IgG1-WT)) | 5.1 | mg/mL |
| NNV024 (Afucosylated NNV023 (IgG1-WT)) | 7.4 | mg/mL |

Human Complement Serum (CTS-006) from Creative Biolabs was used as a source of complement. The C3&C5 removed Normal Human Serum, which has been deprived of essential proteins of the complement cascade, served as a negative control to rule out the complement dependency of antibody-induced cytotoxicity.

### Culturing of Target Cells

The cell lines Raji and Daudi were cultured in RPM11640 supplemented with GlutamaxX (Gibco, Paisley, UK), 10% heat-inactivated FCS (Gibco) and 1% Penicillin-streptomycin mix (Gibco). Cells were incubated in a humidified atmosphere with 5% CO₂ at 37° C. The cell suspensions were usually diluted to a concentration of 0.4 million cells/mL with pre-warmed medium twice a week.

Three days before the start of the CDC experiment, both Raji and Daudi cell lines were diluted to ensure that they were in the exponential phase of growth at the beginning of the experiment.

### Experiment Procedure in Short

On the day of the experiment, the target cells Raji and Daudi were harvested, washed and seeded into two black flat-bottom 96 well plates (CDC plates) at a concentration of approx. 1.5 million cells/ml (37500 cells/well) in 25 μL of serum-free RPMI.

Serial dilutions of the test items (1:25) were prepared in duplicates at three different concentrations: 0.016, 0.4 and 10 μg/mL. In addition, a control solution containing no antibody was prepared. 25 μL of each dilution were added to the CDC plates. To promote cell opsonization, target cells and test antibodies were incubated for 20 min at 37° C. in a humidified incubator.

Afterwards, 50 μL of 25% Human Complement Serum (CTS-006, Creative Biolabs) were added to one of the CDC plates and 50 μL of C3&C5 Removed Normal Human Serum (CTS-054, Creative Biolabs) to the other one. The final serum concentration for the assay was 12.5%. The plates were incubated for two hours at 37° C. in a humidified incubator to promote complement-dependent cell lysis.

To measure cytotoxicity, the cell viability reagent AlamarBlue was added to the plates (1:10 dilution), in all cell-containing wells plus 8 background wells without any cells. Upon overnight incubation (18 hours, 37° C., 5% CO₂), the fluorescent signal derived from resazurin reduction was acquired using the Ascent Fluoroskan fluorometer (excitation: 544 nm; emission:590 nm).

### Data Analysis

The cell-specific viability signal was adjusted by dividing the fluorescence measured in each cell-containing well on the average of the signals from background wells within the same plate (signal/noise ratio).

For both experiments, the average and standard deviation of the duplicates for each concentration of all the test items were calculated. The resulting data set was plotted using a clustered bar plot, with the following axes: signal/noise ratio (AU) vs. Antibody concentration.

### Results

The results obtained from the combination of two biological replicates in both Raji and Daudi cells are described in the next two sections.

### CDC Induction in Raji Cells

The data shown in FIG. 26 represent the CDC effects of the screened antibodies on the target cells Raji.

In 12.5% Human Serum Complement (FIG. 1A), DuoHexabody-CD37 activates CDC prominently, as shown by a strong decrease of cell viability compared to the control (around 40% reduction at 10 μg/mL). The CDC effects of DuoHexabody-CD37 were appreciated already with the lowest concentration (0.016 μg/mL) and were in the same range as the positive control Ofatumumab WT with 0.4 μg/mL of antibody (relative cell viability of 69.1% for DuoHexabody-CD37 vs 70.6% for Ofatumumab WT). At the highest concentration, Ofatumumab WT works slightly better in reducing Raji cell viability (46.6% for Ofatumumab vs 60.6% for DuoHexabody-CD37).

In general, the humanized NNV antibodies tested in this study displayed very modest CDC activation in Raji cells. The best CDC activation was achieved by NNV024, which reduced cell viability by 11% at a concentration of 0.4 μg/mL, and by 20% at 10 μg/mL. NNV024 had a statistically significantly higher CDC effect than NNV023 at both 0.4 μg/mL and 10 μg/mL (p<0.005, t-test). Comparing these results with the CDC activation by the negative control Ofatumumab P329A, a small reduction was observed with 0.4 μg/mL of antibody. At the highest concentration NNV024 activated CDC at a minor extent than Ofatumumab P329A. However, the CDC induction achieved by the negative control antibody might be due to residual C1q binding or cross-linking effects that occurred at high concentrations.

The assessment of the CDC performance of the commercially approved obinutuzumab revealed that also this antibody killed Raji cells in a limited manner. At the concentration of 0.4 μg/mL, almost no reduction of cell viability could be appreciated (98% vs control), while 10 μg/mL of Obinutuzumab activated CDC at rates similar to the ones observed for NNV024. NNV024 had a statistically significantly higher CDC effect than of obinutuzumab at 0.4 μg/mL (p<0.05, t-test).

To confirm that the cytotoxic effects induced by the antibodies in the panel were dependent on the complement cascade, the assay was also performed in 12, 5% human serum deprived of the complement proteins C3 and C5, essential for the formation of membrane attack complex. The results obtained are represented in the bar plots of FIG. 1 B. As expected, no relevant reduction of cell viability was observed with any of the antibodies tested, even at the highest concentration.

### CDC Induction in Daudi Cells

The data shown in FIG. 27 represent the CDC effects of the screened antibodies on the target cells Daudi.

In 12.5% Human Complement Serum, DuoHexabody-CD37 induces very strong cytotoxicity on Daudi cells. In particular, cell survival is reduced by around 90% in presence of both 0.4 μg/mL and 10 μg/mL of antibody (relative cell survival 13, 3% at 0.4 μg/mL and 12, 5% at 10 μg/mL). CDC activation looks more prominent for DuoHexabody-CD37 than for the positive control Ofatumumab WT at the concentration 0.4 μg/mL (relative cell viability of 13, 3% vs 54.8%), while both antibodies drastically reduce cell survival at 10 μg/mL (12.9% for Ofatumumab WT and 12.5% for DuoHexabody-CD37).

NNV024 exhibited modest CDC effects on Daudi cells, by reducing cell survival of around 20-25% at both 0.4 and 10 μg/mL. The CDC activity of NNV024 was statistically significantly higher than of NNV023 (p<0.05, t-test).

The evaluation of the performance of Obinutuzumab in CDC revealed very limited cytotoxicity on Daudi cells, with values similar to the ones showed by the negative control Ofatumumab P329A. It was possible to appreciate a slight reduction of cell viability only at 10 μg/mL (relative cell viability 93%), and no effects at lower antibody concentrations. In contrast with the observations made in Raji cells, NNV024 achieved significantly better CDC levels than Obinutuzumab in Daudi cells (p<0.05, t-test).

As for Raji cells, the ability of the tested antibodies to kill Daudi cells in a complement dependent manner was tested in 12.5% C3&C5 removed human serum. The results shown in FIG. 27B indicate that no relevant reduction of cell viability occurred when complement activation was impaired, confirming the dependence of complement for antibody-induced cytotoxicity.

### Discussion

The results of this study indicated that the biparatopic antibody DuoHexabody-CD37 was able to achieve the best levels of CDC among the tested articles in both of the target cell lines analysed. In particular, it showed dramatic effects on Daudi cells, where cell viability was reduced by around 90% already at the concentration of 0.4 μg/mL. This result is in line with the data reported by Oostindie et al. 2020 [1], which demonstrated that DuoHexaBody-37 had superior CDC activity compared to other CD37 antibodies. This bispecific antibody can deliver better CDC through its improved ability to induce IgG hexamerization via its E430G mutation and dual epitope targeting.

The data shown revealed that the antibodies tested activated CDC in a higher extent in Daudi than in Raji cells. One of the reasons behind this observation might be the higher expression of CD37 in Daudi cells (mention paper/report showing this), which would promote a larger number of antigen-antibody interactions and make these cells more sensitive to the complement cascade. In addition, the results indicated that the NNV candidates worked differently in the two cell lines and suggested that different molecular mechanisms might be prominent for CDC in the two systems. The reasons underlying these differences are not known and might regard the charge and glycan and lipid composition of the plasma membrane.

The NNV candidate that worked best in Raji cells is NNV024. Despite the limited ability to deliver CDC in comparison to DuoHexabody-CD37, the performance is clearly superior to the one of NNV023 and NNV025, which do not present any modification and did not induce any cytotoxicity

### Conclusion

The screening of NNV antibodies showed that, at least in the concentration range tested, the CDC potential of the anti-CD37 humanized antibodies of the present disclosure is lower than the one displayed by Duohexabody-CD37 in both Daudi and Raji cell lines. The performance of NNV024 is similar to the one of obinutuzumab in Raji cells and higher than obinutzumab in Daudi cells.

### Example 12 Therapeutic Effect of NNV024 and Obinutuzumab in SCID Mice Injected with Intravenous Daudi Cells

### Aim

The aim of this study is to compare the therapeutic effect of a single injection of NNV024 with Obinutuzumab in mice with intravenous NHL Daudi model. The example is an update of example 9 and contains the complete dataset of the experiment.

### Materials and Methods

### Test Articles

Vials containing the necessary amounts of NNV024 humanized monoclonal antibody (Nordic Nanovector) (7.4 mg/ml), Obinutuzumab antibody (Gazyvaro, Roche) (5 mg/ml diluted in 0.9% NaCl from 25 mg/ml stock), and 0.9% NaCl were provided by Nordic Nanovector. All the solutions in the shipped vials were sterile filtered and stored at 3-8° C., protected from light. Animals and housing: 50 female CB17-SCID mice, 6-7 weeks of age were ordered from Envigo, France and allowed to acclimate for 5 days prior to study start. The mice will be weighed and earmarked in the acclimation week. The mice were housed in an individually ventilated cages, 5 mice per cage. The nesting material, hideaways, drinking bottles, cage bars lids and lids were autoclaved prior to use. The mice were fed ad libitum with irradiated rodent diet. The animals were maintained with a 12-hour lighting cycle at a room temperature of 21-23° C. and air relative humidity of 40%.

### Cell Culturing

Daudi cells in suspension cells were grown in T75 flasks (Thermo) 25 ml medium/per flask in 37° C., 5% CO₂ humidified incubator. Cells were harvested when the cell concentration was 1-1.5 million cells per ml, viability was minimum 85% at harvest. Cell culture medium RPMI-1640-GlutaMAX (Life Technologies Cat no 72400-021) supplemented with 10% inactivated FBS (Life Technologies cat no 10270), 1% PenStrep (Life Technologies cat no 15140-122) and 1 mM Sodium Pyruvate (Sigma cat no p5280).

Injection of cells: The mice were injected with tumor cells suspended in RPMI medium from a fresh bottle without any supplements within 1.5-2 hours after they have been placed in transport vials.

The cells were kept on ice once placed in the transport vial until injected into the mice. The mice were injected in the lateral tail vein with 10 million cells pr. animal in a volume of 100 μl per mouse.

The animals were specifically observed for 15-20 minutes to register any treatment related adverse effect after the injection.

### Experimental Design

The current study includes 5 study groups, each with 10 animals per group (Table 31). Each mouse will be injected once with 100 μl of the indicated injection solution. This study was a “blinded” study, so the study director and the pathologist did not know what treatment each group received. On day 0 the mice were inoculated intravenously with 10 million Daudi cells, weighed, and randomized according to body weight into 5 groups so that each group had similar average body weight at study start. The next day (day 1) treatment was initiated. Animals were euthanized at one or more of the following humane endpoinds: Hind leg paralysis, overall weight loss of 20% or weight loss of 15% over a period of one week if signs of discomfort, kidney tumors 7-10 mm in diameter or larger, or signs of substantial discomfort.

TABLE 31
Study groups.

| Treatment | Dose μg/mouse/injection |
|---|---|
| Control - Formulation Buffer (0.9% NaCl) | 100 μL |
| NNV024 | 10 μg |
| NNV024 | 50 μg |
| Obinutuzumab | 10 μg |
| Obinutuzumab | 50 μg |

### Results

Four weeks after start of treatment all mice in the control group were euthanized while all the mice in the treatment groups were alive. FIG. 28 shows the survival of the mice in the different treatment groups as a function of time after injection of the treatments. The survival in all groups of ab-treated mice was significantly longer than the survival of the mice in the control group (log rank test, p<0.01). The survival of the mice treated with 50 μg NNV024 was significantly higher than the survival of mice treated with 50 μg and 10 μg obinutuzumab (log rank test p=0.0157 and 0.0119, respectively). The survival of the mice treated with 10 μg NNV024 was not significantly higher than for mice treated with either dose of obinutuzumab. At the end of the study, 118 days after start of treatment, the survival of mice treated with 50 μg or 10 μg NNV024 was 30% and 20%, respectively, while the survival was 0% and 10% for mice treated with 50 or 10 μg obinutuzumab (Table 32).

TABLE 32
Median survival and surviving fraction
118 days after start of treatment.

| Treatment | Median survival* | Survival at day 118 |
|---|---|---|
| Control - Formulation | 23.5 days | 0% |
| Buffer (0.9% NaCl) | | |
| 10 mg NNV024 | 70.5 days | 20% |
| 50 mg NNV024 | 84 days | 30% |
| 10 mg obinutuzumab | 53 days | 10% |
| 50 mg obinutuzumab | 62 days | 0% |

*All treatment groups had significantly higher survival than the control group (p < 0.0001, Kaplan Meier log-rank test (Mantel-Cox)).

The trend seen for the surviving fraction was also seen for body weight of the mice (FIG. 29).

The average weight of mice treated with NNV024 increased with time, while the average weight of mice treated with obinutuzumab started to drop approximately 40 days after start of treatment. 79 days after start of treatment there was 2.3-3.4 g higher average weight of mice in the NNV024 groups than the Obinutuzumab groups.

### Discussion

In example 8 no difference in survival was observed for mice injected intravenously with Daudi lymphoma cells and treated with 1-4 injections of 100 μg NNV024 or obinutuzumab. The ADCC experiments in example 7 and 10 showed that the differences between NNV024 and obinutuzumab was highest for the lowest antibody concentrations tested. Therefore, the therapy experiment of example 8 was repeated in the current study with single injections of 10 or 50 μg of NNV024 or Obinutuzumab. With the lower antibody doses the superior ADCC results seen in example 7 and 10 for NNV024 was confirmed in vivo, indicating that mechanism of action for NNV024 in vivo is induction of ADCC and/or CDC in the tumor cells since the ADCC and CDC effect of NNV024 is higher than of Obinutuzumab while the ADCP effect is lower than for Obinutuzumab.

### Conclusion

The therapeutic efficacy of the NNV024 antibody was statistically significantly higher than of Obinutuzumab when treated with 50 μg antibody/mouse in an intravenous Daudi lymphoma model in SCID mice.

### Example 13 Comparative ADCC Induction of NNV023, NNV024, Duohexabody-CD37 and Obinutuzumab

### Aim

The aim of this study is to compare induction of ADCC by NNV023, NNV024, duohexabody-CD37 and Obinutuzumab in Daudi, Ramos, Raji, Rec-1, U2932, DOHH-2, SU-DHL-4, SU-DHL-6, WSU-DLCL-2, Granta-519, and REH cells.

### Materials and Methods

### Culturing and Preparation of Target Cells

The Daudi, Ramos, Raji, Rec-1, and REH cell lines were obtained from American Type Culture Collection (ATCC, Manassas, VA). The U2932, SU-DHL-4, SU-DHL-6, WSU-DLCL-2 and Granta-519 cell lines were provided by University Medical Center Groningen (UMCG, The Netherlands). DOHH-2 were purchased from German Collection of Microorganisms and Cell Cultures GmbH (DSMZ, Braunschweig, Germany). Authentication of cell lines was performed at Eurofins Genomics Europe Applied Genomics GmbH (Ebersberg, Germany). The genetic characteristics of all tested lines were found to match their characteristics in the databases. All the cell lines were cultured at 37° C. in a humidified 5% CO₂ incubator using RPMI 1640 GlutaMAX (Gibco, Waltham, USA) cell culture medium, except for Granta-519 that was cultured in DMEM high glucose (Gibco). The cell growth media were supplemented with 10% Fetal Bovine Serum and 1% Penicillin-Streptomycin (both from Gibco).

### Experiment Procedure

The target (T) cell lines (Daudi, Ramos, Raji, Rec-1, U2932, DOHH-2, SU-DHL-4, SU-DHL-6, WSU-DLCL-2, Granta-519, and REH) were diluted two days prior the day of the experiment to 0.5×10⁶ cells/mL. On the day of the experiment, they were harvested, extensively washed three times in an excess of the PBS with 0.5% BSA, resuspended in RPMI supplemented with 4% low IgG serum (assay buffer) to concentration 1.0×10⁶ cells/mL and seeded (2.5×10⁴ cells/well) in a white flat bottom 96 well plate (165306, Thermo Fisher). Right after plating the cells, the plates were conditioned to ambient temperature, 21° C. (RT) for 15 min. Serial dilutions (0.001-1 μg/mL in assay buffer) of the test antibodies NNV023, NNV024, obinutuzumab, rituximab, and DuoHexabody-CD37 were added to the wells as triplicates and incubated for 30 min at 21° C. to induce opsonization. Some wells with target cells were left without addition of any Ab. The Promega effector Jurkat cells (E) recombinantly modified to stably express FcγRIIIa-V158 receptors were thawed, resuspended in the assay buffer and added to the target cells in proportion 3:1 (E:T). The system was incubated for 16-18 hours followed by addition of Bio-Glo Luciferase Assay (Promega) in equivolumetric ratio with content of the wells. After 15 min of incubation at RT (21° C.), the luminescence read-out was performed using a Spark microplate reader (TECAN, Switzerland). The values in relative luminescent units (RLU) were obtained for each well containing cells.

### Data Analysis

The value of the background control (the mean RLU of triplicates of cells without Abs) was withdrawn from the signals of all wells of the same plate. The mean RLU and standard deviation of triplicates for each concentration of the test antibody were calculated. Normalized induction of ADCC was calculated by dividing the luminescence values of the treated cells by the mean luminescence of untreated (Ab-free) cells. The resulting data set was plotted using scatter and line plot in the axes: RLU vs. [Ab]μg/mL.

For each test Ab the potency (EC₅₀), efficacy (Eₘₐₓ), and AUC to induce ADCC signaling were calculated by fitting the binding data to the dose-response (agonist, three parameters) nonlinear regression model using GraphPad Prism 9.0. The result for each cell line was plotted in the coordinates: relevant parameter (EC₅₀, Eₘₐₓ, AUC) vs. antibody name. The average value for these parameters was calculated for each Ab and over imposed on the clustered scatter plot. Of note, the signal of DuoHexaBody-CD37 at the highest concentration dipped down which distorts the EC₅₀ assessment and results in an erroneous Hill-slope parameter; therefore, the highest concentration has not been used in the regression analysis and fitting. The upper asymptote for this Ab was constrained to its maximum value obtained.

### Results

The result obtained from a single experiment testing ADCC activation in eleven NHL cell lines is presented in FIGS. 30, 31, 32 and 33.

In vitro ADCC potency was investigated using reporter assays in a panel of eleven cell lines. The lymphoma cell models represented B-NHL subtypes with an unmet therapeutical need, such as Burkitt's lymphoma, GCB- and ABC-DLBCL, and MCL. An acute lymphoblastic leukemia (ALL) cell line, REH, was used as a CD20-/CD37-control [Schneider D, Xiong Y, Wu D, NIIe V, Schmitz S, Haso W, et al. A tandem CD19/CD20 CAR lentiviral vector drives on-target and off-target antigen modulation in leukemia cell lines. J Immunother Cancer. 2017; 5:42.] The degree of induction of ADCC signalling in vitro was dependent on the target cell line. NNV024 Ab displayed overall better ADCC activation (EC₅₀ and AUC) compared to other tested antibodies practically in all cell lines (FIGS. 30, 31, 32 and 33), except for Raji, where the IC₅₀ and AUC values of NNV024 were similar to obinutuzumab. Being fully afucosylated, NNV024 and obinutuzumab, displayed high degree agonism to FcγRIIIa-158V in all cell lines with average Eₘₐₓ being 1.36-fold and 1.32-fold higher than Eₘₐₓ of rituximab. The average potency (EC₅₀) in the ten cell lines for NNV024 (35.5-fold higher EC₅₀ compared to the EC₅₀ of rituximab) prevails the one of obinutuzumab (19.3-fold higher EC₅₀ compared to the EC₅₀ of rituximab).

The other four antibodies displayed only a partial agonism of different degree: the efficacy of NNV023 and rituximab was comparable and only 75% of NNV024. DuoHexabody-CD37 has also displayed a partial agonism to FcγRIIIa-V158 receptors, with the efficacy (Eₘₐₓ) being of just 53% of NNV024 (FIG. 33A). Rituximab displayed the lowest potency (EC₅₀) of all tested antibodies (FIG. 33). The potency of NNV023 was around 7-fold higher than the potency of rituximab (FIG. 33B).

### Discussion

Absence of fucose in the core of the N-glycan is a key factor in enhancing ADCC induction. NNV024 and obinutuzumab were designed to be depleted with the core fucose to maximize complementarity of Fc parts of Ab and FcγRIIIa receptors to boost ADCC function of the Abs. Antibodies manufactured with different technology of afucosylation may have different composition of the N-glycan or different fraction of afucosylated N-glycan. The afucosylated Abs (NNV024 and obinutuzumab) tested in the study were manufactured using different technologies by different manufacturers. Therefore, to understand the observed differences in ADCC for the tested antibodies, a qualitative and quantitative analysis of the glycan content of NNV024 and obinutuzumab is warranted.

### Conclusion

The afucosylated humanized anti-CD37 Ab (NNV024) was found superior in inducing ADCC signaling as compared with the other antibodies tested: obinutuzumab, rituximab, NNV023, and DuoHexabody-CD37.

### Example 14 Plasma Half-Life and Pharmacokinetics of NNV023, NNV024 and NNV025 in Human FcRn Expressing Mice

### Aim

The aim of the experiment was to compare the plasma half-lives (T_{1/2}) and other PK parameters for NNV023, NNV024 and NNV025 with obinutuzumab (anti-CD20, IgG1, afucosylated, Gazyvaro) and a recombinant form of DuoHexabody-CD37 (anti-CD37; Biparatopic IgG1; DuoHexabody-CD37) in the human FcRn transgenic mouse model.

### Materials and Methods

Each antibody (Table 33) was administered by intravenous (i.v) injection to hemizygous human FcRn transgenic mice (Tg32 hemizygous, the Jackson Laboratory (JAX)) (5 animals per group) at a dose of 5 mg/kg. The blood was sampled, and plasma isolated at day 1, 2, 3, 5, 7, 10, 12, 16, 19, 23 and 30 post administration of the antibodies. The plasma concentration of the antibodies was determined by ELISA. The half-life and other PK parameters were calculated using two different methods: (a) the percent remaining Ab in plasma was entered into the exponential decay formula; (b) plasma concentrations were fitted to a non-compartmental (NCA) PK model.

TABLE 33
Test antibodies

| obinutuzumab | Gazyvaro |
|---|---|
| DuoHexabody-CD37 | Biparatopic anti-CD37 mAb (Genmab, |
| | recombinantly produced by NNV) |
| NNV025 | Humanized NNV003 (IgG1-WT) |
| NNV023 | Reduced Immunogenicity risk (IgG-WT) |
| NNV024 | Afucosylated NNV023 (IgG1-WT) |

### Blood Sampling and Plasma Preparation:

1. Hemizygous human FcRn transgenic mice (B6.Cg-Fcgrt^{tm1Dor} Tg(FCGRT)32Dcr/DcrJ) (Tg32 hemizygous) that are knockout for the mouse FcRn heavy chain and express the genomic transgene of the human FcRn heavy chain, under the control of the human FcRn promotor, were used in the plasma half-life/pharmacokinetic study.
2. On day 0, the mice were dosed with 5 mg/kg of the antibodies, diluted in 1× phosphate buffered saline (PBS), by i.v injection.
3. Blood samples (25 μl) were drawn from the retro-orbital sinus on day 1, 2, 3, 5, 7, 10, 12, 16, 19, 23 and 30 post administration of the antibodies.
4. The blood samples were immediately mixed with 1 ml 1% K3-EDTA to prevent coagulation and centrifuged at 17000×g for 5 min at 4° C. to isolate plasma.
5. Isolated plasma was diluted 1:10 in glycerol/PBS solution (1:1) and stored at −20° C. until analysis. JAX recommends analyzing the plasma samples within 6 months of isolation.

### Quantification of Antibodies in Plasma:

1. 96-well EIA/RIA plates were coated with 100 ml of anti-human IgG (whole molecule) antibody from goat diluted to 1 μg/mL in PBS, and incubated overnight at 4° C.
2. The plates were washed four times with PBS containing 0.05% Tween 20 (T) using a HydrospeedA plate washer.
3. The remaining surface area was blocked with PBS/T containing 4% skimmed milk powder (S) for 1 hour (h) at room temperature (RT).
4. The plates were washed four times with PBS/T using a HydrospeedA plate washer.
5. The plasma samples were diluted 1:50, 1:100, 1:200, 1:400, 1:800 and 1:1600 in PBS/T/S, added to the plates and incubated for 1 hour at RT. A 12-point standard curve (STD) (1000.0-0.488 ng/mL) of the antibody variant corresponding to the study group being analyzed was added to the same plate in duplicates.
6. Antibodies captured from plasma samples were detected by an alkaline phosphatase (AP)-conjugated polyclonal anti-human IgG (Fc specific) antibody from goat (diluted 1:5000 in PBS/T/S).
7. The plates were washed four times with PBS/T using a HydrospeedA plate washer.
8. Visualization was performed by addition of p-nitrophenylphosphate (1 mg/mL in diethanolamine buffer).
9. The plates were developed for 20-30 minutes before the 405 nm absorption values were recorded using a TECAN Sunrise plate reader.

### Calculation of Plasma Half-Life and PK Parameters:

Method 1: The absorbance values obtained from the ELISA analysis were interpolated to the standard curves using GraphPad Prism. Values corresponding to the exponential part standard curve were used. The concentration of the antibody samples in plasma at each time point was then calculated by multiplying with sample dilution factor used (Microsoft Excel). The first concentration measured (day 1) was then normalized to 100% and remaining data points were plotted as percent antibody remaining in plasma. Data points from the β-elimination phase were then used to calculate the plasma half-life using the formula:

$${τ}_{1 / 2} = \frac{log ( 0.5 )}{log ( \frac{{A}_{t}}{{A}_{0}} )} \times t$$

where T_{1/2} is the half-life, A_{c} is the amount of antibody remaining, t is the elapsed time, and A₀ and Aₜ are the amount of antibody at day 1 and at t.

Method 2: The antibody concentrations in plasma determined by ELISA were then fitted to a non-compartmental (NCA) PK model using the gPKPDsim PK add-on for MatLAb [Hosseini I, et al. gPKPDSim: a SimBiology((R))-based GUI application for PKPD modeling in drug development. J Pharmacokinet Pharmacodyn. 2018; 45(2):259-75. Epub 2018/01/06.].

### Results

Pharmacokinetic properties of NNV025, NNV023 and NNV024 were compared with obinutuzumab and recombinant DuoHexaBody-CD37 by injecting these mAbs in mice expressing human FcRn.

The percentages of antibodies remaining in plasma, in the absence of competing IgG, were plotted against time and their plasma half-life calculated.

The results indicate that the amino acid substitution (V110 D) introduced to reduce the risk of immunogenicity, significantly increased the plasma half-life of NNV023 (12.0±2.4 days) by more than 4 days compared to NNV025 (7.4±0.8 days), see FIG. 1. Afucosylation of NNV023 led to a more than 3-days reduction in plasma half-life, NNV024 (8.8±0.8 days). Finally, the plasma half-lives of obinutuzumab (4.4±1.3 days) and DuoHexabody-CD37 (4.1±1.2 days) were more than 7 days shorter than that of NNV023 (FIG. 34).

The plasma concentrations of the antibody variants were also fitted to a non-compartmental analysis (NCA) PK model (Table 34). The results revealed a similar hierarchy of parameters between the antibody variants as obtained in the percentage-based analysis above. NNV023 showed a 4-day extended plasma half-life (16.3±5.8 days) compared to NNV025 (12.1±4.7 days) and NNV024 (12.3±6.2 days). The NCA method confirmed that obinutuzumab and DuoHexabody-CD37 were cleared from plasma faster than the other antibody variants. The half-lives of the antibody variants reflected differences in the AUC, clearance, mean residency time (MRT) and volume of distribution at steady state (Vss), (Table 34).

TABLE 34
PK parameters of antibodies in Tg32 hemizygous mice using NCA PK model.

| | NCA PK model | | | | | | |
|---|---|---|---|---|---|---|---|
| | AUC | Cmax | CL | MRT | Vss | Half-life | |
| Group | (μg*d/mL) | (μg/mL) | (mL/d/kg) | (d) | (mL/kg) | NCA (d) | R² |
| NNV025 | 321.5 | 33.2 | 14.4 | 15.5 | 195.5 | 12.1 ± 4.7 | 0.974 |
| NNV023 | 278.6 | 19.9 | 16.4 | 21.7 | 255.1 | 16.3 ± 5.8 | 0.977 |
| NNV024 | 212.1 | 20.0 | 25.1 | 8.6 | 205.5 | 12.3 ± 6.2 | 0.991 |
| obinutuzumab | 708.4¹ | 110.6¹ | 7.3 | 5.9 | 39.2 | 5.4 ± 2.9 | 0.969 |
| DuoHexabody- | 186.9 | 28.68 | 28.2 | 4.3 | 114.9 | 3.6 ± 1.8 | 0.949 |
| CD37 | | | | | | | |

Abbreviations:
AUC—area under the curve, Cmax—peak plasma concentration of Ab, CL—clearance, MRT—mean residency time, Vss—volume of distribution at steady state, d—days.
¹Five times higher concentration was used for obinutuzumab than for the other antibodies due to a technical error. Therefore, the values for Cmax was 5 times higher than for the other antibodies.

### Discussion

The NNV023 antibody, engineered for reduced immunogenicity risk in humans, showed a long plasma half-life and favorable PK parameters in the Tg32 hemizygous mouse model (12.0±2.4 days). Strikingly, this variant showed about 5 days longer plasma half-life than that of humanized NNV025 (7.4±0.8 days), which is a result of only one amino acid substitution difference in the light chain (V110D). As such, this change alters the ability of the antibody to persist in the circulatory system. If this relates to altered engagement of human FcRn in vivo is not known. However, no difference in human FcRn binding and transport properties have been observed, as described in previous reports. Afucosylation (NNV024) resulted in a reduction in plasma half-life (4 days) compared to NNV023. The reduced half-life of NNV024 is in line with a report showing a modest reduction in half-life of afucosylated trastuzumab (anti-HER2; IgG1; Herceptin) [see Junttila T T, et al. Superior in vivo efficacy of afucosylated trastuzumab in the treatment of HER2-amplified breast cancer. Cancer Res. 2010; 70(11):4481-9. Epub 2010/05/21].

### Conclusion

The NNV023 antibody with the V110D mutation in the light chain had longer plasma half-life than NNV025, without the mutation, and also longer than the afucosylated version, NNV024. In addition, NNV024 had longer plasma half-life than obinutuzumab and recombinant DuoHexabody-CD37.

### Example 15 Serum Half-Life of NNV023 and NNV024 Compared with Obinutuzumab in TG32 Hemizygous IVIg Pre-Loaded Mice

### Aim

The aim of the experiment was to compare the plasma half-lives (Ti/2) and other PK parameters for NNV023 and NNV024 with Obinutuzumab (anti-CD20, IgG1, afucosylated, Gazyvaro) in the human FcRn transgenic mouse model TG32 pre-loaded with intravenous immunoglobulin (IVIg) to mimic competition with endogenous antibodies. Mouse IgG does not bind human FcRn and, as such, is not rescued from intracellular degradation. Mouse IgG is therefore cleared rapidly in human FcRn transgenic mice so that they lack representative amounts of endogenous IgG. To account for the lack of competition with endogenous IgG, the human FcRn transgenic mice were pre-loaded with intravenous immunoglobulin (IVIg) in this experiment.

### Materials and Methods

Each antibody was administered by intravenous (i.v.) injection to hemizygous human FcRn transgenic mice (Tg32 hemizygous, the Jackson Laboratory (JAX)) (4-5 animals per group) at a dose of 5 mg/kg (Table 35). The mice were pre-loaded with 250 mg/kg IVIg 2 days before administration of the antibodies. Blood samples were taken, and plasma isolated at day 1, 2, 3, 5, 7, 10, 12, 16, 19, 23 and 30 post administration of the antibodies. The plasma concentration of the antibodies was determined by ELISA before half-life and PK parameters were calculated using two different methods.

TABLE 35
Test antibodies

| obinutuzumab | Gazyvaro |
|---|---|
| NNV023 | Reduced Immunogenicity risk (IgG-WT) |
| NNV024 | Afucosylated NNV023 (IgG1-WT) |

The in vivo plasma half-life collection of samples is described in Table 36. Briefly:
1. Hemizygous human FcRn transgenic mice (B6.Cg-Fcgrt^{tm1Dor} Tg(FCGRT)₃₂Dcr/DcrJ) (Tg32 hemizygous) that are knockout for the mouse FcRn heavy chain and express the genomic transgene of the human FcRn heavy chain, under the control of the human FcRn promotor, were used in the plasma half-life/pharmacokinetic study.
2. On day −2, the mice were pre-loaded with 250 mg/kg IVIg (privigen) by i.v injection.
3. On day 0, the mice were dosed with 5 mg/kg of the antibodies, diluted in 1× phosphate buffered saline (PBS), by i.v injection.
4. Blood samples (25 μl) were drawn from the retro-orbital sinus on day 1, 2, 3, 7, 10, 12, 16, 19, 23 and 30 post administration of the antibodies.
5. The blood samples were immediately mixed with 1 ml 1% K3-EDTA to prevent coagulation and centrifuged at 17000×g for 5 min at 4° C. to isolate plasma.
6. Isolated plasma was diluted 1:10 in glycerol/PBS solution (1:1) and stored at −20° C. until analysis. JAX recommends analyzing the plasma samples within 6 months of isolation.

TABLE 36
Experimental design for the in vivo PK assessment.

| | | | | Pre-dose (IVIg) | | |
|---|---|---|---|---|---|---|
| | | | Dose | on Day -2 (48 h | | |
| | | | (mg/ | before test | | |
| Group | N | Test Articles | kg) | article injection) | Route | Blood collection days |
| 1 | 5 | NNV023 | 5 | 250 mg/kg | IV | 1, 2, 3, 7, 10, 12, 16, 19, 23, 30 |
| 3 | 5 | NNV024 | 5 | 250 mg/kg | IV | 1, 2, 3, 7, 10, 12, 16, 19, 23, 30 |
| 6 | 5 | Obinutuzumab | 4 | 250 mg/kg | IV | 1, 2, 3, 7, 10, 12, 16, 19, 23, 30 |

The ELISA-assay for detection of NNV023, NNV024 and obinutuzumab in plasma from IVIg pre-loaded Tg32 hemizygous mice was performed as follows:
1. 96-well EIA/RIA plates were coated with 100 μl of PBS containing 0.5 μg/mL of the anti-idiotypic Fab AbD34091_Fab (Eurofins GmbH, Puchheim, Germany) or 0.25 μg/mL of anti-idiotypic anti-obinutuzumab mouse monoclonal antibody (A01945, clone 18H8, Genscript, Leiden, The Netherlands) and incubated overnight at 4° C. AbD34091_Fab binds to the CDR region of the four NNV IgGs, while 18H8 binds the CDR portion of obinutuzumab.
2. The plates were washed four times with PBS/Tween20 (T) using a Hydrospeed™ plate washer.
3. The remaining surface area was blocked with PBS/T with 4% skimmed milk powder (S) for 1 hour at RT.
4. The plates were washed four times with PBS/T using a Hydrospeed™ plate washer.
5. Plasma samples were diluted 1:50, 1:100, 1:200, 1:400, 1:800 and 1:1600 in PBS/T/S, added to the plates. In addition, a 12-point standard curve (STD) of the test antibody corresponding to the study group being analyzed (1000-0.488 ng/mL) was added in duplicates.
6. The plates were incubated for 1 hour at RT before washing four times with PBS/T/S using a Hydrospeed™ plate washer.
7. NNV antibodies captured on AbD34091_Fab were detected by adding an AP-conjugated polyclonal anti-human IgG (Fc specific) antibody from goat (diluted 1:5000 in PBS/T/S).
8. Obinutuzumab antibody captured on anti-obinutuzumab antibody (18H8) was detected by adding a second anti-idiotype biotinylated anti-obinutuzumab antibody (16B7) (diluted 1:3000 in PBS/T/S).
9. The plates were incubated for 1 hour at RT before washing four times with PBS/T/S using a Hydrospeed™ plate washer.
10. Bound biotinylated anti-obinutuzumab (16B7) was detected by adding ALP-conjugated streptavidin (diluted 1:5000 in PBS/T/S). The plates were incubated for 1 hour at RT and then washed four times with PBS/T/S using a Hydrospeed™ plate washer.
11. Visualization of both the wells for detection of NNV antibodies and obinutuzumab was performed by addition of p-nitrophenylphosphate (1 mg/mL final concentration in diethanolamine buffer).
12. The plates were developed for 30-40 minutes before the 405 nm absorption values were recorded using a TECAN Sunrise plate reader.

The calculation of plasma half-life and PK parameters were performed as follows:
1. The absorbance values obtained from the ELISA analysis were interpolated to the standard curves using Graphpad Prism. Values corresponding to the exponential part of the standard curve were used. The concentration of the antibody samples in plasma at each time point was then calculated by multiplying for the sample dilution factor used (Microsoft Excel). The first concentration measured (day 1) was then normalized to 100% and the remaining data points were plotted as percent antibody remaining in plasma. The remaining fractions for each tested antibody (percentage of remaining antibody in the plasma) were plotted in function of the time from injection and fitted to a non-linear regression model (two-phase decay).
   Data points from the β-elimination phase were then used to calculate the plasma half-life using the formula:

$${t}_{1 / 2} = \frac{log ( 0.5 )}{log ( \frac{{A}_{t}}{{A}_{0}} )} \times t$$

where t_{1/2} is the half-life, A_{c} is the amount of antibody remaining, and A₀ is the amount of antibody at day 1 and t is the elapsed time.
2. The antibody concentrations in plasma determined by ELISA were then fitted to a non-compartmental (NCA) PK model using the gPKPDsim PK add-on for MatLAb [Hosseini I, et al. gPKPDSim: a SimBiology((R))-based GUI application for PKPD modeling in drug development. J Pharmacokinet Pharmacodyn. 2018; 45(2):259-75. Epub 2018/01/06]. The PK parameters calculated from this analysis were: the Area under the curve (AUC), the maximum concentration after administration (Cmax), clearance (CL), the mean residency time (MRT), the volume of distribution at steady state (Vss) and the half-life (t_{1/2}). 3. Statistical analysis was performed using repeated measurements two-way ANOVA with Geisser-Greenhouse correction in GraphPad Prism. Outlier detection/elimination was performed using Grubb's test in GraphPad Prism.

### Results

Pharmacokinetic properties of NNV023 and NNV024 were compared with obinutuzumab by injecting these mAbs in mice expressing human FcRn. The percentages of antibodies remaining in plasma, in the absence of competing IgG, were plotted against time and their plasma half-life calculated.

The results show that with IVIg pre-loading the amino acid substitution (V110D) introduced to reduce the risk of immunogenicity, did not significantly increase the plasma half-life of NNV023 (5.9±0.9 days) as compared to NNV024 (4.3±1.5 days) (FIG. 35). The average plasma half-lives of NNV023 and NNV024 was 2.5 and 0.9 days, respectively, longer than the one of obinutuzumab (3.4±1.3 days), but this difference was not statistically significant.

The plasma concentrations of the antibody variants were also fitted to a non-compartmental analysis (NCA) PK model (FIG. 36 and Table 37). The NCA analysis showed that the plasma half-life of NNV023 (8.6±4.8 days) and NNV024 (7.4±3.3 days) were 4.7 days and 3.5 days, respectively, longer than for obinutuzumab (3.9±3.0 days). Two-way ANOVA statistical testing revealed that the difference between NNV023 and Obinutuzumab was significant (p-value=0.001), while the difference between Obinutuzumab and NNV024 was deemed as non-significant (p-value=0.07).

TABLE 37
NCA PK parameters of NNV antibodies in
IVIg pre-loaded Tg32 hemizygous mice.

| | | AUC | Cmax | CL | MRT | Vss | Half-life | |
|---|---|---|---|---|---|---|---|---|
| Group | Route | (μg*d/mL) | (μg/mL) | (mL/d/kg) | (d) | (mL/kg) | (d) | R² |
| NNV024 | IV | 262.3 | 44.6 | 19.1 | 8.8 | 169.4 | 7.4 ± 3.3 | 0.94 |
| | Bolus | | | | | | | |
| NNV023 | IV | 425.8 | 55.4 | 11.1 | 10.4 | 118.0 | 8.6 ± 4.8 | 0.97 |
| | Bolus | | | | | | | |
| obin | IV | 148.4 | 29.1 | 42.2 | 5.2 | 182.0 | 3.9 ± 3.0 | 0.94 |
| | Bolus | | | | | | | |

AUC (μg*d/mL) = Area under the curve from the first to the last measured time point.
Cmax (μg/mL) = Maximum concentration after administration.
CL (mL/d/kg) = Clearance.
MRT (d) = Mean residency time (the average time the drug remains in the compartment)
Vss (mL/kg) = Volume of distribution at steady state.
Half-life (d) = The terminal half-life of the drug.
R² = How well the regression for half-life determination approximates the real data points.

### Discussion

The NNV024 and NNV023 antibodies, both engineered for reduced immunogenicity risk in humans and without and with fucose, respectively, showed a long plasma half-life and favorable PK parameters in the Tg32 hemizygous mouse model as compared with obinutuzumab.

### Conclusion

The NNV024 antibody had longer plasma half-life than obinutuzumab.

### Example 16 Therapeutic Effect of NNV024, Duohexabdy-CD37 and Obinutuzumab in Tg Mice Injected with Intravenous Daudi Cells

### Aim

The aim of this study is to compare the therapeutic effect of a single injection of NNV024 with duohexabdy-CD37 and obinutuzumab in Tg mice injected with intravenous Daudi cells. The model chosen for this study is a transgenic strain of SCID mice expressing the human FcRn (SCID FcRn−/− hFcRn (32) Tg), which represent the gold standard for the characterization of human IgG therapeutic candidates. Through the pre-loading of normal human IgG, this model can mimic the competition of IgGs for FcRn that is determinant for plasma half-life. Therefore, using these mice as disease hosts would combine the evaluation of both the pharmacokinetic profile and the therapeutic efficacy of the diverse antibody candidates in vivo.

### Materials and Methods

Vials containing the necessary amounts of NNV024 (7.4 mg/ml) (Nordic Nanovector), Duohexabody-CD37 (9.35 mg/ml) (manufactured in the Nordic Nanovector laboratory), obinutuzumab antibody (Gazyvaro, Roche) (5 mg/ml diluted in 0.9% NaCl from 25 mg/ml stock), and 0.9% NaCl were provided. All solutions were sterile filtered. Normal human Immunoglobin for intravenous injection (100 mg/mL) (IVIg, Privigen, CSL Behring) were used for pre-dosing of the mice. 32 female SCID FcRn−/− hFcRn (32) Tg mice, 5-8 weeks old, were ordered from The Jackson Laboratory (018441-B6.Cg-Fcgrt<tm1 Dcr>Prkdc<scid>Tg(FCGRT)32Dcr/DcrJ (jax.org)). The mice were allowed 5 days of acclimatization prior to study start. The mice were provided with filtered drinking water (Danmil filter, Product code DA25CSSS02 19252077, Rating 0.2 MIC http://danmil.dana5.dk/). Water bottles are changed 3 times a week. The mice were fed ad libitum with irradiated rodent diet (Altromin NIH #31 M—from Brogarden, Denmark). The animals were maintained with a 12-hours lighting cycle at a room temperature of 21-23° C. and air relative humidity of 40%.

Daudi cells were grown in T75 flasks (Thermo) with 25 ml medium/per flask, in 37° C., 5% CO₂ humidified incubator. They will be harvested when 1-1.5 million cells per ml, viability minimum 85% at harvest. Cell culture medium RPMI-1640-GlutaMAX (Life Technologies Cat no 72400-021) supplemented with 10% inactivated FBS (Life Technologies cat no 10270), 1% PenStrep (Life Technologies cat no 15140-122) and 1 mM Sodium Pyruvate (Sigma cat no p5280).

The mice were injected with tumor cells suspended in RPMI medium from a fresh bottle without any supplements. The mice were injected in the lateral tail vein with 10 million cells pr. animal in a volume of 100 μl per mouse. The animals were specifically observed for 15-20 minutes to register any treatment related adverse effects.

The study had 4 study groups, with 8 animals per group (Table 38). On day −1 the animals were weighed and randomized into the study groups according to body weight and age so that each group had the same average body weight and similar distribution of age at study start. Subsequently, serum blood samples were drawn (100 μl whole blood) from all mice and all the animals were pre-dosed with 250 mg/kg normal human IgG (IVIg, Privigen) to mimic competition with endogenous IgG on Fc receptors. On day 0 the mice were inoculated intravenously with 10 million Daudi cells and the next day (day 1), treatment was initiated according to Table 38.

TABLE 38
Study groups.

| | Pre- | Treatment | Number | # |
|---|---|---|---|---|
| Treatment | dosing | dose | of mice | injections |
| 0.9% NaCl | 250 mg/kg | 100 ml | 8 | 1 |
| | IVIg | | | |
| NNV024 | 250 mg/kg | 2.69 mg/kg | 8 | 1 |
| | IVIg | | | |
| Obinutuzumab | 250 mg/kg | 2.69 mg/kg | 8 | 1 |
| | IVIg | | | |
| Duohexabody-CD37 | 250 mg/kg | 2.69 mg/kg | 8 | 1 |
| | IVIg | | | |

Animals were euthanized at one or more of the following humane end-points: Hind leg paralysis, overall weight loss of 20% or weight loss of 15% over a period of one week if signs of discomfort, kidney tumours 7-10 mm in diameter or larger, or signs of substantial discomfort. Prior to euthanasia serum blood samples were collected and the serum stored at −20° C. After the mice have been euthanised necropsy were performed.

### Results

Survival analysis revealed that all treatment groups had statistically significantly better survival as compared to the control group, p=0.0004 (Kaplan Meier: Log-rank (Mantel-Cox Test)) while there was no difference between the treatment groups (Table 39 and FIG. 37). One animal in the NNV024 group did not have a visible tumor during necropsy but histopathology revealed infiltration of Daudi cells in the sacral part of the columna. No tumors were discovered in the groups that received Duohexabody-CD37 and obinutuzumab treatments.

TABLE 39
Median survival of female SCID FcRn−/−hFcRn (32)
Tg mice with i.v. injected Daudi-lymphoma cells (10
million cells) treated with 2.69 mg/kg of NNV024, obinutuzumab
or recombinant DuoHexabody-CD37 or 100 ml NaCl.

| | | | | % Animals |
|---|---|---|---|---|
| | Median | | P-value | without |
| | Survival | % Animals | summary | Daudi cell |
| | (days from | surviving | vs Control | lymphoma |
| Treatment | inoculation) | at day 130 | group | at day 130 |
| NaCl | 76 | 12.5 | — | 12.5 |
| NNV024 | >130 | 100 | 0.0004 | 87.5 |
| Duohexabody- | >130 | 100 | 0.0004 | 100 |
| CD37 | | | | |
| Obinutuzumab | >130 | 100 | 0.0004 | 100 |

FIG. 38 show mean body weights of each treatment group from arrival (day −14) until the study ended (Day 130). The first four and a half weeks all groups had similar weight gain pattern. From day 35 the control group started losing weight as the animals in this group started showing signs of disease. The last mouse that was euthanized in the control group with symptom of disease was euthanized on day 95. After that, one mouse was left and survived without symptoms until the study ended on day 130. The remaining treatment groups all had similar weight gaining patterns with small fluctuations.

### Discussion

The aim of the present study was to investigate the efficacy potential of the humanized CD37-targeting antibody NNV024 and compare it to the CD20-targeting antibody obinutuzumab and to the CD37-targeting biparatopic antibody DuoHexabody-CD37. The results show that at a single treatment of 2.69 mg/kg of antibody there was no detectable difference between the antibodies with respect to survival or bodyweight of the mice.

This result is different from the results of example 9 and 12 where there was a significantly better therapeutic effect of NNV024 than of obinutuzumab. In the current study a different mouse model (transgenic SCID FcRn−/− hFcRn (32) Tg mice) was used and the survival of the control group was about 3 times longer than survival of the control group in in example 9 and 12 where the median survival of the control animals was 23 days, whereas the median survival of the transgenic SCID mice in the current study was 76 days. Therefore, the transgenic mice in the current study appear to be more resistant to the Daudi cells than the CB17 SCID mouse model and perhaps that played a role in the different outcomes. The mice themselves were more resilient in fighting the malignancy than the CB17 SCID mice, therefore any difference between the treatment regimens could have been masked by the long incubation time of the tumor in these mice.

Another factor that was different from the study in example 9 and 12, was that the mice used in the current study were a bit older. The age of the mice in example 9 and 12 was 7-8 weeks at inoculation, whereas the transgenic mice in the current study were 7 (5 mice), 8 (13 mice) and 10 (40 mice) weeks of age at inoculation. The control animals were therefore only about 10-11 weeks of age when they were euthanized in example 9 and 12, but the transgenic control mice in the current study were on average 23 weeks of age when they were euthanized.

### Conclusion

The results show that a single treatment, according to body weight (2.69 mg/kg or 45-60 μg/mouse), with the humanized CD37-targeting antibody NNV024 appear to be equally as effective in treating disseminated B-cell malignancy as the CD20-targeting antibody obinutuzumab and the CD37-targeting biparatopic antibody DuoHexabody-CD37.

### Example 17 Induction of ADCC in Patient-Derived CLL Cells

### Aim

The aim of this example is to compare ADCC induction by NNV024 and NNV023 with obinutuzumab, rituximab and recombinant DuoHexabody-CD37 in patient derived chronic lymphocytic leukemia (CLL) cells.

### Materials and Methods

### ADCC/ADCP Reporter Assay

Peripheral blood mononuclear cells (PBMCs) from the blood of patients suffering from CLL were collected at Oslo University Hospital (OUS) [See Skánland, Sigrid S. Phospho flow cytometry with fluorescent cell barcoding for single cell signaling analysis and biomarker discovery. JoVE (Journal of Visualized Experiments), 2018, 140: e58386.]. The CLL cells constitute 90% of PBMCs of the samples. The samples have been randomly picked from a biobank consisting of CLL samples of patients who had not received any prior treatment against CLL. Thus, the cells were clinically described as treatment naive and carrying the IGVH mutation.

The cells (approx. 1×10⁸) were stored in liquid nitrogen, thawed on the day of the experiment (Day 0) and resuspended in RPMI1640 GlutaMax medium (Gibco) supplemented with 10% v/v heat-inactivated FCS (Gibco), 1% v/v penicillin-streptomycin mix (Gibco), 1× MEM non-essential amino acids (NEAA) and 1× sodium pyruvate. The suspension was then transferred to T25 cell culture flask (VWR, Cat.nr.: 734-2311) and kept for one hour in a humidified atmosphere with 5% CO₂ at 37° C. for recovery after thawing.

Subsequently, all cells were washed twice with 0.5% BSA in PBS, centrifuged (300× g, 5 min, 21° C.) and resuspended in the assay buffer (RPMI-1640 supplemented with 5% (w/v) Low Serum IgG (Promega)) to a final concentration of 1×10⁶ cells/mL. The cell suspension was dispensed (25 μL/well) in each well of a white 96 well plate (Nunclon).

The test antibodies (NNV023, NNV024, rituximab, obinutuzumab, recombinant version of DuoHexabody-CD37) were serially diluted in the assay buffer to cover the concentration range 1×10⁻⁶-1×10⁻⁰ μg/mL. These solutions were dispensed (25 μL/well) in duplicates to the 96 well plates with CLL cells and incubated at 21° C. for 30 min. Several wells were left with no Ab to serve as a background control.

The effector cells, recombinantly modified Jurkat cells carrying ADCC-related FcγIIIa (V158) receptors (Promega, Cat.nr.: G7010) and ADCP-related FcγIIa (H131) (Promega, Cat.nr.: G9991) and FcγIIa (R131) (Promega, Cat.nr.: CS1781 B08) receptors, were prepared as a suspension in the assay buffer with the concentration of 3×10⁶ cells/mL. These cells were dispensed (25 μL/well) into the 96 well plates with the target cells opsonized with the test antibodies. Thus, the target to effector cell ratio was 1:3.

The assay plates were kept for 18-22 hours in a humidified atmosphere with 5% CO₂ at 37° C. After incubation, 75 μL of luciferase substrate (from Promega kit) was added to each well and incubated for 10-25 min. The readout of the luciferase luminescence was performed using Tecan plate luminometer at an acquisition speed 0.5 sec/well.

The luminescence value of each well was background-corrected by withdrawing average background control (no Abs wells) value and normalized between max and min values on the scale from 0 to 1. The result was plotted in the coordinates: “Activation of FcγIIIa-V158 receptors (RLU norm.)” vs. “Log[Ab](ng/mL)”.

### Relative Expression of CD37/CD20 in CLL Samples Determined by Flow Cytometry

Anti-CD20 (10F381 rituximab) FITC-conjugated rabbit/human chimeric Ab (Novus biologicals ref: NBP2-526 46F) and anti-CD37 Alexa648-conjugated lilotomab (manufactured and conjugated in-house) were used as the fluorescent labels for flow cytometry.

First, the saturation titer was established in Daudi and SU-DHL-6 cell lines serving as standards highly expressing CD37 and CD20 on their surface. The saturation titer for rituximab and lilotomab was determined as 10 μg and 8 μg per 10⁶ cells respectively. REH cells were used as negative standard.

To determine the expression of CD37 and CD20 on CLL cells, 2 million cells of each CLL sample or cell line (Daudi, SU-DHL-6, REH, and CLL of a patient) were harvested and adjusted to 1×10⁶ cells/mL in 2 mL. Cells were washed once with 6 mL PBS with 0.5% BSA and pelleted by centrifugation (400×g, 5 min, 4° C.), resuspended in 2 mL cold PBS with 0.5% BSA. The cell suspensions were dispensed to a 96 well-plate (100 μL/well) in triplicates.

The fluorescently labeled rituximab and lilotomab were added to the cells in the concentrations respective to their saturation titers: 10 μg/10⁶ cells of FITC-rituximab and 8 μg/10⁶ cells of Alexa648-lilotomab. Cells to where no fluorescent Ab was added were used as a background (autofluorescence) control. The plate was incubated for 60 minutes on ice, before 3 washes with cold PBS was performed. The cells were resuspended in 150 μL PBS with 0.5% BSA for flow cytometry analysis.

Flow cytometry analysis was carried out on Guava easyCyte HT12 (Merck). FSC/SSC dotplot was used to gate cells into FSC H/FSC A dotplot for singlet discrimination. The singlet region was gated into FITC (CD20) or Alexa647 (CD37) channel for fluorescent readout.

The average median fluorescent intensities from RTX and HH1-Lilotomab were calculated from the fluorescent readout of the selected channel subtracting the median fluorescent intensity from the sample without antibody.

### Results

### ADCC Reporter Assay

Activation of FcγIIIa (V158) receptors sensed by the ADCC reporter assay is shown in FIG. 39. The results indicate that NNV024 is a strong activator of ADCC in patient-derived CLL cells. The ADCC signaling induced by 1 μg/mL of NNV024 in all four patient CLL samples is around 3-fold times higher than of 1 μg/mL of obinutuzumab. The fully fucosylated NNV023 induced ADCC on a par with DuoHexabody-CD37. Ability of rituximab to induce ADCC in the CLL patient samples was the lowest of all tested Abs.

### Flow Cytometry

The CLL from patient blood express 8-9 times more CD37 compared to CD20 (FIG. 40). The expression of both antigens is very similar in the four randomly picked CLL treatment naive samples.

### Discussion

NNV024 showed 3 times stronger ADCC activation than obinutuzumab, Duohexabody-CD37 and NNV023 at 1 mg/ml in 4 patient-derived CLL samples. The difference between NNV024 and obinutuzumab can be explained by the 8-9-fold times stronger expression of CD37 than of CD20, while the difference between NNV024 and NNV023 and DuoHexabody-CD37 can be explained by the afucosylation of NNV024.

### Conclusion

The afucosylated anti-CD37 humanized NNV024 greatly outperforms, the fucosylated NNV023, rituximab and DuoHexabody-CD37 as well as obinutuzumab in ability to induce ADCC-signaling in patient-derived CLL cells. If well tolerated, this Ab can be used as a replacement of therapeutical anti-CD20 monoclonal Abs as a B-cell depleting agent.

1. An antibody, antibody fragment or antibody derivative thereof, which comprises:
a) a heavy chain variable domain (VH) comprising VH-CDR1, VH-CDR2 and VH-CDR3, and
b) a light chain variable domain (VL) comprising VL-CDR1, VL-CDR2 and VL-CDR3, wherein,
c) the heavy chain variable domain (VH) comprises the amino acid sequence of SEQ ID NO: 29 and
d) the light chain variable domain (VL) comprises the amino acid sequence of SEQ ID NO: 8 [light chain of H02871],
wherein, according to SEQ TD NO: 8, position 13 is A or T, position 43 is A or S, position 49 is Y or N, position 71 is F or Y, position 78 is M or L, position 106 is I, M, or V, position 110 is V or D,
i. the light chain VL-CDR1 comprises the amino acid sequence ASQDVST,
ii. the light chain VL-CDR2 comprises the amino acid sequence WA, and
iii. the light chain VL-CDR3 comprises the amino acid sequence HYSTP.

2.-62. (canceled)

63. The antibody, antibody fragment or antibody derivative thereof according to claim 1, wherein the light chain variable domain (VL) comprises the amino acid sequence of any one of SEQ ID NOs: 24 or 72.

64. The antibody, antibody fragment or antibody derivative thereof according to claim 1, wherein the antibody fragment is a fragment selected from the group consisting of a Fab, Fab′, scFV, F(ab′)2, F(ab)2, F(ab)3 and scFv-Fc fragment.

65. The antibody, antibody fragment or antibody derivative thereof according to claim 1, wherein the antibody fragment is a minibody, diabody, triabody, or tetrabody.

66. The antibody, antibody fragment or antibody derivative thereof according to claim 65, wherein the glycosylation of said antibody, antibody fragment or antibody derivative thereof is fucose deficient.

67. A nucleic acid sequence encoding an antibody, antibody fragment or antibody derivative thereof according to claim 1.

68. A host cell comprising one or more nucleic acid sequence(s) according to claim 67.

69. An antibody, antibody fragment or antibody derivative thereof, drug conjugate that binds to human CD37 comprising:
an antibody, antibody fragment or antibody derivative thereof according to claim 1,
a linker, and
a drug selected from the group consisting of a toxin, a radioisotope, an anticancer drug, a cytotoxic drug and a cytostatic drug.

70. The antibody, antibody fragment or antibody derivative thereof drug conjugate according to claim 69, wherein drug is a radionuclide, selected from the group consisting of ²¹¹At, ²¹³Bi, ²¹²Bi, ²¹²Pb, ²²⁵Ac, ²²⁷Th, ¹⁶¹Tb, ⁹⁰Y, ¹⁸⁶Re, ¹⁸⁸Re, ¹⁹⁹Au, ¹⁹⁴Ir, ¹⁶⁶Ho, ¹⁵⁹Gd, ¹⁵³Sm, ¹⁴⁹Pm, ¹⁴²Pr, ¹¹¹Ag, ¹⁰⁹Pd, ⁷⁷As, ⁶⁴Cu, ⁶⁷Cu, ⁴⁷Sc, and ¹⁷⁷Lu.

71. A pharmaceutical composition comprising, as the active ingredient, one or more antibody/antibodies, antibody fragment(s) or antibody derivative(s) thereof according to claim 1, and a pharmaceutically acceptable carrier.

72. A method for producing an antibody, antibody fragment or antibody derivative thereof, the method comprising:
introducing into a mammalian host cell one or more nucleic acid nucleic acid sequences encoding the antibody, antibody fragment or antibody derivative thereof of claim 1,
culturing said host cell in a suitable media,
recovering said antibody, antibody fragment or antibody derivative thereof from the culturing broth, and
purifying the antibody, antibody fragment or antibody derivative thereof.

73. A method of inhibiting cancer and/or inflammatory disease(s) and/or autoimmune diseases, comprising administering the antibody, antibody fragment or antibody derivative thereof according to claim 1 to a subject in need thereof.

74. A pharmaceutical composition comprising the antibody, antibody fragment or antibody derivative thereof according to claim 1.

75. A kit for the production of an antibody fragment or antibody derivative thereof, drug conjugate comprising:
two or more vials, wherein one vial contains a conjugate comprising a drug linked to a linker, and one vial comprising an antibody fragment or antibody derivative thereof according to claim 1, and
optionally instructions for preparing said antibody-drug conjugate.

76. An antibody, antibody fragment or antibody derivative thereof, conjugate that binds to human CD37 comprising:
an antibody, antibody fragment or antibody derivative thereof according to claim 1,
a linker, and
a compound enriched in one or more isotopes selected from the group consisting of 11C, 13N, 15O, 18F, 64Cu and 89Zr.
